(19) United States
(12) Patent Application Publication
(10) Pub. No.: US 2025/0325542 A1
(43) Pub. Date: Oct. 23, 2025

(54) INHIBITORS OF ANTIMOCROBIAL RESISTANCE AND METHOS USING SAME

(71) Applicant: Baylor College of Medicine, Houston, TX (US)

(72) Inventors: Timothy Palzkill, Houston, TX (US); Martin Matzuk, Houston, TX (US); Suhyeorn Park, Houston, TX (US); Srinivas Chamakuri, Houston, TX (US)

(21) Appl. No.: 19/101,711

(22) PCT Filed: Jun. 23, 2023
(86) PCT No.: PCT/US2023/068963

§ 371 Date: Feb. 6, 2025

Related U.S. Application Data
(60) Provisional application No. 63/355,481, filed on Jun. 24, 2022.

(51) Int. Cl. A61K 31/506; A61K 45/06; C07D 239/28

(52) U.S. Cl. CPC A61K 31/506; A61K 45/06; C07D 239/28

(57) ABSTRACT

In one aspect, the present disclosure relates to compounds which inhibit one or more β-lactamases and/or penicillin binding proteins (PBPs), and pharmaceutical compositions thereof. In another aspect, the present disclosure provides a method of treating, preventing, and/or ameliorating a bacterial infection in a subject, the method comprising administering to the subject a therapeutically effective amount of at least one compound of the present disclosure and/or at least one pharmaceutical composition of the present disclosure. In certain embodiments, the β-lactamase is selected from the group consisting of NDM-1, KPC-2, and OXA-48. In certain embodiments, the PBP is PBP-3.

### CROSS-REFERENCE TO RELATED APPLICATIONS

The present application claims priority under 35 U.S.C. § 119(e) to U.S. Provisional Patent Application No. 63/355,481, filed Jun. 24, 2022, which application is hereby incorporated herein by reference in its entirety.

### STATEMENT REGARDING FEDERALLY SPONSORED RESEARCH OR DEVELOPMENT

This invention was made with government support under A1143832 awarded by the National Institutes of Health. The government has certain rights in the invention.

### BACKGROUND OF THE DISCLOSURE

Antimicrobial resistant infections account for 2.8 million infections and 35,000 deaths in the US alone (CDC). Growing resistance to conventional treatments are thus a rising public health threat. β-lactam antibiotics are the most commonly prescribed of all antibacterial agents, representing about 65% of antibiotic sales or 15 billion dollars. There are four families of β-lactam antibiotics: penicillins, carbapenems, cephalosporins, and monobactams. The function of each class of β-lactam antibiotics relies on the 4-membered β-lactam ring pharmacophore, with each class being distinguished by one or more substituents appended to the ring and/or the stereochemistry of these substituents. While penicillins, the first discovered family of β-lactam antibiotics, remain in widespread use, other families of β-lactam antibiotics (e.g. carbapenems) have been employed to enhance to the spectrum of action and fight against increasing antibiotic resistance. Carbapenems are especially less susceptible to resistance due to stability to β-lactamase-mediated breakdown.

The onset of resistance against β-lactam antibiotics is largely attributed to hydrolysis by β-lactamase enzymes that are produced by bacteria. β-lactamases are divided into four different classes (i.e., classes A, B, C, and D) according to amino acid sequence homology. Further, classes A, C, and D comprise an active site serine required for catalysis, whereas members of class B are zinc metalloenzymes, comprising a Zn(II) center required for catalysis.

Among the increasing number of β-lactamases, carbapenemases poses a greater threat by effectively catalyzing the hydrolysis of the β-lactam ring of carbapenems. The most prevalent carbapenemases worldwide include KPC-2 (i.e., class A), NDM and VIM (i.e. class C), and OXA-48 (i.e., class D) β-lactamases. KPC-2 is the most frequently encountered carbapenemase in carbapenem-resistant Enterobacteriaceae (CRE) strains from clinics worldwide.

In order to fight the growing frequency of β-lactamase-mediated resistance against β-lactam antibiotics, there have been increasing efforts to develop β-lactamase inhibitors. These inhibitors do not have antibacterial activity alone, but instead inhibit β-lactamases to block hydrolysis and restore the efficacy of D-lactam antibiotics such as amoxicillin.

Thus, there is a need in the art for β-lactamase and/or penicillin binding protein (PBP) inhibitors and methods of use thereof, alone, or in combination with one or more antibiotics (e.g., β-lactam antibiotics). The present disclosure addresses this need.

### BRIEF SUMMARY OF THE INVENTION

The present disclosure provides certain compounds of Formula (I), (II), (III), and (IV), or salts, solvates, stereoisomers, isotopologues, or tautomers thereof, or any mixtures thereof, wherein the substituents in (I), (II), (III), and (IV) are defined elsewhere herein:

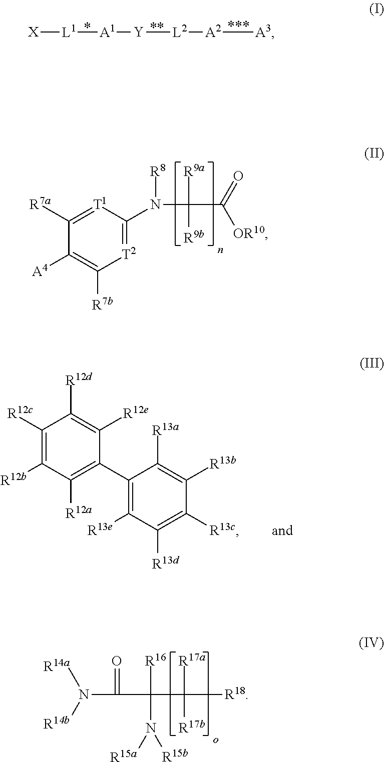

The present disclosure further provides pharmaceutical compositions comprising at least one compound of the present disclosure and a pharmaceutically acceptable carrier.

The present disclosure further provides methods of treating, preventing, and/or ameliorating a bacterial infection in a subject, the method comprising administering to the subject a therapeutically effective amount of at least one compound of the present disclosure and/or at least one pharmaceutical composition of the present disclosure.

The present disclosure further provides methods of inhibiting a β-lactamase and/or penicillin binding protein (PBP) in a subject, the method comprising administering to the subject a therapeutically effective amount of at least one compound of the present disclosure and/or at least one pharmaceutical composition of the present disclosure.

### BRIEF DESCRIPTION OF THE FIGURES

The drawings illustrate generally, by way of example, but not by way of limitation, various embodiments of the present application.

FIG. 1A provides a schematic overview of DNA encoded library screening. During cycle 1, the amine of the headpiece, the initial DNA strand with free amine as the functional group for chemical reactions, is reacted with functional group 1 (FG1) on cycle 1 building blocks, followed by cycle 1 DNA ligation onto the headpiece. This is pooled and split to the correct number of cycle 2 building blocks. The process is repeated where functional group 2 (FG2) on cycle 1 building blocks is reacted with functional group 3 (FG3) on cycle 2 building blocks, and DNA strands are ligated. The fully constructed library is pooled and incubated with a His-tagged protein. The binders are enriched by washing out the non-binders and repeating the binding-washing cycle 2-3 times. The binders are then identified by deep sequencing the DNA barcode tags. Finally, the hit compounds are synthesized in the absence of the DNA tag and assayed to identify the lead compounds. FIG. 1B shows a model compound in a β-lactamase focused library. The synthesized DNA-encoded library (DEL) contains a small molecule that comprises a linker (C1a) and two building blocks (C1b and C2) that end in a carboxylate or tetrazole functional group. The small molecule is built on a core DNA structure headpiece where DNA barcodes for each building block are added.

FIG. 2A: hydrolyzed meropenem is shown bound in the active site of NDM-1 (PDB: 5N0H), a class B β-lactamase. The carboxylate-binding pocket includes residues such as K211, which forms two H-bonding interactions with the carboxylic acid on meropenem. The carboxylic acid also coordinates with Zn2 and a water molecule present inside the carboxylate-binding pocket. FIG. 2B: hydrolyzed meropenem is bound in the active site of OXA-48 (PDB: 6PT1), a class D β-lactamase. Meropenem is covalently linked to the catalytic residue S70. The carboxylic acid in the carboxylate-binding pocket forms H-bonding interactions with R250, T209, and S118.

FIG. 3A provides non-limiting, exemplary DECL building blocks. C1 includes acid-halides, ester-protected di-acids, N-boc-protected di-amines, aldehyde-amines, and aldehyde-amines. C2 includes amino-acids, boronic acid-acids, aldehyde-acids, and esters. FIG. 3B shows affinity selection results. Analysis of selection results for NDM-1 β-lactamase as the target. The enrichment score (AC-zscore-n) of selection against NTC (γ-axis) is plotted against selection against NDM-1 (x-axis). FIG. 3C: the structure of the most enriched compound CDD-2335 is extrapolated from the deep sequencing analysis data.

FIG. 4A: a total of 4 hit compounds from OXA-48 screening were synthesized in the absence of the DNA and the linker (C1a), wherein C1b and C2 are highlighted. CDD-2746 (FIG. 4B) and CDD-2801 (FIG. 4C) were synthesized in the addition of the linker as well as in the absence of the methyl amide for SAR studies. X-ray crystal structure of OXA-48 in complex with CDD-2725 (PDB ID: 8SQF) (FIG. 4D) and CDD-2801 (PDB ID: 8SQG) (FIG. 4E). Both inhibitors bind in a similar binding pose with C1 positioned in the carboxylate-binding pocket and C2 pointing towards R214.

FIG. 5A: three hit compounds from NDM-1 screening were synthesized in the absence of the DNA and the linker (C1a), wherein C1b and C2 building blocks are highlighted. CDD-2376 is the most potent inhibitor with a low-micromolar inhibition constant. FIG. 5B: CDD-2376 was synthesized with the addition of the linker as well as in the absence of C1b to determine the lead compound necessary for further SAR study. Truncating the molecule eliminates inhibition activity. FIG. 5C: zinc-concentration dependent experiment was conducted to determine the mechanism of inhibition of CDD-2376. The inhibition constant at low (1 μM) and high (10 μM) concentrations of zinc had a minor difference, suggesting its inhibition through binding inside the active site instead of stripping the zinc away from the active site.

FIG. 6 provides the structure of CDD-2376.

FIG. 7 provides accumulation data in bacteria of tetracycline (positive control), clindamycin (negative control), and the NDM-1 inhibitors. CDD-2376 has the lowest amount of accumulation in bacteria, followed by CDD-2653, then CDD-2616. Accumulation of CDD-2616 is similar to the tetracycline positive control.

FIGS. 8A-8B provide molecular docking studies of hit compounds to the active site of NDM-1. Compounds CDD-2376 (FIG. 8A) and CDD-2616 (FIG. 8B) were docked inside the active site of a crystal structure of NDM-1 (PDB: 4EYL). The carboxylic acid group present in each of compounds CDD-2376 and CDD-2616 were positioned between the two zinc ions of NDM-1, and each form a H-bond with Asn220, which residue is known to interact with the hydrolyzed β-lactam ring. Additionally, the hydroxyl substituent adjacent to the carboxylic acid group binds to Lys211 inside the acid-binding pocket.

FIG. 9 provides a synthetic scheme for the preparation of qDOS38_1.

FIGS. 10A-10B provide synthetic schemes for the preparation of qDOS38_2.

### DETAILED DESCRIPTION OF THE INVENTION

β-lactam antibiotics represent about 65% of all antibiotic sales or 15 billion dollars per year worldwide. However, antibiotic resistance is an increasing threat to conventional therapies. The Centers for Disease Control and Prevention (CDC) has reported at least 2.8 million infections and 35,000 deaths related to antibiotic resistance every year in the US.

Clavulanate and avibactam are commonly prescribed inhibitors along with sulbactam, tazobactam, relebactam, and vaborbactam. Clavulanate, sulbactam, and tazobactam, however, comprise a β-lactam moiety, which increases their susceptibility to hydrolysis by β-lactamase enzymes, thereby limiting their utility.

Avibactam and relebactam have a diazabicyclooctane (DBO) structure, which can react with β-lactamase to generate an acyl-enzyme intermediate by covalent bond formation with the enzyme active site. However, unlike compounds with a β-lactam core, this reaction is reversible, and avibactam and relebactam can recyclize back to the intact drug molecule. Vaborbactam has a cyclic boronic acid core that covalently interacts with the catalytic serine residue inside the β-lactamase active site. Similar to avibactam and relebactam, the cyclic boronate core is not hydrolyzed and thus is an effective inhibitor of β-lactamase enzymes.

Vaborbactam and relebactam were discovered to inhibit class A carbapenemases such as KPC enzymes and are used in combination with carbapenems. Although avibactam is effective against KPC-2, a clinical isolate containing a KPC-2 variant with a D179Y mutation and some laboratory mutations, including the S130G mutation, were discovered to increase resistance to avibactam. There are currently no clinically available inhibitors for any class B β-lactamases including NDM-1. There is only one inhibitor, avibactam, which is effective against OXA-48. Relebactam and vaborbactam are ineffective against OXA-48.

Although there are increasing efforts to create novel carbapenemase inhibitors, many currently existing drug discovery efforts are a mere optimization of the three known pharmacophores, β-lactam, DBO, and boronate. Furthermore, all of the available inhibitors containing such pharmacophores are covalent inhibitors that attach to the nucleophilic serine in the active site of β-lactamase enzymes. While there has been some successful drug discovery against KPC-2, NDM-1, and OXA-48 β-lactamases using fragment-based drug discovery and virtual screening, most of the compounds that are past the pre-clinical phase contain known pharmacophores.

DNA-encoded library (DEL) technology was first proposed by Brenner and Lemer in 1992 as a new method of creating a diverse chemical library while utilizing DNA sequence as a barcode for identification of compounds subsequent to screening. Conventional high-throughput screening methods are relatively expensive and only explore a limited amount of chemical space. This limits the wide usage of chemical libraries in academic settings and is challenging for pharmaceutical companies to maintain large chemical screening libraries. In contrast, DEL uses significantly fewer resources and reagents and thus is receiving considerable attention among pharmaceutical companies and more widely in the drug discovery field.

DEL technology takes advantage of combinatorial synthesis and a DNA barcoding system to screen a wide range of chemical space while efficiently identifying hit compounds by DNA sequencing. DELs are created using split-and-pool combinatorial synthesis. The libraries are designed to contain drug-like molecules that are made out of building blocks with diverse chemical and biophysical properties.

The starting DNA-containing headpiece is split into a number of wells, each containing its own building block. After the attachment of the first building block, the contents of the wells are pooled and evenly split again into wells, each with a compound from the second set of building blocks. Upon addition of the second building block, a DNA fragment encoding the building block is ligated to the molecules in each well. This process is repeated for two or three cycles until the desired library is synthesized. The split-and-pool method allows synthesis of all possible combinations of building blocks in the most efficient manner. These molecules are covalently attached to a unique synthetic DNA barcode sequence that enables the identification of molecules that bind to a target after affinity enrichment steps. The population of candidate hit molecules enriched in a binding selection to the target protein is identified by next-generation sequencing of their associated DNA barcodes. Normalized Z-scores are used in calculating an enrichment score for each compound compared to a non-target control.

In addition to the pharmacophore β-lactam ring, all β-lactam antibiotics have a carboxylate group that significantly contributes to the binding energy of the molecules inside the active site. The carboxylate binding pocket is adjacent to but separate from the catalytic residues in the active site and is present in all four classes of β-lactamase enzymes. Other functional groups, such as sulfonic acid and tetrazole, are also known to bind in the same pocket. In the conventional creation of DELs, it is common to eschew these functional groups to avoid generating libraries of compounds that are overly hydrophilic. One skilled in the art of medicinal chemistry may often consider drug-like molecules having a middle range of hydrophobicity and lipophilicity ideal, as such properties favor permeation through a cell membrane while also being water-soluble.

For targeting Gram-negative bacteria, however, a drug molecule can also penetrate through outer membrane porins, thus the hydrophilicity can be increased, and, indeed, β-lactam antibiotics are hydrophilic molecules. Thus, the present disclosure describes the first effort to create a β-lactamase-focused DEL containing a core functionality with a known affinity for β-lactamases. It has been hypothesized that such an approach greatly increases the likelihood of identifying compounds which bind strongly one or more of the three classes of β-lactamase enzymes, as well as other enzymes containing a carboxylate binding pocket.

Thus, in one aspect, the present disclosure relates to the discovery of new small molecule inhibitors of β-lactamase enzymes including, but not limited to NDM-1, KPC-2, and OXA-48, as well as bacterial penicillin binding proteins (PBPs), using DNA-encoded library (DEL) technology.

Reference will now be made in detail to certain embodiments of the disclosed subject matter, examples of which are illustrated in part in the accompanying drawings. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference.

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

### Definitions

The term “about” as used herein can allow for a degree of variability in a value or range, for example, within 10%, within 5%, or within 1% of a stated value or of a stated limit of a range, and includes the exact stated value or range.

The term “acyl” as used herein refers to a group containing a carbonyl moiety wherein the group is bonded via the carbonyl carbon atom. The carbonyl carbon atom is bonded to a hydrogen forming a “formyl” group or is bonded to another carbon atom, which can be part of an alkyl, aryl, aralkyl cycloalkyl, cycloalkylalkyl, heterocyclyl, heterocyclylalkyl, heteroaryl, heteroarylalkyl group or the like. An acyl group can include 0 to about 12, 0 to about 20, or 0 to about 40 additional carbon atoms bonded to the carbonyl group. An acyl group can include double or triple bonds within the meaning herein. An acryloyl group is an example of an acyl group. An acyl group can also include heteroatoms within the meaning herein. A nicotinoyl group (pyridyl-3-carbonyl) is an example of an acyl group within the meaning herein. Other examples include acetyl, benzoyl, phenylacetyl, pyridylacetyl, cinnamoyl, and acryloyl groups and the like. When the group containing the carbon atom that is bonded to the carbonyl carbon atom contains a halogen, the group is termed a “haloacyl” group. An example is a trifluoroacetyl group.

The term “alkenyl” as used herein refers to straight and branched chain and cyclic alkyl groups as defined herein, except that at least one double bond exists between two carbon atoms. Thus, alkenyl groups have from 2 to 40 carbon atoms, or 2 to about 20 carbon atoms, or 2 to 12 carbon atoms or, in some embodiments, from 2 to 8 carbon atoms. Examples include, but are not limited to vinyl, —CH═C═CCH₂, —CH═CH(CH₃), —CH═C(CH₃)₂, —C(CH₃)═CH₂, —C(CH₃)═CH(CH₃), —C(CH₂CH₃)═CH₂, cyclohexenyl, cyclopentenyl, cyclohexadienyl, butadienyl, pentadienyl, and hexadienyl among others.

The term “alkoxy” as used herein refers to an oxygen atom connected to an alkyl group, including a cycloalkyl group, as are defined herein. Examples of linear alkoxy groups include but are not limited to methoxy, ethoxy, propoxy, butoxy, pentyloxy, hexyloxy, and the like. Examples of branched alkoxy include but are not limited to isopropoxy, sec-butoxy, tert-butoxy, isopentyloxy, isohexyloxy, and the like. Examples of cyclic alkoxy include but are not limited to cyclopropyloxy, cyclobutyloxy, cyclopentyloxy, cyclohexyloxy, and the like. An alkoxy group can include about 1 to about 12, about 1 to about 20, or about 1 to about 40 carbon atoms bonded to the oxygen atom, and can further include double or triple bonds, and can also include heteroatoms. For example, an allyloxy group or a methoxyethoxy group is also an alkoxy group within the meaning herein, as is a methylenedioxy group in a context where two adjacent atoms of a structure are substituted therewith.

The term “alkyl” as used herein refers to straight chain and branched alkyl groups and cycloalkyl groups having from 1 to 40 carbon atoms, 1 to about 20 carbon atoms, 1 to 12 carbons or, in some embodiments, from 1 to 8 carbon atoms. Examples of straight chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, t-butyl, neopentyl, isopentyl, and 2,2-dimethylpropyl groups. As used herein, the term “alkyl” encompasses n-alkyl, isoalkyl, and anteisoalkyl groups as well as other branched chain forms of alkyl. Representative substituted alkyl groups can be substituted one or more times with any of the groups listed herein, for example, amino, hydroxy, cyano, carboxy, nitro, thio, alkoxy, and halogen groups.

The term “alkylene” or “alkylenyl” as used herein refers to a bivalent saturated aliphatic radical (e.g., —CH₂—, —CH₂CH₂—, and —CH₂CH₂CH₂—, inter alia). In certain embodiments, the term may be regarded as a moiety derived from an alkene by opening of the double bond or from an alkane by removal of two hydrogen atoms from the same (e.g., —CH₂—) different (e.g., —CH₂CH₂—) carbon atoms.

The term “alkynyl” as used herein refers to straight and branched chain alkyl groups, except that at least one triple bond exists between two carbon atoms. Thus, alkynyl groups have from 2 to 40 carbon atoms, 2 to about 20 carbon atoms, or from 2 to 12 carbons or, in some embodiments, from 2 to 8 carbon atoms. Examples include, but are not limited to —C≡CH, —C≡C(CH₃), —C≡C(CH₂CH₃), —CH₂C≡CH, —CH₂C≡C(CH₃), and —CH₂C≡C(CH₂CH₃) among others.

The term “amine” as used herein refers to primary, secondary, and tertiary amines having, e.g., the formula N(group)₃ wherein each group can independently be H or non-H, such as alkyl, aryl, and the like. Amines include but are not limited to R—NH₂, for example, alkylamines, arylamines, alkylarylamines; R₂NH wherein each R is independently selected, such as dialkylamines, diarylamines, aralkylamines, heterocyclylamines and the like; and R₃N wherein each R is independently selected, such as trialkylamines, dialkylarylamines, alkyldiarylamines, triarylamines, and the like. The term “amine” also includes ammonium ions as used herein.

The term “amino group” as used herein refers to a substituent of the form —NH₂, —NHR, —NR₂, —NR₃⁺, wherein each R is independently selected, and protonated forms of each, except for —NR₃⁺, which cannot be protonated. Accordingly, any compound substituted with an amino group can be viewed as an amine. An “amino group” within the meaning herein can be a primary, secondary, tertiary, or quaternary amino group. An “alkylamino” group includes a monoalkylamino, dialkylamino, and trialkylamino group.

The term “aralkyl” as used herein refers to alkyl groups as defined herein in which a hydrogen or carbon bond of an alkyl group is replaced with a bond to an aryl group as defined herein. Representative aralkyl groups include benzyl and phenylethyl groups and fused (cycloalkylaryl)alkyl groups such as 4-ethyl-indanyl. Aralkenyl groups are alkenyl groups as defined herein in which a hydrogen or carbon bond of an alkyl group is replaced with a bond to an aryl group as defined herein.

The term “aryl” as used herein refers to cyclic aromatic hydrocarbon groups that do not contain heteroatoms in the ring. Thus aryl groups include, but are not limited to, phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, and naphthyl groups. In some embodiments, aryl groups contain about 6 to about 14 carbons in the ring portions of the groups. Aryl groups can be unsubstituted or substituted, as defined herein. Representative substituted aryl groups can be mono-substituted or substituted more than once, such as, but not limited to, a phenyl group substituted at any one or more of 2-, 3-, 4-, 5-, or 6-positions of the phenyl ring, or a naphthyl group substituted at any one or more of 2- to 8-positions thereof.

The term “cycloalkyl” as used herein refers to cyclic alkyl groups such as, but not limited to, cyclopropyl, cyclobutyl, cyclopentyl, cyclohexyl, cycloheptyl, and cyclooctyl groups. In some embodiments, the cycloalkyl group can have 3 to about 8-12 ring members, whereas in other embodiments the number of ring carbon atoms range from 3 to 4, 5, 6, or 7. Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like. Cycloalkyl groups also include rings that are substituted with straight or branched chain alkyl groups as defined herein. Representative substituted cycloalkyl groups can be mono-substituted or substituted more than once, such as, but not limited to, 2,2-, 2,3-, 2,4- 2,5- or 2,6-disubstituted cyclohexyl groups or mono-, di- or tri-substituted norbornyl or cycloheptyl groups, which can be substituted with, for example, amino, hydroxy, cyano, carboxy, nitro, thio, alkoxy, and halogen groups. The term “cycloalkenyl” alone or in combination denotes a cyclic alkenyl group.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “ameliorated” if the severity of a symptom of the disease or disorder, the frequency with which such a symptom is experienced by a patient, or both, is reduced.

As used herein, the terms “effective amount,” “pharmaceutically effective amount” and “therapeutically effective amount” refer to a nontoxic but sufficient amount of an agent to provide the desired biological result. That result may be reduction and/or alleviation of the signs, symptoms, or causes of a disease, or any other desired alteration of a biological system. An appropriate therapeutic amount in any individual case may be determined by one of ordinary skill in the art using routine experimentation.

The terms “halo,” “halogen,” or “halide” group, as used herein, by themselves or as part of another substituent, mean, unless otherwise stated, a fluorine, chlorine, bromine, or iodine atom.

The term “haloalkyl” group, as used herein, includes mono-halo alkyl groups, poly-halo alkyl groups wherein all halo atoms can be the same or different, and per-halo alkyl groups, wherein all hydrogen atoms are replaced by halogen atoms, such as fluoro. Examples of haloalkyl include trifluoromethyl, 1,1-dichloroethyl, 1,2-dichloroethyl, 1,3-dibromo-3,3-difluoropropyl, perfluorobutyl, and the like.

The term “heteroaryl” as used herein refers to aromatic ring compounds containing 5 or more ring members, of which, one or more is a heteroatom such as, but not limited to, N, O, and S; for instance, heteroaryl rings can have 5 to about 8-12 ring members. A heteroaryl group is a variety of a heterocyclyl group that possesses an aromatic electronic structure. A heteroaryl group designated as a C2-heteroaryl can be a 5-ring with two carbon atoms and three heteroatoms, a 6-ring with two carbon atoms and four heteroatoms and so forth. Likewise a C4-heteroaryl can be a 5-ring with one heteroatom, a 6-ring with two heteroatoms, and so forth. The number of carbon atoms plus the number of heteroatoms sums up to equal the total number of ring atoms. Heteroaryl groups include, but are not limited to, groups such as pyrrolyl, pyrazolyl, triazolyl, tetrazolyl, oxazolyl, isoxazolyl, thiazolyl, pyridinyl, thiophenyl, benzothiophenyl, benzofuranyl, indolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Heteroaryl groups can be unsubstituted, or can be substituted with groups as is discussed herein. Representative substituted heteroaryl groups can be substituted one or more times with groups such as those listed herein.

Additional examples of aryl and heteroaryl groups include but are not limited to phenyl, biphenyl, indenyl, naphthyl (1-naphthyl, 2-naphthyl), N-hydroxytetrazolyl, N-hydroxytriazolyl, N-hydroxyimidazolyl, anthracenyl (1-anthracenyl, 2-anthracenyl, 3-anthracenyl), thiophenyl (2-thienyl, 3-thienyl), furyl (2-furyl, 3-furyl), indolyl, oxadiazolyl, isoxazolyl, quinazolinyl, fluorenyl, xanthenyl, isoindanyl, benzhydryl, acridinyl, thiazolyl, pyrrolyl (2-pyrrolyl), pyrazolyl (3-pyrazolyl), imidazolyl (1-imidazolyl, 2-imidazolyl, 4-imidazolyl, 5-imidazolyl), triazolyl (1,2,3-triazol-1-yl, 1,2,3-triazol-2-yl 1,2,3-triazol-4-yl, 1,2,4-triazol-3-yl), oxazolyl (2-oxazolyl, 4-oxazolyl, 5-oxazolyl), thiazolyl (2-thiazolyl, 4-thiazolyl, 5-thiazolyl), pyridyl (2-pyridyl, 3-pyridyl, 4-pyridyl), pyrimidinyl (2-pyrimidinyl, 4-pyrimidinyl, 5-pyrimidinyl, 6-pyrimidinyl), pyrazinyl, pyridazinyl (3-pyridazinyl, 4-pyridazinyl, 5-pyridazinyl), quinolyl (2-quinolyl, 3-quinolyl, 4-quinolyl, 5-quinolyl, 6-quinolyl, 7-quinolyl, 8-quinolyl), isoquinolyl (1-isoquinolyl, 3-isoquinolyl, 4-isoquinolyl, 5-isoquinolyl, 6-isoquinolyl, 7-isoquinolyl, 8-isoquinolyl), benzo[b]furanyl (2-benzo[b]furanyl, 3-benzo[b]furanyl, 4-benzo[b]furanyl, 5-benzo[b]furanyl, 6-benzo[b]furanyl, 7-benzo[b]furanyl), 2,3-dihydro-benzo[b]furanyl (2-(2,3-dihydro-benzo[b]furanyl), 3-(2,3-dihydro-benzo[b]furanyl), 4-(2,3-dihydro-benzo[b]furanyl), 5-(2,3-dihydro-benzo[b]furanyl), 6-(2,3-dihydro-benzo[b]furanyl), 7-(2,3-dihydro-benzo[b]furanyl), benzo[b]thiophenyl (2-benzo[b]thiophenyl, 3-benzo[b]thiophenyl, 4-benzo[b]thiophenyl, 5-benzo[b]thiophenyl, 6-benzo[b]thiophenyl, 7-benzo[b]thiophenyl), 2,3-dihydro-benzo[b]thiophenyl, (2-(2,3-dihydro-benzo[b]thiophenyl), 3-(2,3-dihydro-benzo[b]thiophenyl), 4-(2,3-dihydro-benzo[b]thiophenyl), 5-(2,3-dihydro-benzo[b]thiophenyl), 6-(2,3-dihydro-benzo[b]thiophenyl), 7-(2,3-dihydro-benzo[b]thiophenyl), indolyl (1-indolyl, 2-indolyl, 3-indolyl, 4-indolyl, 5-indolyl, 6-indolyl, 7-indolyl), indazole (1-indazolyl, 3-indazolyl, 4-indazolyl, 5-indazolyl, 6-indazolyl, 7-indazolyl), benzimidazolyl (1-benzimidazolyl, 2-benzimidazolyl, 4-benzimidazolyl, 5-benzimidazolyl, 6-benzimidazolyl, 7-benzimidazolyl, 8-benzimidazolyl), benzoxazolyl (1-benzoxazolyl, 2-benzoxazolyl), benzothiazolyl (1-benzothiazolyl, 2-benzothiazolyl, 4-benzothiazolyl, 5-benzothiazolyl, 6-benzothiazolyl, 7-benzothiazolyl), carbazolyl (1-carbazolyl, 2-carbazolyl, 3-carbazolyl, 4-carbazolyl), 5H-dibenz[b,f]azepine (5H-dibenz[b,f]azepin-1-yl, 5H-dibenz[b,f]azepine-2-yl, 5H-dibenz[b,f]azepine-3-yl, 5H-dibenz[b,f]azepine-4-yl, 5H-dibenz[b,f]azepine-5-yl), 10,11-dihydro-5H-dibenz[b,f]azepine (10,11-dihydro-5H-dibenz[b,f]azepine-1-yl, 10,11-dihydro-5H-dibenz[b,f]azepine-2-yl, 10,11-dihydro-5H-dibenz[b,f]azepine-3-yl, 10,11-dihydro-5H-dibenz[b,f]azepine-4-yl, 10,11-dihydro-5H-dibenz[b,f]azepine-5-yl), and the like.

The term “heteroarylalkyl” as used herein refers to alkyl groups as defined herein in which a hydrogen or carbon bond of an alkyl group is replaced with a bond to a heteroaryl group as defined herein.

The term “heteroarylene” or “heteroarylenyl” as used herein refers to a bivalent heteroaryl radical (e.g., 2,4-pyridylene). In certain embodiments, the term may be regarded as a divalent radical formed by the removal of two hydrogen atoms from one or more rings of a heteroaryl moiety, wherein the hydrogen atoms may be removed from the same or different rings, preferably the same ring.

The term “heterocyclylalkyl” as used herein refers to alkyl groups as defined herein in which a hydrogen or carbon bond of an alkyl group as defined herein is replaced with a bond to a heterocyclyl group as defined herein. Representative heterocyclyl alkyl groups include, but are not limited to, furan-2-yl methyl, furan-3-yl methyl, pyridine-3-yl methyl, tetrahydrofuran-2-yl ethyl, and indol-2-yl propyl.

The term “heterocyclyl” as used herein refers to aromatic and non-aromatic ring compounds containing three or more ring members, of which one or more is a heteroatom such as, but not limited to, N, O, and S. Thus, a heterocyclyl can be a cycloheteroalkyl, or a heteroaryl, or if polycyclic, any combination thereof. In some embodiments, heterocyclyl groups include 3 to about 20 ring members, whereas other such groups have 3 to about 15 ring members. A heterocyclyl group designated as a C2-heterocyclyl can be a 5-ring with two carbon atoms and three heteroatoms, a 6-ring with two carbon atoms and four heteroatoms and so forth. Likewise a C4-heterocyclyl can be a 5-ring with one heteroatom, a 6-ring with two heteroatoms, and so forth. The number of carbon atoms plus the number of heteroatoms equals the total number of ring atoms. A heterocyclyl ring can also include one or more double bonds. A heteroaryl ring is an embodiment of a heterocyclyl group. The phrase “heterocyclyl group” includes fused ring species including those that include fused aromatic and non-aromatic groups. For example, a dioxolanyl ring and a benzdioxolanyl ring system (methylenedioxyphenyl ring system) are both heterocyclyl groups within the meaning herein. The phrase also includes polycyclic ring systems containing a heteroatom such as, but not limited to, quinuclidyl. Heterocyclyl groups can be unsubstituted, or can be substituted as discussed herein. Heterocyclyl groups include, but are not limited to, pyrrolidinyl, piperidinyl, piperazinyl, morpholinyl, pyrrolyl, pyrazolyl, triazolyl, tetrazolyl, oxazolyl, isoxazolyl, thiazolyl, pyridinyl, thiophenyl, benzothiophenyl, benzofuranyl, dihydrobenzofuranyl, indolyl, dihydroindolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Representative substituted heterocyclyl groups can be mono-substituted or substituted more than once, such as, but not limited to, piperidinyl or quinolinyl groups, which are 2-, 3-, 4-, 5-, or 6-substituted, or disubstituted with groups such as those listed herein.

The term “hydrocarbon” or “hydrocarbyl” as used herein refers to a molecule or functional group that includes carbon and hydrogen atoms. The term can also refer to a molecule or functional group that normally includes both carbon and hydrogen atoms but wherein all the hydrogen atoms are substituted with other functional groups.

As used herein, the term “hydrocarbyl” refers to a functional group derived from a straight chain, branched, or cyclic hydrocarbon, and can be alkyl, alkenyl, alkynyl, aryl, cycloalkyl, acyl, or any combination thereof. Hydrocarbyl groups can be shown as (Cₐ-C_{b})hydrocarbyl, wherein a and b are integers and mean having any of a to b number of carbon atoms. For example, (C₁-C₄)hydrocarbyl means the hydrocarbyl group can be methyl (C₁), ethyl (C₂), propyl (C₃), or butyl (C₄), and (C₀-C_{b})hydrocarbyl means in certain embodiments there is no hydrocarbyl group.

The term “independently selected from” as used herein refers to referenced groups being the same, different, or a mixture thereof, unless the context clearly indicates otherwise. Thus, under this definition, the phrase “X¹, X², and X³ are independently selected from noble gases” would include the scenario where, for example, X¹, X², and X³ are all the same, where X¹, X², and X³ are all different, where X¹ and X² are the same but X³ is different, and other analogous permutations.

The term “monovalent” as used herein refers to a substituent connecting via a single bond to a substituted molecule. When a substituent is monovalent, such as, for example, F or Cl, it is bonded to the atom it is substituting by a single bond.

The term “organic group” as used herein refers to any carbon-containing functional group. Examples can include an oxygen-containing group such as an alkoxy group, aryloxy group, aralkyloxy group, oxo(carbonyl) group; a carboxyl group including a carboxylic acid, carboxylate, and a carboxylate ester; a sulfur-containing group such as an alkyl and aryl sulfide group; and other heteroatom-containing groups. Non-limiting examples of organic groups include OR, OOR, OC(O)N(R)₂, CN, CF₃, OCF₃, R, C(O), methylenedioxy, ethylenedioxy, N(R)₂, SR, SOR, SO₂R, SO₂N(R)₂, SO₃R, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, C(═NOR)R, and substituted or unsubstituted (C₁-C₁₀₀)hydrocarbyl, wherein R can be hydrogen (in examples that include other carbon atoms) or a carbon-based moiety, and wherein the carbon-based moiety can be substituted or unsubstituted.

The terms “patient,” “subject,” or “individual” are used interchangeably herein, and refer to any animal, or cells thereof whether in vitro or in situ, amenable to the methods described herein. In a non-limiting embodiment, the patient, subject or individual is a human.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained.

As used herein, the language “pharmaceutically acceptable salt” refers to a salt of the administered compounds prepared from pharmaceutically acceptable non-toxic acids or bases, including inorganic acids or bases, organic acids or bases, solvates, hydrates, or clathrates thereof.

Suitable pharmaceutically acceptable acid addition salts may be prepared from an inorganic acid or from an organic acid. Examples of inorganic acids include hydrochloric, hydrobromic, hydriodic, nitric, carbonic, sulfuric (including sulfate and hydrogen sulfate), and phosphoric acids (including hydrogen phosphate and dihydrogen phosphate). Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, malonic, saccharin, fumaric, pyruvic, aspartic, glutamic, benzoic, anthranilic, 4-hydroxybenzoic, phenylacetic, mandelic, embonic (pamoic), methanesulfonic, ethanesulfonic, benzenesulfonic, pantothenic, trifluoromethanesulfonic, 2-hydroxyethanesulfonic, p-toluenesulfonic, sulfanilic, cyclohexylaminosulfonic, stearic, alginic, β-hydroxybutyric, salicylic, galactaric and galacturonic acid.

Suitable pharmaceutically acceptable base addition salts of compounds described herein include, for example, ammonium salts, metallic salts including alkali metal, alkaline earth metal and transition metal salts such as, for example, calcium, magnesium, potassium, sodium and zinc salts. Pharmaceutically acceptable base addition salts also include organic salts made from basic amines such as, for example, N,N′-dibenzylethylene-diamine, chloroprocaine, choline, diethanolamine, ethylenediamine, meglumine (N-methylglucamine) and procaine. All of these salts may be prepared from the corresponding compound by reacting, for example, the appropriate acid or base with the compound.

As used herein, the term “pharmaceutically acceptable carrier” or “pharmaceutically acceptable excipient” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound described herein within or to the patient such that it may perform its intended function. Typically, such compounds are carried or transported from one organ, or portion of the body, to another organ, or portion of the body. Each carrier must be “acceptable” in the sense of being compatible with the other ingredients of the formulation, including the compound(s) described herein, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound(s) described herein, and are physiologically acceptable to the patient. Supplementary active compounds may also be incorporated into the compositions. The “pharmaceutically acceptable carrier” may further include a pharmaceutically acceptable salt of the compound(s) described herein. Other additional ingredients that may be included in the pharmaceutical compositions used with the methods or compounds described herein are known in the art and described, for example in Remington's Pharmaceutical Sciences (Genaro, Ed., Mack Publishing Co., 1985, Easton, PA), which is incorporated herein by reference.

The term “phenylene” or “phenylenyl” as used herein refers to a bivalent phenyl radical (e.g., 1,4-phenylene). In certain embodiments, the term may be regarded as a divalent radical formed by the removal of two hydrogen atoms from a benzene moiety.

The term “room temperature” as used herein refers to a temperature of about 15° C. to 28° C.

The term “solvent” as used herein refers to a liquid that can dissolve a solid, liquid, or gas. Non-limiting examples of solvents are silicones, organic compounds, water, alcohols, ionic liquids, and supercritical fluids.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%. The term “substantially free of” as used herein can mean having none or having a trivial amount of, such that the amount of material present does not affect the material properties of the composition including the material, such that the composition is about 0 wt % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less. The term “substantially free of” can mean having a trivial amount of, such that a composition is about 0 wt % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less, or about 0 wt %.

The term “substituted” as used herein in conjunction with a molecule or an organic group as defined herein refers to the state in which one or more hydrogen atoms contained therein are replaced by one or more non-hydrogen atoms. The term “functional group” or “substituent” as used herein refers to a group that can be or is substituted onto a molecule or onto an organic group. Examples of substituents or functional groups include, but are not limited to, a halogen (e.g., F, Cl, Br, and I); an oxygen atom in groups such as hydroxy groups, alkoxy groups, aryloxy groups, aralkyloxy groups, oxo(carbonyl) groups, carboxyl groups including carboxylic acids, carboxylates, and carboxylate esters; a sulfur atom in groups such as thiol groups, alkyl and aryl sulfide groups, sulfoxide groups, sulfone groups, sulfonyl groups, and sulfonamide groups; a nitrogen atom in groups such as amines, hydroxyamines, nitriles, nitro groups, N-oxides, hydrazides, azides, and enamines; and other heteroatoms in various other groups. Non-limiting examples of substituents that can be bonded to a substituted carbon (or other) atom include F, Cl, Br, I, OR, OC(O)N(R)₂, CN, NO, NO₂, ONO₂, azido, CF₃, OCF₃, R, O (oxo), S (thiono), C(O), S(O), methylenedioxy, ethylenedioxy, N(R)₂, SR, SOR, SO₂R, SO₂N(R)₂, SO₃R, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, and C(═NOR)R, wherein R can be hydrogen or a carbon-based moiety; for example, R can be hydrogen, (C₁-C₁₀₀) hydrocarbyl, alkyl, acyl, cycloalkyl, aryl, aralkyl, heterocyclyl, heteroaryl, or heteroarylalkyl; or wherein two R groups bonded to a nitrogen atom or to adjacent nitrogen atoms can together with the nitrogen atom or atoms form a heterocyclyl.

A “therapeutic” treatment is a treatment administered to a subject who exhibits signs of pathology, for the purpose of diminishing or eliminating those signs.

The terms “treat,” “treating” and “treatment,” as used herein, means reducing the frequency or severity with which symptoms of a disease or condition are experienced by a subject by virtue of administering an agent or compound to the subject.

### Compounds

In one aspect, the present disclosure provides a compound of formula (I), or a salt, solvate, stereoisomer, isotopologue, or tautomer thereof, or any mixtures thereof:

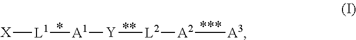

wherein:
R^{1a} and R^{1b} are each independently selected from the group consisting of H, optionally substituted C₁-C₆ alkyl, optionally substituted C₃-C₁₂ cycloalkyl, optionally substituted C₇-C₁₂ aralkyl, optionally substituted C₂-C₁₂ heterocyclyl, optionally substituted C₆-C₁₂ aryl, and C(═O)(optionally substituted C₁-C₆ alkyl);
A¹ and A² are each independently selected from the group consisting of a bond, optionally substituted phenylenyl and optionally substituted C₂-C₉ heteroarylenyl;
A³ is selected from the group consisting of optionally substituted C₆-C₁₂ aryl and optionally substituted C₂-C₁₂ heteroaryl;
L¹ and L² are each independently selected from the group consisting of a bond, optionally substituted C₁-C₃ alkylenyl, —C(═O), —C(═O)(optionally substituted C₁-C₃ alkylenyl)-O—, —C(═O)(optionally substituted C₁-C₃ alkylenyl)-, (optionally substituted C₁-C₃ alkylenyl)C(═O)—, —C(═O)(optionally substituted C₁-C₃ alkylenyl)C(═O)—, optionally substituted C₃-C₈ heterocycloalkylenyl, —C(═O)(optionally substituted C₃-C₈ heterocycloalkylenyl)(optionally substituted C₁-C₃ alkylenyl)C(═O)—, and —C(═O)(optionally substituted C₃-C₈ cycloalkylenyl)C(═O)—;
R² is selected from the group consisting of H and optionally substituted C₁-C₆ alkyl,
   wherein R² can combine with A² to form an optionally substituted C₃-C₈ heterocycloalkyl;
X is selected from the group consisting of N(R^{1a})(R^{1b}) and optionally substituted C₂-C₁₂ heterocyclyl;
Y is selected from the group consisting of a bond, —N(R²)—, —(CH₂)₁₋₃N(R²)—**, —O—, and —S—;
* indicates a bond between L¹ and A¹;
** indicates a bond between Y and L²;
*** indicates a bond between A² and A3; and
each occurrence of R^{A} and R^{B} is independently selected from the group consisting of H, —C(═O)(optionally substituted C₁-C₆ alkyl), optionally substituted C₁-C₆ alkyl, optionally substituted C₃-C₈ cycloalkyl, optionally substituted C₇-C₁₂ aralkyl, optionally substituted phenyl, optionally substituted naphthyl, and optionally substituted heteroaryl.

In certain embodiments, A¹ is a bond. In certain embodiments, A² is a bond. In certain embodiments, L¹ is a bond. In certain embodiments, X is

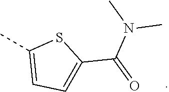

In certain embodiments, Y is a bond or —NH—.

In certain embodiments, the compound of formula (I) is a compound of formula (Ia):

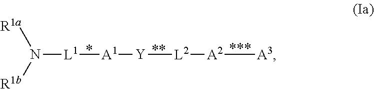

wherein:
A¹ and A² are each independently selected from the group consisting of optionally substituted phenylenyl and optionally substituted C₂-C₉ heteroarylenyl;
A³ is optionally substituted C₆-C₁₂ aryl;
L¹ and L² are each independently selected from the group consisting of bond, optionally substituted C₁-C₃ alkylenyl, —C(═O), —C(═O)(optionally substituted C₁-C₃ alkylenyl)-O—, —C(═O)(optionally substituted C₁-C₃ alkylenyl)-, (optionally substituted C₁-C₃ alkylenyl)C(═O)—, —C(═O)(optionally substituted C₁-C₃ alkylenyl)C(═O)—, optionally substituted C₃-C₈ heterocycloalkylenyl, —C(═O)(optionally substituted C₃-C₈ heterocycloalkylenyl)(optionally substituted C₁-C₃ alkylenyl)C(═O)—, and —C(═O)(optionally substituted C₃-C₈ cycloalkylenyl)C(═O)—;
R^{1a} and R^{1b} are each independently selected from the group consisting of H, optionally substituted C₁-C₆ alkyl, optionally substituted C₃-C₁₂ cycloalkyl, optionally substituted C₇-C₁₂ aralkyl, optionally substituted C₂-C₁₂ heterocyclyl, optionally substituted C₆-C₁₂ aryl, and C(═O)(optionally substituted C₁-C₆ alkyl);
R² is selected from the group consisting of H and optionally substituted C₁-C₆ alkyl,
   wherein R² can combine with A² to form an optionally substituted C₃-C₈ heterocycloalkyl;
Y is selected from the group consisting of —N(R²)—, —(CH₂)₁₋₃N(R²)—**, —O—, and —S—;
* indicates a bond between L¹ and A¹;
** indicates a bond between Y and L²;
*** indicates a bond between A² and A³; and
each occurrence of R^{A} and R^{B} is independently selected from the group consisting of H, —C(═O)(optionally substituted C₁-C₆ alkyl), optionally substituted C₁-C₆ alkyl, optionally substituted C₃-C₈ cycloalkyl, optionally substituted C₇-C₁₂ aralkyl, optionally substituted phenyl, optionally substituted naphthyl, and optionally substituted heteroaryl.

In certain embodiments, A¹ is selected from the group consisting of:

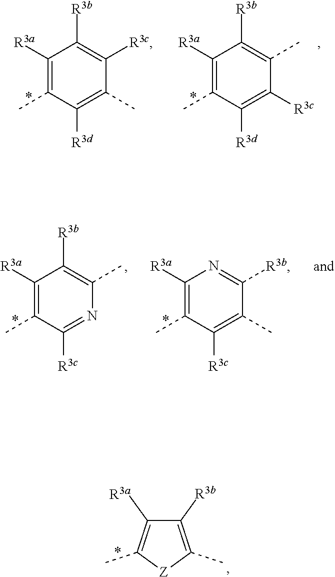

wherein:
R^{3a}, R^{3b}, R^{3c}, and R^{3d}, if present, are each independently selected from the group consisting of H, halogen, OR^{A}, C(═O)OR^{A}, CN, NO₂, optionally substituted C₁-C₆ alkyl, and optionally substituted C₃-C₈ cycloalkyl.

In certain embodiments, at least one selected from R^{3a}, R^{3b}, R^{3c}, and R^{3d}, is H. In certain embodiments, at least two selected from R^{3a}, R^{3b}, R^{3c} and R^{3d} are H. In certain embodiments, at least three selected from R^{3a}, R^{3b}, R^{3c}, and R^{3d} are H.

In certain embodiments, A¹ is

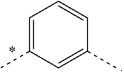

In certain embodiments, A¹ is

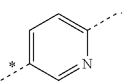

In certain embodiments, A¹ is

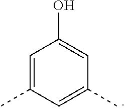

In certain embodiments, A¹ is

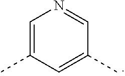

In certain embodiments, A¹ is

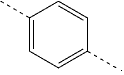

In certain embodiments, A¹ is

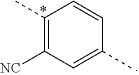

In certain embodiments, A¹ is

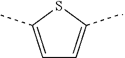

In certain embodiments, A² is selected from the group consisting of:

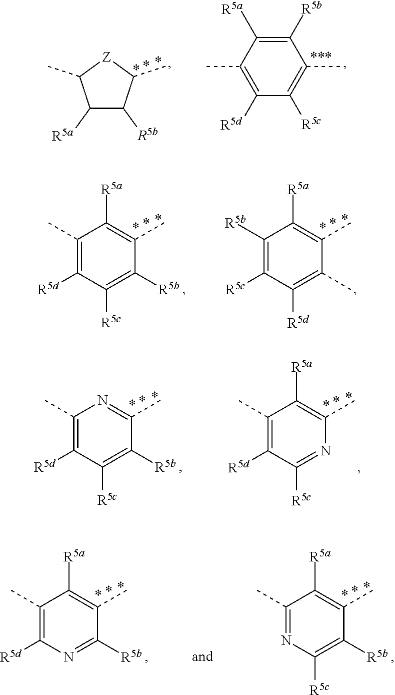

wherein:
Z is selected from the group consisting of O, S, and NR⁴;
R⁴ is selected from the H and optionally substituted C₁-C₆ alkyl; and
R^{5a}, R^{5b}, R^{5c}, and R^{5d}, if present, are each independently selected from the group consisting of H, halogen, OR^{A}, C(═O)OR^{A}, CN, NO₂, optionally substituted C₁-C₆ alkyl, and optionally substituted C₃-C₈ cycloalkyl.

In certain embodiments, at least one selected from R^{5a}, R^{5b}, R^{5c}, and R^{5d} is H. In certain embodiments, at least two selected from R^{5a}, R^{5b}, R^{5c}, and R^{5d} are H. In certain embodiments, at least three selected from R^{5a}, R^{5b}, R^{5c}, and R^{5d} are H.

In certain embodiments A² is

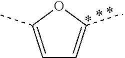

In certain embodiments, A² is

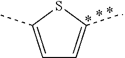

In certain embodiments, A² is

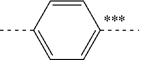

In certain embodiments, A² is

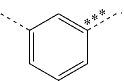

In certain embodiments, A² is

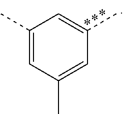

In certain embodiments, A² is

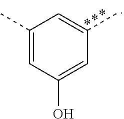

In certain embodiments, A² is

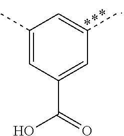

In certain embodiments, A² is

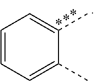

In certain embodiments, A² is

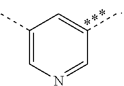

In certain embodiments, A² is

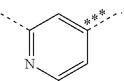

In certain embodiments, A² is

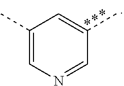

In certain embodiments, A² is

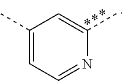

In certain embodiments, A³ is selected from the group consisting of:

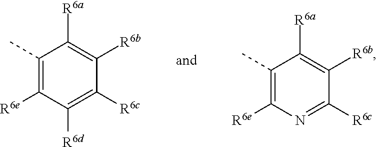

wherein:
R^{6a}, R^{6b}, R^{6c}, R^{6d}, and R^{6e}, if present, are each independently selected from the group consisting of H, halogen, OR^{A}, C(═O)OR^{A}, (optionally substituted C₁-C₆ alkylenyl)C(═O)OR^{A}, CN, NO₂, optionally substituted C₁-C₆ alkyl, and optionally substituted C₃-C₈ cycloalkyl.

In certain embodiments, R^{6a} is H. In certain embodiments, R^{6a} is C(═O)OH. In certain embodiments, R^{6a} is OH. In certain embodiments, R^{6a} is CH₂C(═O)OH. In certain embodiments, R^{6a} is OMe. In certain embodiments, R^{6a} is OBn. In certain embodiments, R^{6b} is H. In certain embodiments, R^{6b} is C(═O)OH. In certain embodiments, R^{6b} is OH. In certain embodiments, R^{6b} is CH₂C(═O)OH. In certain embodiments, R^{6b} is OMe. In certain embodiments, R^{6b} is OBn. In certain embodiments, R^{6c} is H. In certain embodiments, R^{6c} is C(═O)OH. In certain embodiments, R^{6c} is OH. In certain embodiments, R^{6c} is CH₂C(═O)OH. In certain embodiments, R^{6c} is OMe. In certain embodiments, R^{6c} is OBn. In certain embodiments, R^{6d} is H. In certain embodiments, R^{6d} is C(═O)OH. In certain embodiments, R^{6d} is OH. In certain embodiments, R^{6d} is CH₂C(═O)OH. In certain embodiments, R^{6d} is OMe. In certain embodiments, R^{6d} is OBn. In certain embodiments, R^{6e} is H. In certain embodiments, R^{6e} is C(═O)OH. In certain embodiments, R^{6e} is OH. In certain embodiments, R^{6e} is CH₂C(═O)OH. In certain embodiments, R^{6e} is OMe. In certain embodiments, R^{6c} is OBn.

In certain embodiments, at least one selected from R^{6a}, R^{6b}, R^{6c}, R^{6d}, and R^{6e} is H. In certain embodiments, at least two selected from R^{6a}, R^{6b}, R^{6c}, R^{6d}, and R^{6e} are H. In certain embodiments, at least three selected from R^{6a}, R^{6b}, R^{6c}, R^{6d}, and R^{6e} are H. In certain embodiments, at least four selected from R^{6a}, R^{6b}, R^{6c}, R^{6d}, and R^{6e} are H.

In certain embodiments, at least one selected from R^{6a}, R^{6b}, R^{6c}, R^{6d}, and R^{6e} is C(═O)OH. In certain embodiments, at least one selected from R^{6a}, R^{6b}, R^{6c}, R^{6d}, and R^{6e} is CH₂C(═O)OH. In certain embodiments, at least one selected from R^{6a}, R^{6b}, R^{6c}, R^{6d}, and R^{6e} is OH.

In certain embodiments, A³ is

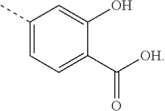

In certain embodiments, A³ is

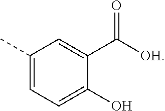

In certain embodiments, A³ is

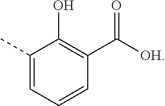

In certain embodiments, A³ is

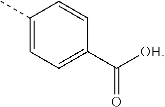

In certain embodiments, A³ is

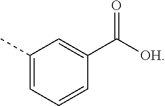

In certain embodiments,

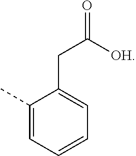

In certain embodiments, A³ is

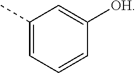

In certain embodiments, A³ is

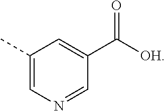

In certain embodiments, A³ is

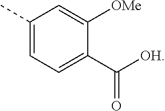

In certain embodiments, A³ is

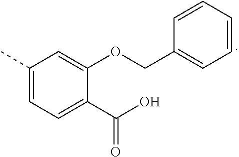

In certain embodiments, L¹ is —CH₂—. In certain embodiments, L¹ is —CH₂C(═O)—*. In certain embodiments, L¹ is —CH₂CH₂C(═O)—*. In certain embodiments, L¹ is —C(═O)CH₂CH₂O—*. In certain embodiments, L¹ is

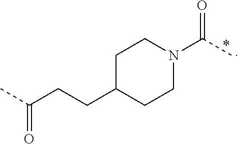

In certain embodiments, L¹ is

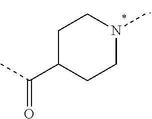

In certain embodiments, L¹ is

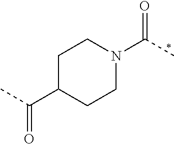

In certain embodiments, L² is —CH₂—. In certain embodiments, L² is —C(═O)—. In certain embodiments, L² is **—C(═O)CH₂O—.

In certain embodiments, R^{1a} is H. In certain embodiments, R^{1a} is CH₃. In certain embodiments, R^{1a} is C(═O)CH₃. In certain embodiments, R^{1a} is

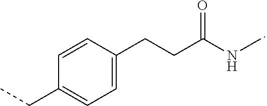

In certain embodiments, R^{1b} is H. In certain embodiments, R^{1b} is

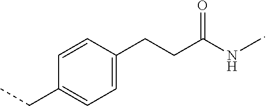

In certain embodiments, R^{1a} is H and R^{1b} is H. In certain embodiments, R^{1a} is CH₃ and R^{1b} is H. In certain embodiments, R^{1a} is C(═O)CH₃ and R^{1b} is H. In certain embodiments, R^{1a} is

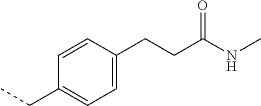

### and R^{1b} is H.

In certain embodiments, R² is H.

In certain embodiments, Y is —NH—. In certain embodiments, Y is —CH₂NH—**. In certain embodiments, Y is —(CH₂)₃NH—**.

In certain embodiments, the compound is selected from the group consisting of
2-hydroxy-4-(5-(((3-((methylamino)methyl)phenyl)amino)methyl)furan-2-yl)benzoic acid;
2-(4′-(((3-(((4-(3-(methylamino)-3-oxopropyl)benzyl)amino)methyl)phenyl)amino)methyl)-[1,1′-biphenyl]-2-yl)acetic acid;
5-((((5-(dimethylcarbamoyl)thiophen-2-yl)methyl)amino)methyl)-2-hydroxybenzoic acid;
5-(((5-(acetamidomethyl)pyridin-2-yl)amino)methyl)-2-hydroxybenzoic acid;
2-hydroxy-4-(5-(((3-(((4-(3-(methylamino)-3-oxopropyl)benzyl)amino)methyl)phenyl)amino)methyl)furan-2-yl)benzoic acid;
2-hydroxy-4-(5-((methylamino)methyl)furan-2-yl)benzoic acid;
4-(5-(((3-((methylamino)methyl)phenyl)amino)methyl)furan-2-yl)benzoic acid;
3-(5-(((3-((methylamino)methyl)phenyl)amino)methyl)furan-2-yl)phenol;
2-hydroxy-5-(5-(((3-((methylamino)methyl)phenyl)amino)methyl)furan-2-yl)benzoic acid;
2-hydroxy-3-(5-(((3-((methylamino)methyl)phenyl)amino)methyl)furan-2-yl)benzoic acid;
3-(5-(((3-((methylamino)methyl)phenyl)amino)methyl)furan-2-yl)benzoic acid;
4-(5-(((3-(aminomethyl)phenyl)amino)methyl)furan-2-yl)-2-hydroxybenzoic acid;
3-(5-(((3-(aminomethyl)phenyl)amino)methyl)furan-2-yl)-2-hydroxybenzoic acid;
4-(5-(((3-(aminomethyl)phenyl)amino)methyl)thiophen-2-yl)-2-hydroxybenzoic acid;
4′-(((3-(aminomethyl)phenyl)amino)methyl)-3-hydroxy-[1,1′-biphenyl]-4-carboxylic acid;
3′-(((3-(aminomethyl)phenyl)amino)methyl)-3-hydroxy-[1,1′-biphenyl]-4-carboxylic acid;
5-(4-(((3-aminophenyl)amino)methyl)phenyl)nicotinic acid;
5-(((3-(aminomethyl)phenyl)amino)methyl)-3′-hydroxy-[1,1′-biphenyl]-3,4′-dicarboxylic acid;
4-(6-(((3-(aminomethyl)phenyl)amino)methyl)pyridin-2-yl)-2-hydroxybenzoic acid;
3′-(((3-(aminomethyl)phenyl)amino)methyl)-3-hydroxy-5′-methyl-[1,1′-biphenyl]-4-carboxylic acid;
3′-(((3-(aminomethyl)phenyl)amino)methyl)-3,5′-dihydroxy-[1,1′-biphenyl]-4-carboxylic acid;
3′-(((3-(acetamidomethyl)phenyl)amino)methyl)-3-hydroxy-[1,1′-biphenyl]-4-carboxylic acid;
3′-((3-(aminomethyl)phenyl)carbamoyl)-3-hydroxy-[1,1′-biphenyl]-4-carboxylic acid;
4-(2-(3-(aminomethyl)phenyl)isoindolin-5-yl)-2-hydroxybenzoic acid;
3-hydroxy-3′-(((3-((methylamino)methyl)phenyl)amino)methyl)-[1,1′-biphenyl]-4-carboxylic acid;
3-hydroxy-3′-(((3-(2-(methylamino)-2-oxoethyl)phenyl)amino)methyl)-[1,1′-biphenyl]-4-carboxylic acid;
3′-(((3-(aminomethyl)-5-hydroxyphenyl)amino)methyl)-3-hydroxy-[1,1′-biphenyl]-4-carboxylic acid;
3′-(((5-(aminomethyl)pyridin-3-yl)amino)methyl)-3-hydroxy-[1,1′-biphenyl]-4-carboxylic acid;
4-(5-(((3-(aminomethyl)phenyl)amino)methyl)pyridin-3-yl)-2-hydroxybenzoic acid;
4-(2-(((3-(aminomethyl)phenyl)amino)methyl)pyridin-4-yl)-2-hydroxybenzoic acid;
4-(4-(((3-(aminomethyl)phenyl)amino)methyl)pyridin-2-yl)-2-hydroxybenzoic acid;
3′-methoxy-5-(2-(methylamino)-2-oxoethoxy)-[1,1′-biphenyl]-3,4′-dicarboxylic acid;
3′-(benzyloxy)-5-(2-(methylamino)-2-oxoethoxy)-[1,1′-biphenyl]-3,4′-dicarboxylic acid;
3′-(benzyloxy)-5-(2-((4-(3-(methylamino)-3-oxopropyl)phenyl)amino)-2-oxoethoxy)-[1,1′-biphenyl]-3,4′-dicarboxylic acid;
2′-(((3-(3-aminophenyl)propyl)amino)methyl)-[1,1′-biphenyl]-3-carboxylic acid;
2′-(((3-(3-((4-(3-(methylamino)-3-oxopropyl)benzyl)amino)phenyl)propyl)amino)methyl)-[1,1′-biphenyl]-3-carboxylic acid;
2′-(((3-cyano-4-(4-(dimethylcarbamoyl)piperidin-1-yl)benzyl)amino)methyl)-[1,1′-biphenyl]-3-carboxylic acid;
2-hydroxy-5-((((5-(4-(3-(methylamino)-3-oxopropyl)piperidine-1-carbonyl)thiophen-2-yl)methyl)amino)methyl)benzoic acid; and
2-hydroxy-5-(((5-((4-(methylcarbamoyl)cyclohexane-1-carboxamido)methyl)pyridin-2-yl)amino)methyl)benzoic acid.

In certain embodiments, the compound is not 3′-(((3-(acetamidomethyl)phenyl)amino)methyl)-3-hydroxy-[1,1′-biphenyl]-4-carboxylic acid.

In another aspect, the present disclosure provides a compound of formula (II), or a salt, solvate, stereoisomer, isotopologue, or tautomer thereof, or any mixtures thereof:

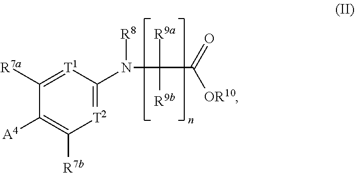

wherein:
A⁴ is selected from the group consisting of optionally substituted C₆-C₁₀ aryl and optionally substituted C₂-C₉ heteroaryl;
R^{7a}, R^{7b}, R^{7c}, and R^{7d}, if present, are each independently selected from the group consisting of H, halogen, OR^{A}, C(═O)OR^{A}, CN, NO₂, optionally substituted C₁-C₆ alkyl, and optionally substituted C₃-C₈ cycloalkyl;
R⁸ is selected from the group consisting of H and optionally substituted C₁-C₆ alkyl;
each occurrence of R^{9a} and R^{9b} is independently selected from the group consisting of H and optionally substituted C₁-C₆ alkyl;
R¹⁰ is selected from the group consisting of H, optionally substituted C₁-C₆ alkyl, optionally substituted C₃-C₁₂ cycloalkyl, optionally substituted C₇-C₁₂ aralkyl, optionally substituted C₂-C₁₂ heterocyclyl, optionally substituted C₆-C₁₂ aryl;
T¹ is selected from the group consisting of N and CR^{7c};
T² is selected from the group consisting of N and CR^{7d};
n is an integer selected from the group consisting of 1, 2, and 3; and
each occurrence of R^{A} and R^{B} is independently selected from the group consisting of H, —C(═O)(optionally substituted C₁-C₆ alkyl), optionally substituted C₁-C₆ alkyl, optionally substituted C₃-C₈ cycloalkyl, optionally substituted C₇-C₁₂ aralkyl, optionally substituted phenyl, optionally substituted naphthyl, and optionally substituted heteroaryl.

In certain embodiments, A⁴ is selected from the group consisting of:

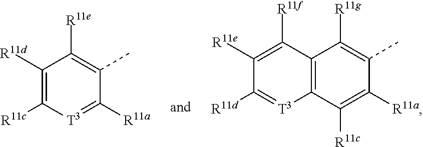

wherein:
R^{11a}, R^{11b}, R^{11c}, R^{11d}, R^{11e}, R^{11f}, and R^{11g}, if present, are each independently selected from the group consisting of H, halogen, OR^{A}, O(optionally substituted C₁-C₃ alkylenyl)C(═O)OR^{A}, O(optionally substituted C₁-C₃ alkylenyl)C(═O)N(R^{A})(R^{B}), C(═O)R^{A}, C(═O)OR^{A}, C(═O)N(R^{A})(R^{B}), CN, NO₂, optionally substituted C₁-C₆ alkyl, and optionally substituted C₃-C₈ cycloalkyl; and
T³ is selected from the group consisting of N and C^{11b}.

In certain embodiments, A⁴ is

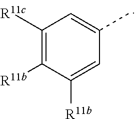

In certain embodiments, A⁴ is

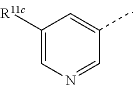

In certain embodiments, A⁴ is

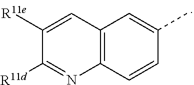

In certain embodiments, R^{11a} is H. In certain embodiments, R^{11a} is Me. In certain embodiments, R^{11a} is OMe. In certain embodiments, R^{11a} is F. In certain embodiments, R^{11a} is C(═O)NH(Me). In certain embodiments, R^{11a} is C(═O)H. In certain embodiments, R^{11a} is OCH₂C(═O)OH. In certain embodiments, R^{11a} is OCH₂C(═O)NMe₂.

In certain embodiments, R^{11b} is H. In certain embodiments, R^{11b} is Me. In certain embodiments, R^{11b} is OMe. In certain embodiments, R^{11b} is F. In certain embodiments, R^{11b} is C(═O)NH(Me). In certain embodiments, R^{11b} is C(═O)H. In certain embodiments, R^{11b} is OCH₂C(═O)OH. In certain embodiments, R^{11b} is OCH₂C(═O)NMe₂.

In certain embodiments, R^{11c} is H. In certain embodiments, R^{11c} is Me. In certain embodiments, R^{11c} is OMe. In certain embodiments, R^{11c} is F. In certain embodiments, R^{11c} is C(═O)NH(Me). In certain embodiments, R^{11c} is C(═O)H. In certain embodiments, R^{11c} is OCH₂C(═O)OH. In certain embodiments, R^{11c} is OCH₂C(═O)NMe₂.

In certain embodiments, R^{11d} is H. In certain embodiments, R^{11d} is Me. In certain embodiments, R^{11d} is OMe. In certain embodiments, R^{11d} is F. In certain embodiments, R^{11d} is C(═O)NH(Me). In certain embodiments, R^{11d} is C(═O)H. In certain embodiments, R^{11d} is OCH₂C(═O)OH. In certain embodiments, R^{11d} is OCH₂C(═O)NMe₂.

In certain embodiments, R^{11e} is H. In certain embodiments, R^{11e} is Me. In certain embodiments, R^{11e} is OMe. In certain embodiments, R^{11e} is F. In certain embodiments, R^{11e} is C(═O)NH(Me). In certain embodiments, R^{11e} is C(═O)H. In certain embodiments, R^{11e} is OCH₂C(═O)OH. In certain embodiments, R^{11e} is OCH₂C(═O)NMe₂.

In certain embodiments, R^{11f} is H. In certain embodiments, R^{11f} is Me. In certain embodiments, R^{11f} is OMe. In certain embodiments, R^{11f} is F. In certain embodiments, R^{11f} is C(═O)NH(Me). In certain embodiments, R^{11f} is C(═O)H. In certain embodiments, R^{11f} is OCH₂C(═O)OH. In certain embodiments, R^{11f} is OCH₂C(═O)NMe₂.

In certain embodiments, R^{11g} is H. In certain embodiments, R^{11g} is Me. In certain embodiments, R^{11g} is OMe. In certain embodiments, R^{11g} is F. In certain embodiments, R^{11g} is C(═O)NH(Me). In certain embodiments, R^{11g} is C(═O)H. In certain embodiments, R^{11g} is OCH₂C(═O)OH. In certain embodiments, R^{11g} is OCH₂C(═O)NMe₂.

In certain embodiments, A⁴ is

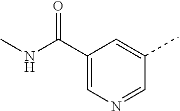

In certain embodiments, A⁴ is

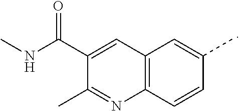

In certain embodiments, A⁴ is

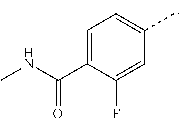

In certain embodiments, A⁴ is

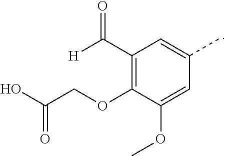

In certain embodiments, A⁴ is

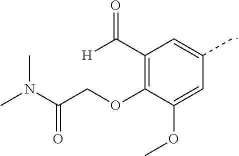

In certain embodiments, at least one of the following applies:
(a) T¹ is N;
(b) T² is N;
(c) R^{7a} is H; and
(d) R^{7b} is H.

In certain embodiments, R⁸ is H.

In certain embodiments, R^{9a} is H and R^{9b} is H.

In certain embodiments, n is 1.

In certain embodiments, R¹⁰ is H.
In certain embodiments, the compound is selected from the group consisting of
(5-(5-(methylcarbamoyl)pyridin-3-yl)pyrimidin-2-yl)glycine;
(5-(2-methyl-3-(methylcarbamoyl)quinolin-6-yl)pyrimidin-2-yl)glycine;
(5-(3-fluoro-4-(methylcarbamoyl)phenyl)pyrimidin-2-yl)glycine;
(5-(4-(2-(dimethylamino)-2-oxoethoxy)-3-formyl-5-methoxyphenyl)pyrimidin-2-yl)glycine; and
(5-(4-(carboxymethoxy)-3-formyl-5-methoxyphenyl)pyrimidin-2-yl)glycine.

In another aspect, the present disclosure provides a compound of formula (III), or a salt, solvate, stereoisomer, isotopologue, or tautomer thereof, or any mixtures thereof:

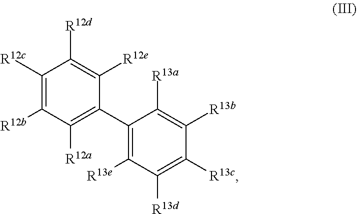

wherein:
R^{12a}, R^{12b}, R^{12c}, R^{12d}, and R^{12e} are each independently selected from the group consisting of H, halogen, OR^{A}, C(═O)R^{A}, C(═O)OR^{A}, C(═O)N(R^{A})(R^{B}), CN, NO₂, optionally substituted C₁-C₆ alkyl, and optionally substituted C₃-C₈ cycloalkyl,
   wherein at least one selected from the group consisting of R^{12a}, R^{12b}, R^{12c}, R^{12d}, and R^{12e} is C(═O)OH;
R^{13a}, R^{13b}, R^{13c}, R^{13d}, and R^{13e} are each independently selected from the group consisting of H, halogen, OR^{A}, C(═O)R^{A}, C(═O)OR^{A}, C(═O)N(R^{A})(R^{B}), CN, NO₂, optionally substituted C₁-C₆ alkyl, and optionally substituted C₃-C₈ cycloalkyl,
   wherein at least one selected from the group consisting of R^{13a}, R^{13b}, R^{13c}, R^{13d}, and R^{13e} is C(═O)OH; and
each occurrence of R^{A} and R^{B} is independently selected from the group consisting of H, —C(═O)(optionally substituted C₁-C₆ alkyl), optionally substituted C₁-C₆ alkyl, optionally substituted C₃-C₈ cycloalkyl, optionally substituted C₇-C₁₂ aralkyl, optionally substituted phenyl, optionally substituted naphthyl, and optionally substituted heteroaryl.

In certain embodiments, R^{12a} is H. In certain embodiments, R^{12a} is OH. In certain embodiments, R^{12a} is C(═O)OH. In certain embodiments, R^{12b} is H. In certain embodiments, R^{12b} is OH. In certain embodiments, R^{12b} is C(═O)OH. In certain embodiments, R^{12c} is H. In certain embodiments, R^{12c} is OH. In certain embodiments, R^{12c} is C(═O)OH. In certain embodiments, R^{12d} is H. In certain embodiments, R^{12d} is OH. In certain embodiments, R^{12d} is C(═O)OH. In certain embodiments, R^{12e} is H. In certain embodiments, R^{12e} is OH. In certain embodiments, R^{12e} is C(═O)OH.

In certain embodiments, R^{13a} is H. In certain embodiments, R^{13a} is OMe. In certain embodiments, R^{13a} is OCH₂Ph. In certain embodiments, R^{13a} is C(═O)OH. In certain embodiments, R^{13b} is H. In certain embodiments, R^{13b} is OMe. In certain embodiments, R^{13b} is OCH₂Ph. In certain embodiments, R^{13b} is C(═O)OH. In certain embodiments, R^{13c} is H. In certain embodiments, R^{13c} is OMe. In certain embodiments, R^{13c} is OCH₂Ph. In certain embodiments, R^{13c} is C(═O)OH. In certain embodiments, R^{13d} is H. In certain embodiments, R^{13d} is OMe. In certain embodiments, R^{13d} is OCH₂Ph. In certain embodiments, R^{13d} is C(═O)OH. In certain embodiments, R^{13e} is H. In certain embodiments, R^{13e} is OMe. In certain embodiments, R^{13e} is OCH₂Ph. In certain embodiments, R^{13e} is C(═O)OH.

In certain embodiments, the compound is:

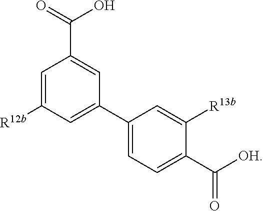

In certain embodiments, the compound is selected from the group consisting of:
5-hydroxy-3′-methoxy-[1,1′-biphenyl]-3,4′-dicarboxylic acid; and
3′-(benzyloxy)-5-hydroxy-[1,1′-biphenyl]-3,4′-dicarboxylic acid.

In another aspect, the present disclosure provides a compound of formula (IV), or a salt, solvate, stereoisomer, isotopologue, or tautomer thereof, or any mixtures thereof:

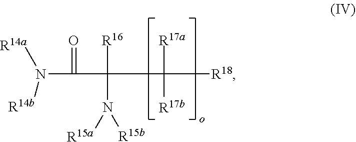

R^{14a} and R^{14b} are each independently selected from the group consisting of H, optionally substituted C₁-C₆ alkyl, optionally substituted C₃-C₈ cycloalkyl, and optionally substituted C₇-C₁₂ aralkyl;
R^{15a} and R^{15b} are each independently selected from the group consisting of H and optionally substituted C₁-C₆ alkyl;
R¹⁶ is selected from the group consisting of H and optionally substituted C₁-C₆ alkyl;
R^{17a} and R^{17b} are each independently selected from the group consisting of H and optionally substituted C₁-C₆ alkyl;
R¹⁸ is optionally substituted aryl;
is an integer selected from the group consisting of 0, 1, 2, and 3; and
each occurrence of R^{A} and R^{B} is independently selected from the group consisting of H, —C(═O)(optionally substituted C₁-C₆ alkyl), optionally substituted C₁-C₆ alkyl, optionally substituted C₃-C₈ cycloalkyl, optionally substituted C₇-C₁₂ aralkyl, optionally substituted phenyl, optionally substituted naphthyl, and optionally substituted heteroaryl.

In certain embodiments, R^{14a} is H. In certain embodiments, R^{14a} is CH₂CH₂Ph.

In certain embodiments, R^{14b} is H. In certain embodiments, R^{14b} is CH₂CH₂Ph.

In certain embodiments, R^{15a} and R^{15b} are each independently H.

In certain embodiments, R¹⁶ is H.

In certain embodiments, R^{17a} and R^{17b} are each independently H.

In certain embodiments, o is 1.

In certain embodiments, R¹⁸ is phenyl optionally substituted with at least one hydroxyl. In certain embodiments, R¹⁸ is

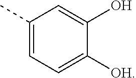

In certain embodiments, the compound is 2-amino-3-(3,4-dihydroxyphenyl)-N-phenethylpropanamide.

In certain embodiments, each occurrence of alkyl, cycloalkyl, aralkyl, alkylenyl, phenylenyl, heteroarylenyl, heterocyclyl, heteroaryl, phenyl, naphthyl, and aryl is independently optionally substituted with at least one substituent C₁-C₆ alkyl, C₃-C₈ cycloalkyl, C₁-C₆ hydroxyalkyl, halogen, CN, NO₂ OR^{I}, N(R^{I})(R^{II}), C₁-C₆ alkoxy, C₃-C₈ cycloalkoxy, C₁-C₆ haloalkoxy, C₃-C₈ halocycloalkoxy, phenyl, heteroaryl, heterocyclyl, (C₁-C₆ alkylenyl)C(═O)N(R^{I})(R^{II}), (C₁-C₆ alkylenyl)C(═O)OR^{I}, O(C₁-C₃ alkylenyl)C(═O)OR^{II}, O(C₁-C₃ alkylenyl)C(═O)N(R^{I})(R^{II}), C(═O)R^{I}, C(═O)OR^{I}, OC(═O)R^{I}, OC(═O)OR^{I}, SR^{I}, S(═O)R^{I}, S(═O)₂R^{I}, S(═O)₂N(R^{I})(R^{II}), S(═O)₂NR^{I}C(═O)NHR^{II}, N(R^{I})S(═O)₂R^{II}, N(R^{I})C(═O)R^{II}, and C(═O)NR^{I}R^{II}, wherein R^{I} and R^{II} is independently selected from the group consisting of H, —C(═O)(C₁-C₆ alkyl), C₁-C₆ alkyl, C₃-C₈ cycloalkyl, C₇-C₁₂ aralkyl, phenyl, naphthyl, and heteroaryl.

TABLE 1
Exemplary compounds

| Compoud | Structure |
|---|---|
| 1 CDD-2308 | 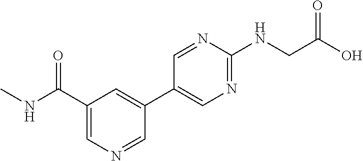 [SMILES: CNC(=O)c1cncc(-c2cnc(NCC(=O)O)nc2)c1] |
| | (5-(5-(methylcarbamoyl)pyridin-3-yl)pyrimidin-2-yl)glycine |
| 2 CDD-2310 | 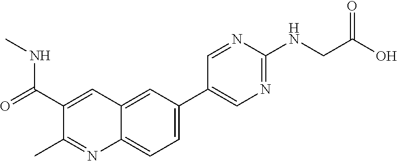 [SMILES: CNC(=O)c1cc2cc(-c3cnc(NCC(=O)O)nc3)ccc2nc1C] |
| | (5-(2-methyl-3-(methylcarbamoyl)quinolin-6-yl)pyrimidin-2- |
| | yl)glycine |
| 3 CDD-2309 | 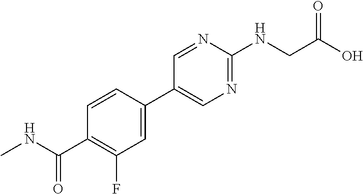 [SMILES: CNC(=O)c1ccc(-c2cnc(NCC(=O)O)nc2)cc1F] |
| | (5-(3-fluoro-4-(methylcarbamoyl)phenyl)pyrimidin-2-yl)glycine |
| 4 CDD-2312 | 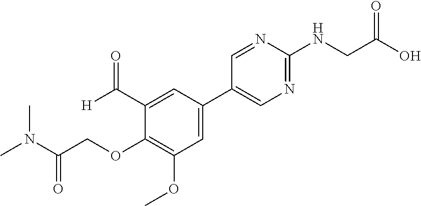 [SMILES: COc1cc(-c2cnc(NCC(=O)O)nc2)cc(C=O)c1OCC(=O)N(C)C] |
| | (5-(4-(2-(dimethylamino)-2-oxoethoxy)-3-formyl-5- |
| | methoxyphenyl)pyrimidin-2-yl)glycine |
| 5 CDD-2311 | 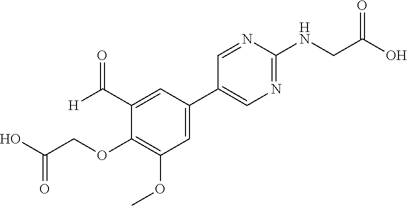 [SMILES: COc1cc(-c2cnc(NCC(=O)O)nc2)cc(C=O)c1OCC(=O)O] |
| | (5-(4-(carboxymethoxy)-3-formyl-5-methoxyphenyl)pyrimidin-2- |
| | yl)glycine |
| 6 CDD-2724 | 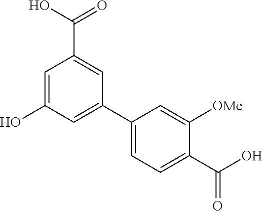 [SMILES: COc1cc(-c2cc(O)cc(C(=O)O)c2)ccc1C(=O)O] |
| | 5-hydroxy-3′-methoxy-[1,1′-biphenyl]-3,4′-dicarboxylic acid |
| 7 CDD-2725 | 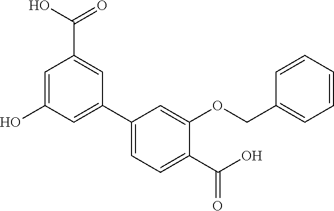 [SMILES: O=C(O)c1cc(O)cc(-c2ccc(C(=O)O)c(OCc3ccccc3)c2)c1] |
| | 3′-(benzyloxy)-5-hydroxy-[1,1′-biphenyl]-3,4′-dicarboxylic acid |
| 8 CDD-2376 | 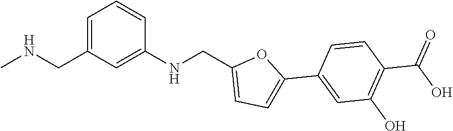 [SMILES: CNCc1cccc(NCc2ccc(-c3ccc(C(=O)O)c(O)c3)o2)c1] |
| | 2-hydroxy-4-(5-(((3- |
| | ((methylamino)methyl)phenyl)amino)methyl)furan-2-yl)benzoic acid |
| 9 CDD-2336 | 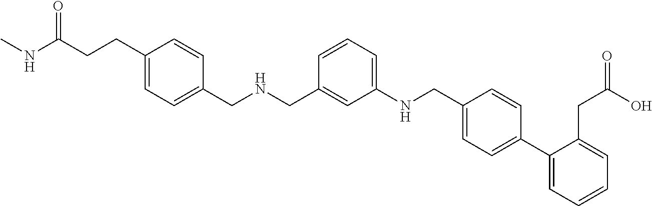 [SMILES: CNC(=O)CCc1ccc(CNCc2cccc(NCc3ccc(-c4ccccc4CC(=O)O)cc3)c2)cc1] |
| | 2-(4′-(((3-(((4-(3-(methylamino)-3- |
| | oxopropyl)benzyl)amino)methyl)phenyl)amino)methyl)-[1,1′- |
| | biphenyl]-2-yl)acetic acid |
| 10 CDD-2350 | 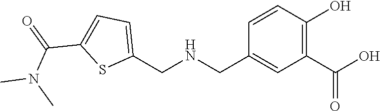 [SMILES: CN(C)C(=O)c1ccc(CNCc2ccc(O)c(C(=O)O)c2)s1] |
| | 5-((((5-(dimethylcarbamoyl)thiophen-2-yl)methyl)amino)methyl)-2- |
| | hydroxybenzoic acid |
| 11 CDD-2373 | 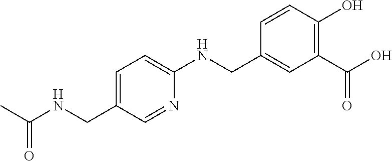 [SMILES: CC(=O)NCc1ccc(NCc2ccc(O)c(C(=O)O)c2)nc1] |
| | 5-(((5-(acetamidomethyl)pyridin-2-yl)amino)methyl)-2- |
| | hydroxybenzoic acid |
| 12 CDD-2335 | 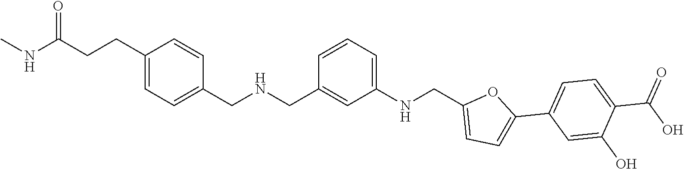 [SMILES: CNC(=O)CCc1ccc(CNCc2cccc(NCc3ccc(-c4ccc(C(=O)O)c(O)c4)o3)c2)cc1] |
| | 2-hydroxy-4-(5-(((3-(((4-(3-(methylamino)-3- |
| | oxopropyl)benzyl)amino)methyl)phenyl)amino)methyl)furan-2- |
| | yl)benzoic acid |
| 13 CDD-2363 | 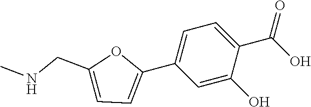 [SMILES: CNCc1ccc(-c2ccc(C(=O)O)c(O)c2)o1] |
| | 2-hydroxy-4-(5-((methylamino)methyl)furan-2-yl)benzoic acid |
| 14 CDD-2463 | 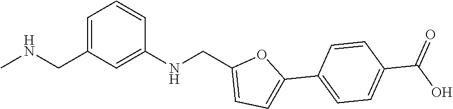 [SMILES: CNCc1cccc(NCc2ccc(-c3ccc(C(=O)O)cc3)o2)c1] |
| | 4-(5-(((3-((methylamino)methyl)phenyl)amino)methyl)furan-2- |
| | yl)benzoic acid |
| 15 CDD-2508 | 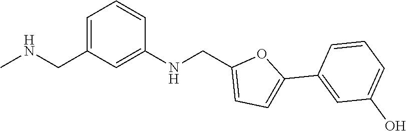 [SMILES: CNCc1cccc(NCc2ccc(-c3cccc(O)c3)o2)c1] |
| | 3-(5-(((3-((methylamino)methyl)phenyl)amino)methyl)furan-2- |
| | yl)phenol |
| 16 CDD-2510 | 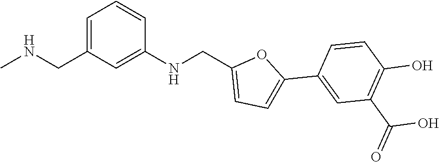 [SMILES: CNCc1cccc(NCc2ccc(-c3ccc(O)c(C(=O)O)c3)o2)c1] |
| | 2-hydroxy-5-(5-(((3- |
| | ((methylamino)methyl)phenyl)amino)methyl)furan-2-yl)benzoic acid |
| 17 CDD-2522 | 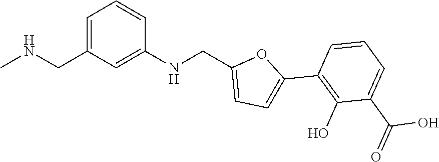 [SMILES: CNCc1cccc(NCc2ccc(-c3cccc(C(=O)O)c3O)o2)c1] |
| | 2-hydroxy-3-(5-(((3- |
| | ((methylamino)methyl)phenyl)amino)methyl)furan-2-yl)benzoic acid |
| 18 CDD-2602 | 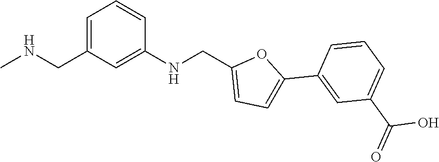 [SMILES: CNCc1cccc(NCc2ccc(-c3cccc(C(=O)O)c3)o2)c1] |
| | 3-(5-(((3-((methylamino)methyl)phenyl)amino)methyl)furan-2- |
| | yl)benzoic acid |
| 19 CDD-2511 | 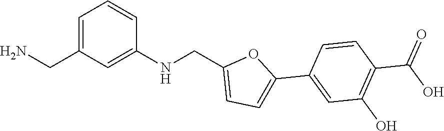 [SMILES: NCc1cccc(NCc2ccc(-c3ccc(C(=O)O)c(O)c3)o2)c1] |
| | 4-(5-(((3-(aminomethyl)phenyl)amino)methyl)furan-2-yl)-2- |
| | hydroxybenzoic acid |
| 20 CDD-2567 | 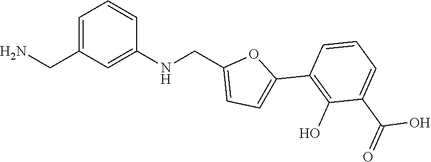 [SMILES: NCc1cccc(NCc2ccc(-c3cccc(C(=O)O)c3O)o2)c1] |
| | 3-(5-(((3-(aminomethyl)phenyl)amino)methyl)furan-2-yl)-2- |
| | hydroxybenzoic acid |
| 21 CDD-2600 | 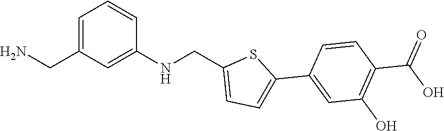 [SMILES: NCc1cccc(NCc2ccc(-c3ccc(C(=O)O)c(O)c3)s2)c1] |
| | 4-(5-(((3-(aminomethyl)phenyl)amino)methyl)thiophen-2-yl)-2- hydroxybenzoic acid |
| 22 CDD-2615 | 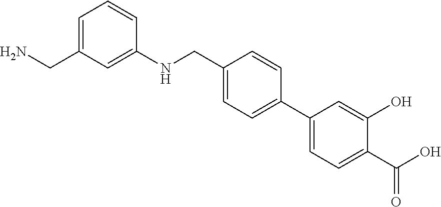 [SMILES: NCc1cccc(NCc2ccc(-c3ccc(C(=O)O)c(O)c3)cc2)c1] |
| | 4′-(((3-(aminomethyl)phenyl)amino)methyl)-3-hydroxy-[1,1′- |
| | biphenyl]-4-carboxylic acid |
| 23 CDD-2616 | 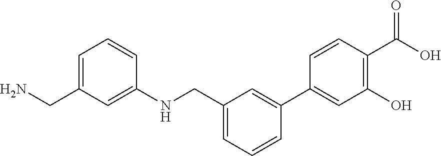 [SMILES: NCc1cccc(NCc2cccc(-c3ccc(C(=O)O)c(O)c3)c2)c1] |
| | 3′-(((3-(aminomethyl)phenyl)amino)methyl)-3-hydroxy-[1,1′- |
| | biphenyl]-4-carboxylic acid |
| 24 CDD-2396 | 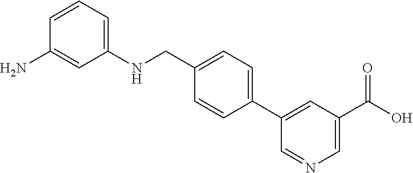 [SMILES: Nc1cccc(NCc2ccc(-c3cncc(C(=O)O)c3)cc2)c1] |
| | 5-(4-(((3-aminophenyl)amino)methyl)phenyl)nicotinic acid |
| 25 CDD-2507 | 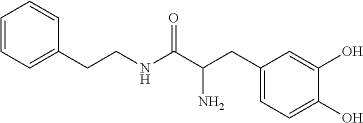 [SMILES: NC(Cc1ccc(O)c(O)c1)C(=O)NCCc1ccccc1] |
| | 2-amino-3-(3,4-dihydroxyphenyl)-N-phenethylpropanamide |
| 26 CDD-2897 | 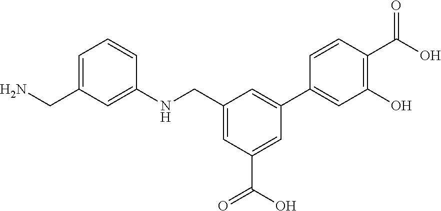 [SMILES: NCc1cccc(NCc2cc(C(=O)O)cc(-c3ccc(C(=O)O)c(O)c3)c2)c1] |
| | 5-(((3-(aminomethyl)phenyl)amino)methyl)-3'-hydroxy-[1,1′- |
| | biphenyl]-3,4′-dicarboxylic acid |
| 27 CDD-2653 | 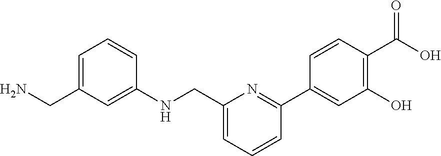 [SMILES: NCc1cccc(NCc2cccc(-c3ccc(C(=O)O)c(O)c3)n2)c1] |
| | 4-(6-(((3-(aminomethyl)phenyl)amino)methyl)pyridin-2-yl)-2- |
| | hydroxybenzoic acid |
| 28 CDD-2997 | 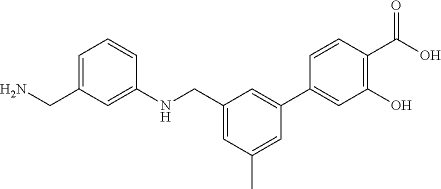 [SMILES: Cc1cc(CNc2cccc(CN)c2)cc(-c2ccc(C(=O)O)c(O)c2)c1] |
| | 3′-(((3-(aminomethyl)phenyl)amino)methyl)-3-hydroxy-5′-methyl- |
| | [1,1′-bipheny]]-4-carboxylic acid |
| 29 CDD-3061 | 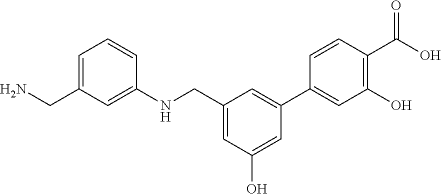 [SMILES: NCc1cccc(NCc2cc(O)cc(-c3ccc(C(=O)O)c(O)c3)c2)c1] |
| | 3′-(((3-(aminomethyl)phenyl)amino)methyl)-3,5′-dihydroxy-[1,1′- |
| | biphenyl]-4-carboxylic acid |
| 30 CDD-2919 | 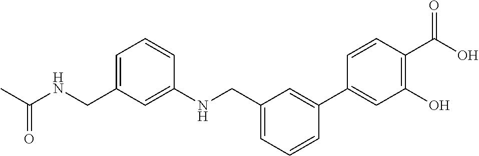 [SMILES: CC(=O)NCc1cccc(NCc2cccc(-c3ccc(C(=O)O)c(O)c3)c2)c1] |
| | 3′-(((3-(acetamidomethyl)phenyl)amino)methyl)-3-hydroxy-[1,1′- |
| | biphenyl]-4-carboxylic acid |
| 31 CDD-2907 | 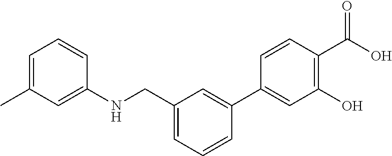 [SMILES: Cc1cccc(NCc2cccc(-c3ccc(C(=O)O)c(O)c3)c2)c1] |
| | 3-hydroxy-3′-((m-tolylamino)methyl)-[1,1′-biphenyl]-4-carboxylic acid |
| 32 CDD-2985 | 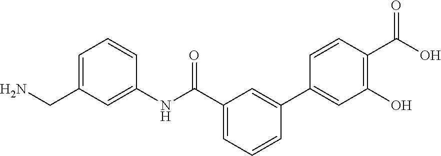 [SMILES: NCc1cccc(NC(=O)c2cccc(-c3ccc(C(=O)O)c(O)c3)c2)c1] |
| | 3′-((3-(aminomethyl)phenyl)carbamoyl)-3-hydroxy-[1,1′-biphenyl]-4- |
| | carboxylic acid |
| 33 CDD-3080 | 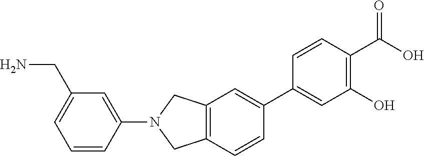 [SMILES: NCc1cccc(N2Cc3ccc(-c4ccc(C(=O)O)c(O)c4)cc3C2)c1] |
| | 4-(2-(3-(aminomethyl)phenyl)isoindolin-5-yl)-2-hydroxybenzoic acid |
| 34 CDD-2906 | 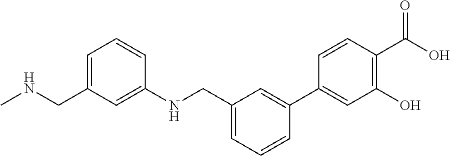 [SMILES: CNCc1cccc(NCc2cccc(-c3ccc(C(=O)O)c(O)c3)c2)c1] |
| | 3-hydroxy-3′-(((3-((methylamino)methyl)phenyl)amino)methyl)-[1,1′- |
| | biphenyl]-4-carboxylic acid |
| 35 CDD-2920 | 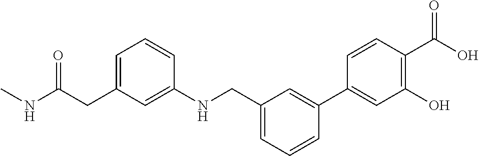 [SMILES: CNC(=O)Cc1cccc(NCc2cccc(-c3ccc(C(=O)O)c(O)c3)c2)c1] |
| | 3-hydroxy-3′-(((3-(2-(methylamino)-2- oxoethyl)phenyl)amino)methyl)-[1,1′-biphenyl]-4-carboxylic acid |
| 36 CDD-2974 | 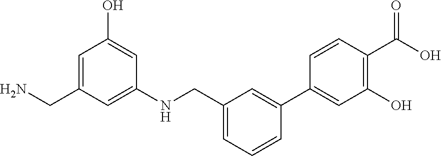 [SMILES: NCc1cc(O)cc(NCc2cccc(-c3ccc(C(=O)O)c(O)c3)c2)c1] |
| | 3′-(((3-(aminomethyl)-5-hydroxyphenyl)amino)methyl)-3-hydroxy- |
| | [1,1′-biphenyl]-4-carboxylic acid |
| 37 CDD-2975 | 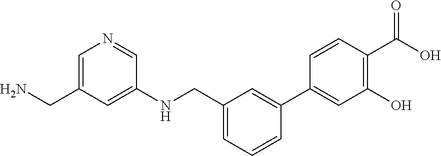 [SMILES: NCc1cncc(NCc2cccc(-c3ccc(C(=O)O)c(O)c3)c2)c1] |
| | 3′-(((5-(aminomethyl)pyridin-3-yl)amino)methyl)-3-hydroxy-[1,1′- |
| | biphenyl]-4-carboxylic acid |
| 38 CDD-2998 | 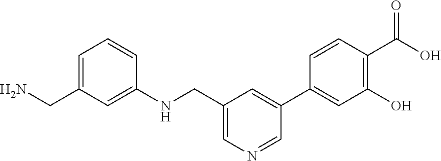 [SMILES: NCc1cccc(NCc2cncc(-c3ccc(C(=O)O)c(O)c3)c2)c1] |
| | 4-(5-(((3-(aminomethyl)phenyl)amino)methyl)pyridin-3-yl)-2- |
| | hydroxybenzoic acid |
| 39 CDD-3059 | 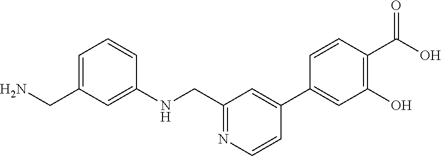 [SMILES: NCc1cccc(NCc2cc(-c3ccc(C(=O)O)c(O)c3)ccn2)c1] |
| | 4-(2-(((3-(aminomethyl)phenyl)amino)methyl)pyridin-4-yl)-2- |
| | hydroxybenzoic acid |
| 40 CDD-3060 | 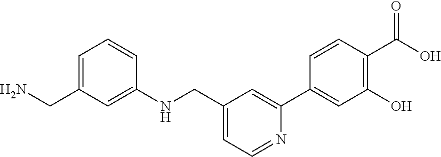 [SMILES: NCc1cccc(NCc2ccnc(-c3ccc(C(=O)O)c(O)c3)c2)c1] |
| | 4-(4-(((3-(aminomethyl)phenyl)amino)methyl)pyridin-2-yl)-2- |
| | hydroxybenzoic acid |
| 41 CDD-2746 | 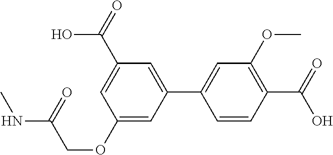 [SMILES: CNC(=O)COc1cc(C(=O)O)cc(-c2ccc(C(=O)O)c(OC)c2)c1] |
| | 3′-methoxy-5-(2-(methylamino)-2-oxoethoxy)-[1,1′-biphenyl]-3,4′- |
| | dicarboxylic acid |
| 42 CDD-2747 | 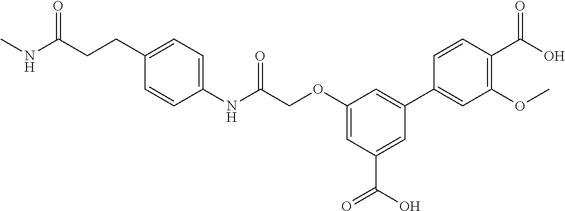 |
| | 3′-methoxy-5-(2-((4-(3-(methylamino)-3-oxopropyl)phenyl)amino)-2- |
| | oxoethoxy)-[1,1′-biphenyl]-3,4′-dicarboxylic acid |
| 43 CDD-2801 | 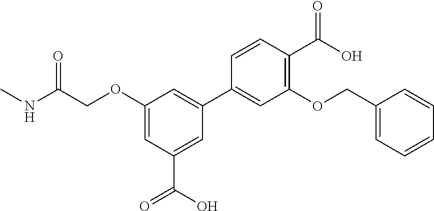 [SMILES: CNC(=O)COc1cc(C(=O)O)cc(-c2ccc(C(=O)O)c(OCc3ccccc3)c2)c1] |
| | 3′-(benzyloxy)-5-(2-(methylamino)-2-oxoethoxy)-[1,1′-biphenyl]-3,4′- |
| | dicarboxylic acid |
| 44 CDD-2802 | 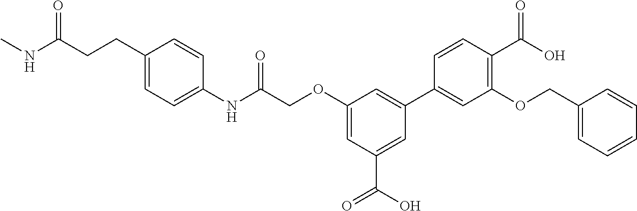 [SMILES: CNC(=O)CCc1ccc(NC(=O)COc2cc(C(=O)O)cc(-c3ccc(C(=O)O)c(OCc4ccccc4)c3)c2)cc1] |
| | 3′-(benzyloxy)-5-(2-((4-(3-(methylamino)-3-oxopropyl)phenyl)amino)- |
| | 2-oxoethoxy)-[1,1′-biphenyl]-3,4′-dicarboxylic acid |
| 45 CDD-2820 | 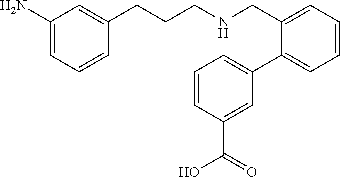 [SMILES: Nc1cccc(CCCNCc2ccccc2-c2cccc(C(=O)O)c2)c1] |
| | 2′-(((3-(3-aminophenyl)propyl)amino)methyl)-[1,1′-biphenyl]-3- |
| | carboxylic acid |
| 46 CDD-2819 | 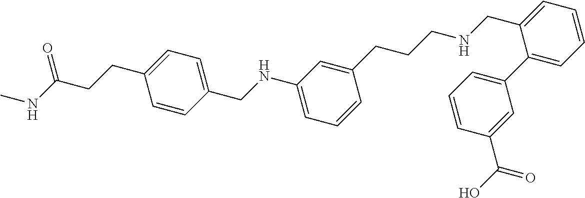 [SMILES: CNC(=O)CCc1ccc(CNc2cccc(CCCNCc3ccccc3-c3cccc(C(=O)O)c3)c2)cc1] |
| | 2′-(((3-(3-((4-(3-(methylamino)-3- |
| | oxopropyl)benzyl)amino)phenyl)propyl)amino)methyl)-[1,1′- |
| | biphenyl]-3-carboxylic acid |
| 47 CDD-2836 | 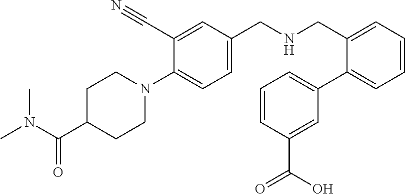 [SMILES: CN(C)C(=O)C1CCN(c2ccc(CNCc3ccccc3-c3cccc(C(=O)O)c3)cc2C#N)CC1] |
| | 2′-(((3-cyano-4-(4-(dimethylcarbamoyl)piperidin-1- |
| | yl)benzyl)amino)methyl)-[1,1′-biphenyl]-3-carboxylic acid |
| 48 CDD-2371 | 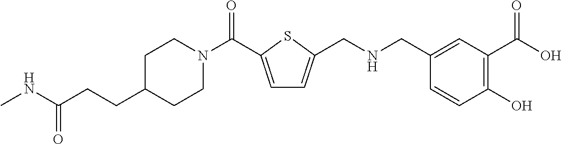 [SMILES: CNC(=O)CCC1CCN(C(=O)c2ccc(CNCc3ccc(O)c(C(=O)O)c3)s2)CC1] |
| | 2-hydroxy-5-((((5-(4-(3-(methylamino)-3-oxopropyl)piperidine-1- |
| | carbonyl)thiophen-2-yl)methyl)amino)methyl)benzoic acid |
| 49 CDD-2395 | 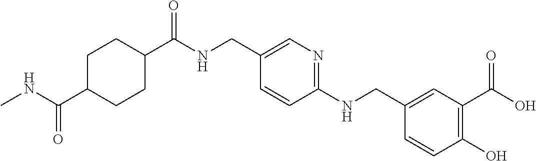 [SMILES: CNC(=O)C1CCC(C(=O)NCc2ccc(NCc3ccc(O)c(C(=O)O)c3)nc2)CC1] |
| | 2-hydroxy-5-(((5-((4-(methylcarbamoyl)cyclohexane-1- |
| | carboxamido)methyl)pyridin-2-yl)amino)methyl)benzoic acid |

The compounds described herein can possess one or more stereocenters, and each stereocenter can exist independently in either the (R) or (S) configuration. In certain embodiments, compounds described herein are present in optically active or racemic forms. It is to be understood that the compounds described herein encompass racemic, optically-active, regioisomeric and stereoisomeric forms, or combinations thereof that possess the therapeutically useful properties described herein. Preparation of optically active forms is achieved in any suitable manner, including by way of non-limiting example, by resolution of the racemic form with recrystallization techniques, synthesis from optically-active starting materials, chiral synthesis, or chromatographic separation using a chiral stationary phase. In certain embodiments, a mixture of one or more isomer is utilized as the therapeutic compound described herein. In other embodiments, compounds described herein contain one or more chiral centers. These compounds are prepared by any means, including stereoselective synthesis, enantioselective synthesis and/or separation of a mixture of enantiomers and/or diastereomers. Resolution of compounds and isomers thereof is achieved by any means including, by way of non-limiting example, chemical processes, enzymatic processes, fractional crystallization, distillation, and chromatography.

The methods and formulations described herein include the use of N-oxides (if appropriate), crystalline forms (also known as polymorphs), solvates, amorphous phases, and/or pharmaceutically acceptable salts of compounds having the structure of any compound(s) described herein, as well as metabolites and active metabolites of these compounds having the same type of activity. Solvates include water, ether (e.g., tetrahydrofuran, methyl tert-butyl ether) or alcohol (e.g., ethanol) solvates, acetates and the like. In certain embodiments, the compounds described herein exist in solvated forms with pharmaceutically acceptable solvents such as water, and ethanol. In other embodiments, the compounds described herein exist in unsolvated form.

In certain embodiments, the compound(s) described herein can exist as tautomers. All tautomers are included within the scope of the compounds presented herein.

In certain embodiments, compounds described herein are prepared as prodrugs. A “prodrug” refers to an agent that is converted into the parent drug in vivo. In certain embodiments, upon in vivo administration, a prodrug is chemically converted to the biologically, pharmaceutically or therapeutically active form of the compound. In other embodiments, a prodrug is enzymatically metabolized by one or more steps or processes to the biologically, pharmaceutically or therapeutically active form of the compound.

In certain embodiments, sites on, for example, the aromatic ring portion of compound(s) described herein are susceptible to various metabolic reactions. Incorporation of appropriate substituents on the aromatic ring structures may reduce, minimize or eliminate this metabolic pathway. In certain embodiments, the appropriate substituent to decrease or eliminate the susceptibility of the aromatic ring to metabolic reactions is, by way of example only, a deuterium, a halogen, or an alkyl group.

Compounds described herein also include isotopically-labeled compounds wherein one or more atoms is replaced by an atom having the same atomic number, but an atomic mass or mass number different from the atomic mass or mass number usually found in nature.

Examples of isotopes suitable for inclusion in the compounds described herein include and are not limited to ²H, ³H, ¹¹C, ¹³C, ¹⁴C, ³⁶Cl, ¹⁸F, ¹²³I, ¹²⁵I, ¹³N, ¹⁵N, ¹⁵O, ¹⁷O, ¹⁸O, ³²P, and ³⁵S. In certain embodiments, isotopically-labeled compounds are useful in drug and/or substrate tissue distribution studies. In other embodiments, substitution with heavier isotopes such as deuterium affords greater metabolic stability (for example, increased in vivo half-life or reduced dosage requirements). In yet other embodiments, substitution with positron emitting isotopes, such as ¹¹C, ¹⁸F, ¹⁵O, and ¹³N, is useful in Positron Emission Topography (PET) studies for examining substrate receptor occupancy. Isotopically-labeled compounds are prepared by any suitable method or by processes using an appropriate isotopically-labeled reagent in place of the non-labeled reagent otherwise employed.

In certain embodiments, the compounds described herein are labeled by other means, including, but not limited to, the use of chromophores or fluorescent moieties, bioluminescent labels, or chemiluminescent labels.

The compounds described herein, and other related compounds having different substituents are synthesized using techniques and materials described herein and as described, for example, in Fieser & Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), Larock's Comprehensive Organic Transformations (VCH Publishers Inc., 1989), March, Advanced Organic Chemistry 4^{th} Ed., (Wiley 1992); Carey & Sundberg, Advanced Organic Chemistry 4th Ed., Vols. A and B (Plenum 2000, 2001), and Green & Wuts, Protective Groups in Organic Synthesis 3rd Ed., (Wiley 1999) (all of which are incorporated by reference for such disclosure). General methods for the preparation of compound as described herein are modified by the use of appropriate reagents and conditions, for the introduction of the various moieties found in the formula as provided herein.

Compounds described herein are synthesized using any suitable procedures starting from compounds that are available from commercial sources, or are prepared using procedures described herein.

In certain embodiments, reactive functional groups, such as hydroxyl, amino, imino, thio or carboxy groups, are protected in order to avoid their unwanted participation in reactions. Protecting groups are used to block some or all of the reactive moieties and prevent such groups from participating in chemical reactions until the protective group is removed. In other embodiments, each protective group is removable by a different means. Protective groups that are cleaved under totally disparate reaction conditions fulfill the requirement of differential removal.

In certain embodiments, protective groups are removed by acid, base, reducing conditions (such as, for example, hydrogenolysis), and/or oxidative conditions. Groups such as trityl, dimethoxytrityl, acetal and t-butyldimethylsilyl are acid labile and are used to protect carboxy and hydroxy reactive moieties in the presence of amino groups protected with Cbz groups, which are removable by hydrogenolysis, and Fmoc groups, which are base labile. Carboxylic acid and hydroxy reactive moieties are blocked with base labile groups such as, but not limited to, methyl, ethyl, and acetyl, in the presence of amines that are blocked with acid labile groups, such as t-butyl carbamate, or with carbamates that are both acid and base stable but hydrolytically removable.

In certain embodiments, carboxylic acid and hydroxy reactive moieties are blocked with hydrolytically removable protective groups such as the benzyl group, while amine groups capable of hydrogen bonding with acids are blocked with base labile groups such as Fmoc. Carboxylic acid reactive moieties are protected by conversion to simple ester compounds as exemplified herein, which include conversion to alkyl esters, or are blocked with oxidatively-removable protective groups such as 2,4-dimethoxybenzyl, while co-existing amino groups are blocked with fluoride labile silyl carbamates.

Allyl blocking groups are useful in the presence of acid- and base-protecting groups since the former are stable and are subsequently removed by metal or pi-acid catalysts. For example, an allyl-blocked carboxylic acid is deprotected with a palladium-catalyzed reaction in the presence of acid labile t-butyl carbamate or base-labile acetate amine protecting groups. Yet another form of protecting group is a resin to which a compound or intermediate is attached. As long as the residue is attached to the resin, that functional group is blocked and does not react. Once released from the resin, the functional group is available to react.

Typically blocking/protecting groups may be selected from allyl, benzyl (Bn), benzyloxycarbonyl (Cbz), allyloxycarbonyl (Alloc), methyl, ethyl, t-butyl, t-butyldimethylsilyl (TBDMS), 2-(trimethylsilyl)ethoxycarbonyl (Teoc), t-butyloxycarbonyl (Boc), para-methoxybenzyl (PMB), triphenylmethyl (trityl), acetyl, and fluorenylmethoxycarbonyl (FMOC).

Other protecting groups, plus a detailed description of techniques applicable to the creation of protecting groups and their removal are described in Greene & Wuts, Protective Groups in Organic Synthesis, 3rd Ed., John Wiley & Sons, New York, NY, 1999, and Kocienski, Protective Groups, Thieme Verlag, New York, NY, 1994, which are incorporated herein by reference for such disclosure.

The present disclosure provides certain exemplary methods for the preparation of compounds of Formula (I), for example, including but not limited to the methods depicted in Schemes 1-7.

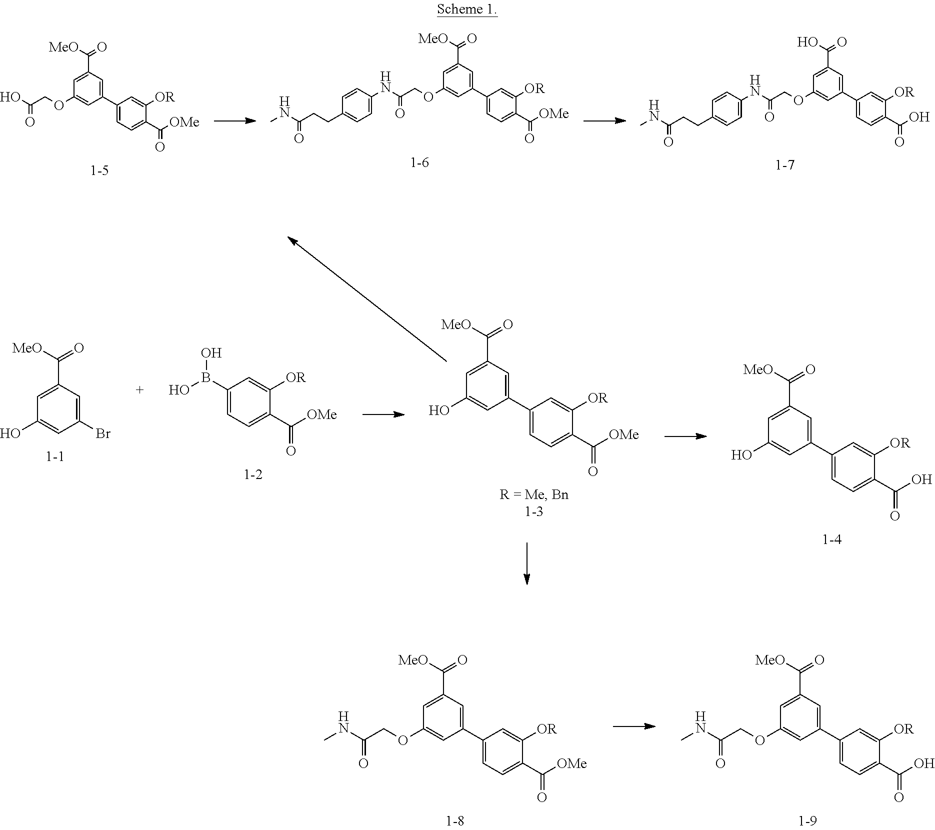

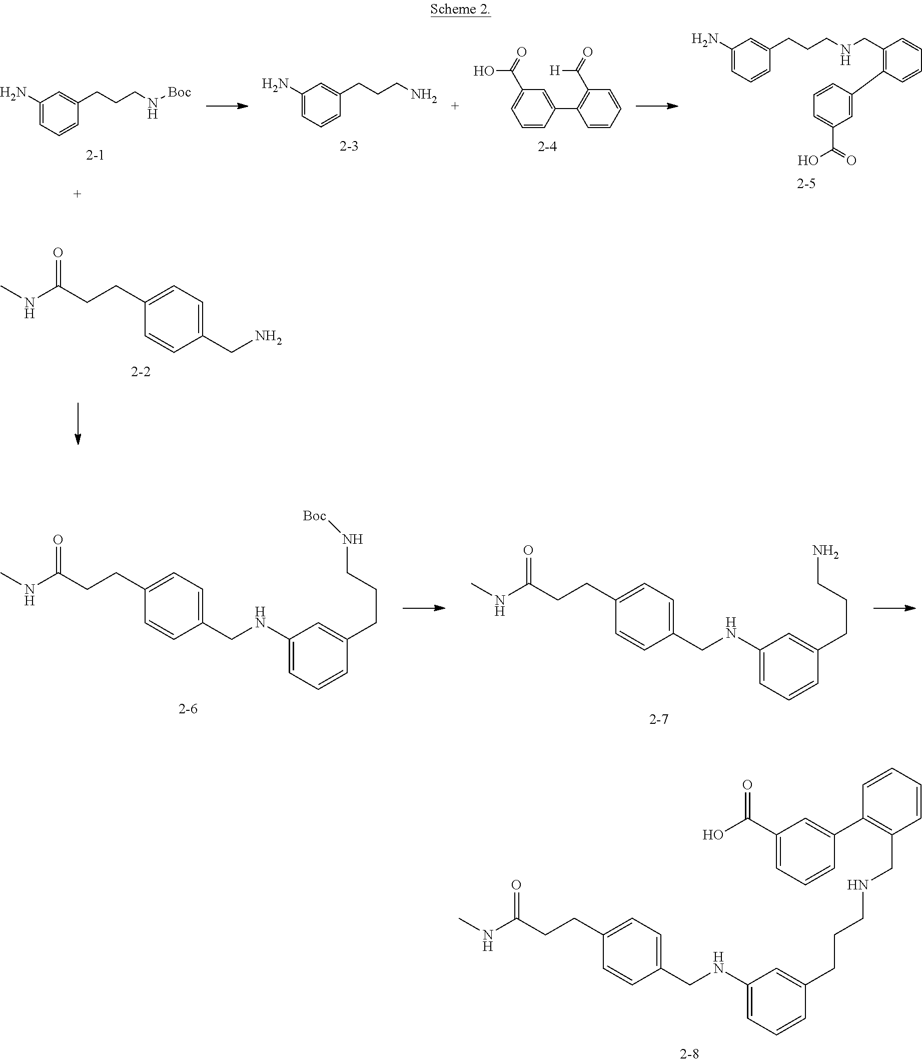

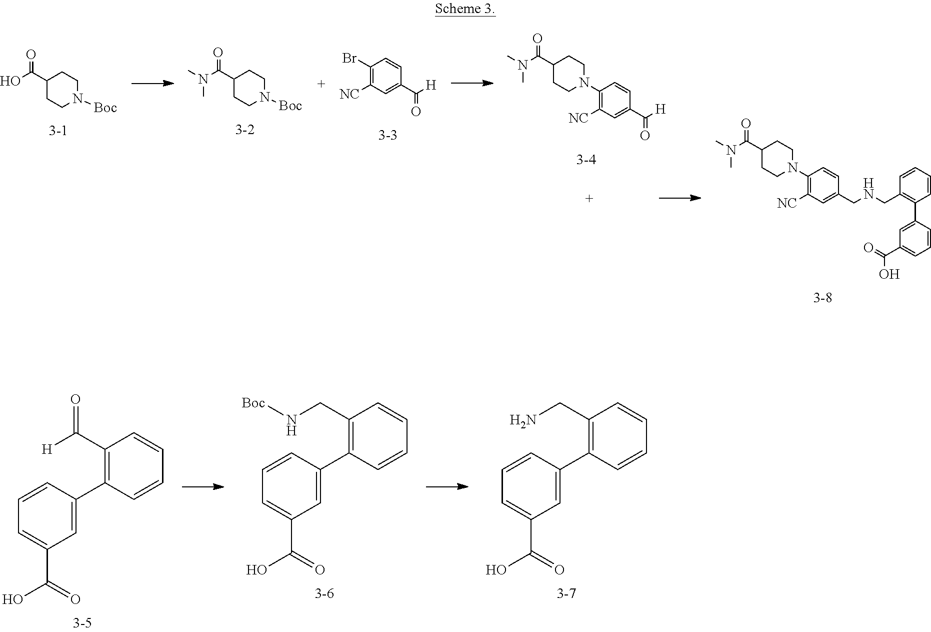

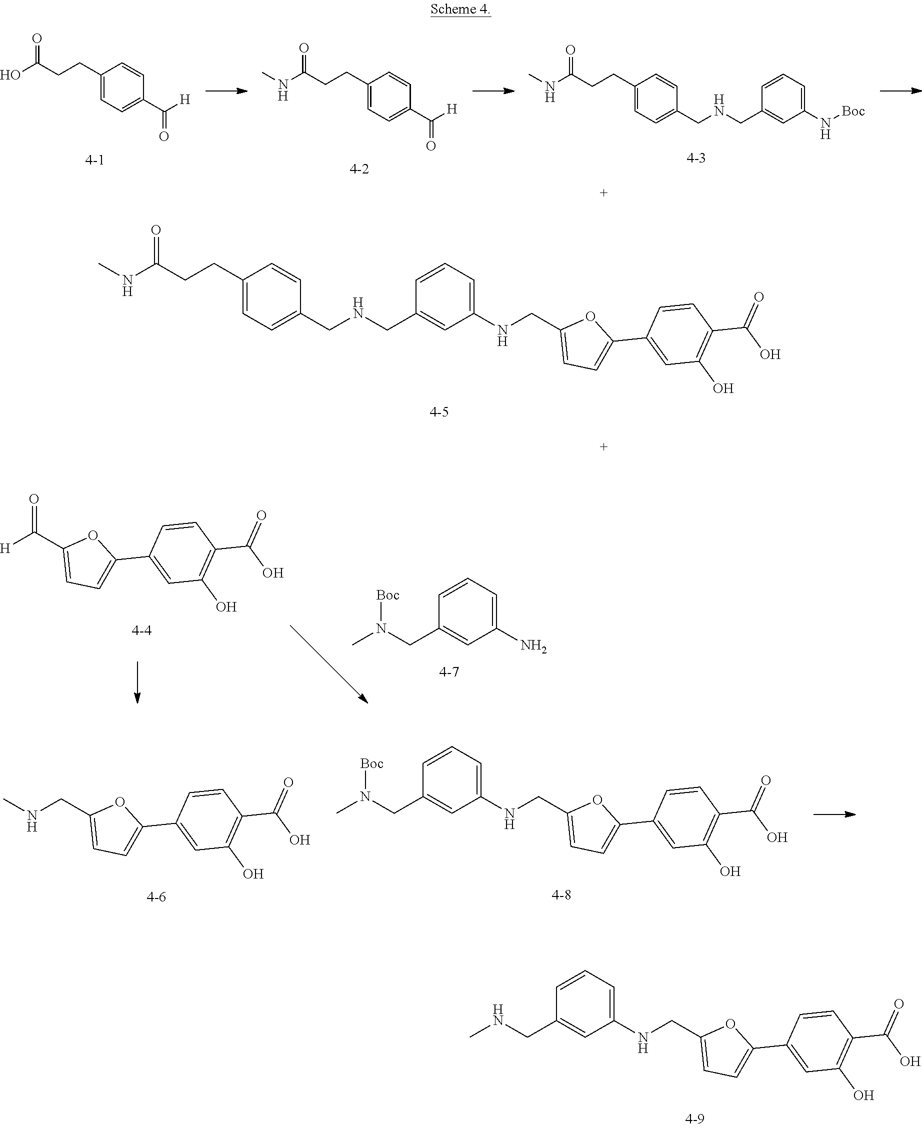

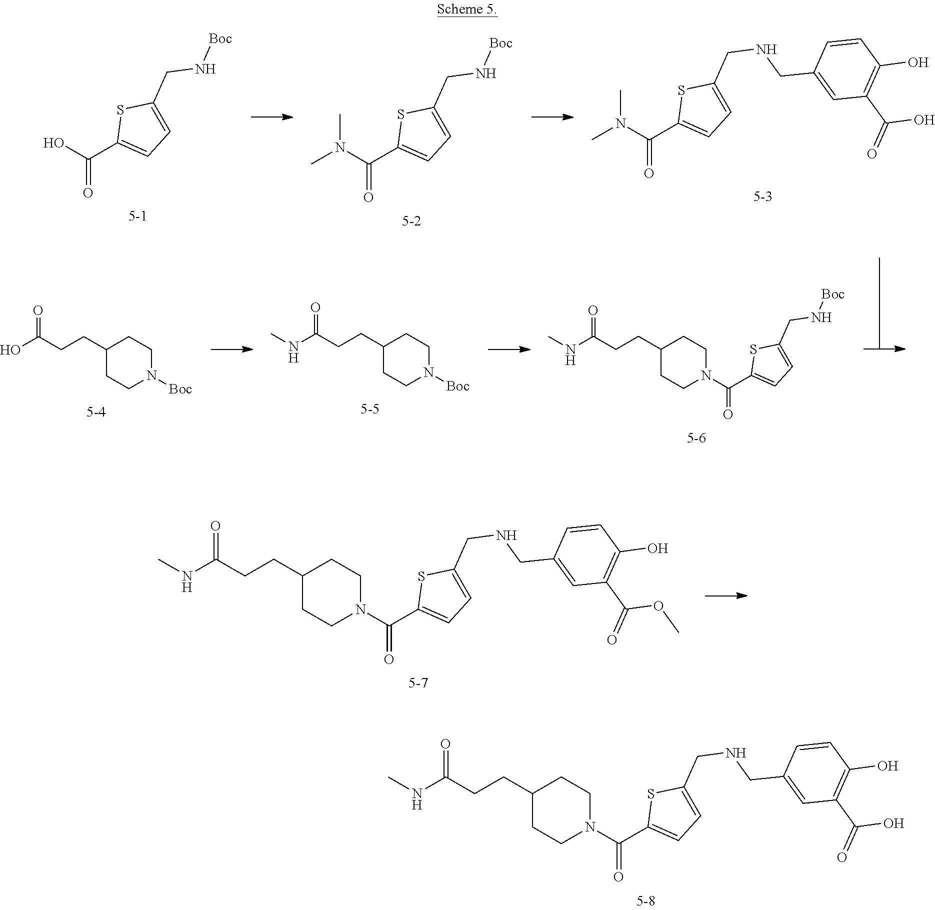

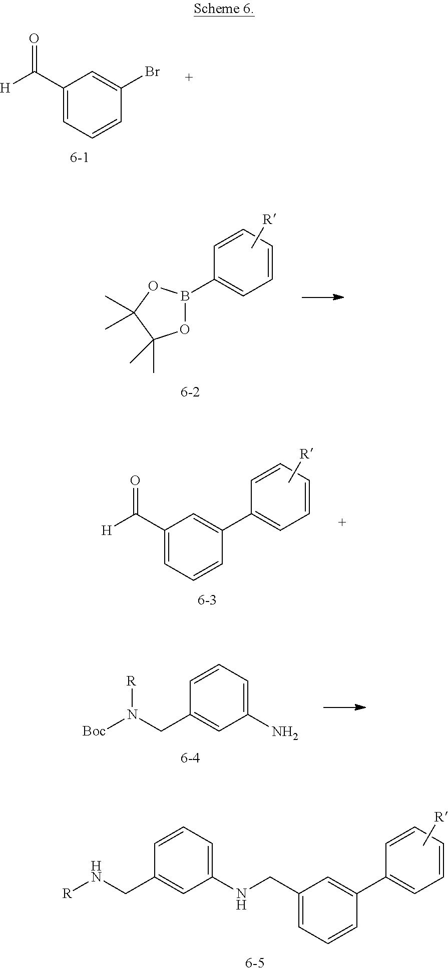

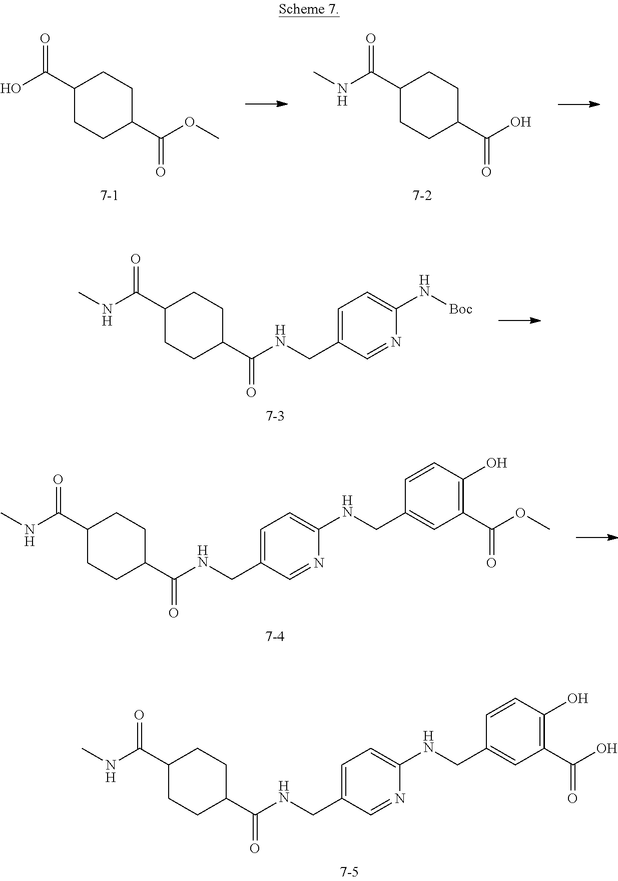

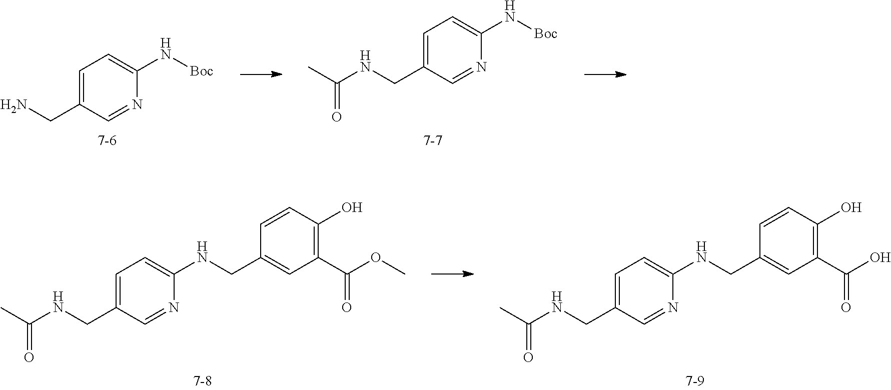 [SMILES: CC(C)(C)OC(=O)Nc1ccc(CN)cn1 ; CC(=O)NCc1ccc(NC(=O)OC(C)(C)C)nc1 ; COC(=O)c1cc(CNc2ccc(CNC(C)=O)cn2)ccc1O ; CC(=O)NCc1ccc(NCc2ccc(O)c(C(=O)O)c2)nc1]

### Compositions

In one aspect, the present disclosure provides a pharmaceutical composition comprising the compound of the present disclosure and a pharmaceutically acceptable carrier.

The present disclosure further provides a pharmaceutical composition comprising at least one compound of the present disclosure and a pharmaceutically acceptable carrier.

In certain embodiments, the pharmaceutical composition further comprises at least one additional antibiotic agent.

In certain embodiments, the at least one additional antibiotic agent is one or more selected from the group consisting of benzathine, benzylpenicillin (penicillin G), benzathine penicillin G, benzathine penicillin V, phenoxymethylpenicillin (penicillin V), procaine penicillin, pheneticillin, cloxacillin, dicloxacillin, flucloxacillin, methicillin, nafcillin, oxacillin, temocillin, amoxicillin, ampicillin, mecillinam, piperacillin, carbenicillin, ticarcillin, azlocillin, mezlocillin, cefazolin, cephalexin, cephalosporin C, cephalothin, cefapirin, cefuroxime, cefaclor, cefprozil, cefaclor, cefamandole, cefuroxime, cefotetan, cefoxitin, cefixime, cefotaxime, cefpodoxime, ceftazidime, ceftriaxone, cefdinir, cefepime, cepirome, ceftaroline, ceftobiprole, biapenem, doripenem, ertapenem, faropenem, Imipenem, meropenem, panipenem, razupenem, tebipenem, thienamycin, aztreonam, tigemonam, nocardicin A, and tabtoxinine.

The compositions containing the compound(s) described herein include a pharmaceutical composition comprising at least one compound as described herein and at least one pharmaceutically acceptable carrier. In some embodiments, the pharmaceutical composition comprises Kolliphor EL, and aqueous buffer, or a combination thereof. In certain embodiments, the aqueous buffer comprises phosphate buffered saline (PBS). In some embodiments, the aqueous buffer comprises 1×PBS. In certain embodiments, the pharmaceutical composition comprises about 5%, about 10%, about 15%, about 20%, about 25%, about 30%, about 35%, or about 40% Kolliphor EL. In some embodiments, the pharmaceutical composition comprises about 20% Kolliphor EL in 1×PBS.

In certain embodiments, the composition is formulated for an administration route such as oral or parenteral, for example, transdermal, transmucosal (e.g., sublingual, lingual, (trans)buccal, (trans)urethral, vaginal (e.g., trans- and perivaginally), (intra)nasal and (trans)rectal, intravesical, intrapulmonary, intraduodenal, intragastrical, intrathecal, subcutaneous, intramuscular, intradermal, intra-arterial, intravenous, intrabronchial, inhalation, and topical administration. In some embodiments, the composition is formulated as a pill, tablet, gelcap, or capsule for oral administration.

### Methods

The present disclosure further provides a method of treating, preventing, and/or ameliorating a bacterial infection in a subject, the method comprising administering to the subject a therapeutically effective amount of at least one compound of any one of claims 1-47 and/or at least one pharmaceutical composition of any one of claims 48-50.

In certain embodiments, the subject is further administered at least one additional antibiotic agent. In certain embodiments, the subject is co-administered the at least one compound and/or composition and the at least one additional antibiotic agent. In certain embodiments, the at least one compound and/or composition and the at least one additional antibiotic agent are coformulated.

In certain embodiments, the at least one additional antibiotic agent is one or more selected from the group consisting of benzathine, benzylpenicillin (Penicillin G), benzathine penicillin G, benzathine penicillin V, phenoxymethylpenicillin (penicillin V), procaine penicillin, pheneticillin, cloxacillin, dicloxacillin, flucloxacillin, methicillin, nafcillin, oxacillin, temocillin, amoxicillin, ampicillin, mecillinam, piperacillin, carbenicillin, ticarcillin, azlocillin, mezlocillin, cefazolin, cephalexin, cephalosporin C, cephalothin, cefapirin, cefuroxime, cefaclor, cefprozil, cefaclor, cefamandole, cefuroxime, cefotetan, cefoxitin, cefixime, cefotaxime, cefpodoxime, ceftazidime, ceftriaxone, cefdinir, cefepime, cepirome, ceftaroline, ceftobiprole, biapenem, doripenem, ertapenem, faropenem, Imipenem, meropenem, panipenem, razupenem, tebipenem, thienamycin, aztreonam, tigemonam, nocardicin A, and tabtoxinine.

The present disclosure further provides a method of inhibiting a β-lactamase and/or penicillin binding protein (PBP) in a subject, the method comprising administering to the subject a therapeutically effective amount of at least one compound of the present disclosure and/or at least one pharmaceutical composition of the present disclosure.

In certain embodiments, the β-lactamase is a class A β-lactamase. In certain embodiments, the β-lactamase is a class B β-lactamase. In certain embodiments, the β-lactamase is a class C β-lactamase. In certain embodiments, the β-lactamase is a class D (β-lactamase.

In certain embodiments, the class A β-lactamase is KPC. In certain embodiments, the KPC is KPC-2. In certain embodiments, KPC-2 is inhibited in a subject with administration of any one of compounds 1-5. In certain embodiments, the class B β-lactamase is NDM. In certain embodiments, the NDM is NDM-1. In certain embodiments, NDM-1 is inhibited in a subject with administration of any one of compounds 8-23. In certain embodiments, the class B β-lactamase is VIM. In certain embodiments, the class D β-lactamase is OXA-48. In certain embodiments, OXA-48 is inhibited in a subject with administration of compound 6 or 7.

In certain embodiments, the penicillin binding protein (PBP) is PBP-3. In certain embodiments, PBP-3 is inhibited in a subject with administration of 24 or 25.

In certain embodiments, administration of a compound of formula (I) is suitable for treatment, prevention, and/or amelioration of a bacterial infection in the absence of an additional antibiotic agent. In certain embodiments, administration of a compound of formula (II) is suitable for treatment, prevention, and/or amelioration of a bacterial infection in the absence of an additional antibiotic agent. In certain embodiments, administration of a compound of formula (III) is suitable for treatment, prevention, and/or amelioration of a bacterial infection in the absence of an additional antibiotic agent. In certain embodiments, administration of a compound of formula (IV) is suitable for treatment, prevention, and/or amelioration of a bacterial infection in the absence of an additional antibiotic agent.

In certain embodiments, the subject is further administered at least one additional antibiotic agent. In certain embodiments, the subject is co-administered the at least one compound and/or composition and the at least one additional antibiotic agent. In certain embodiments, the at least one compound and/or composition and the at least one additional antibiotic agent are coformulated.

In certain embodiments, the at least one additional antibiotic agent is one or more selected from the group consisting of benzathine, benzylpenicillin (penicillin G), benzathine penicillin G, benzathine penicillin V, phenoxymethylpenicillin (penicillin V), procaine penicillin, pheneticillin, cloxacillin, dicloxacillin, flucloxacillin, methicillin, nafcillin, oxacillin, temocillin, amoxicillin, ampicillin, mecillinam, piperacillin, carbenicillin, ticarcillin, azlocillin, mezlocillin, cefazolin, cephalexin, cephalosporin C, cephalothin, cefapirin, cefuroxime, cefaclor, cefprozil, cefaclor, cefamandole, cefuroxime, cefotetan, cefoxitin, cefixime, cefotaxime, cefpodoxime, ceftazidime, ceftriaxone, cefdinir, cefepime, cepirome, ceftaroline, ceftobiprole, biapenem, doripenem, ertapenem, faropenem, Imipenem, meropenem, panipenem, razupenem, tebipenem, thienamycin, aztreonam, tigemonam, nocardicin A, and tabtoxinine.

In certain embodiments, the subject is a mammal. In certain embodiments, the mammal is a human.

### Administration/Dosage/Formulations

The regimen of administration may affect what constitutes an effective amount. The therapeutic formulations may be administered to the subject either prior to or after the onset of the disease or disorder. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions described herein to a patient, preferably a mammal, more preferably a human, may be carried out using known procedures, at dosages and for periods of time effective to treat the disease or disorder in the patient. An effective amount of the therapeutic compound necessary to achieve a therapeutic effect may vary according to factors such as the state of the disease or disorder in the patient; the age, sex, and weight of the patient; and the ability of the therapeutic compound to treat the disease or disorder in the patient. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound described herein is from about 1 and 5,000 mg/kg of body weight/per day. One of ordinary skill in the art would be able to study the relevant factors and make the determination regarding the effective amount of the therapeutic compound without undue experimentation.

Actual dosage levels of the active ingredients in the pharmaceutical compositions described herein may be varied so as to obtain an amount of the active ingredient that is effective to achieve the desired therapeutic response for a particular patient, composition, and mode of administration, without being toxic to the patient.

In particular, the selected dosage level depends upon a variety of factors including the activity of the particular compound employed, the time of administration, the rate of excretion of the compound, the duration of the treatment, other drugs, compounds or materials used in combination with the compound, the age, sex, weight, condition, general health and prior medical history of the patient being treated, and like factors well, known in the medical arts.

A medical doctor, e.g., physician or veterinarian, having ordinary skill in the art may readily determine and prescribe the effective amount of the pharmaceutical composition required. For example, the physician or veterinarian could start doses of the compounds described herein employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical vehicle. The dosage unit forms of the compound(s) described herein are dictated by and directly dependent on (a) the unique characteristics of the therapeutic compound and the particular therapeutic effect to be achieved, and (b) the limitations inherent in the art of compounding/formulating such a therapeutic compound.

In certain embodiments, the compositions described herein are formulated using one or more pharmaceutically acceptable excipients or carriers. In certain embodiments, the pharmaceutical compositions described herein comprise a therapeutically effective amount of a compound described herein and a pharmaceutically acceptable carrier.

The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, it is preferable to include isotonic agents, for example, sugars, sodium chloride, or polyalcohols such as mannitol and sorbitol, in the composition. Prolonged absorption of the injectable compositions may be brought about by including in the composition an agent which delays absorption, for example, aluminum monostearate or gelatin.

In certain embodiments, the compositions described herein are administered to the patient in dosages that range from one to five times per day or more. In other embodiments, the compositions described herein are administered to the patient in range of dosages that include, but are not limited to, once every day, every two, days, every three days to once a week, and once every two weeks. It is readily apparent to one skilled in the art that the frequency of administration of the various combination compositions described herein varies from individual to individual depending on many factors including, but not limited to, age, disease or disorder to be treated, gender, overall health, and other factors. Thus, administration of the compounds and compositions described herein should not be construed to be limited to any particular dosage regime and the precise dosage and composition to be administered to any patient is determined by the attending physician taking all other factors about the patient into account.

The compound(s) described herein for administration may be in the range of from about 1 μg to about 10,000 mg, about 20 μg to about 9,500 mg, about 40 μg to about 9,000 mg, about 75 μg to about 8,500 mg, about 150 μg to about 7,500 mg, about 200 μg to about 7,000 mg, about 350 μg to about 6,000 mg, about 500 μg to about 5,000 mg, about 750 μg to about 4,000 mg, about 1 mg to about 3,000 mg, about 10 mg to about 2,500 mg, about 20 mg to about 2,000 mg, about 25 mg to about 1,500 mg, about 30 mg to about 1,000 mg, about 40 mg to about 900 mg, about 50 mg to about 800 mg, about 60 mg to about 750 mg, about 70 mg to about 600 mg, about 80 mg to about 500 mg, and any and all whole or partial increments therebetween.

In some embodiments, the dose of a compound described herein is from about 1 mg and about 2,500 mg. In some embodiments, a dose of a compound described herein used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in some embodiments, a dose of a second compound as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In certain embodiments, a composition as described herein is a packaged pharmaceutical composition comprising a container holding a therapeutically effective amount of a compound described herein, alone or in combination with a second pharmaceutical agent; and instructions for using the compound to treat, or reduce one or more symptoms of a disease or disorder in a patient.

Formulations may be employed in admixtures with conventional excipients, i.e., pharmaceutically acceptable organic or inorganic carrier substances suitable for oral, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., other analgesic agents.

Routes of administration of any of the compositions described herein include oral, nasal, rectal, intravaginal, parenteral, buccal, sublingual or topical. The compounds for use in the compositions described herein can be formulated for administration by any suitable route, such as for oral or parenteral, for example, transdermal, transmucosal (e.g., sublingual, lingual, (trans)buccal, (trans)urethral, vaginal (e.g., trans- and perivaginally), (intra)nasal and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions described herein are not limited to the particular formulations and compositions that are described herein.

### Oral Administration

For oral application, particularly suitable are tablets, dragees, liquids, drops, suppositories, or capsules, caplets and gelcaps. The compositions intended for oral use may be prepared according to any method known in the art and such compositions may contain one or more agents selected from the group consisting of inert, non-toxic pharmaceutically excipients that are suitable for the manufacture of tablets. Such excipients include, for example an inert diluent such as lactose; granulating and disintegrating agents such as cornstarch; binding agents such as starch; and lubricating agents such as magnesium stearate. The tablets may be uncoated or they may be coated by known techniques for elegance or to delay the release of the active ingredients. Formulations for oral use may also be presented as hard gelatin capsules wherein the active ingredient is mixed with an inert diluent.

For oral administration, the compound(s) described herein can be in the form of tablets or capsules prepared by conventional means with pharmaceutically acceptable excipients such as binding agents (e.g., polyvinylpyrrolidone, hydroxypropylcellulose or hydroxypropyl methylcellulose); fillers (e.g., cornstarch, lactose, microcrystalline cellulose or calcium phosphate); lubricants (e.g., magnesium stearate, talc, or silica); disintegrates (e.g., sodium starch glycollate); or wetting agents (e.g., sodium lauryl sulphate). If desired, the tablets may be coated using suitable methods and coating materials such as OPADRY™ film coating systems available from Colorcon, West Point, Pa. (e.g., OPADRY™ OY Type, OYC Type, Organic Enteric OY-P Type, Aqueous Enteric OY-A Type, OY-PM Type and OPADRY™ White, 32K18400). Liquid preparation for oral administration may be in the form of solutions, syrups or suspensions. The liquid preparations may be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl p-hydroxy benzoates or sorbic acid).

### Parenteral Administration

For parenteral administration, the compounds as described herein may be formulated for injection or infusion, for example, intravenous, intramuscular or subcutaneous injection or infusion, or for administration in a bolus dose and/or continuous infusion. Suspensions, solutions or emulsions in an oily or aqueous vehicle, optionally containing other formulatory agents such as suspending, stabilizing and/or dispersing agents may be used.

Sterile injectable forms of the compositions described herein may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1, 3-butanediol.

Among the acceptable vehicles and solvents that may be employed are water, Ringer's solution and isotonic sodium chloride solution. Sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically acceptable oils, such as olive oil or castor oil, especially in their polyoxyethylated versions. These oil solutions or suspensions may also contain a long-chain alcohol diluent or dispersant, such as Ph. Helv or similar alcohol.

### Additional Administration Forms

Additional dosage forms suitable for use with the compound(s) and compositions described herein include dosage forms as described in U.S. Pat. Nos. 6,340,475; 6,488,962; 6,451,808; 5,972,389; 5,582,837; and 5,007,790. Additional dosage forms suitable for use with the compound(s) and compositions described herein also include dosage forms as described in U.S. Patent Applications Nos. 20030147952; 20030104062; 20030104053; 20030044466; 20030039688; and 20020051820. Additional dosage forms suitable for use with the compound(s) and compositions described herein also include dosage forms as described in PCT Applications Nos. WO 03/35041; WO 03/35040; WO 03/35029; WO 03/35177; WO 03/35039; WO 02/96404; WO 02/32416; WO 01/97783; WO 01/56544; WO 01/32217; WO 98/55107; WO 98/11879; WO 97/47285; WO 93/18755; and WO 90/11757.

### Controlled Release Formulations and Drug Delivery Systems

In certain embodiments, the formulations described herein can be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release which is longer that the same amount of agent administered in bolus form.

For sustained release, the compounds may be formulated with a suitable polymer or hydrophobic material which provides sustained release properties to the compounds. As such, the compounds for use with the method(s) described herein may be administered in the form of microparticles, for example, by injection or in the form of wafers or discs by implantation.

In some cases, the dosage forms to be used can be provided as slow or controlled-release of one or more active ingredients therein using, for example, hydropropylmethyl cellulose, other polymer matrices, gels, permeable membranes, osmotic systems, multilayer coatings, microparticles, liposomes, or microspheres or a combination thereof to provide the desired release profile in varying proportions. Suitable controlled-release formulations known to those of ordinary skill in the art, including those described herein, can be readily selected for use with the pharmaceutical compositions described herein. Thus, single unit dosage forms suitable for oral administration, such as tablets, capsules, gelcaps, and caplets that are adapted for controlled-release are encompassed by the compositions and dosage forms described herein.

Most controlled-release pharmaceutical products have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time. Advantages of controlled-release formulations include extended activity of the drug, reduced dosage frequency, and increased patient compliance. In addition, controlled-release formulations can be used to affect the time of onset of action or other characteristics, such as blood level of the drug, and thus can affect the occurrence of side effects.

Most controlled-release formulations are designed to initially release an amount of drug that promptly produces the desired therapeutic effect, and gradually and continually release of other amounts of drug to maintain this level of therapeutic effect over an extended period of time. In order to maintain this constant level of drug in the body, the drug must be released from the dosage form at a rate that will replace the amount of drug being metabolized and excreted from the body.

Controlled-release of an active ingredient can be stimulated by various inducers, for example pH, temperature, enzymes, water, or other physiological conditions or compounds. The term “controlled-release component” is defined herein as a compound or compounds, including, but not limited to, polymers, polymer matrices, gels, permeable membranes, liposomes, or microspheres or a combination thereof that facilitates the controlled-release of the active ingredient. In some embodiments, the compound(s) described herein are administered to a patient, alone or in combination with another pharmaceutical agent, using a sustained release formulation. In some embodiments, the compound(s) described herein are administered to a patient, alone or in combination with another pharmaceutical agent, using a sustained release formulation.

The term delayed release is used herein in its conventional sense to refer to a drug formulation that provides for an initial release of the drug after some delay following drug administration and that mat, although not necessarily, includes a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

### Dosing

The therapeutically effective amount or dose of a compound described herein depends on the age, sex and weight of the patient, the current medical condition of the patient and the progression of the disease or disorder in the patient being treated. The skilled artisan is able to determine appropriate dosages depending on these and other factors.

A suitable dose of a compound described herein can be in the range of from about 0.01 mg to about 5,000 mg per day, such as from about 0.1 mg to about 1,000 mg, for example, from about 1 mg to about 500 mg, such as about 5 mg to about 250 mg per day. The dose may be administered in a single dosage or in multiple dosages, for example from 1 to 4 or more times per day. When multiple dosages are used, the amount of each dosage may be the same or different. For example, a dose of 1 mg per day may be administered as two 0.5 mg doses, with about a 12-hour interval between doses.

It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on.

In the case wherein the patient's status does improve, upon the doctor's discretion the administration of the compound(s) described herein is optionally given continuously; alternatively, the dose of drug being administered is temporarily reduced or temporarily suspended for a certain length of time (i.e., a “drug holiday”). The length of the drug holiday optionally varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday includes from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, is reduced to a level at which the improved disease is retained. In certain embodiments, patients require intermittent treatment on a long-term basis upon any recurrence of symptoms and/or infection.

The compounds described herein can be formulated in unit dosage form. The term “unit dosage form” refers to physically discrete units suitable as unitary dosage for patients undergoing treatment, with each unit containing a predetermined quantity of active material calculated to produce the desired therapeutic effect, optionally in association with a suitable pharmaceutical carrier. The unit dosage form may be for a single daily dose or one of multiple daily doses (e.g., about 1 to 4 or more times per day). When multiple daily doses are used, the unit dosage form may be the same or different for each dose.

Toxicity and therapeutic efficacy of such therapeutic regimens are optionally determined in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index, which is expressed as the ratio between LD₅₀ and ED₅₀. The data obtained from cell culture assays and animal studies are optionally used in formulating a range of dosage for use in human. The dosage of such compounds lies preferably within a range of circulating concentrations that include the ED₅₀ with minimal toxicity. The dosage optionally varies within this range depending upon the dosage form employed and the route of administration utilized.

Those skilled in the art will recognize, or be able to ascertain using no more than routine experimentation, numerous equivalents to the specific procedures, embodiments, claims, and examples described herein. Such equivalents are considered to be within the scope of this disclosure and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that wherever values and ranges are provided herein, all values and ranges encompassed by these values and ranges, are meant to be encompassed within the scope of the present disclosure. Moreover, all values that fall within these ranges, as well as the upper or lower limits of a range of values, are also contemplated by the present application.

The following examples further illustrate aspects of the present disclosure. However, they are in no way a limitation of the teachings or disclosure of the present disclosure as set forth herein.

### EXAMPLES

Various embodiments of the present application can be better understood by reference to the following Examples which are offered by way of illustration. The scope of the present application is not limited to the Examples given herein.

### Materials and Methods

### Materials

All DNA materials were obtained from LGC Biosearch Technologies. T4 DNA ligase was obtained from Enzymatics (Qiagen). All chemical building blocks were commercially purchased and stored in acetonitrile or DMSO solutions (100 or 200 mM) at −80° C. All buffers were prepared in-house using bio-grade reagents and high-purity solvents.

### Procedure for DNA Analysis

DECL materials were assayed on a Vanquish UHPLC system with a Thermo DNAPac RP column (2.1×50 mm, 4 μm) attached to an LTQ XL ion trap mass spectrometer (ThermoFisher Scientific). The injection volume was 5 μL that contained 50-200 pmol of DNA analyte. The mobile phase was 15 mM TEA/100 mM HFIP in water/methanol. Mass spectra were acquired in the full scan negative-ion mode over the mass range 500-2000 and m/z patterns were deconvoluted using ProMass software.

### General Procedure for Ethanol Precipitation and DNA Reconstruction

Based on a theoretical solution volume n, n/20 volume of a 5 M NaCl stock solution was added and the solution was gently mixed. Absolute ethanol (4n volume, 80% v/v final ethanol concentration) was added, thoroughly mixed, and stored at −20° C. overnight to precipitate any DNA material. The resulting slurry was centrifuged (>10,000 g for 1 h) and the supernatant was decanted out. An additional volume n of chilled 75% ethanol (v/v) was added, and the centrifugation-decantation process was repeated. After removal of the supernatant, the pellet was dried (in open air or under a gentle vacuum within a speed-vac) and then reconstituted in neutral water or buffer (to a concentration of ˜1 mM; assessed by optical density measurements).

### General Procedure for DNA Ligation

The final concentration of the reaction mixture was set at 0.24 mM for C1, C2, and C3 ligation. For DNA tagging, the final concentration was 0.1 mM, and for closing primer ligation, it was set at 0.05 mM. To the DNA piece (1 equiv), a slightly higher equivalent of codon pieces was added (eg. 1.1 equiv for C1, 1.2 equiv for C2, 1.3 equiv for C3) so that there was an excess of codons compared to the larger DNA piece. Total volume (V) was calculated using the final concentration and DNA mol amount. V/10 of HEPES buffer, V/100˜150 ligase, and the rest of the volume of H₂O were premixed before adding to the DNA-codon mixture. For C1 ligation, the reaction was monitored with LC-MS. For the rest of the cycles, the ligation was monitored using 10% TBE gel or 6% TBE gel containing urea.

### Protein Expression and Purification

His-tagged-NDM-1 and His-tagged-OXA-48 were expressed from a pET28a vector in E. coli BL21 (DE3) cells. The cells were grown in LB broth (Luria-Bertani) supplemented with 25 μg/mL kanamycin at 37° C. until mid-log-phase (OD600=0.8). The expression of the His-tagged-NDM-1 or His-tagged-OXA-48 protein was then induced by adding a final concentration of 0.5 mM isopropyl β-D-1-thiogalactopyranoside (IPTG) and shaking at 18° C. for 20 h. The cell pellet was collected by centrifugation (5,000 g), then re-suspended in the lysis buffer (NDM-1: 20 mM HEPES, 500 mM NaCl, 20 mM imidazole, pH 7.4; OXA-48: 50 mM HEPES, pH 7.4) and lysed using a French press followed by sonication. After centrifugation (10,000 g), the supernatant of the cell lysate was filtered (0.45 μm filter) and loaded onto a HisTrap™ FF column (GE Healthcare). The His-tagged-NDM-1 or His-tagged-OXA-48 was eluted with an imidazole gradient in the lysis buffer. The His-tag was removed by incubation with TEV protease (1:50) for 12 h at 4° C. The His-tagged TEV protease was removed by incubation with TALON Metal Affinity Resin. NDM-1 or OXA-48 in the column flow through fraction was further purified by gel-filtration chromatography using a Superdex 75 GL 16/600 sizing column (GE Healthcare) with 20 mM HEPES, 100 mM NaCl, 50 μM ZnSO4, pH 7.4 as the running buffer for NDM-1, and 50 mM HEPES, pH 7.4 as the running buffer for OXA-48. Fractions containing NDM-1 or OXA-48 were pooled and concentrated using 10 kDa MWCO Amicon Ultra-15 Centrifugal Filter Units (MilliporeSigma, Burlington, MA) and were examined by SDS-PAGE. The final protein concentration was determined with a DU800 spectrophotometer (Beckman Coulter) by measuring absorbance at 280 nm. NDM-1 and OXA-48 were stored at −80° C. freezer until use.

### Synthesis of the Carboxylate-Binding Pocket-Targeting Library

All materials were purchased from commercial sources. All buffers were prepared in-house using bio-grade reagents and high-purity solvents. All chemical and ligation reactions were monitored using a UHPLC system with an LTQ XL ion trap mass spectrometer (ThermoFisher Scientific) or 10% or 6% TBE gels. Detailed synthetic procedures for each library are described elsewhere herein in greater detail.

Affinity Selection with a DNA-Encoded Chemical Library

DNA-encoded chemical libraries (DECLs) were mixed with 1 M or 5 M of His-OXA-48 and 0.2 M or 1 μM His-NDM-1, in 200 μL of selection buffer (20 mM HEPES pH 7.5, 134 mM potassium acetate, 10 mM imidazole, 1 mM TCEP, 8 mM sodium acetate, 0.8 mM magnesium acetate, 1 mM CHAPS, 4 mM sodium chloride and, 0.1 mg/ml Sheared Salmon Sperm DNA). The same library pool without protein was incubated under the same conditions as a non-target control (NTC) to assess the background binding of DECL molecules to the affinity resin. Before incubation, 1 μL of library molecules were set aside for quantitation using quantitative PCR (qPCR). Ni-NTA magnetic beads (50 μL) were pre-washed with selection buffer and the target protein-library mixture was added for affinity selection. The magnetic beads were washed with 500 μL of selection buffer to remove unbound DECL molecules. Bound compounds were eluted by incubating the beads with 100 L of selection buffer at 80° C. for 10 min. 1 μL of elution material was set aside for quantitation by qPCR, and the remaining volume was subjected to an additional round of affinity selection with fresh protein as mentioned above. After 2 or 3 rounds of selection, qPCR was performed on recovered DECL material after each round of selection to monitor the total amount of DNA tags remaining. This information was used to guide both the selection process and sequencing preparation. An appropriate number of PCR cycles was used to amplify the DNA and added DNA sequences compatible with Illumina sequencing flow cells. The PCR product was purified using Agencourt AMPure XP SPRI beads (Agencourt, Danvers, MA) according to the manufacturer's instructions and then quantitated on an Agilent BioAnalyzer (Santa Clara, CA) using a high-sensitivity DNA kit. The final concentration of amplicon for each sample was pooled at 4 nM concentration. The final concentration of 1.8 μM of library pool samples was loaded onto an Illumina Next-Seq 500 sequencer (San Diego, CA) at the Genomic and RNA Profiling Core Facility at Baylor College of Medicine. Selection output data were processed in-house.

### Determination of Inhibition Activity

The inhibition activity of NDM-1 inhibitors was assessed in 50 mM HEPES (pH 7.4) supplemented with 10 μg/mL BSA and 10 μM ZnSO₄. Water used for preparing the buffer was treated with Chelex 100 resin (200-400 mesh) to remove zinc ions. The rate of substrate (imipenem) hydrolysis by the NDM-1 enzyme was monitored using a Tecan plate reader or a DU800 spectrophotometer (Beckman Coulter) equipped with a thermostatically controlled cell by following the absorbance change of imipenem at 300 nm (Δε300 nm=−9000 M⁻¹ cm⁻¹). Cuvettes with 0.2 or 1 cm path lengths were used, depending on the substrate concentration. The K_{M} value (65 M) for NDM-1 was determined under the initial-rate condition at a constant NDM-1 concentration (2 nM) by fitting the initial velocity (vₒ) at a range of imipenem concentrations (0-600 μM) to the Michaelis-Menten equation using GraphPad Prism9. The inhibition assays were performed at a constant NDM-1 concentration (2 nM) and a constant imipenem concentration (40 μM) below the K_{M} value. The inhibitors were tested up to 100 μM (0-100 μM). Inhibitors were incubated with the NDM-1 enzyme for 10 min at room temperature before mixing with the substrate to start the hydrolysis reaction. The initial velocities at different inhibitor concentrations were fit to the Morrison Tight Binding equation⁶² to determine the inhibition constant (Kᵢ) using GraphPad Prism9. Kinetic parameters were averaged from at least two independent determinations.

OXA-48 inhibitors were tested for their inhibition activity by examining OXA-48 mediated hydrolysis of a colorimetric substrate, nitrocefin, using a Tecan plate reader. The inhibition assays were performed at a constant OXA-48 concentration (0.1 nM) and a constant nitrocefin concentration (30 μM), which is below the KM value. The inhibitors were tested up to 100 μM (0-100 μM). Inhibitors were incubated with OXA-48 enzyme for 10 min at room temperature before mixing with a substrate to start the hydrolysis reaction. The rate constants at different inhibitor concentrations were fit to the Morrison Tight Binding equation to determine the inhibition constant (Ki) using GraphPad Prism9. Kinetic parameters were averaged from at least two independent determinations.

### Minimum Inhibitory Concentration (MIC) Measurement

NDM-1 inhibitors with Ki values below 10 μM were tested for their ability to restore the susceptibility of E. coli expressing NDM-1 to imipenem by determining the MIC in LB media. The MIC was defined as the lowest concentration of antibiotic that inhibits visible growth of the bacterial culture. The NDM-1 gene was cloned into plasmid pTP470 which encodes chloramphenicol resistance. The expression of NDM-1 was under the control of the IPTG-inducible trc promoter. The pTP470 plasmid encoding NDM-1 was transformed into E. coli XL-1 blue by electroporation. The MIC was determined by serial two-fold dilutions of imipenem (0.5-8 μg/mL) in LB broth, with 0 μg/mL imipenem as control. Different concentrations of inhibitors (0, 8, 16, 32, 64, 128, μg/mL) were added to each level of imipenem LB media. A 1:105 dilution of saturated overnight bacterial culture of NDM-1-expressing E. coli XL-1 blue was mixed with the combinations of imipenem-inhibitor concentrations at a final volume of 0.2 mL in 96-well plates with glass beads in each well to facilitate mixing and aeration. The mixtures were incubated for 18 h at 37° C. with shaking at 180 rpm. The bacterial growth was recorded by reading the absorbance of the cultures at 600 nM (OD600) with a Tecan microplate reader.

### X-Ray Crystallography

The crystallization was carried out using the hanging-drop vapor-diffusion method using an automated Mosquito robot. 0.1 μL of purified OXA-48 protein (7.5 mg/mL) was mixed with 0.1 μL of reservoir solution and then equilibrated against 70 μL reservoir solution at room temperature. Crystals of OXA-48 were obtained under the condition containing 25% (v/v) PEG 550 MME, 0.1 M TRIS-HCl pH 8.5. OXA-48 crystals were soaked with 5 mM inhibitor for 2 h at room temperature to obtain OXA-48 structure in complex with the inhibitor. The soaked crystals were flash cooled in liquid nitrogen with 15% glycerol as a cryoprotectant. Diffraction data were collected at the Advanced light source, Berkeley Center for Structural Biology in the context of the Collaborative Crystallography Program on beamline 8.2.2.

Diffraction data were processed using iMosfim and the CCP4 suite software. Molecular replacement was performed using Phaser using the OXA-48 apo structure (PDB ID: 3HBR or 6P96) as a search model. Crystallography Object-Oriented Toolkit (COOT) program was used to fit the model to electron density, and the REFMAC5 and phenix.refine programs were used for refinement. The atomic coordinates for the structure of OXA-48 bound to CDD-2725 (PDB ID: 8SQF) and CDD-2801 (PDB ID: 8SQG) have been deposited in the Protein Data Bank (PDB). Panels containing crystal structures were created using the UCSF Chimera program. The omit maps were created using the Polder map program in Phenix, with the ligand (CDD-2725 or CDD-2801) and bicarbonate atoms omitted.

### Accumulation Assay

The accumulation assays of compounds CDD-2376, CDD-2616, and CDD-2653 were performed in triplicates by following the protocol from Ritcher et al. Tetracycline and clindamycin served as the positive and negative control, respectively. Briefly, the overnight culture of E. coli NDM-1/XL-1B was inoculated into fresh Luria Bertani (LB) broth (1:100 v/v) and incubated at 37° C. with shaking to an optical density (OD600) of 0.55-0.60. The bacteria were pelleted by centrifugation and washed with 1× phosphate-buffered saline (PBS) before being re-suspended in PBS (28 times concentrated). The colony-forming units (CFUs) were determined by a calibration curve. Re-suspended cells were aliquoted into 1.5 mL Eppendorf tubes and recovered at 37° C. for 5 min. The compounds and controls were added with a final concentration of 50 μM. The mixtures were incubated at 37° C. for 10 min and the OD600 values were recorded to note the growth changes. To collect the cell pellets, 800 μL bacteria-compound mixtures were layered onto 700 μL silicone oil (9:1 v/v, AR20/Sigma High Temperature, pre-cooled at −80° C.) and bacteria were pelleted through the silicone oil by centrifugation. Cell pellets were re-suspended in 200 μL H₂O and lysed using three freeze-thaw cycles of 3 min in liquid nitrogen followed by 3 min in a 65° C. water bath. Supernatants were collected after centrifuging and the cell debris was re-suspended in 100 μL methanol. The mixtures from cell debris were then centrifuged, and the methanol supernatants were combined with the supernatants from the last step before LC-MS analysis.

Two NL of supernatant was injected into a Thermo TSQ Quantis MS coupled with a Thermo Vanish UHPLC for analysis. The Phenomenex Luna C18 column (1.0 mm×50 mm, 1.7 μm) was used for resolving tested compounds with the column temperature being kept constant at 40° C. The mobile phase system was (A) water (containing 0.1% formic acid)-(B) acetonitrile (containing 0.1% formic acid), with a flow rate of 0.15 mL/min. The gradient elution program was as follows: 0-0.5 min, 2% B; 0.5-2 min, 2%-95% B; 2-4 min, 95% B; 4-4.3 min, 95%-2% B; 4.3-5 min, 2% B. The MS was operated in a positive selected reaction monitoring mode with electrospray ionization. The LC-MS data were acquired from 1.5 min to 4 min and processed by Thermo Tracefinder software. The compound concentrations in the samples were calculated based on their corresponding standard curves. The standard curves with good linearity (R2>0.99) were established using linear regression between peak area and concentration (0.03-30 μM) with a weight of 1/x2

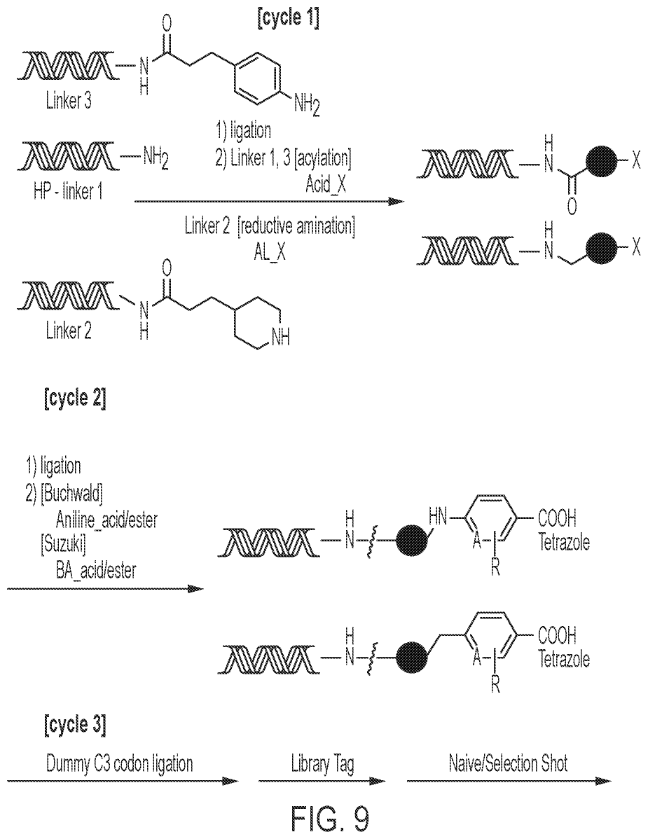

Detailed Procedure for qDOS38_1 Synthesis (FIG. 9)

### Procedure for Cycle 1

To the headpiece (1.5 μmol, 1,500 μL, 1.0 mM in H₂O, 1 equiv) was added the pH9.5 phosphate buffer (600 mol, 2,400 μL, 250 mM in H₂O, 400 equiv). N-boc protected amino acid linkers (150 mol, 750 μL, 200 mM in AcCN, 100 equiv), HATU (150 μmol, 750 μL, 200 mM in DMA, 100 equiv), and DIPEA (150 μmol, 750 μL, 200 mM in AcCN, 100 equiv) were pre-incubated for 15 min prior to being added to the DNA-buffer mixture. The reaction mixture was left overnight at room temperature (room temperature) prior to EtOH precipitation. The N-boc group was deprotected by adding 1.5× the volume of pH 9.5 phosphate buffer (2,250 L) to reconstituted DNA-linker (1,500 μL) and heating it at 80° C. for 24-48 h. The reaction was monitored using LC-MS. The three different linker molecules were evenly split into 1,264 wells. After C1 ligation, the reconstituted DNA-linked amines were subjected to reductive amination or acylation. For reductive amination, the DNA-linked diamine (8 nmol, 8 μL, 1.0 mM in H₂O, 1 equiv) was mixed with pH 5.8 MES buffer (3,200 nmol, 12.8 μL, 250 mM in H₂O, 400 equiv), aldehyde-halide (800 nmol, 4 μL, 200 mM in AcCN, 100 equiv), acetonitrile (8 μL, 30% v/v at final concentration of 0.2 mM), water (3.2 μL, rest of the volume), followed by NaCNBH₃ (800 nmol, 4 μL, 200 mM in H2O, 100 equiv). The mixture was incubated at 35° C. overnight. After monitoring the reaction with LCMS, another 1 equiv of NaCNBH₃ was added to each well and heated to 35° C. overnight before EtOH precipitation. For acylation, DNA-linked amine (8 nmol, 8 μL, 1.0 mM in H₂O, 1 equiv) was mixed with pH9.5 phosphate buffer (3,200 nmol, 12.8 μL, 250 mM in H2O, 400 equiv), acid-halide (800 nmol, 4 μL, 200 mM in AcCN, 100 equiv), HATU (800 nmol, 4 NL, 200 mM in DMA, 100 equiv), and DIPEA (800 nmol, 4 μL, 200 mM in AcCN, 100 equiv), or DMTMM (800 nmol, 4 μL, 200 mM in H₂O, 100 equiv), and acetonitrile (4 L) instead of HATU and DIPEA. The reaction mixture was left at room temperature overnight before EtOH precipitation. All wells were monitored by LC-MS. A total of 750 wells were pooled, removing any wells that had less than 40% yield of the desired product. The pooled material was subjected to an additional round of EtOH precipitation and removal of any material that was not water soluble after reconstitution. The final concentration of C1 pooled material was 1.58 mM (3,000 μL).

### Procedure for Cycle 2

The pooled material was split into 305 wells followed by C2 ligation using standard procedure. The ligated material was subjected to Buchwald and Suzuki coupling. For Buchwald coupling reaction, the DNA-linked halide (15.8 nmol, 15.8 μL, 1.0 mM in H₂O, 1 equiv) was mixed with CsOH (15,800 nmol, 7.9 μL, 2,000 mM in H₂O, 1,000 equiv), DMA (5.53 μL, 50% v/v at final concentration of 0.25 mM), aniline-acid, aniline-ester, or aniline-tetrazole (7,900 nmol, 19.75 μL, 400 mM in DMA, 500 equiv), sodium ascorbate (316 nmol, 7.9 μL, 40 mM in H2O, 20 equiv), followed by PEPPSI-iHept (31.6 nmol, 6.32 μL, 5 mM in DMA, 2 equiv). The reaction mixture was heated to 95° C. for 15 min and then let cooled down to room temperature. Sodium cysteine (1,580 nmol, 7.9 μL, 200 mM in H₂O with 1.8× NaOH, 100 equiv) was then added to the mixture as a metal scavenger and heated to 80° C. for another 15 min before EtOH precipitation. For Suzuki coupling, the DNA-linked halide (15.8 nmol, 15.8 μL, 1.0 mM in H₂O, 1 equiv) was mixed with CsOH (6.320 nmol, 10.53 μL, 600 mM in H₂O, 400 equiv), boronic acid-acid, boronic acid-ester, or boronic acid-tetrazole (1,580 nmol, 7.9 μL, 200 mM in 1:1 H2O:dioxane, 100 equiv), followed by SPhos-Pd-G2 (31.6 nmol, 3.16 μL, 10 mM DMA, 2 equiv). The reaction mixture was heated to 80° C. for 15 min and then let cooled down to room temperature. Then, sodium cysteine (1,580 nmol, 7.9 μL, 200 mM in H₂O with 1.8× NaOH, 100 equiv) was added to the reaction and heated to 80° C. for 15 min before EtOH precipitation. The reaction was monitored using spike-in (3.95 nmol, 25% of DNA mol amount) that was inserted into well A6 of each plate. Those particular wells contained 1.25× of all the other reaction conditions. Some wells were also used to monitor reaction using the headpiece and a previously validated substrate instead of using the pooled material and a substrate from the library building block. All 305 wells were pooled, followed by another round of EtOH precipitation and removal of any material that was not soluble in water upon reconstitution. The final concentration was 0.96 mM (3,000 μL).

### Preparation of DNA Samples for Selection Experiments (Cycle 3)

The pooled material was split into 96 wells for C3 codon ligation to match the overhang of the ID tag. There was no reaction done for C3. The 96 wells were pooled, followed by EtOH precipitation. The pooled material was split into 6 0.5 mL 30 k amicon filters and washed 4 times (4,000 rpm, 40 min) to remove some un-ligated codons. The final pooled concentration was 1.4 mM (2,000 μL). The pooled material was subjected to hydrolysis so that any esters reveal the acid form, which is important for the binding activity to β-lactamases. The pooled material was split into 6 1.5 mL Eppendorf tubes (400 μL), and pH 9.5 buffer (600 NL, 1.5× volume) was added. The reaction mixture was heated at 80° C. for 2 hr prior to EtOH precipitation. The reaction was monitored using spike-in.

The library-specific ID was ligated to the C3 pooled material. After EtOH precipitation, the materials were filtered again using a 30 k amicon filter 3 times (3,500 rpm, 40 min). The ID-tagged material (3 nmol for naïve sequencing, and 9 nmol for selection) for closing primer ligation. The final materials were filtered through a 3 k amicon filter 5 times (3,500 rpm, 20 min) to remove any residual ATP in the reaction mixture.

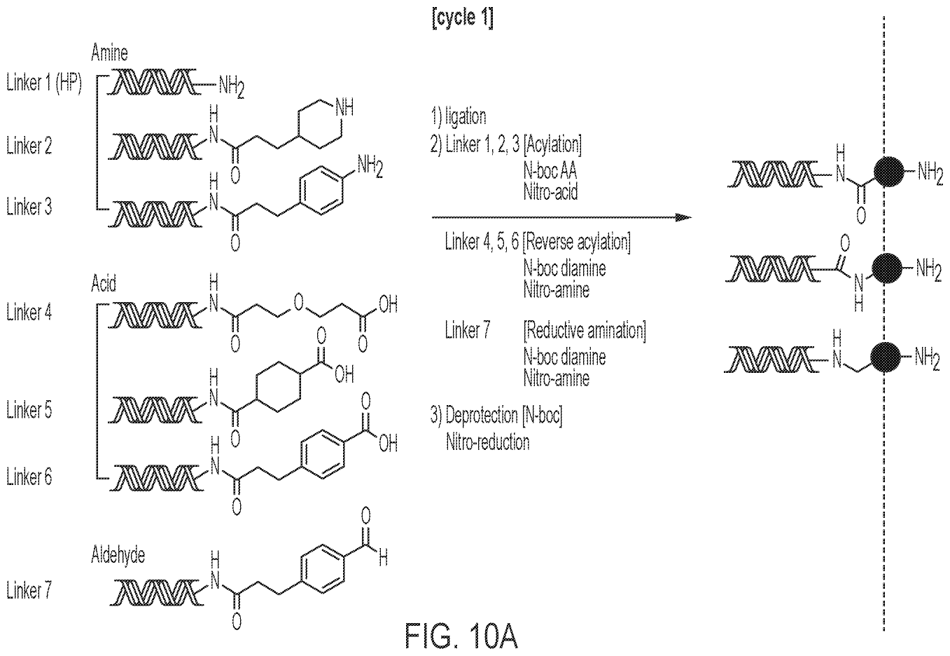

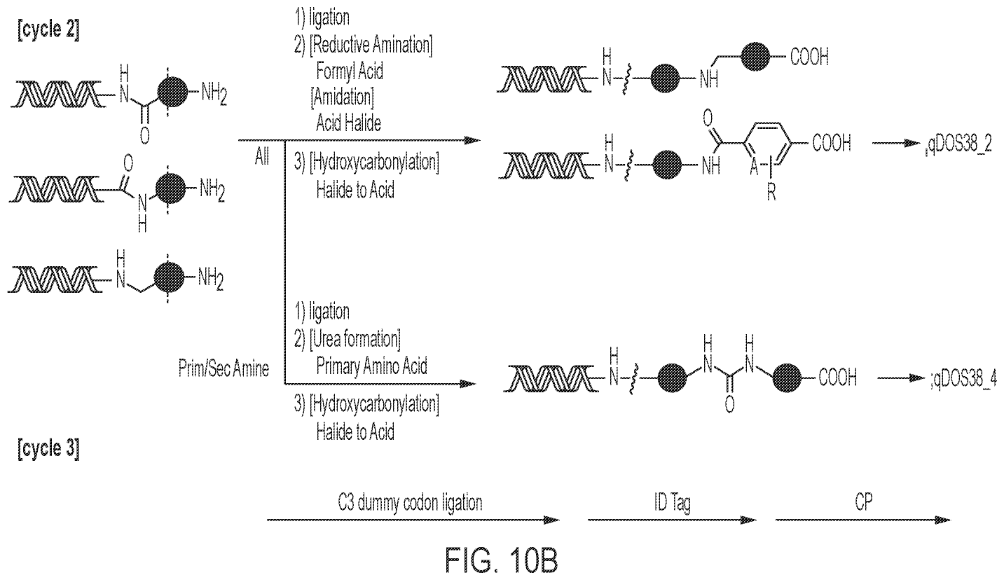

Detailed Procedure for qDOS38_2 Synthesis (FIGS. 10A-10B)

### Procedure for Cycle 1

To the headpiece (1.5 μmol, 1,500 μL, 1.0 mM in H2O, 1 equiv) was added the N-boc protected amino acid linkers, di-acid or acid-ester linkers, or acid-aldehyde linker (150 gmol, 750 μL, 200 mM in AcCN, 100 equiv), pH 9.5 phosphate buffer (600 μmol, 2,400 μL, 250 mM in H2O, 400 equiv), HATU (150 mol, 750 μL, 200 mM in DMA, 100 equiv), and DIPEA (150 mol, 750 μL, 200 mM in AcCN, 100 equiv). The reaction mixture was left overnight at room temperature (room temperature) prior to EtOH precipitation. The N-boc group or ester was deprotected by adding 1.5× the volume of pH 9.5 phosphate buffer (2,250 μL) to reconstituted DNA-linker (1,500 L) and heating it at 80° C. for 24-48 h. The reaction was monitored using LC-MS. The three different linker molecules were evenly split into 2,014 wells. The DNA-linked amines were subjected to acylation. The DNA-linked amine (8 nmol, 8 μL, 1.0 mM in H₂O, 1 equiv) was mixed with pH 9.5 phosphate buffer (3,200 nmol, 12.8 μL, 250 mM in H₂O, 400 equiv), N-boc amino acid or nitro acid (800 nmol, 4 μL, 200 mM in AcCN, 100 equiv), DMTMM (800 nmol, 4 μL, 200 mM in H₂O, 100 equiv), and acetonitrile (4 μL). The reaction mixture was left at room temperature overnight before EtOH precipitation. The DNA-linked acids were subjected to reverse acylation. The DNA-linked acid (8 nmol, 8 μL, 1.0 mM in H₂O, 1 equiv) was mixed with pH5.8 MES buffer (3,200 nmol, 12.8 μL, 250 mM in H₂O, 400 equiv), N-boc diamine or nitro amine (800 nmol, 4 μL, 200 mM in AcCN, 100 equiv), DMTMM (800 nmol, 4 μL, 200 mM in H₂O, 100 equiv), and acetonitrile (4 μL). The reaction mixture was left at room temperature overnight before EtOH precipitation. The DNA-linked aldehyde was subjected to reductive amination. The DNA-linked aldehyde (8 nmol, 8 μL, 1.0 mM in H₂O, 1 equiv) was mixed with pH 5.8 MES buffer (3,200 nmol, 12.8 μL, 250 mM in H₂O, 400 equiv), N-boc diamine or nitro-amine (800 nmol, 4 μL, 200 mM in AcCN, 100 equiv), acetonitrile (8 μL, 30% v/v at final concentration of 0.2 mM), water (3.2 μL, rest of the volume), followed by NaCNBH₃ (800 nmol, 4 μL, 200 mM in H₂O, 100 equiv). The mixture was incubated at 35° C. overnight. After monitoring the reaction with LCMS, another 1 equiv of NaCNBH₃ was added to each well and heated to 35° C. overnight before EtOH precipitation. All wells were monitored with LCMS.

The nitro-group was reduced and N-boc was deprotected. For nitro reduction, the DNA-linked nitro (8 nmol, 8 μL, 1.0 mM in H₂O, 1 equiv) was mixed with NaOH (4,000 nmol, 8 μL, 500 mM in H₂O, 500 equiv), B2(OH)₄ (1,200 nmol, 12 μL, 100 mM in H₂O, 150 equiv), and EtOH (15 μL, 30% v/v at final concentration of 0.2 mM). The reaction mixture was left overnight at room temperature. For N-boc deprotection, the DNA-linked material (8 nmol, 16 NL) was added with pH 9.5 phosphate buffer (24 μL, 1.5× of DNA volume) and heated to 80° C. for 24 hr. All wells were monitored with LC-MS and all wells with less than 40% desired product were omitted from ligation. After C1 ligation, a total of 1170 wells were pooled, containing primary amines, secondary amines, and anilines as a terminal functional group. The pooled material was subjected to an additional round of EtOH precipitation and removal of any material that was not water soluble after reconstitution. The final concentration of C1 pooled material was 1.03 mM (4,500 μL).

### Procedure for Cycle 2

The pooled material was split into 528 wells followed by C2 ligation using standard procedure. The ligated material was subjected to acylation with acid halides and reductive amination with formyl acids. All the reaction conditions are as previously described. The reaction was monitored using spike-in (2.19 nmol, 25% of DNA mol amount) that was inserted into well A6 of each plate. Those particular wells contained 1.25× of all the other reaction conditions. Some wells were also used to monitor reaction using the headpiece and a previously validated substrate instead of using the pooled material and a substrate from the library building block. The acylation products were further subjected to hydroxycarbonylation in order to convert the terminal halide into a carboxylate group. To the DNA-linked halide (8.76 nmol, 10 μL, 0.876 mM in H₂O, 1 equiv) was added CsOH (3,504 nmol, 8.76 μL, 400 mM in H₂O, 400 equiv), Mo(CO)6 (876 nmol, 4.38 μL, 200 mM in MiPO, 100 equiv), followed by SPhos-Pd-G2 (105.12 nmol, 5.26 μL, 20 mM in MiPO, 12 equiv). The reaction mixture was heated to 80° C. for 15 min. Then, sodium cysteine (876 nmol, 4.38 μL, 200 mM in H₂O with 1.8× NaOH, 100 equiv) was added. These reactions were also monitored using the appropriate spike-in and previously validated substrate. All 528 wells were pooled, followed by another round of EtOH precipitation and removal of any material that was not soluble in water upon reconstitution. The final concentration was 0.89 mM (4,500 L).

### Preparation of DNA Samples for Selection Experiments (Cycle 3)

The procedures after C3 codon ligation is as described for qDOS38_1 synthesis.

Detailed Procedure for qDOS38_4 Synthesis

### Procedure for Cycle 1

The procedure until the C1 reaction is the same as the qDOS38_2 synthesis described above.

### Procedure for Cycle 2

Instead of pooling all the wells, only primary and secondary amines were pooled. A total of 1044 wells were pooled from C1. The pooled material was subjected to an additional round of EtOH precipitation and removal of any material that was not water soluble after reconstitution. The final concentration of C1 pooled material was 1.05 mM (2,625 μL).

The pooled material was split into 146 wells and C2 codons were ligated. The reconstituted material was subjected to urea formation. The amino acid building block (1,780 nmol, 8.9 μL, 200 mM in AcCN, 100 equiv) was pre-incubated with DSC (1,780 nmol, 8.9 μL, 200 mM in AcCN, 100 equiv) for 2 hours before adding it to the mixture of DNA-linked amine (17.8 nmol, 17 μL, 0.96 mM in H₂O, 1 equiv), pH 9.5 phosphate buffer (7,120 nmol, 28.48 μL, 250 mM in H₂O, 400 equiv), and TEA (2,670 nmol, 13.35 μL, 200 mM in AcCN, 150 equiv). The reaction mixture was left at room temperature overnight. The reaction was monitored using spike-in and previously validated substrate. All 146 wells were pooled and were subjected to EtOH precipitation and removal of any material that was insoluble in water upon reconstitution. The final concentration of C2 pooled material was 1.02 mM (2,500 uL).

### Preparation of DNA Samples for Selection Experiments (Cycle 3)

The procedures after C3 codon ligation is as described for qDOS38_1 synthesis.

### Instrumentations

All chemical reagents were purchased from commercial sources and used without further purification. Solvents were purchased as either anhydrous grade products in sealed containers or reagent grade and used as received. Flash column chromatography was performed using an automated chromatography instrument with an appropriately sized silica column. Thin layer chromatography was performed on silica gel 60 F254 plates (E. Merck). Non-UV active compounds were visualized on TLC using one of the following stains: KMnO4, ninhydrin, p-anisaldehyde, 2,4-DNP, or bromocresol green. LC-MS used for data collection was 1290 Infinity Series liquid chromatography with a 6150 mass spectrometer system (Agilent). The solid phase column was Agilent Eclipse Plus C18, 2.1 mm×50 mm (8 μm), and the mobile phase solvents were A: 0.05% formic acid in water and B: 5% water in acetonitrile. Peak detection was done at 254 nm and 230 nm. The HRMS data were acquired on a Thermo Q Exactive Orbitrap MS coupled with a Thermo Vanish UHPLC. The Zorbax XDB-C18 (4.6 mm×50 mm, 3 μm) was used for separation and kept at 40° C. The flow rate used was 0.3 mL/min with a gradient ranging from 2% to 95% aqueous acetonitrile containing 0.1% formic acid in a 7-minute run. Q Exactive MS was operated in positive mode with electrospray ionization. The lock mass at m/z 371.1012 for positive mode was used as a reference ion during acquisition.

NMR spectra were collected using a Bruker 600 MHz or 151 MHz NMR for ¹H and ¹³C respectively. All NMR chemical shifts were referenced to the residual non-deuterated solvent as an internal standard. Signal multiplicities are described using the following abbreviations: s=singlet, d=doublet, t=triplet, b=broad, quar=quartet, quin=quintet, m=multiplet, v=very; abbreviations are combined, e.g. vbs=very broad singlet.

### Example 1: β-Lactamase-Focused DNA-Encoded Library Synthesis

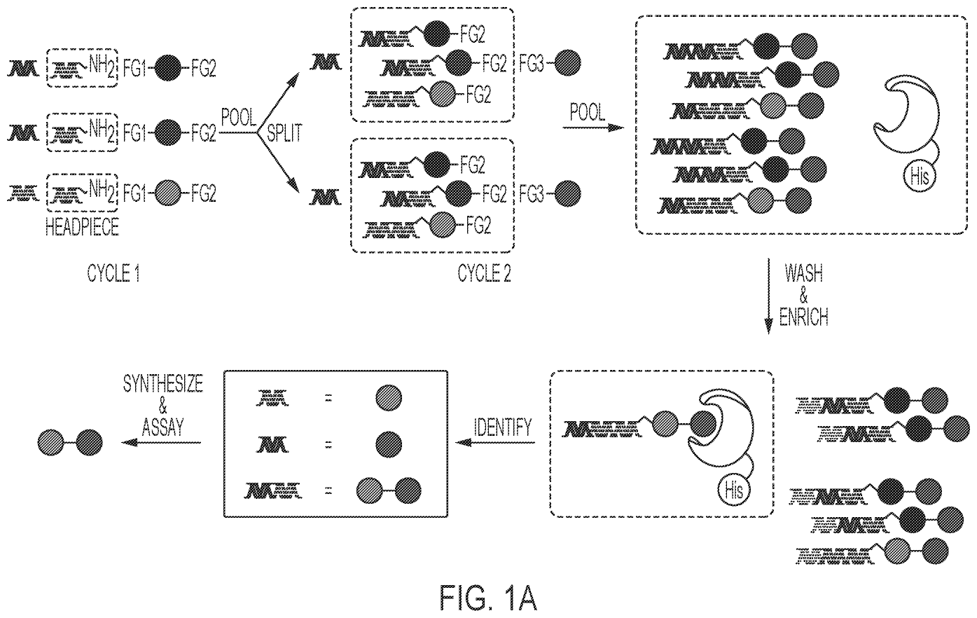

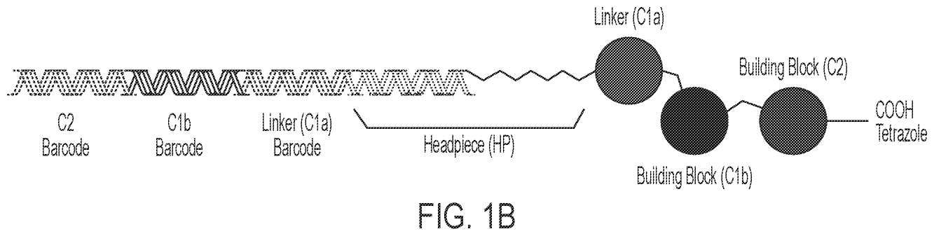

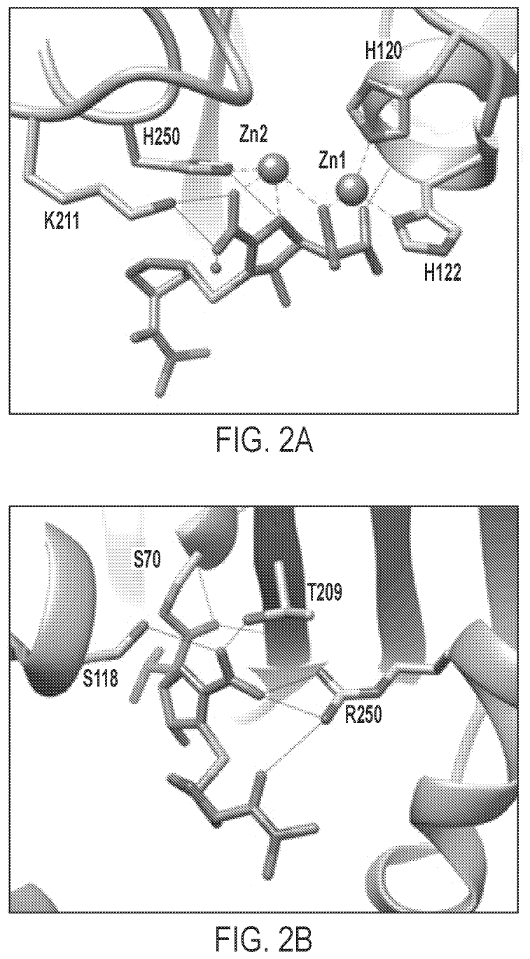

The β-lactamase-focused library containing carboxylic acids or tetrazoles as a core component has been synthesized. The synthesized molecules contain a core DNA scaffold, or headpiece (HP), onto which the remaining components were added (FIGS. 1A-1B). The headpiece consists of 17 nucleotide base pairs with a staggered end for ligation of barcode DNA sections, 4 polyethylene glycol units as well as the attachment point to which the linker and building blocks are added. This library contained two cycles of addition of diverse building blocks, denoted cycle 1 (C1) and cycle 2 (C2). Cycle 1 synthesis also includes the addition of linker molecules denoted as C1a to add more space between the HP and the actual library molecule. Linkers are thought to affect compound binding affinity and can provide a different topological approach towards the active site. C1b and C2 are building blocks that were added to make up the full molecule, where the C2 included the carboxylate handle at the end. Since two cycles were included, diversity was introduced by increasing the number of different chemical reactions used to combine the two building blocks.

The β-lactamase focused library was divided into three sub-libraries named qDOS38_1, qDOS38_2, and qDOS38_4, based on functional groups added post-cycle 1. qDOS38_3 was omitted during library synthesis due to its small size and inconsistent cycle 1 reaction. The functional groups post-cycle 1 were halides, amines (e.g., primary, secondary, and tertiary amines), and primary and secondary amines only, respectively. Various chemical reactions were performed to create C—C bond and C—N(e.g., amine, amide, or urea) between C1 and C2. When pooled at the end of the cycle, qDOS38_1 contained 228,750 compounds, qDOS_2 contained 617,760 compounds, and qDOS38_4 contained 152,146 compounds (i.e., approximately 1 million compounds total). The DNA barcodes from the naïve library were subjected to next generation DNA sequencing for comparison with sequencing data obtained after affinity selections.

### Example 2: Screening Against Different Enzymes and Identification of Hit Molecules

His-tagged enzymes were immobilized onto magnetic Ni-NTA (i.e., Ni(II) nitriloacetate) agarose beads and then incubated with the focused library. A parallel experiment excluding any enzyme on the Ni-NTA beads was conducted as a non-target control (NTC). Incubated magnetic beads were isolated by physical methods using a magnet and the non-binders left in solution were removed. A total of three rounds of selection was performed to fully enrich for specific binders. Between each round of selection, the total amount of DNA remaining was assessed using quantitative-PCR. Lastly, the DNA tags were amplified using PCR and deep sequenced by NGS for identification of the tags. Deep sequencing results were compared to the naïve library to calculate the enrichment score and further compared to the enrichment score of the NTC experiment to eliminate any non-specific binders.

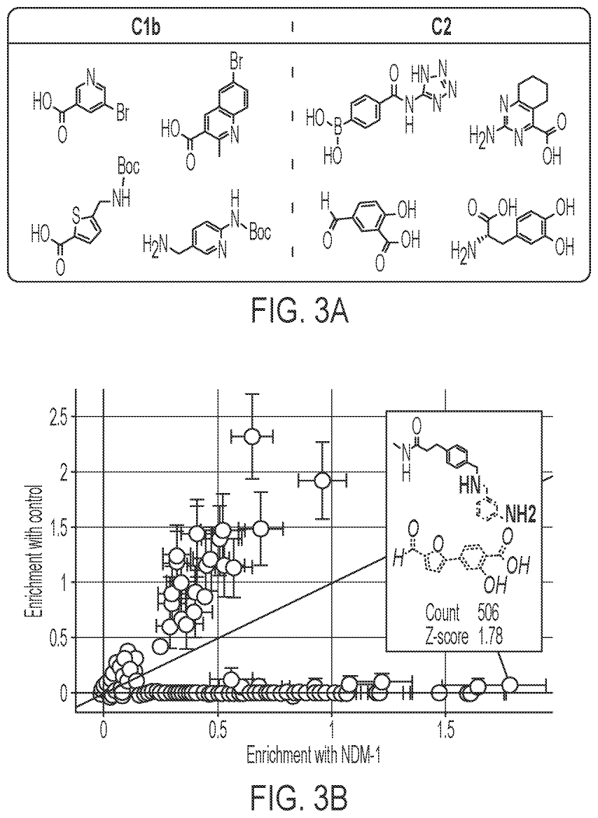

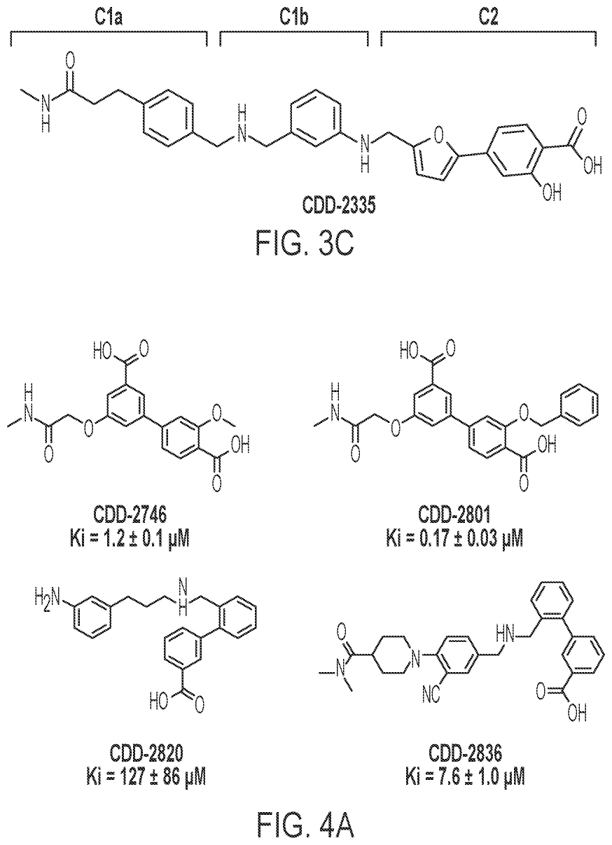

The analyzed data from deep sequencing was visualized on a 2-D plot of the enrichment scores of NTC on the γ-axis and against target protein on the x-axis (FIG. 3B). Individual circles represent a single molecule whose identity is revealed by C1 and C2 structures separately. The complete molecular information can be then gathered with the knowledge of library production route. Compounds that show any enrichment with the NTC were disregarded as non-specific binders. Compounds along the x-axis with higher enrichment scores were selected for further testing. These compounds were identified, resynthesized off-DNA, and subjected to further inhibition testing against the target enzymes.

### Example 3: Chemical Synthesis (i.e., Off-DNA Synthesis)

### General Procedure A: Amide Coupling

A mixture of amine (1 eq) and acid (0.8-1.2 eq) was dissolved in DMF (˜0.1M). HATU (1.5 eq) and DIPEA (2 eq) were added into the reaction mixture and stirred at r.t. for 3-15 h. Reaction was monitored by thin-layer chromatography (TLC) and quenched with sodium bicarbonate and extracted with ethyl acetate. The organic layer was dried over anhydrous sodium sulfate and dried in vacuo. The crude product was purified by normal phase silica gel chromatography using a hexanes/ethyl acetate or dichloromethane/methanol gradient. Product fractions were collected and dried in vacuo, yielding the desired product.

### General Procedure B: Reductive Amination

A mixture of aldehyde (1 eq) and amine (1.1-1.5 eq) was dissolved in DCM or MeOH (˜0.1 M) depending on their solubility and polarity. A small amount of sodium sulfate was added to the reaction mixture to remove any water generated from the reaction. A drop of acetic acid was added to catalyze the imine formation. The reaction mixture was stirred at r.t. for 1 h then STAB (1.5 eq) was added into the mixture and stirred at r.t. overnight. Reaction was monitored by TLC and quenched with sodium bicarbonate and extracted with ethyl acetate. If the product was too polar to be extracted, extraction step was skipped and just filtered using a 0.45 μm PTFE syringe filter to yield the crude product.

### General Procedure C: Suzuki Coupling

In a dry, high-pressure microwave reaction vial, halide (bromo- or iodo-, 1 eq), boronic acid or boronic acid pinacol ester (1.2 eq), potassium carbonate (1.5 eq), and Pd(dppf)Cl₂·DCM (0.1 eq) were added. The mixture was dissolved in 1,4-dioxane (˜0.5 M) and added 20% v/v H₂O. The reaction vial was purged under nitrogen gas and rapidly stirred at 110° C. for 30-60 min. Reaction was monitored by TLC and quenched with sodium bicarbonate and extracted with ethyl acetate. The organic layer was dried over anhydrous sodium sulfate and dried in vacuo. The crude product was purified by normal phase silica gel chromatography using a hexanes/ethyl acetate or dichloromethane/methanol gradient. Product fractions were collected and dried in vacuo, yielding the desired product.

### General Procedure D: Boc Deprotection

Prepared compound was dissolved in 1:3 TFA:DCM solution and stirred for 2-15 h at r.t. Reaction mixture neutralized with trimethylamine and dried under vacuum to obtain crude material. Reaction was monitored by TLC or LC-MS. Upon completion, the reaction mixture was dried in vacuo to obtain the crude material. The crude product was purified by HPLC or reverse phase C18 gel chromatography using a water/methanol or water/acetonitrile gradient. Product fractions were collected and dried in vacuo, yielding the desired product.

For acid-sensitive compound, the compound was dissolved in 1:1 1,4-dioxane:HCl solution in dioxane (4 M). The reaction mixture was stirred for 1-2 h at r.t. Reaction mixture was neutralized with 1N sodium bicarbonate and dried under vacuum. When dissolved in MeOH, any salt was filtered using a 0.45 μm PTFE syringe filter. Reaction was monitored by TLC or LC-MS. Upon completion, the reaction mixture was neutralized with TN sodium bicarbonate and dried in vacuo. The crude product was purified by HPLC or reverse phase C18 gel chromatography using a water/methanol or water/acetonitrile gradient. Product fractions were collected and dried in vacuo, yielding the desired product.

### General Procedure E: Ester Hydrolysis

Prepared compound was dissolved in 75% v/v THF and added 25% v/v LiOH solution in H₂O (TM, 3-5 eq). The reaction mixture was stirred at r.t. for 12-72 h. For slower reactions, 5% v/v MeOH was added to facilitate the hydrolysis reaction. Reaction mixture was neutralized with 1N HCl and dried under vacuum. Reaction was monitored by TLC or LC-MS. Upon completion, the reaction mixture was neutralized with 1N HCl and dried in vacuo. The crude product was purified by HPLC or reverse phase C18 gel chromatography using a water/methanol or water/acetonitrile gradient. Product fractions were collected and dried in vacuo, yielding the desired product.

### Synthesis of (5-(5-(methylcarbamoyl)pyridin-3-yl)pyrimidin-2-yl)glycine (CDD-2308)

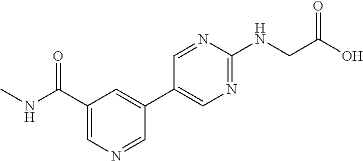 [SMILES: CNC(=O)c1cncc(-c2cnc(NCC(=O)O)nc2)c1]

The general procedure for Suzuki coupling was followed with 25 mg (0.116 mmol) 5-bromo-N-methylnicotinamide and 53 mg (0.174 mmol) ethyl (5-(4,4,5,5-tetramethyl-1,3,2-dioxaborolan-2-yl)pyrimidin-2-yl)glycinate in 1 mL 1,4-dioxane. The finished reaction was dried and purified using a normal phase chromatography with ethyl acetate/hexane followed by methanol/DCM gradient. 40 mg (55% yield) of product was isolated. 20 mg (0.0635 mmol) of isolated product was subjected to hydrolysis of ester using the general procedure described above. The reaction mixture was purified using reverse phase chromatography. Product fractions were dried in vacuo to yield 7.5 mg (41%) final product. ¹H NMR (600 MHz, MeOD) δ 8.90 (s, 2H), 8.66 (s, 2H), 8.40 (t, J=2.0 Hz, 1H), 3.97 (s, 2H), 2.97 (s, 3H).

### Synthesis of (5-(3-fluoro-4-(methylcarbamoyl)phenyl)pyrimidin-2-yl)glycine (CDD-2309)

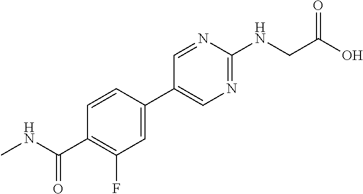 [SMILES: CNC(=O)c1ccc(-c2cnc(NCC(=O)O)nc2)cc1F]

The general procedure for amide coupling was followed using 100 mg (0.376 mmol) 2-fluoro-4-iodobenzoic acid and 226 μM (0.451 mmol) methylamine (2M in THF). Extracted portion was dried in vacuo, then purified by normal phase chromatography using an ethyl acetate/hexane gradient. Product fractions were dried in vacuo to yield 104 mg (99%) of 2-fluoro-4-iodo-N-methylbenzamide as a white solid.

40 mg (0.143 mmol) 2-fluoro-4-iodo-N-methylbenzamide was reacted with 66 mg (0.215 mmol) ethyl (5-(4,4,5,5-tetramethyl-1,3,2-dioxaborolan-2-yl)pyrimidin-2-yl)glycinate following the general procedure of Suzuki coupling. Extracted portion was dried in vacuo, then purified by normal phase chromatography using an ethyl acetate/hexane gradient.

Product fractions were dried in vacuo to yield 34.4 mg (73%) of product. 20 mg (0.0602 mmol) of the product was reacted further using the general procedure for hydrolysis. Reaction mixture was dried and purified using reverse phase chromatography yielding 6.2 mg (34%) of final product. ¹H NMR (600 MHz, MeOD) δ 8.65 (s, 2H), 7.83 (t, J=7.9 Hz, 1H), 7.49 (dd, J=24.1, 10.1 Hz, 2H), 4.13 (s, 2H), 2.95 (s, 3H).

### Synthesis of (5-(2-methyl-3-(methylcarbamoyl)quinolin-6-yl)pyrimidin-2-yl)glycine (CDD-2310)

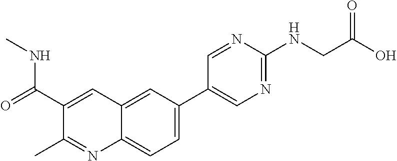 [SMILES: CNC(=O)c1cc2cc(-c3cnc(NCC(=O)O)nc3)ccc2nc1C]

The general procedure for amide coupling was followed using 70 mg (0.263 mmol) 6-bromo-2-methylquinoline-3-carboxylic acid and 166 μM (0.332 mmol) methylamine (2M in THF). Extracted portion was dried in vacuo, then purified by normal phase chromatography using an ethyl acetate/hexane gradient. Product fractions were dried in vacuo to yield 70 mg (95%) of 6-bromo-N,2-dimethylquinoline-3-carboxamide.

40 mg (0.143 mmol) 6-bromo-N,2-dimethylquinoline-3-carboxamide was reacted with 66 mg (0.215 mmol) ethyl (5-(4,4,5,5-tetramethyl-1,3,2-dioxaborolan-2-yl)pyrimidin-2-yl)glycinate following the general procedure of Suzuki coupling. Extracted portion was dried in vacuo, then purified by normal phase chromatography using an ethyl acetate/hexane gradient. Product fractions were dried in vacuo to yield 11 mg (20%) of product. 10 mg (0.0264 mmol) of the product was reacted further using the general procedure for hydrolysis. Reaction mixture was dried and purified using reverse phase chromatography yielding 2.4 mg (26%) of final product. ¹H NMR (600 MHz, MeOD) δ 8.73 (s, 1H), 8.64 (s, 1H), 8.36 (s, 1H), 8.13 (s, 1H), 8.04 (s, 1H), 7.82 (t, J=7.9 Hz, 1H), 7.48 (dd, J=23.6, 10.2 Hz, 1H), 4.35 (d, J=267.6 Hz, 2H), 2.96 (d, J=23.8 Hz, 3H), 2.77 (s, 2H).

### Synthesis of 5-(4-(2-(dimethylamino)-2-oxoethoxy)-3-formyl-5-methoxyphenyl)pyrimidin-2-yl)glycine) (CDD-2311)

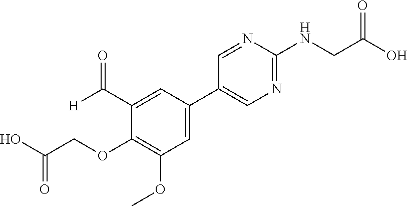 [SMILES: COc1cc(-c2cnc(NCC(=O)O)nc2)cc(C=O)c1OCC(=O)O]

40 mg (0.127 mmol) 2-(4-bromo-2-formyl-6-methoxyphenoxy)-N,N-dimethylacetamide was reacted with 58 mg (0.215 mmol) ethyl (5-(4,4,5,5-tetramethyl-1,3,2-dioxaborolan-2-yl)pyrimidin-2-yl)glycinate following the general procedure of Suzuki coupling. Extracted portion was dried in vacuo, then purified by normal phase chromatography using an ethyl acetate/hexane gradient. Product fractions were dried in vacuo to yield 35 mg (67%) of product. 20 mg (0.0481 mmol) of the product was reacted further using the general procedure for hydrolysis overnight. Reaction mixture was dried and purified using reverse phase chromatography yielding 2.4 mg (13.8%) of final product. ¹H NMR (600 MHz, MeOD) δ 10.57 (s, 1H), 8.58 (s, 2H), 7.14 (d, J=18.4 Hz, 1H), 4.15 (s, 2H), 3.97 (s, 2H), 3.92 (s, 3H).

### Synthesis of (5-(4-(carboxymethoxy)-3-formyl-5-methoxyphenyl)pyrimidin-2-yl)glycine

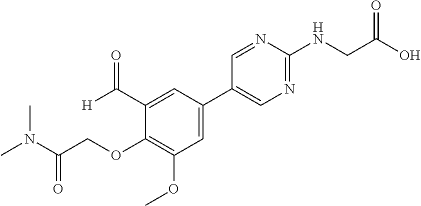 [SMILES: COc1cc(-c2cnc(NCC(=O)O)nc2)cc(C=O)c1OCC(=O)N(C)C]

Synthesis of CDD-2311 was followed exactly but hydrolysis reaction was only conducted for 1 h instead of overnight. Reaction mixture was dried and purified using reverse phase chromatography yielding 3.4 mg (18.2%) of final product. ¹H NMR (600 MHz, MeOD) δ 10.53 (s, 1H), 8.59 (s, 2H), 7.49 (d, J=20.7 Hz, 1H), 7.31-7.07 (m, 1H), 5.83 (s, 1H), 5.01 (s, 1H), 4.11 (s, 2H), 3.96 (d, J=32.8 Hz, 3H), 3.05 (d, J=10.1 Hz, 3H), 2.98 (d, J=27.1 Hz, 3H).

### Synthesis of 2-hydroxy-4-(5-(((3-(((4-(3-(methylamino)-3-oxopropyl)benzyl)amino)methyl)phenyl)amino)methyl)furan-2-yl)benzoic acid (CDD-2335)

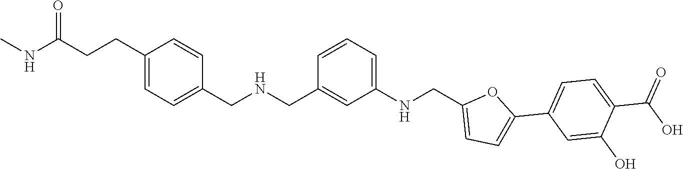 [SMILES: CNC(=O)CCc1ccc(CNCc2cccc(NCc3ccc(-c4ccc(C(=O)O)c(O)c4)o3)c2)cc1]

The general procedure for amide coupling was followed using 100 mg (0.562 mmol) 3-(4-formylphenyl)propanoic acid and 337 μM (0.674 mmol) methylamine (2M in THF). Extracted portion was dried in vacuo, then purified by normal phase chromatography using an ethyl acetate/hexane gradient. Product fractions were dried in vacuo to yield 138.8 mg (65%) of 3-(4-formylphenyl)-N-methylpropanamide as a white solid.

The general procedure for reductive amination was followed using 43 mg (0.225 mmol) 3-(4-formylphenyl)-N-methylpropanamide and 55 mg (0.251 mmol) tert-butyl (3-(aminomethyl)phenyl)carbamate in DCM. Extracted portion was dried, then purified by normal phase chromatography using an ethyl acetate/hexane followed by methanol/DCM gradient. Product fractions were dried in vacuo to yield 52.2 mg (58%) of the product as a white solid.

50 mg (0.126 mmol) of product was Boc-deprotected using TFA procedure stated above. The crude mixture of deprotected product was immediately used in next reaction without further purification.

The general procedure for reductive amination was followed with deprotected product and 35 mg (0.151 mmol) 4-(5-formylfuran-2-yl)-2-hydroxybenzoic acid. Reaction mixture was dried, then purified by reverse phase chromatography using a methanol/water followed. Product fractions were dried in vacuo to yield 10.42 mg (16%) of CDD-2335 as a white solid. ¹H NMR (600 MHz, MeOD) δ 7.77 (d, J=8.1 Hz, 1H), 7.28 (d, J=7.7 Hz, 2H), 7.22 (d, J=7.9 Hz, 2H), 7.21-7.18 (m, 1H), 7.02-7.00 (m, 2H), 6.81 (d, J=8.3 Hz, 1H), 6.78 (s, 1H), 6.70 (d, J=7.4 Hz, 1H), 6.64 (d, J=3.2 Hz, 1H), 6.29 (d, J=3.1 Hz, 1H), 4.37 (s, 2H), 4.07 (d, J=5.0 Hz, 4H), 2.88 (t, J=7.7 Hz, 2H), 2.65 (s, 3H), 2.43 (t, J=7.7 Hz, 2H). ¹³C NMR (151 MHz, MeOD) δ 153.61, 151.77, 149.09, 148.61, 142.41, 129.58, 129.55, 129.51, 128.80, 128.77, 117.72, 114.29, 113.37, 112.98, 112.72, 110.21, 110.10, 108.73, 108.36, 106.75, 50.96, 50.10, 48.17, 40.22, 36.98, 31.03, 24.87. Calc.'d m/z [M+H](C30H32N3O5+): 514.59; Obs.'d m/z: 514.35

### Synthesis of 2-(4′-(((3-(((4-(3-(methylamino)-3-oxopropyl)benzyl)amino)methyl)phenyl)amino)methyl)-[1,1′-biphenyl]-2-yl)acetic acid (CDD-2336)

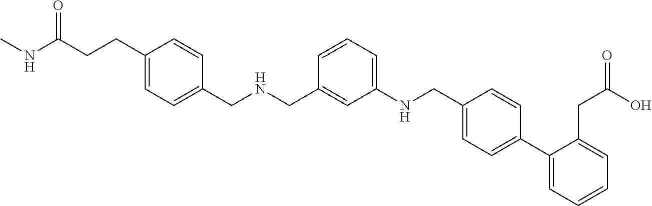 [SMILES: CNC(=O)CCc1ccc(CNCc2cccc(NCc3ccc(-c4ccccc4CC(=O)O)cc3)c2)cc1]

Same procedure was as the synthesis of CDD-2445 was used until Boc-deprotection step. 10 mg (0.0252 mmol) Boc-deprotected product was reacted with 7.3 mg (0.03024 mmol) 2-(4′-formyl-[1,1′-biphenyl]-2-yl)acetic acid following the general procedure of reductive amination. The filtered reaction mixture was dried, then purified by reverse phase chromatography using a methanol/water followed. Product fractions were dried in vacuo to yield 2.6 mg (35%) of CDD-2336. ¹H NMR (600 MHz, MeOD) δ 7.46 (d, J=6.9 Hz, 1H), 7.37 (d, J=7.0 Hz, 3H), 7.33-7.19 (m, 10H), 7.14 (t, J=8.6 Hz, 2H), 6.75 (d, J=8.0 Hz, 1H), 6.60 (d, J=5.8 Hz, 1H), 6.56 (s, 1H), 4.40 (s, 2H), 3.99 (d, J=58.0 Hz, 4H), 2.91 (t, J=7.6 Hz, 2H), 2.65 (s, 3H), 2.45 (t, J=7.7 Hz, 2H).

### Synthesis of 5-((((5-(dimethylcarbamoyl)thiophen-2-yl)methyl)amino)methyl)-2-hydroxybenzoic acid (CDD-2350)

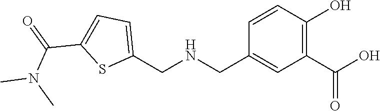 [SMILES: CN(C)C(=O)c1ccc(CNCc2ccc(O)c(C(=O)O)c2)s1]

The general procedure for amide coupling was followed using 30 mg (0.117 mmol) 5-(((tert-butoxycarbonyl)amino)methyl)thiophene-2-carboxylic acid and 70 μM (0.140 mmol) dimethylamine (2M in THF). Extracted portion was dried in vacuo, then purified by normal phase chromatography using an ethyl acetate/hexane gradient. Product fractions were dried in vacuo to yield 24.5 mg (73.5%) of tert-butyl ((5-(dimethylcarbamoyl)thiophen-2-yl)methyl)carbamate.

20 mg (0.0702 mmol) of product was Boc-deprotected using TFA procedure stated above for 2 h. The crude mixture of deprotected product was immediately used in next reaction without further purification. The general procedure for reductive amination was followed using product from Boc-deprotection and 14 mg (0.0842 mmol) 5-formyl-2-hydroxybenzoic acid in methanol. Reaction mixture was dried, then purified by reverse phase chromatography using a methanol/water followed. Product fractions were dried in vacuo to yield 2.5 mg (10%) of CDD-2350. ¹H NMR (600 MHz, MeOD) δ 7.89 (s, 1H), 7.42 (s, 1H), 7.37 (s, 1H), 7.25 (s, 1H), 6.86 (d, J=7.5 Hz, 1H), 4.57 (s, 1H), 4.42 (s, 2H), 4.13 (s, 2H), 3.19 (d, J=87.6 Hz, 6H).

### Synthesis of 2-hydroxy-4-(5-((methylamino)methyl)furan-2-yl)benzoic acid (CDD-2363)

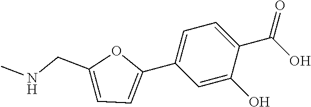 [SMILES: CNCc1ccc(-c2ccc(C(=O)O)c(O)c2)o1]

The general procedure for reductive amination was followed using 8.8 mg (0.0379 mmol) 4-(5-formylfuran-2-yl)-2-hydroxybenzoic acid and methylamine (26 μL, 0.0517 mmol, 2M in THF) in 1 mL MeOH. After reaction was completed, the reaction mixture was purified using reverse phase chromatography. Product fractions dried in vacuo yielding 1.4 mg (15%) of the final product. ¹H NMR (600 MHz, DMSO) δ 8.94 (s, 2H), 7.68 (d, J=7.8 Hz, 1H), 7.03 (s, 1H), 6.98 (d, J=7.6 Hz, 1H), 6.92 (s, 1H), 6.67 (s, 1H), 4.24 (s, 2H), 2.60 (d, J=16.3 Hz, 3H). Calc.'d m/z [M+H]⁺: 248.0923; Obs.'d: 248.0915

### Synthesis of 5-(((5-(acetamidomethyl)pyridin-2-yl)amino)methyl)-2-hydroxybenzoic acid (CDD-2373)

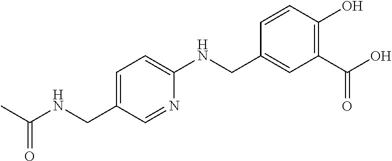 [SMILES: CC(=O)NCc1ccc(NCc2ccc(O)c(C(=O)O)c2)nc1]

50 mg (0.224 mmol) tert-butyl (5-(aminomethyl)pyridin-2-yl)carbamate was dissolved in 4 mL DCM and cooled down to 0° C. To the solution, 78 μL (0.448 mmol, 2 eq) of DIPEA was added then 21 mg (0.269 mmol, 1.2 eq) solution of acetyl chloride in 1 mL DCM was added dropwise. The reaction mixture was stirred at 0° C. for 1 h before extraction with ethyl acetate. Extracted fraction was dried and purified using normal phase chromatography over ethyl acetate/hexane gradient to yield 41 mg (69%) of tert-butyl (5-(acetamidomethyl)pyridin-2-yl)carbamate.

20 mg (0.0755 mmol) tert-butyl (5-(acetamidomethyl)pyridin-2-yl)carbamate was deprotected using Boc-deprotection reaction described in the general procedure with TFA. After reaction was complete, the crude material was directly used to react with 20 mg (0.109 mmol) methyl 5-formyl-2-hydroxybenzoate in 1 mL THF. To the reaction mixture, 35 μL (0.118 mmol, 1.3 eq) Ti(IV)isopropoxide was added. The reaction was purged under N₂ and stirred at 60° C. for 1 h before adding 8 mg (0.1818 mmol, 2 eq) NaBH₄. The reaction was stirred overnight at r.t. Reaction was monitored using LC-MS and purified using reverse phase chromatography to yield 0.5 mg (2%) of the reaction product. The reaction product was hydrolyzed following the general procedure. The reacted material was purified using reverse phase chromatography to yield 0.8 mg (99%) of the reaction product. ¹H NMR (600 MHz, DMSO) δ 8.13 (d, J=15.6 Hz, 1H), 7.84 (d, J=1.4 Hz, 1H), 7.61 (d, J=2.0 Hz, 1H), 7.24 (td, J=8.5, 2.2 Hz, 1H), 7.07 (dd, J=8.2, 2.2 Hz, 1H), 6.80 (t, J=5.8 Hz, 1H), 6.53 (d, J=8.2 Hz, 1H), 6.43 (d, J=8.5 Hz, 1H), 4.29 (d, J=5.8 Hz, 2H), 4.02 (d, J=5.5 Hz, 2H), 1.80 (s, 3H).

### Synthesis of 2-hydroxy-4-(5-(((3-((methylamino)methyl)phenyl)amino)methyl)furan-2-yl)benzoic acid (CDD-2376)

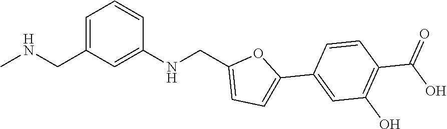 [SMILES: CNCc1cccc(NCc2ccc(-c3ccc(C(=O)O)c(O)c3)o2)c1]

The general procedure for reductive amination was followed using 25 mg (0.103 mmol) tert-butyl (3-aminobenzyl)(methyl)carbamate and 20 mg (0.0862 mmol) 4-(5-formylfuran-2-yl)-2-hydroxybenzoic acid in 1.5 mL MeOH. After reaction was completed, the reaction mixture was dried in vacuo to get the crude product. The crude material was immediately used in Boc-deprotection reaction with TFA as described above. Reaction mixture was dried and followed by reverse phase chromatography using a methanol/water gradient. Product fractions were dried in vacuo to yield 6.42 mg (17%) of CDD-2376 as a white solid. ¹H NMR (600 MHz, MeOD) δ 9.59 (s, 1H), 7.80 (d. J=8.5 Hz, 1H), 7.20 (t, J=7.8 Hz, 1H), 7.05-7.04 (m, 2H), 6.83-6.79 (m, 1H), 6.79 (s, 1H), 6.70 (d, J=7.5 Hz, 1H), 6.68 (d, J=3.4 Hz, 1H), 6.31-6.31 (m, 1H), 4.37 (s, 2H), 4.05 (s, 2H), 2.67 (s, 3H). Calc.'d m/z [M+H](C20H21N2O4+): 353.39; Obs.'d m/z:353.25

### Synthesis of 4-(5-(((3-((methylamino)methyl)phenyl)amino)methyl)furan-2-yl)benzoic acid (CDD-2463)

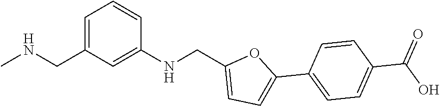 [SMILES: CNCc1cccc(NCc2ccc(-c3ccc(C(=O)O)cc3)o2)c1]

The general procedure for reductive amination was followed using 20 mg (0.047 mmol) tert-butyl (3-aminobenzyl)(methyl)carbamate and 20 mg (0.0932 mmol) 4-(5-formylfuran-2-yl)benzoic acid in 1 mL MeOH. The extracted material was dried to isolate the crude material. The crude material was immediately used in Boc-deprotection reaction with TFA as described above. Reaction mixture was dried and followed by reverse phase chromatography using a methanol/water gradient. Product fractions were dried in vacuo to yield 0.94 mg (3%) of CDD-2463. ¹H NMR (600 MHz, MeOD) δ 9.60 (s, 1H), 7.96 (m, 3H), 7.62 (d, J=6.6 Hz, 2H), 7.21 (t, J=7.8 Hz, 1H), 6.82 (d, J=8.2 Hz, 1H), 6.78 (s, 1H), 6.72 (d, J=3.2 Hz, 1H), 6.70 (d, J=7.4 Hz, 1H), 6.34 (d, J=3.1 Hz, 1H), 4.40 (s, 2H), 4.03 (s, 2H), 2.65 (s, 3H).

### Synthesis of (3-(5-(((3-((methylamino)methyl)phenyl)amino)methyl)furan-2-yl)phenol (CDD-2508)

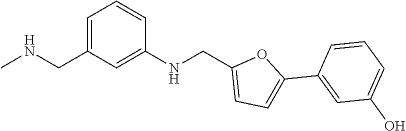 [SMILES: CNCc1cccc(NCc2ccc(-c3cccc(O)c3)o2)c1]

The general procedure for Suzuki coupling was followed with 40 mg (0.228 mmol) 5-bromofuran-2-carbaldehyde and 37 mg (0.274 mmol) (3-hydroxyphenyl)boronic acid. The extracted material was purified using normal phase chromatography under ethyl acetate/hexane gradient. Product fractions were collected to yield 37 mg (88%) of reaction product. 30 mg (0.159 mmol) of the reaction product was reacted with 37 mg (0.159 mmol) tert-butyl (3-aminobenzyl)(methyl)carbamate following the general procedure for reductive amination. The extracted material was purified using normal phase chromatography under ethyl acetate/hexane gradient. Product fractions were collected to yield 41 mg (63%) of reaction product. 20 mg (0.049 mmol) of the reaction product was Boc-deprotected using the general procedure with HCl for 2.5 h. Reaction was monitored using LC-MS and purified using HPLC under water/methanol gradient. Product fractions were collected and dried in vacuo to yield 6 mg (40%) of the title compound. ¹H NMR (600 MHz, MeOD) δ 7.15 (t, J=7.8 Hz, 1H), 7.09 (dd, J=7.2, 2.9 Hz, 2H), 7.06 (s, 1H), 6.70 (s, 1H), 6.65 (d, J=7.9 Hz, 2H), 6.62 (d, J=7.4 Hz, 1H), 6.58 (d, J=2.3 Hz, 1H), 6.29 (d, J=2.0 Hz, 1H), 4.35 (s, 2H), 3.59 (s, 2H), 2.33 (s, 3H).

### Synthesis of 2-hydroxy-5-(5-(((3-((methylamino)methyl)phenyl)amino)methyl)furan-2-yl)benzoic acid (CDD-2510)

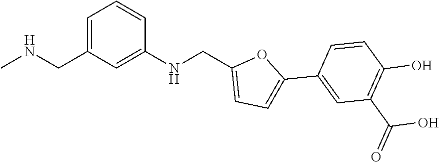 [SMILES: CNCc1cccc(NCc2ccc(-c3ccc(O)c(C(=O)O)c3)o2)c1]

The general procedure for Suzuki coupling was followed with 40 mg (0.228 mmol) 5-bromofuran-2-carbaldehyde and 76 mg (0.273 mmol) methyl 2-hydroxy-5-(4,4,5,5-tetramethyl-1,3,2-dioxaborolan-2-yl)benzoate. The extracted material was purified using normal phase chromatography under ethyl acetate/hexane gradient. Product fractions were collected to yield 38 mg (68%) of reaction product. 15.6 mg (0.0634 mmol) of the reaction product was reacted with 15 mg (0.0813 mmol) tert-butyl (3-aminobenzyl)(methyl)carbamate following the general procedure for reductive amination. The extracted material was purified using normal phase chromatography under ethyl acetate/hexane gradient. Product fractions were collected to yield 26 mg (87%) of reaction product.

20 mg (0.049 mmol) of the reaction product was hydrolyzed using the general procedure as described above. After neutralization, the product was filtered in 1,4-dioxane and proceeded onto Boc-deprotected using the general procedure with HCl for 1.5 h. Reaction was monitored using LC-MS and purified using HPLC under water/methanol gradient. Product fractions were collected and dried in vacuo to yield 2.3 mg (16%) of CDD-2510. ¹H NMR (600 MHz, MeOD) δ 8.10 (d, J=1.8 Hz, 1H), 7.54 (dd, J=8.5, 2.0 Hz, 1H), 7.19 (t, J=7.8 Hz, 1H), 6.87 (s, 1H), 6.79 (d, J=8.5 Hz, 2H), 6.70 (d, J=7.4 Hz, 1H), 6.45 (d, J=3.2 Hz, 1H), 6.27 (d, J=3.2 Hz, 1H), 4.37 (s, 2H), 4.07 (s, 2H), 2.67 (s, 3H).

### Synthesis of 4-(5-(((3-(aminomethyl)phenyl)amino)methyl)furan-2-yl)-2-hydroxybenzoic acid (CDD-2511)

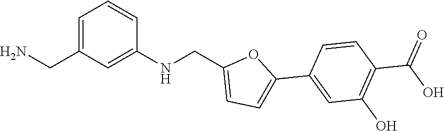 [SMILES: NCc1cccc(NCc2ccc(-c3ccc(C(=O)O)c(O)c3)o2)c1]

The general procedure for reductive amination was followed using 21 mg (0.0948 mmol) tert-butyl (3-aminobenzyl)carbamate and 13.5 mg (0.0582 mmol) 4-(5-formylfuran-2-yl)-2-hydroxybenzoic acid in 1.5 mL MeOH. The reaction was purified using HPLC without any extraction. Product fractions were collected to yield 7.6 mg (30%) of reaction product. 7 mg (0.16 mmol) of reaction product was subjected to Boc-deprotection reaction with HCl as described above. Reaction mixture was purified using HPLC. Product fractions were dried in vacuo to yield 2.3 mg (42%) of CDD-2511. ¹H NMR (600 MHz, MeOD) δ 7.82 (d, J=7.7 Hz, 1H), 7.20 (t, J=7.8 Hz, 1H), 7.11 (s, 2H), 6.81-6.74 (m, 3H), 6.69 (d, J=7.3 Hz, 1H), 6.35 (d, J=2.8 Hz, 1H), 4.41 (s, 2H), 3.99 (s, 2H).

### Synthesis of 2-hydroxy-3-(5-(((3-((methylamino)methyl)phenyl)amino)methyl)furan-2-yl)benzoic acid (CDD-2522)

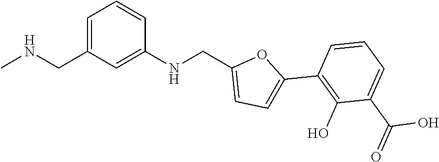 [SMILES: CNCc1cccc(NCc2ccc(-c3cccc(C(=O)O)c3O)o2)c1]

The general procedure for Suzuki coupling was followed with 40 mg (0.228 mmol) 5-bromofuran-2-carbaldehyde and 60 mg (0.216 mmol) methyl 2-hydroxy-3-(4,4,5,5-tetramethyl-1,3,2-dioxaborolan-2-yl)benzoate. The extracted material was purified using normal phase chromatography under ethyl acetate/hexane gradient. Product fractions were collected to yield 30 mg (56%) of reaction product. 15 mg (0.0610 mmol) of the reaction product was hydrolyzed using the general procedure as described above for 48 h. The reaction was neutralized and purified using reverse phase chromatography under water/methanol gradient. Product fractions were collected to yield 6.5 mg (46%) of reaction product 6.5 mg (0.0278 mmol) of the reaction product was reacted with 6.6 mg (0.0278 mmol) tert-butyl (3-aminobenzyl)(methyl)carbamate following the general procedure for reductive amination. The extracted material was purified using HPLC. Product fractions were collected then Boc-deprotected using the general procedure with HCl for 1.5 h. Reaction was monitored using LC-MS and purified using HPLC under water/methanol gradient. Product fractions were collected and dried in vacuo to yield 2.5 mg (25%) of CDD-2522. ¹H NMR (600 MHz, MeOD) δ 7.75 (ddd, J=15.9, 7.7, 1.6 Hz, 2H), 7.20 (t, J=7.8 Hz, 1H), 6.93 (d, J=3.2 Hz, 1H), 6.82 (dd, J=8.2, 1.5 Hz, 1H), 6.77 (dd, J=15.4, 7.7 Hz, 2H), 6.69 (d, J=7.5 Hz, 1H), 6.30 (d, J=3.2 Hz, 1H), 4.39 (s, 2H), 4.02 (s, 2H), 2.63 (s, 3H).

### Synthesis of 3-(5-(((3-(aminomethyl)phenyl)amino)methyl)furan-2-yl)-2-hydroxybenzoic acid (CDD-2567)

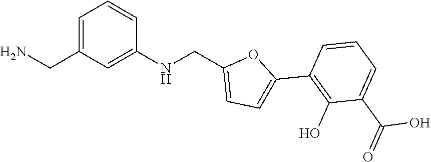 [SMILES: NCc1cccc(NCc2ccc(-c3cccc(C(=O)O)c3O)o2)c1]

Same procedure was followed for CDD-2522 for Suzuki coupling. 14 mg (0.0569 mmol) Suzuki product was reacted with 15 mg (0.0683 mmol) tert-butyl (3-aminobenzyl)carbamate in 1 mL DCM. The extracted material was purified using normal phase chromatography over ethyl acetate/hexane gradient to yield 26 mg (99%) of reaction product.

26 mg (0.0581 mmol) reaction product was hydrolyzed using the general procedure as described above. The reaction material was purified with HPLC to yield 17.5 mg (69%) of reaction product. This was then Boc-deprotected following the general procedure with HCl for 1.5 h. The reaction material was purified with HPLC to yield 8.8 mg (66%) of CDD-2567. ¹H NMR (600 MHz, DMSO) δ 8.07 (s, 3H), 7.65 (dd, J=18.0, 7.4 Hz, 2H), 7.13 (t, J=7.8 Hz, 1H), 6.95 (d, J=3.1 Hz, 1H), 6.76-6.67 (m, 3H), 6.63 (d, J=7.5 Hz, 1H), 6.37 (d, J=3.0 Hz, 1H), 6.33 (s, 1H), 4.32 (s, 2H), 3.89 (s, 2H).

### Synthesis of 4-(5-(((3-(aminomethyl)phenyl)amino)methyl)thiophen-2-yl)-2-hydroxybenzoic acid (CDD-2600)

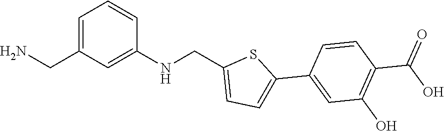 [SMILES: NCc1cccc(NCc2ccc(-c3ccc(C(=O)O)c(O)c3)s2)c1]

The general procedure for Suzuki coupling was followed with 30 mg (0.144 mmol) 5-bromothiophene-2-carbaldehyde and 50 mg (0.180 mmol) methyl 2-hydroxy-4-(4,4,5,5-tetramethyl-1,3,2-dioxaborolan-2-yl)benzoate. The extracted material was purified using normal phase chromatography under ethyl acetate/hexane gradient. Product fractions were collected to yield 26 mg (70%) of reaction product. 20 mg (0.0763 mmol) of the reaction product was reacted with 20 mg (0.0916 mmol) tert-butyl (3-aminobenzyl)carbamate following the general procedure for reductive amination. The extracted material was purified using normal phase chromatography under ethyl acetate/hexane gradient. Product fractions were collected to yield 24 mg (68%) of reaction product.

24 mg (0.0513 mmol) of the reaction product was hydrolyzed using the general procedure as described above for 48 h. The reaction was neutralized and purified using HPLC. Product fractions were collected to yield 3 mg (13%) of reaction product. 3 mg (0.00654 mmol) of the reaction product was Boc-deprotected using the general procedure with HCl for 1.5 h. Reaction was monitored using LC-MS and purified using HPLC under water/methanol gradient. Product fractions were collected and dried in vacuo to yield 0.3 mg (15%) of CDD-2600. ¹H NMR (600 MHz, DMSO) δ 7.61 (d, J=8.4 Hz, 1H), 7.28 (d, J=3.5 Hz, 1H), 7.08 (t, J=7.7 Hz, 1H), 7.01 (d, J=3.2 Hz, 1H), 6.79 (d, J=6.4 Hz, 2H), 6.69 (s, 1H), 6.60 (d, J=6.5 Hz, 2H), 6.38 (t, J=5.9 Hz, 1H), 4.44 (d, J=5.9 Hz, 2H), 3.80 (s, 2H).

### Synthesis of 3-(5-(((3-((methylamino)methyl)phenyl)amino)methyl)furan-2-yl)benzoic acid (CDD-2602)

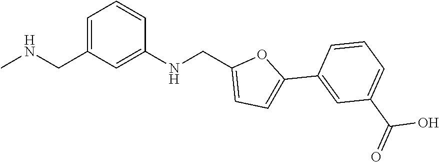 [SMILES: CNCc1cccc(NCc2ccc(-c3cccc(C(=O)O)c3)o2)c1]

200 mg (1.143 mmol) 3-boronobenzoic acid was reacted with 725 μL (5.714 mmol, 5 eq) TMSCl in 5 mL MeOH for 2 h. The reaction mixture was dried, neutralized with sodium bicarbonate and extracted with ethyl acetate. Without any further purification, (3-(methoxycarbonyl)phenyl)boronic acid was collected as white solid (197 mg, 91%).

The general procedure for Suzuki coupling was followed with 65 mg (0.343 mmol) (3-(methoxycarbonyl)phenyl)boronic acid and 50 mg (0.286 mmol) 5-bromofuran-2-carbaldehyde.

The extracted material was purified using normal phase chromatography under ethyl acetate/hexane gradient. Product fractions were collected to yield 67.5 mg (97%) of reaction product. 50 mg (0.205 mmol) of the reaction product was reacted with 58 mg (0.246 mmol) tert-butyl (3-aminobenzyl)carbamate following the general procedure for reductive amination. The extracted material was purified using normal phase chromatography under ethyl acetate/hexane gradient. Product fractions were collected to yield 30.5 mg (32%) of reaction product.

30 mg (0.0647 mmol) of the reaction product was hydrolyzed using the general procedure as described above overnight. The reaction was neutralized and purified using HPLC. Product fractions were collected to yield 21.5 mg (76%) of reaction product. 20 mg (0.0459 mmol) of the reaction product was Boc-deprotected using the general procedure with HCl for 2 h. Reaction was monitored using LC-MS and purified using HPLC under water/methanol gradient. Product fractions were collected and dried in vacuo to yield 7 mg (46%) of CDD-2602. ¹H NMR (600 MHz, MeOD) δ 8.18 (s, 1H), 7.80 (d, J=7.6 Hz, 1H), 7.65 (d, J=7.7 Hz, 1H), 7.34 (t, J=7.7 Hz, 1H), 7.18 (t, J=7.8 Hz, 1H), 6.87 (s, 11H), 6.78 (dd, J=8.2, 1.3 Hz, 1H), 6.70 (d, J=7.4 Hz, 1H), 6.65 (d, J=3.2 Hz, 1H), 6.30 (d, J=3.1 Hz, 1H), 4.37 (s, 2H), 4.06 (s, 2H), 2.66 (s, 3H).

### Synthesis of 4′-(((3-(aminomethyl)phenyl)amino)methyl)-3-hydroxy-[1,1′-biphenyl]-4-carboxylic acid (CDD-2615)

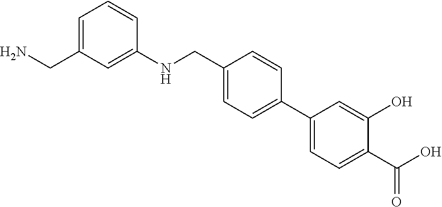 [SMILES: NCc1cccc(NCc2ccc(-c3ccc(C(=O)O)c(O)c3)cc2)c1]

The general procedure for Suzuki coupling was followed with 60 mg (0.324 mmol) 4-bromobenzaldehyde and 100 mg (0.389 mmol) methyl 2-hydroxy-4-(4,4,5,5-tetramethyl-1,3,2-dioxaborolan-2-yl)benzoate. The extracted material was purified using normal phase chromatography under ethyl acetate/hexane gradient. Product fractions were collected to yield 78.4 mg (94.5%) of reaction product. 50 mg (0.195 mmol) of the reaction product was reacted with 52 mg (0.234 mmol) tert-butyl (3-aminobenzyl)carbamate following the general procedure for reductive amination. The extracted material was purified using normal phase chromatography under ethyl acetate/hexane gradient. Product fractions were collected to yield 60 mg (66%) of reaction product.

50 mg (0.112 mmol) of the reaction product was hydrolyzed using the general procedure as described above overnight. The reaction was neutralized and purified using HPLC. Product fractions were collected to yield 19 mg (37.5%) of reaction product. 18 mg (0.0402 mmol) of the reaction product was Boc-deprotected using the general procedure with HCl for 2 h. Reaction was monitored using LC-MS and purified using HPLC under water/methanol gradient. Product fractions were collected and dried in vacuo to yield 0.7 mg (5%) of CDD-2615. ¹H NMR (600 MHz, MeOD) δ 10.03 (s, 1H), 8.55 (s, 1H), 8.01-7.83 (m, 3H), 7.60 (m, 2H), 7.46 (m, 2H), 7.13 (s, 1H), 7.02 (d, J=6.6 Hz, 2H), 6.68-6.53 (m, 3H), 4.37 (m, 2H), 4.11 (s, 1H), 3.85 (s, 1H).

### Synthesis of 3′-(((3-(aminomethyl)phenyl)amino)methyl)-3-hydroxy-[1,1′-biphenyl]-4-carboxylic acid (CDD-2616)

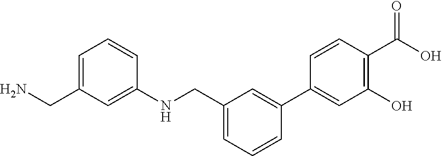 [SMILES: NCc1cccc(NCc2cccc(-c3ccc(C(=O)O)c(O)c3)c2)c1]

The general procedure for Suzuki coupling was followed with 60 mg (0.324 mmol) 3-bromobenzaldehyde and 100 mg (0.389 mmol) methyl 2-hydroxy-4-(4,4,5,5-tetramethyl-1,3,2-dioxaborolan-2-yl)benzoate. The extracted material was purified using normal phase chromatography under ethyl acetate/hexane gradient. Product fractions were collected to yield 73.7 mg (89%) of reaction product. 50 mg (0.195 mmol) of the reaction product was reacted with 52 mg (0.234 mmol) tert-butyl (3-aminobenzyl)carbamate following the general procedure for reductive amination. The extracted material was purified using normal phase chromatography under ethyl acetate/hexane gradient. Product fractions were collected to yield 59 mg (66%) of reaction product.

50 mg (0.112 mmol) of the reaction product was hydrolyzed using the general procedure as described above overnight. The reaction was neutralized and purified using HPLC. Product fractions were collected to yield 33 mg (66%) of reaction product. 30 mg (0.0669 mmol) of the reaction product was Boc-deprotected using the general procedure with HCl for 2 h. Reaction was monitored using LC-MS and purified using HPLC under water/methanol gradient. Product fractions were collected and dried in vacuo to yield 9.5 mg (41%) of CDD-2616. ¹H NMR (600 MHz, MeOD) δ 7.86 (d, J=7.8 Hz, 1H), 7.61 (s, 1H), 7.48 (d, J=7.3 Hz, 1H), 7.40-7.33 (m, 2H), 7.14 (t, J=7.8 Hz, 1H), 6.99 (d, J=8.7 Hz, 2H), 6.68 (d, J=7.5 Hz, 2H), 6.64 (d, J=7.4 Hz, 1H), 4.42 (s, 2H), 3.91 (s, 2H).

### Synthesis of 5-(4-(((3-aminophenyl)amino)methyl)phenyl)nicotinic acid (CDD-2396)

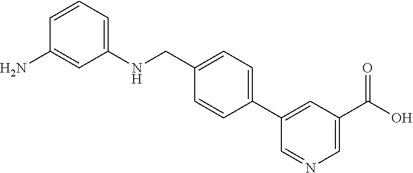 [SMILES: Nc1cccc(NCc2ccc(-c3cncc(C(=O)O)c3)cc2)c1]

The general procedure for reductive amination was followed using 50 mg (0.24 mmol) tert-butyl (3-aminophenyl)carbamate and 60 mg (0.26 mmol) 5-(4-formylphenyl)nicotinic acid in 2 mL MeOH. The crude material was purified on normal phase chromatography over methanol/DCM gradient without extraction. The product fractions were collected and were used in Boc-deprotection reaction with TFA as described above. Reaction mixture was dried and followed by reverse phase chromatography using a methanol/water gradient. Product fractions were dried in vacuo to yield 1.5 mg (2%) of CDD-2396. ¹H NMR (600 MHz, MeOD) δ 9.05 (d, J=1.7 Hz, 1H), 8.91 (d, J=2.1 Hz, 1H), 8.56 (t, J=2.0 Hz, 1H), 7.65 (d, J=8.2 Hz, 2H), 7.52 (d, J=8.1 Hz, 2H), 6.90 (t, J=3.9 Hz, 1H), 6.21-6.11 (m, 3H), 4.37 (s, 2H).

### Synthesis of 2-amino-3-(3,4-dihydroxyphenyl)-N-phenethylpropanamide (CDD-2507)

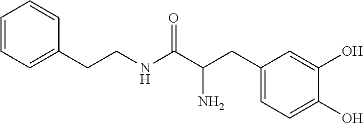 [SMILES: NC(Cc1ccc(O)c(O)c1)C(=O)NCCc1ccccc1]

The general procedure for amide coupling was followed using 50 mg (0.413 mmol) 2-phenylethan-1-amine and 147 mg (0.496 mmol) 2-((tert-butoxycarbonyl)amino)-3-(3,4-dihydroxyphenyl)propanoic acid. Extracted portion was dried in vacuo, then purified by normal phase chromatography using an ethyl acetate/hexane gradient. Product fractions were dried in vacuo to yield 98.4 mg (60%) of the reaction product. 10 mg (0.025 mmol) of reaction product was Boc-deprotected using TFA procedure stated above for 2 h. The crude material was dried and purified using reverse phase chromatography using water/methanol gradient. Product fractions were dried in vacuo to yield 1.5 mg (20.5%) of CDD-2507. ¹H NMR (600 MHz, MeOD) δ 7.28 (t, J=7.5 Hz, 2H), 7.18 (dd, J=17.7, 7.4 Hz, 3H), 6.74 (d, J=8.0 Hz, 1H), 6.68 (d, J=1.3 Hz, 1H), 6.54 (dd, J=8.0, 1.4 Hz, 1H), 3.82 (t, J=7.2 Hz, 1H), 3.55-3.47 (m, 1H), 3.36-3.33 (m, 1H), 2.94 (dd, J=13.9, 7.0 Hz, 1H), 2.81 (dd, J=13.8, 7.7 Hz, 1H), 2.72 (ddt, J=21.1, 13.8, 6.9 Hz, 2H).

### Synthesis of 5-hydroxy-3′-methoxy-[1,1′-biphenyl]-3,4′-dicarboxylic acid (CDD-2724)

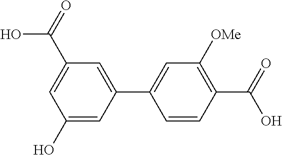 [SMILES: COc1cc(-c2cc(O)cc(C(=O)O)c2)ccc1C(=O)O]

### Step 1: dimethyl 5-hydroxy-3′-methoxy-[1,1′-biphenyl]-3,4′-dicarboxylate

The intermediate title compound was prepared according to general procedure C. Molecular Formula: C17H1606. ¹H NMR (600 MHz, CDCl₃) δ 7.88 (d, J=8.0 Hz, 1H), 7.85-7.82 (m, 1H), 7.56-7.54 (m, 1H), 7.29-7.27 (m, 1H), 7.17 (dd, J=8.0, 1.3 Hz, 1H), 7.12 (bs, 1H), 3.95 (s, 3H), 3.94 (s, 3H), 3.93 (s, 3H).

### Step 2: 5-hydroxy-3′-methoxy-[1,1′-biphenyl]-3,4′-dicarboxylic acid (CDD-2724)

The title compound was prepared from intermediate dimethyl 5-hydroxy-3′-methoxy-[1,1′-biphenyl]-3,4′-dicarboxylate following general procedure E. Molecular Formula: C₁₅H₁₂O₆. ¹H NMR (600 MHz, DMSO) δ 12.89 (bs, 1H), 10.09 (bs, 1H), 8.02 (s, 1H), 7.72 (d, J=7.9 Hz, 1H), 7.66 (s, 1H), 7.41 (bs, 1H), 7.34 (bs, 1H), 7.28 (s, 1H), 7.24 (d, J=8.0 Hz, 1H), 3.91 (s, 3H). ¹³C NMR (151 MHz, DMSO) δ 167.65, 167.50, 159.09, 158.54, 144.75, 141.42, 133.38, 131.96, 129.78, 120.92, 118.96, 118.91, 118.74, 116.20, 111.18, 63.29, 56.34. Calc.'d m/z [M+H]⁺: 289.0712; Obs.'d: 289.0699.

### Synthesis of 3′-(benzyloxy)-5-hydroxy-[1,1′-biphenyl]-3,4′-dicarboxylic acid (CDD-2725)

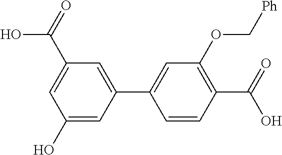 [SMILES: O=C(O)c1cc(O)cc(-c2ccc(C(=O)O)c(OCc3ccccc3)c2)c1]

### Step 1: dimethyl 3′-(benzyloxy)-5-hydroxy-[1,1′-biphenyl]-3,4′-dicarboxylate

The intermediate title compound was prepared according to general procedure C. Molecular Formula: C₂₃H₂₀O₆. ¹H NMR (600 MHz, CDCl₃) δ 7.92 (d, J=8.3 Hz, 1H), 7.81 (s, 1H), 7.55-7.51 (m, 3H), 7.40 (t, J=7.5 Hz, 2H), 7.32 (t, J=7.3 Hz, 1H), 7.23 (s, 1H), 7.22-7.19 (m, 2H), 5.25 (s, 2H), 3.95 (s, 3H), 3.93 (s, 3H).

### Step 2: 3′-(benzyloxy)-5-hydroxy-[1,1′-biphenyl]-3,4′-dicarboxylic acid (CDD-2725)

The title compound was prepared from intermediate dimethyl 3′-(benzyloxy)-5-hydroxy-[1,1′-biphenyl]-3,4′-dicarboxylate following general procedure E. Molecular Formula: C₂₁H₁₆O₆. ¹H NMR (600 MHz, DMSO) δ 12.89 (bs, 1H), 10.15 (bs, 1H), 8.01 (s, 1H), 7.75 (d, J=8.0 Hz, 1H), 7.65 (s, 1H), 7.55 (d, J=7.5 Hz, 2H), 7.44-7.38 (m, 4H), 7.34-7.30 (m, 2H), 7.26 (d, J=8.0 Hz, 1H), 5.33 (s, 2H). ¹³C NMR (151 MHz, DMSO) δ 167.67, 167.59, 158.55, 158.00, 144.59, 141.31, 137.60, 131.95, 129.78, 128.82, 128.09, 127.63, 119.22, 118.98, 118.68, 116.22, 112.74, 70.17, 63.29. Calc.'d m/z [M+H]⁺: 365.1025; Obs.'d: 365.1019.

### Synthesis of 3′-methoxy-5-(2-(methylamino)-2-oxoethoxy)-[1,1′-biphenyl]-3,4′-dicarboxylic acid (CDD-2746)

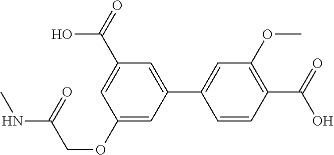 [SMILES: CNC(=O)COc1cc(C(=O)O)cc(-c2ccc(C(=O)O)c(OC)c2)c1]

### Step 1: dimethyl 3′-methoxy-5-(2-(methylamino)-2-oxoethoxy)-[1,1′-biphenyl]-3,4′-dicarboxylate

In a reaction vial, 1a (50 mg, 0.158 mmol), 2-bromo-N-methylacetamide (96 mg, 0.633 mmol), and potassium carbonate (167 mg, 0.79 mmol) were dissolved in 1 mL acetone. The reaction mixture was heated to 60° C. and rapidly stirred for 2 hr. The reaction was monitored with TLC (Rf=0.05 at 40% ethyl acetate/hexanes). Upon completion, the reaction mixture was diluted in ethyl acetate and washed with brine. The organic layer was dried over anhydrous sodium sulfate and dried in vacuo. The crude product was purified by normal phase silica gel chromatography using a hexanes/ethyl acetate gradient. Product fractions were eluted at 60-100% ethyl acetate/hexanes and were very streaky. Product fractions were collected and dried in vacuo, yielding the desired product (60.7 mg, 99%). Molecular Formula: C₂₀H₂₁NO₇. ¹H NMR (600 MHz, CDCl₃) δ 7.97-7.94 (m, 1H), 7.90 (d, J=8.0 Hz, 1H), 7.61-7.59 (m, 1H), 7.36-7.33 (m, 1H), 7.20 (dd, J=8.0, 1.2 Hz, 1H), 7.15 (bs, 1H), 4.61 (s, 2H), 4.00 (s, 3H), 3.96 (s, 3H), 3.92 (s, 3H), 2.95 (d, J=4.9 Hz, 3H), 1.57 (s, 2H).

### Step 2: 3′-methoxy-5-(2-(methylamino)-2-oxoethoxy)-[1,1′-biphenyl]-3,4′-dicarboxylic acid (CDD-2746)

In a microwave vial, the intermediate (15 mg, 0.0388 mmol) and triethylamnine (33 μL, 0.233 mmol) were dissolved in 1 mL acetonitrile. Lithium bromide (101 mg, 1.16 mmol) was quickly dissolved in 200 μL water and added to the reaction mixture. The reaction mixture was heated to 90° C. overnight. The reaction was monitored using LC-MS. Upon completion, the reaction mixture was filtered with acetonitrile and dissolved back in methanol. The crude product was purified with HPLC to yield the pure product (6.5 mg, 47%). Molecular Formula: C₁₈H₁₇NO₇. ¹H NMR (600 MHz, DMSO) δ 8.11 (bd, J=4.2 Hz, 1H), 7.81 (s, 1H), 7.61 (d, J=7.7 Hz, 1H), 7.49 (s, 1H), 7.32 (s, 1H), 7.26-7.20 (m, 2H), 4.56 (s, 2H), 3.89 (s, 3H), 2.67 (d, J=4.6 Hz, 3H). ¹³C NMR (151 MHz, DMSO) δ 168.53, 159.55, 158.29, 118.91, 116.63, 115.89, 110.95, 99.98, 84.64, 67.63, 56.21, 46.12, 25.83, 11.83. Calc.'d m/z [M+H]+: 360.1083; Obs.'d: 360.1076.

### Synthesis of 3′-methoxy-5-(2-((4-(3-(methylamino)-3-oxopropyl)phenyl)amino)-2-oxoethoxy)-[1,1′-biphenyl]-3,4′-dicarboxylic acid (CDD-2747)

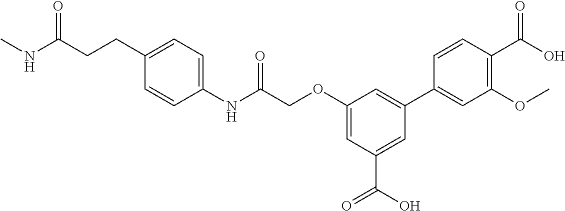

### Step 1: 3-(4-aminophenyl)-N-methylpropanamide

The intermediate title compound was prepared according to general procedure A with 3-(4-aminophenyl)propanoic acid (200 mg, 1.21 mmol) and 2M methylamine in THF (730 μL, 1.46 mmol). The crude product was used in the next reaction without further purification. Molecular Formula: C₁₀H₁₄N₂O. ¹H NMR (600 MHz, CDCl₃) δ 6.97 (d, J=8.2 Hz, 2H), 6.62 (d, J=8.3 Hz, 2H), 2.84 (t, J=7.7 Hz, 2H), 2.75 (d, J=4.8 Hz, 3H), 2.43-2.38 (m, 2H).

### Step 2: dimethyl 5-(2-(tert-butoxy)-2-oxoethoxy)-3′-methoxy-[1,1′-biphenyl]-3,4′-dicarboxylate

In a reaction vial, the intermediate (50 mg, 0.158 mmol), tert-butyl 2-bromoacetate (62 mg, 0.316 mmol), and potassium carbonate (100 mg, 0.474 mmol) were dissolved in 1 mL acetone. The reaction mixture was heated to 60° C. and rapidly stirred for 3 hr. The reaction was monitored with TLC (Rf=0.45 at 40% ethyl acetate/hexanes). Upon completion, the reaction mixture was diluted in ethyl acetate and washed with brine. The organic layer was dried over anhydrous sodium sulfate and dried in vacuo. The crude product was purified by normal phase silica gel chromatography using a hexanes/ethyl acetate gradient. Product fractions were eluted at 20% ethyl acetate/hexanes. Product fractions were collected and dried in vacuo, yielding the desired product (white oil, 60.8 mg, 89%). Molecular Formula: C₂₃H₂₆O₈. ¹H NMR (600 MHz, CDCl₃) δ 7.93-7.91 (m, 1H), 7.89 (d, J=8.0 Hz, 1H), 7.54-7.51 (m, 1H), 7.40-7.38 (m, 1H), 7.20 (dd, J=8.0, 1.3 Hz, 1H), 7.17-7.14 (m, 1H), 4.62 (s, 2H), 3.98 (s, 3H), 3.95 (s, 3H), 3.91 (s, 3H), 1.51 (s, 9H).

### Step 3: dimethyl 3′-methoxy-5-(2-((4-(3-(methylamino)-3-oxopropyl)phenyl)amino)-2-oxoethoxy)-[1,1′-biphenyl]-3,4′-dicarboxylate

General experimental procedure D was followed with TFA to remove tert-butyl group. No further purification was done. Subsequently, general experimental procedure A was followed with the linker (3-(4-aminophenyl)-N-methylpropanamide) (20 mg, 0.112 mmol) and the intermediate (35 mg, 0.093 mmol). Molecular Formula: C₂₉H₃₀N₂O₈. ¹H NMR (600 MHz, CDCl₃) δ 8.22 (s, 1H), 7.99-7.97 (m, 1H), 7.91 (d, J=8.0 Hz, 1H), 7.69-7.66 (m, 11H), 7.52 (d, J=8.3 Hz, 2H), 7.43-7.40 (m, 11H), 7.23-7.18 (m, 3H), 7.16 (d, J=0.7 Hz, 1H), 5.31 (s, 1H), 4.72 (s, 2H), 4.00 (s, 3H), 3.98 (s, 3H), 3.93 (s, 3H), 2.96 (t, J=7.6 Hz, 2H), 2.77 (d, J=4.8 Hz, 3H), 2.45 (t, J=7.6 Hz, 2H), 2.05 (s, 2H).

### Step 4: 3′-methoxy-5-(2-((4-(3-(methylamino)-3-oxopropyl)phenyl)amino)-2-oxoethoxy)-[1,1′-biphenyl]-3,4′-dicarboxylic acid (CDD-2747)

The title compound was prepared from the intermediate following general procedure E. General experimental procedure E was followed. Molecular Formula: C₂₇H₂₆N₂O₈. ¹H NMR (600 MHz, DMSO) δ 10.08 (s, 1H), 7.85 (s, 1H), 7.74 (d, J=7.9 Hz, 2H), 7.58 (s, 2H), 7.54 (d, J=8.3 Hz, 2H), 7.33 (s, 1H), 7.31 (d, J=8.0 Hz, 1H), 7.14 (d, J=8.3 Hz, 2H), 4.85 (s, 2H), 3.90 (s, 3H), 2.75 (t, J=7.7 Hz, 2H), 2.32 (t, J=7.8 Hz, 2H). Calc.'d m/z [M+H]⁺: 507.1767; Obs.'d: 507.1760

### Synthesis of 3′-(benzyloxy)-5-(2-(methylamino)-2-oxoethoxy)-[1,1′-biphenyl]-3,4′-dicarboxylic acid (CDD-2801)

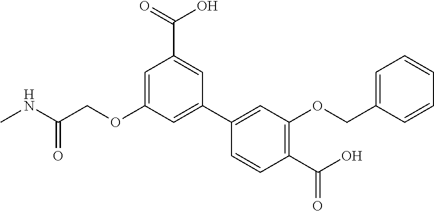 [SMILES: CNC(=O)COc1cc(C(=O)O)cc(-c2ccc(C(=O)O)c(OCc3ccccc3)c2)c1]

### Step 1: dimethyl 3′-(benzyloxy)-5-(2-(methylamino)-2-oxoethoxy)-[1,1′-biphenyl]-3,4′-dicarboxylate

In a reaction vial, dimethyl 3′-(benzyloxy)-5-hydroxy-[1,1′-biphenyl]-3,4′-dicarboxylate (50 mg, 0.128 mmol), 2-bromo-N-methylacetamide (58 mg, 0.383 mmol), and potassium carbonate (108 mg, 0.510 mmol) were dissolved in 1 mL acetone. The reaction mixture was heated to 60° C. and rapidly stirred for 2 hr. The reaction was monitored with TLC (Rf=0.08 at 40% ethyl acetate/hexanes). Upon completion, the reaction mixture was diluted in ethyl acetate and washed with brine. The organic layer was dried over anhydrous sodium sulfate and dried in vacuo. The crude product was purified by normal phase silica gel chromatography using a hexanes/ethyl acetate gradient. Product fractions were eluted at 50% ethyl acetate/hexanes. Product fractions were collected and dried in vacuo, yielding the desired product (52.3 mg, 89%). Molecular Formula: C₂₆H₂₅NO₇. ¹H NMR (600 MHz, CDCl₃) δ 7.93 (d, J=8.0 Hz, 1H), 7.92-7.90 (m, 1H), 7.61-7.58 (m, 1H), 7.54 (d, J=7.4 Hz, 2H), 7.42 (t, J=7.5 Hz, 2H), 7.34 (t, J=7.4 Hz, 1H), 7.28-7.27 (m, 1H), 7.23-7.19 (m, 2H), 6.60 (s, 1H), 5.28 (s, 2H), 4.59 (s, 2H), 3.96 (s, 3H), 3.93 (s, 3H), 2.95 (d, J=4.9 Hz, 3H).

### Step 2: 3′-(benzyloxy)-5-(2-(methylamino)-2-oxoethoxy)-[1,1′-biphenyl]-3,4′-dicarboxylic acid (CDD-2801)

In a microwave vial, the intermediate (15 mg, 0.0324 mmol) and triethylamnine (30 μL, 0.190 mmol) were dissolved in 1 mL acetonitrile. Lithium bromide (141 mg, 1.6 mmol) was quickly dissolved in 200 μL water and added to the reaction mixture. The reaction mixture was heated to 90° C. overnight. The reaction was monitored using LC-MS. Upon completion, the reaction mixture was dried in vacuo and purified with HPLC to yield the pure product (8 mg, 57%). Molecular Formula: C₂₄H₂₁NO₇. ¹H NMR (600 MHz, DMSO) δ 8.09 (bd, J=3.9 Hz, 1H), 7.76 (s, 1H), 7.56 (d, J=7.5 Hz, 2H), 7.42 (s, 1H), 7.36 (t, J=7.5 Hz, 2H), 7.34-7.26 (m, 2H), 7.15 (s, 1H), 7.13-7.09 (m, 2H), 5.19 (s, 2H), 4.50 (s, 2H), 2.67 (d, J=4.5 Hz, 3H). ¹³C NMR (151 MHz, DMSO) δ 168.31, 143.30, 141.77, 141.41, 139.52, 139.51, 138.03, 128.17, 127.41, 120.60, 118.66, 113.84, 113.81, 113.53, 113.50, 112.31, 99.55, 69.92, 67.13, 25.40. Calc.'d m/z [M+H]⁺: 436.1396; Obs.'d: 436.1388.

### Synthesis of 3′-(benzyloxy)-5-(2-((4-(3-(methylamino)-3-oxopropyl)phenyl)amino)-2-oxoethoxy)-[1,1′-biphenyl]-3,4′-dicarboxylic acid (CDD-2802)

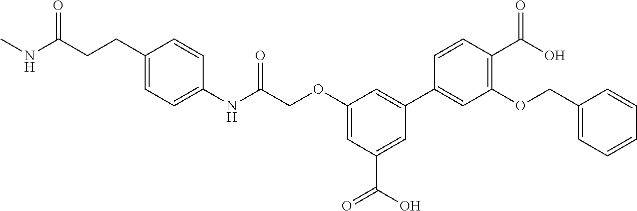 [SMILES: CNC(=O)CCc1ccc(NC(=O)COc2cc(C(=O)O)cc(-c3ccc(C(=O)O)c(OCc4ccccc4)c3)c2)cc1]

### Step 1: dimethyl 3′-(benzyloxy)-5-(2-(tert-butoxy)-2-oxoethoxy)-[1,1′-biphenyl]-3,4′-dicarboxylate

In a reaction vial, dimethyl 3′-(benzyloxy)-5-hydroxy-[1,1′-biphenyl]-3,4′-dicarboxylate (50 mg, 0.128 mmol), tert-butyl 2-bromoacetate (50 mg, 0.255 mmol), and potassium carbonate (81 mg, 0.383 mmol) were dissolved in 1 mL acetone. The reaction mixture was heated to 60° C. and rapidly stirred for 3 hr. The reaction was monitored with TLC (Rf=0.75 at 40% ethyl acetate/hexanes). Upon completion, the reaction mixture was diluted in ethyl acetate and washed with brine. The organic layer was dried over anhydrous sodium sulfate and dried in vacuo. The crude product was purified by normal phase silica gel chromatography using a hexanes/ethyl acetate gradient. Product fractions were eluted at 15% ethyl acetate/hexanes. Product fractions were collected and dried in vacuo, yielding the desired product (white oil, 63.8 mg, 99%). Molecular Formula: C₂₉H₃₀O₈. ¹H NMR (600 MHz, CDCl₃) δ 7.92 (d, J=8.4 Hz, 1H), 7.88 (s, 1H), 7.54 (d, J=7.5 Hz, 2H), 7.53 (d, J=1.1 Hz, 1H), 7.41 (t, J=7.6 Hz, 2H), 7.35-7.31 (m, 2H), 7.23-7.21 (m, 2H), 5.26 (s, 2H), 4.61 (s, 2H), 3.95 (s, 3H), 3.93 (s, 3H), 1.51 (s, 9H).

### Step 2: dimethyl 3′-(benzyloxy)-5-(2-((4-(3-(methylamino)-3-oxopropyl)phenyl)amino)-2-oxoethoxy)-[1,1′-biphenyl]-3,4′-dicarboxylate

General experimental procedure D was followed with TFA to remove tert-butyl group. No further purification was done. Subsequently, general experimental procedure A was followed with the linker (3-(4-aminophenyl)-N-methylpropanamide) (21 mg, 0.118 mmol) and the intermediate (44.5 mg, 0.0988 mmol). Molecular Formula: C₃₅H₃₄N₂O₈. ¹H NMR (600 MHz, CDCl₃) δ 11.27 (s, 1H), 8.12 (s, 1H), 7.84 (d, J=8.2 Hz, 1H), 7.65-7.59 (m, 2H), 7.47 (d, J=8.3 Hz, 2H), 7.20-7.17 (m, 3H), 7.16-7.13 (m, 1H), 6.97 (d, J=7.3 Hz, 2H), 6.89-6.85 (m, 1H), 6.82 (d, J=8.2 Hz, 1H), 5.35 (bs, 1H), 4.38 (s, 1H), 3.99 (s, 3H), 3.98-3.95 (m, 1H), 3.90 (s, 2H), 3.87-3.84 (m, 1H), 2.95 (t, J=7.6 Hz, 2H), 2.77 (d, J=4.8 Hz, 3H), 2.44 (t, J=7.6 Hz, 2H).

### Step 3: 3′-(benzyloxy)-5-(2-((4-(3-(methylamino)-3-oxopropyl)phenyl)amino)-2-oxoethoxy)-[1,1′-biphenyl]-3,4′-dicarboxylic acid (CDD-2802)

The title compound was prepared from the intermediate following general procedure E. Molecular Formula: C₃₃H₃₀N₂O₈. ¹H NMR (600 MHz, DMSO) δ 13.01 (bs, 1H), 9.99 (s, 1H), 7.73 (bd, J=4.2 Hz, 1H), 7.69 (s, 1H), 7.52-7.48 (m, 3H), 7.34 (s, 1H), 7.16-7.08 (m, 4H), 7.07-7.01 (m, 2H), 6.87 (d, J=7.4 Hz, 2H), 6.64 (s, 1H), 5.02 (s, 1H), 4.65 (s, 2H), 3.88 (s, 2H), 3.24 (s, 1H), 2.75 (t, J=7.7 Hz, 2H), 2.54 (d, J=4.4 Hz, 3H), 2.32 (t, J=7.8 Hz, 2H). Calc.'d m/z [M+H]⁺: 583.2080; Obs.'d: 583.2073.

### Synthesis of 2′-(((3-(3-aminophenyl)propyl)amino)methyl)-[1,1′-biphenyl]-3-carboxylic acid (CDD-2820)

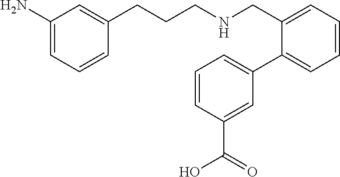 [SMILES: Nc1cccc(CCCNCc2ccccc2-c2cccc(C(=O)O)c2)c1]

Tert-butyl (3-(3-aminophenyl)propyl)carbamate was prepared following general procedure D using TFA. The crude product was used in the next reaction without further purification. General experimental procedure B was followed using the crude amine product and 2′-formyl-[1,1′-biphenyl]-3-carboxylic acid. Molecular Formula: C₂₃H₂₄N₂O₂. ¹H NMR (600 MHz, DMSO) δ 7.97 (s, 1H), 7.89 (d, J=7.5 Hz, 1H), 7.53 (d, J=7.5 Hz, 1H), 7.43 (t, J=7.5 Hz, 1H), 7.39-7.32 (m, 2H), 7.30 (t, J=7.2 Hz, 1H), 7.22 (d, J=7.3 Hz, 1H), 6.87 (t, J=7.7 Hz, 1H), 6.41 (d, J=7.8 Hz, 1H), 6.27 (d, J=7.1 Hz, 1H), 6.14 (t, J=5.4 Hz, 1H), 5.94 (s, 1H), 4.07 (d, J=5.4 Hz, 2H), 2.73 (t, J=7.3 Hz, 2H), 2.38-2.33 (m, 2H), 1.73-1.65 (m, 2H). Calc.'d m/z [M+H]⁺: 361.1916; Obs.'d: 361.1911

### Synthesis of 2′-(((3-(3-((4-(3-(methylamino)-3-oxopropyl)benzyl)amino)phenyl)propyl)amino)methyl)-[1,1′-biphenyl]-3-carboxylic acid (CDD-2819)

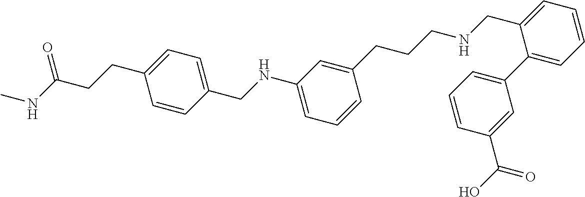 [SMILES: CNC(=O)CCc1ccc(CNc2cccc(CCCNCc3ccccc3-c3cccc(C(=O)O)c3)c2)cc1]

### Step 1: tert-butyl (3-(3-((4-(3-(methylamino)-3-oxopropyl)benzyl)amino)phenyl)propyl)carbamate

The title compound was prepared according to general procedure B using 3-(4-aminophenyl)-N-methylpropanamide and tert-butyl (3-(3-aminophenyl)propyl)carbamate. Molecular Formula: C₂₅H₃₅N₃O₃. ¹H NMR (600 MHz, CDCl₃) δ 7.27 (d, J=7.9 Hz, 2H), 7.16 (d, J=7.9 Hz, 2H), 7.07 (t, J=7.7 Hz, 1H), 6.53 (d, J=7.4 Hz, 1H), 6.46 (d, J=8.0 Hz, 1H), 6.42 (s, 1H), 5.52 (bs, 1H), 4.53 (bs, 1H), 4.27 (s, 2H), 3.13-3.07 (m, 2H), 2.95 (t, J=7.7 Hz, 2H), 2.76 (d, J=4.8 Hz, 3H), 2.53 (t, J=7.6 Hz, 2H), 2.45 (t, J=7.8 Hz, 2H), 1.78-1.71 (m, 2H), 1.44 (s, 9H).

### Step 2: 2′-(((3-(3-((4-(3-(methylamino)-3-oxopropyl)benzyl)amino)phenyl)propyl)amino)methyl)-[1,1′-biphenyl]-3-carboxylic acid (CDD-2819)

The title compound was prepared from the intermediate using general experimental procedure D with TFA. The crude product was used in the next reaction without further purification. General procedure B was followed to yield the final product. Molecular Formula: C₃₄H₃₇N₃O₃. ¹H NMR (600 MHz, DMSO) δ 7.91 (s, 1H), 7.81 (s, 1H), 7.58-7.50 (m, 2H), 7.49-7.45 (m, 1H), 7.36 (t, J=7.3 Hz, 1H), 7.32 (t, J=7.2 Hz, 1H), 7.22 (d, J=7.4 Hz, 3H), 7.11 (d, J=7.8 Hz, 2H), 6.88 (t, J=7.7 Hz, 1H), 6.38 (s, 1H), 6.32 (d, J=7.8 Hz, 1H), 6.28 (d, J=7.4 Hz, 1H), 4.16 (s, 2H), 3.56 (s, 2H), 3.16 (s, 1H), 2.77-2.73 (m, 2H), 2.62-2.60 (m, 1H), 2.54 (bd, J=4.4 Hz, 3H), 2.39-2.35 (m, 4H), 2.33-2.30 (m, 2H), 1.59-1.51 (m, 2H). Calc.'d m/z [M+H]⁺: 536.2913; Obs.'d: 536.2922.

### Synthesis of 2′-(((3-cyano-4-(4-(dimethylcarbamoyl)piperidin-1-yl)benzyl)amino)methyl)-[1,1′-biphenyl]-3-carboxylic acid (CDD-2836)

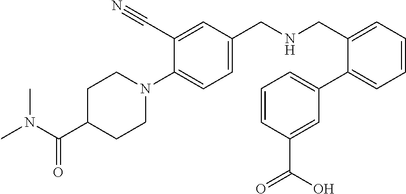 [SMILES: CN(C)C(=O)C1CCN(c2ccc(CNCc3ccccc3-c3cccc(C(=O)O)c3)cc2C#N)CC1]

### Step 1: tert-butyl 4-(dimethylcarbamoyl)piperidine-1-carboxylate

General experimental procedure A was followed with 1-(tert-butoxycarbonyl)piperidine-4-carboxylic acid (200 mg, 0.873 mmol) and 2M dimethylamine in THF (524 μL, 1.05 mmol). The crude product was used in the next reaction without further purification. Molecular Formula: C₁₃H₂₄N₂O₃. ¹H NMR (600 MHz, MeOD) δ 4.15-4.09 (m, 4H), 3.37 (s, 1H), 3.15 (s, 3H), 2.94 (s, 3H), 2.03 (s, 2H), 1.71 (d, J=12.9 Hz, 2H), 1.61-1.55 (m, 2H), 1.48 (s, 9H).

### Step 2: 1-(2-cyano-4-formylphenyl)-N,N-dimethylpiperidine-4-carboxamide

Tert-butyl 4-(dimethylcarbamoyl)piperidine-1-carboxylate was deprotected following general experimental procedure D with TFA. Then, in a microwave vial, the deprotected linker (50 mg, 0.321 mmol) was dissolved in 1 mL 1,4-dioxane followed by 2-bromo-5-formylbenzonitrile (101 mg, 0.481 mmol), potassium tert-butoxide (72 mg, 0.642 mmol), and BrettPhos Pd G4 (30 mg, 0.0321 mmol). The reaction mixture was thoroughly degassed under N₂ gas and heated to 100° C. overnight. The reaction was monitored with TLC (Rf=0.1 at 70% ethyl acetate/hexanes). The reaction mixture was filtered using a 0.45 μm PTFE syringe filter and washed with brine and ethyl acetate. The organic layer was dried over anhydrous sodium sulfate and dried in vacuo. The crude product was purified by normal phase silica gel chromatography using a hexanes/ethyl acetate gradient. Product fractions were eluted at 100% ethyl acetate. Product fractions were collected and dried in vacuo, yielding the desired product (yellow oil, 50 mg, 55%). Molecular Formula: C₁₆H₁₉N₃O₂. ¹H NMR (800 MHz, CDCl₃) δ 9.80 (s, 1H), 8.00 (d, J=2.0 Hz, 1H), 7.90 (dd, J=8.8, 2.1 Hz, 1H), 7.02 (d, J=8.8 Hz, 1H), 3.98 (dt, J=12.9, 3.2 Hz, 2H), 3.21-3.16 (m, 2H), 3.09 (s, 3H), 2.97 (s, 3H), 2.81 (tt, J=10.4, 4.1 Hz, 1H), 2.04 (qd, J=11.2, 3.7 Hz, 2H), 1.89 (dd, J=13.8, 3.4 Hz, 2H).

### Step 3: 2′-(((tert-butoxycarbonyl)amino)methyl)-[1,1′-biphenyl]-3-carboxylic acid

2′-formyl-[1,1′-biphenyl]-3-carboxylic acid (50 mg, 0.221 mmol) and tert-butyl carbamate (77 mg, 0.664 mmol) were dissolved in 1:3 dichloromethane:acetonitrile solution (1 mL:3 mL), followed by slow addition of triethylsilane (105 μL, 0.664 mmol). The reaction mixture was stirred vigorously as trifluoroacetic acid (34 μL, 0.442 mmol) was slowly added dropwise. The reaction mixture was left stirring at room temperature overnight. The reaction was monitored by TLC (Rf=0.25 at 10% methanol/dichloromethane). The product Rf measured on TLC was indistinguishable from starting material but did not stain with DNP when the starting material did stain with DNP. The reaction mixture was diluted with ethyl acetate and washed with brine. The organic layer was dried over anhydrous sodium sulfate and dried in vacuo. The crude product was purified by normal phase silica gel chromatography using a methanol/dichloromethane gradient. Product fractions were eluted at 10% methanol/dichloromethane. Product fractions were collected and dried in vacuo, yielding the desired product (35 mg, 48%). Molecular Formula: C₁₉H₂₁NO₄. ¹H NMR (600 MHz, CDCl₃) δ 8.07 (bs, 1H), 8.03 (s, 1H), 7.51 (d, J=16.2 Hz, 2H), 7.45 (d, J=6.0 Hz, 1H), 7.40-7.35 (m, 1H), 7.34-7.30 (m, 1H), 7.23 (d, J=5.4 Hz, 1H), 5.03 (d, J=394.9 Hz, 1H), 4.23 (d, J=44.6 Hz, 2H), 1.38 (d, J=38.7 Hz, 9H).

### Step 4: 2′-(((3-cyano-4-(4-(dimethylcarbamoyl)piperidin-1-yl)benzyl)amino)methyl)-[1,1′-biphenyl]-3-carboxylic acid (CDD-2836)

The title compound was prepared by deprotection of the intermediate according to general procedure D using TFA. The crude product was subjected to general procedure B without purification to obtain the final product. Molecular Formula: C₃₀H₃₂N₄O₃. ¹H NMR (600 MHz, DMSO) δ 7.84-7.78 (m, 2H), 7.57 (d, J=7.6 Hz, 1H), 7.54 (d, J=1.5 Hz, 1H), 7.42 (d, J=8.4 Hz, 1H), 7.34 (td, J=7.5, 1.3 Hz, 1H), 7.30 (td, J=7.4, 1.2 Hz, 1H), 7.27-7.23 (m, 2H), 7.18 (d, J=7.4 Hz, 1H), 7.04 (d, J=8.5 Hz, 1H), 5.02 (s, 1H), 3.54 (d, J=12.4 Hz, 3H), 3.44 (d, J=11.8 Hz, 2H), 3.05 (s, 3H), 2.85-2.75 (m, 6H), 2.52-2.51 (m, 1H), 1.77-1.71 (m, 4H). Calc.'d m/z [M+H]⁺: 497.2553; Obs.'d: 497.2545.

### Synthesis of 2-hydroxy-5-((((5-(4-(3-(methylamino)-3-oxopropyl)piperidine-1-carbonyl)thiophen-2-yl)methyl)amino)methyl)benzoic acid (CDD-2371)

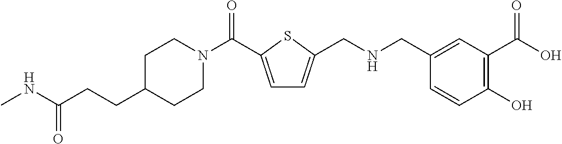 [SMILES: CNC(=O)CCC1CCN(C(=O)c2ccc(CNCc3ccc(O)c(C(=O)O)c3)s2)CC1]

### Step 1: tert-butyl 4-(3-(methylamino)-3-oxopropyl)piperidine-1-carboxylate

The title compound was prepared according to general procedure A. Molecular Formula: C₁₄H₂₆N₂O₃. ¹H NMR (600 MHz, CDCl₃) δ 5.83 (bs, 1H), 4.04 (bs, 2H), 2.77 (d, J=4.8 Hz, 3H), 2.63 (bs, 2H), 2.17 (t, J=7.8 Hz, 2H), 1.62 (d, J=12.8 Hz, 2H), 1.56 (s, 2H), 1.42 (s, 9H), 1.06 (ddd, J=24.6, 12.5, 4.4 Hz, 2H).

### Step 2: tert-butyl ((5-(4-(3-(methylamino)-3-oxopropyl)piperidine-1-carbonyl)thiophen-2-yl)methyl)carbamate

The title compound was prepared according to general procedure D using TFA to protect the intermediate without further purification. General experimental procedure A was subsequently followed. Molecular Formula: C₂₀H₃₁N₃O₄S. ¹H NMR (600 MHz, MeOD) δ 7.90 (s, 1H), 7.32-7.27 (m, 1H), 7.19 (d, J=3.6 Hz, 1H), 6.93 (d, J=3.7 Hz, 1H), 4.51-4.27 (m, 4H), 3.37-3.30 (m, 1H), 3.01 (vbs, 2H), 2.72-2.69 (m, 3H), 2.23 (t, J=7.4 Hz, 2H), 1.80 (d, J=12.7 Hz, 2H), 1.59 (t, J=5.8 Hz, 3H), 1.45 (s, 9H), 1.19 (qd, J=13.1, 4.2 Hz, 2H).

### Step 3: methyl 2-hydroxy-5-((((5-(4-(3-(methylamino)-3-oxopropyl)piperidine-1-carbonyl)thiophen-2-yl)methyl)amino)methyl)benzoate

The title compound was prepared according to general procedure D using TFA to deprotect the intermediate without further purification. General experimental procedure B was subsequently followed. Molecular Formula: C₂₄H₃₁N₃O₅S. ¹H NMR (600 MHz, MeOD) δ 7.83 (d, J=2.1 Hz, 1H), 7.48 (dd, J=8.5, 2.2 Hz, 1H), 7.22 (d, J=3.6 Hz, 1H), 6.96 (d, J=3.6 Hz, 1H), 6.92 (d, J=8.5 Hz, 1H), 4.39 (vbs, 2H), 3.95 (s, 3H), 3.93 (s, 2H), 3.71 (s, 2H), 3.00 (vbs, 2H), 2.71 (s, 3H), 2.23 (t, J=7.4 Hz, 2H), 1.81 (d, J=12.7 Hz, 2H), 1.63-1.56 (m, 3H), 1.23-1.15 (m, 2H).

### Step 4: 2-hydroxy-5-((((5-(4-(3-(methylamino)-3-oxopropyl)piperidine-1-carbonyl)thiophen-2-yl)methyl)amino)methyl)benzoic acid (CDD-2371)

The title compound was prepared from the intermediate according to general procedure E. General experimental procedure E was followed. Molecular Formula: C₂₃H₂₉N₃O₅S. ¹H NMR (600 MHz, DMSO) δ 9.27 (vbs, 1H), 7.90 (s, 1H), 7.79-7.71 (m, 1H), 7.30-7.27 (m, 1H), 7.27-7.18 (m, 2H), 6.67 (d, J=8.2 Hz, 1H), 4.33 (s, 2H), 4.15 (bm, 2H), 3.97 (s, 2H), 2.93 (vbs, 2H), 2.55 (d, J=4.5 Hz, 3H), 2.08 (t, J=7.3 Hz, 2H), 1.70 (d, J=12.3 Hz, 2H), 1.52-1.41 (m, 3H), 1.12-1.01 (m, 2H). ¹³C NMR (151 MHz, DMSO) δ 172.41, 171.29, 163.40, 161.65, 138.61, 133.52, 132.21, 129.33, 128.46, 120.04, 115.94, 76.39, 50.17, 48.58, 44.06, 40.06, 39.52, 34.97, 32.50, 31.76, 31.68, 25.42. Calc.'d m/z [M+H]⁺: 460.1906; Obs.'d: 460.1900.

### Synthesis of 2-hydroxy-5-(((5-((4-(methylcarbamoyl)cyclohexane-1-carboxamido)methyl)pyridin-2-yl)amino)methyl)benzoic acid (CDD-2395)

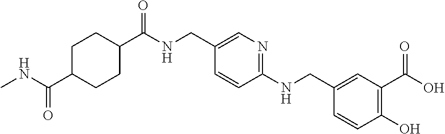 [SMILES: CNC(=O)C1CCC(C(=O)NCc2ccc(NCc3ccc(O)c(C(=O)O)c3)nc2)CC1]

### Step 1: 4-(methylcarbamoyl)cyclohexane-1-carboxylic acid

General experimental procedure A was followed with 4-(methoxycarbonyl)cyclohexane-1-carboxylic acid and 2M methylamine in THF. Then, general procedure E was followed to hydrolyze the ester. Molecular Formula: C₉H₁₅NO₃. ¹H NMR (600 MHz, CDCl₃) δ 4.97-4.91 (m, 4H), 4.66 (s, 4H), 2.75-2.65 (m, 3H), 1.94 (dd, J=90.5, 10.3 Hz, 1H), 1.54-1.35 (m, 1H).

### Step 2: tert-butyl (5-((4-(methylcarbamoyl)cyclohexane-1-carboxamido)methyl)pyridin-2-yl)carbamate

The title compound was prepared following general procedure A. Molecular Formula: C₂₀H₃₀N₄O₄. ¹H NMR (600 MHz, MeOD) δ 8.13 (d, J=1.8 Hz, 1H), 7.80 (d, J=8.6 Hz, 1H), 7.64 (dd, J=8.6, 2.4 Hz, 1H), 4.29 (s, 2H), 2.69 (s, 3H), 2.26-2.12 (m, 2H), 1.86 (td, J=9.4, 3.7 Hz, 4H), 1.53 (s, 9H), 1.50 (dd, J=9.9, 5.2 Hz, 3H).

### Step 3: 2-hydroxy-5-(((5-((4-(methylcarbamoyl)cyclohexane-1-carboxamido)methyl)pyridin-2-yl)amino)methyl)benzoic acid (CDD-2395)

The title compound was prepared following general procedure D using TFA to deprotect the intermediate without further purification. General experimental procedures B, then E were subsequently followed. Molecular Formula: C₂₃H₂₈N₄O₅. ¹H NMR (600 MHz, MeOD) δ 7.86-7.76 (m, 2H), 7.42-7.24 (m, 2H), 6.85-6.73 (m, 1H), 6.53 (dd, J=25.6, 8.6 Hz, 1H), 4.51-3.89 (m, 4H), 2.69 (s, 3H), 2.22-2.13 (m, 2H), 1.85 (d, J=8.5 Hz, 4H), 1.55-1.44 (m, 4H). Calc.'d m/z [M+H]⁺: 441.2138; Obs.'d: 441.2130.

### Synthesis of 4-(5-(((3-(aminomethyl)phenyl)amino)methyl)pyridin-3-yl)-2-hydroxybenzoic acid (CDD-2653)

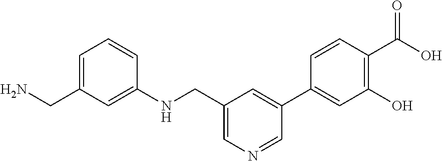 [SMILES: NCc1cccc(NCc2cncc(-c3ccc(C(=O)O)c(O)c3)c2)c1]

### Step 1: methyl 4-(6-formylpyridin-2-yl)-2-hydroxybenzoate

The title compound was prepared according to general procedure C. Molecular Formula: C₁₄H₁₁NO₄. ¹H NMR (600 MHz, CDCl₃) δ 10.84 (s, 1H), 10.17 (s, 1H), 8.01-7.94 (m, 4H), 7.71 (d, J=1.3 Hz, 1H), 7.66 (dd, J=8.3, 1.6 Hz, 1H), 4.00 (s, 3H).

### Step 2: methyl 4-(6-(((3-(((tert-butoxycarbonyl)amino)methyl)phenyl)amino)methyl)pyridin-2-yl)-2-hydroxybenzoate

The title compound was prepared from the intermediate according to general procedure B. Molecular Formula: C₂₆H₂₉N₃O₅. ¹H NMR (600 MHz, CDCl₃) δ 10.82 (s, 1H), 7.94 (d, J=8.3 Hz, 1H), 7.73 (t, J=7.7 Hz, 1H), 7.66-7.62 (m, 2H), 7.58 (dd, J=8.3, 1.4 Hz, 1H), 7.30 (d, J=7.6 Hz, 1H), 7.14 (t, J=8.0 Hz, 1H), 6.67-6.63 (m, 2H), 6.60 (d, J=7.5 Hz, 1H), 4.85 (s, 1H), 4.52 (s, 2H), 4.28-4.19 (m, 1H), 3.98 (s, 3H), 1.45 (s, 10H).

### Step 3: 4-(5-(((3-(aminomethyl)phenyl)amino)methyl)pyridin-3-yl)-2-hydroxybenzoic acid (CDD-2653)

The title compound was prepared from the intermediate according to general procedure D using HCl, followed by general procedure E. Molecular Formula: C₂₀H₁₉N₃O₃. ¹H NMR (600 MHz, DMSO) δ 7.91 (s, 3H), 7.80-7.71 (m, 3H), 7.39-7.32 (m, 2H), 7.26 (d, J=7.3 Hz, 1H), 7.09 (t, J=7.8 Hz, 1H), 6.74 (s, 1H), 6.62 (dd, J=14.3, 7.5 Hz, 2H), 6.51 (t, J=6.0 Hz, 1H), 4.44 (d, J=6.1 Hz, 2H), 3.86 (s, 2H). Calc.'d m/z [M+H]⁺: 350.1505; Obs.'d: 350.1489.

### Synthesis of 5-(((3-(aminomethyl)phenyl)amino)methyl)-3′-hydroxy-[1,1′-biphenyl]-3,4′-dicarboxylic acid (CDD-2897)

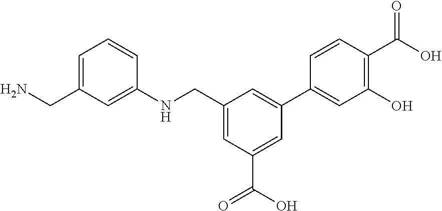 [SMILES: NCc1cccc(NCc2cc(C(=O)O)cc(-c3ccc(C(=O)O)c(O)c3)c2)c1]

### Step 1: dimethyl 5-formyl-3′-hydroxy-[1,1′-biphenyl]-3,4′-dicarboxylate

The title compound was prepared according to general procedure C. Molecular Formula: C₁₇H₁₄O₆. ¹H NMR (600 MHz, CDCl₃) δ 10.86 (s, 1H), 10.14 (s, 1H), 8.55 (d, J=1.7 Hz, 2H), 8.32 (t, J=1.6 Hz, 1H), 7.95 (d, J=8.2 Hz, 1H), 7.29 (d, J=1.7 Hz, 1H), 7.19 (dd, J=8.2, 1.8 Hz, 1H), 4.00 (s, 3H), 4.00 (s, 3H).

### Step 2: dimethyl 3′-hydroxy-5-(((3-((methylamino)methyl)phenyl)amino)methyl)-[1,1′-biphenyl]-3,4′-dicarboxylate

The title compound was prepared according to general procedure B. Molecular Formula: C₂₅H₂₆N₂O₅. ¹H NMR (600 MHz, CDCl₃) δ 10.82 (s, 1H), 8.20 (s, 1H), 8.07 (s, 1H), 7.90 (d, J=8.3 Hz, 1H), 7.81 (s, 1H), 7.22 (d, J=1.7 Hz, 1H), 7.15-7.13 (m, 1H), 7.13-7.10 (m, 3H), 6.69 (d, J=7.6 Hz, 2H), 6.65 (s, 3H), 6.62 (d, J=7.9 Hz, 2H), 6.59 (s, 1H), 6.53 (dd, J=8.0, 1.9 Hz, 1H), 4.79 (s, 2H), 4.44 (s, 2H), 4.23 (d, J=5.3 Hz, 6H), 3.98 (s, 3H), 3.95 (s, 3H), 2.17 (s, 1H), 2.04 (s, 7H), 1.46 (s, 17H), 1.44 (s, 9H).

### Step 3: 5-(((3-(aminomethyl)phenyl)amino)methyl)-3′-hydroxy-[1,1′-biphenyl]-3,4′-dicarboxylic acid (CDD-2897)

The title compound was prepared according to general procedure D using TFA, followed by general procedure E. Molecular Formula: C₂₂H₂₀N₂O₅. ¹H NMR (600 MHz, DMSO) δ 7.99 (s, 1H), 7.91 (s, 1H), 7.82 (s, 1H), 7.72 (d, J=6.4 Hz, 1H), 7.08 (t, J=7.3 Hz, 1H), 6.92-6.83 (m, 2H), 6.72 (s, 1H), 6.64-6.52 (m, 3H), 4.40 (s, 2H), 3.86 (s, 2H). Calc.'d m/z [M+H]⁺: 393.1450; Obs.'d: 393.1447

### Synthesis of 3-hydroxy-3′-(((3-((methylamino)methyl)phenyl)amino)methyl)-[1,1′-biphenyl]-4-carboxylic acid (CDD-2906)

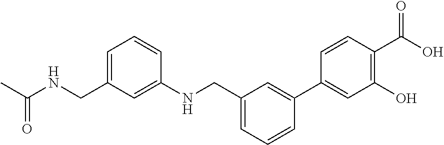 [SMILES: CC(=O)NCc1cccc(NCc2cccc(-c3ccc(C(=O)O)c(O)c3)c2)c1]

### Step 1: methyl 3′-(((3-(((tert-butoxycarbonyl)(methyl)amino)methyl)phenyl)amino)methyl)-3-hydroxy-[1,1′-biphenyl]-4-carboxylate

The title compound was prepared according to general procedure B. Molecular Formula: C₂₈H₃₂N₂O₅. ¹H NMR (600 MHz, CDCl₃) δ 10.80 (s, 1H), 7.87 (d, J=8.3 Hz, 1H), 7.62 (s, 1H), 7.52 (d, J=7.2 Hz, 1H), 7.44-7.38 (m, 2H), 7.19 (d, J=1.7 Hz, 1H), 7.14 (t, J=7.8 Hz, 1H), 7.11 (dd, J=8.3, 1.7 Hz, 1H), 6.70-6.51 (m, 3H), 4.38 (s, 2H), 4.34 (d, J=15.9 Hz, 2H), 3.97 (s, 3H), 2.76 (d, J=33.5 Hz, 3H), 1.46 (d, J=15.2 Hz, 9H).

### Step 2: 3-hydroxy-3′-(((3-((methylamino)methyl)phenyl)amino)methyl)-[1,1′-biphenyl]-4-carboxylic acid (CDD-2906)

The title compound was prepared from the intermediate according to general procedure D using TFA, followed by general procedure E. Molecular Formula: C₂₂H₂₂N₂O₃. ¹H NMR (600 MHz, DMSO) δ 8.00 (s, 1H), 7.68 (d, J=7.8 Hz, 1H), 7.61 (s, 1H), 7.47 (d, J=7.7 Hz, 1H), 7.37 (t, J=7.6 Hz, 1H), 7.31 (d, J=7.6 Hz, 1H), 7.09 (t, J=7.8 Hz, 1H), 6.83 (dd, J=9.6, 1.7 Hz, 2H), 6.68 (s, 1H), 6.62 (dd, J=8.1, 1.6 Hz, 1H), 6.58 (d, J=7.4 Hz, 1H), 6.51 (t, J=6.1 Hz, 1H), 4.34 (d, J=6.1 Hz, 2H), 3.91 (s, 2H), 2.48 (s, 3H). Calc.'d m/z [M+H]⁺: 363.1709; Obs.'d: 363.1702.

### Synthesis of 3-hydroxy-3′-((m-tolylamino)methyl)-[1,1′-biphenyl]-4-carboxylic acid (CDD-2907)

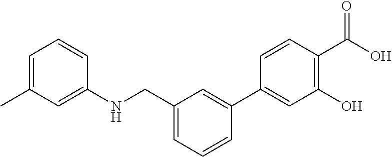 [SMILES: Cc1cccc(NCc2cccc(-c3ccc(C(=O)O)c(O)c3)c2)c1]

### Step 1: methyl 3-hydroxy-3′-((m-tolylamino)methyl)-[1,1′-biphenyl]-4-carboxylate

The title compound was prepared according to general procedure B. Molecular Formula: C₂₂H₂₁NO₃. ¹H NMR (600 MHz, CDCl₃) δ 10.81 (s, 1H), 7.88 (d, J=8.3 Hz, 1H), 7.63 (s, 1H), 7.52 (dt, J=6.8, 2.0 Hz, 1H), 7.44-7.39 (m, 2H), 7.21 (d, J=1.7 Hz, 1H), 7.12 (dd, J=8.3, 1.8 Hz, 1H), 7.08 (t, J=7.7 Hz, 1H), 6.59 (d, J=7.4 Hz, 1H), 6.53 (s, 1H), 6.50 (d, J=8.0 Hz, 1H), 4.39 (s, 2H), 3.97 (s, 3H), 2.28 (s, 3H).

### Step 2: 3-hydroxy-3′-((m-tolylamino)methyl)-[1,1′-biphenyl]-4-carboxylic acid (CDD-2907)

The title compound was prepared from the intermediate according to general procedure E. Molecular Formula: C₂₁H₁₉NO₃. ¹H NMR (600 MHz, DMSO) δ 7.85 (d, J=8.0 Hz, 1H), 7.71 (s, 1H), 7.58-7.55 (m, 1H), 7.44-7.38 (m, 2H), 7.23-7.18 (m, 2H), 6.91 (t, J=7.7 Hz, 1H), 6.44 (s, 1H), 6.41-6.37 (m, 1H), 6.33 (d, J=7.4 Hz, 1H), 4.32 (s, 2H), 2.15 (s, 3H). Calc.'d m/z [M+H]⁺: 334.1443; Obs.'d: 334.1432.

### Synthesis of 3′-(((3-(acetamidomethyl)phenyl)amino)methyl)-3-hydroxy-[1,1′-biphenyl]-4-carboxylic acid (CDD-2919)

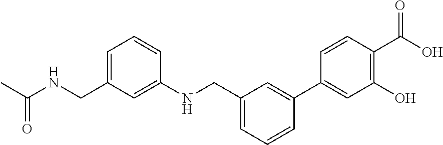 [SMILES: CC(=O)NCc1cccc(NCc2cccc(-c3ccc(C(=O)O)c(O)c3)c2)c1]

### Step 1: tert-butyl (3-(acetamidomethyl)phenyl)carbamate

Tert-butyl (3-(aminomethyl)phenyl)carbamate (150 mg, 0.676 mmol) and diisopropylethylamine (235 μL, 1.35 mmol) were dissolved in 5 mL dichloromethane and chilled to 0° C. While stirring, acyl chloride (64 mg, 0.811 mmol) was added dropwise. The reaction was stirred at 0° C. for 1 hr. and monitored by TLC. The reaction mixture was diluted with ethyl acetate and washed with concentrated sodium bicarbonate. The organic layer was dried over anhydrous sodium sulfate and dried in vacuo. The crude product was purified by normal phase silica gel chromatography using a hexanes/ethyl acetate gradient. Product fractions were eluted at 70% ethyl acetate. Product fractions were collected and dried in vacuo, yielding the desired product (166 mg, 93%). Molecular Formula: C₁₄H₂₀N₂O₃. ¹H NMR (600 MHz, CDCl₃) δ 7.37 (s, 1H), 7.24 (d, J=7.6 Hz, 1H), 7.21 (d, J=8.0 Hz, 1H), 6.96 (d, J=7.3 Hz, 1H), 6.49 (s, 1H), 5.76 (s, 1H), 4.40 (d, J=5.6 Hz, 2H), 2.02 (s, 3H), 1.51 (s, 9H).

### Step 2: methyl 3′-(((3-(acetamidomethyl)phenyl)amino)methyl)-3-hydroxy-[1,1′-biphenyl]-4-carboxylate

The intermediate was deprotected using general experimental procedure D with TFA without further purification. Then, general experimental procedure B was followed. Chemical Formula: C₂₄H₂₄N₂O₄. ¹H NMR (600 MHz, CDCl₃) δ 10.81 (s, 1H), 7.88 (d, J=8.2 Hz, 1H), 7.61 (s, 1H), 7.53 (d, J=7.6 Hz, 1H), 7.43 (t, J=7.6 Hz, 1H), 7.39 (d, J=7.6 Hz, 1H), 7.19 (d, J=1.6 Hz, 1H), 7.15-7.10 (m, 2H), 6.63 (d, J=7.5 Hz, 1H), 6.59-6.55 (m, 2H), 4.40 (s, 2H), 4.34 (d, J=5.6 Hz, 2H), 3.99 (d, J=1.9 Hz, 1H), 3.97 (s, 3H), 1.99 (s, 3H).

### Step 3: 3′-(((3-(acetamidomethyl)phenyl)amino)methyl)-3-hydroxy-[1,1′-biphenyl]-4-carboxylic acid (CDD-2919)

The title compound was prepared according to general procedure E. Molecular Formula: C₂₃H₂₂N₂O₄. ¹H NMR (600 MHz, DMSO) δ 8.20 (t, J=5.7 Hz, 1H), 7.82 (d, J=8.6 Hz, 1H), 7.69 (s, 1H), 7.55 (d, J=7.4 Hz, 1H), 7.43-7.36 (m, 2H), 7.18-7.12 (m, 2H), 6.97 (t, J=7.8 Hz, 1H), 6.55-6.51 (m, 1H), 6.45 (dd, J=8.1, 1.7 Hz, 1H), 6.40 (d, J=7.5 Hz, 1H), 5.76 (s, 1H), 4.31 (s, 2H), 4.09 (d, J=5.9 Hz, 2H), 1.81 (s, 3H). ¹³C NMR (151 MHz, DMSO) δ 148.67, 140.09, 131.94, 130.78, 128.90, 128.78, 125.80, 125.20, 121.22, 121.16, 120.54, 115.00, 114.56, 111.51, 110.58, 99.51, 89.62, 46.40, 42.35, 39.52, 22.53. Calc.'d m/z [M+H]⁺: 391.1658; Obs.'d: 391.1645.

### Synthesis of 3-hydroxy-3′-(((3-(2-(methylamino)-2-oxoethyl)phenyl)amino)methyl)-[1,1′-biphenyl]-4-carboxylic acid (CDD-2920)

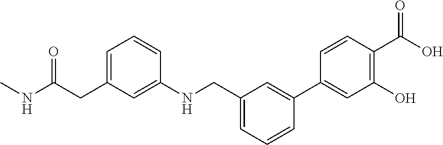 [SMILES: CNC(=O)Cc1cccc(NCc2cccc(-c3ccc(C(=O)O)c(O)c3)c2)c1]

### Step 1: tert-butyl (3-(2-(methylamino)-2-oxoethyl)phenyl)carbamate

The title compound was prepared according to general procedure A using 2-(3-((tert-butoxycarbonyl)amino)phenyl)acetic acid and 2M methylamine in THF. Molecular Formula: C₁₄H₂₀N₂O₃. ¹H NMR (600 MHz, MeOD) δ 7.34 (s, 1H), 7.28 (d, J=8.1 Hz, 1H), 7.20 (t, J=7.8 Hz, 1H), 6.93-6.90 (m, 1H), 3.45 (s, 2H), 2.71 (s, 3H), 1.51 (s, 9H).

### Step 2: methyl 3-hydroxy-3′-(((3-(2-(methylamino)-2-oxoethyl)phenyl)amino)methyl)-[1,1′-biphenyl]-4-carboxylate

The title compound was prepared by deprotection of the intermediate using general procedure D with TFA without further purification, and subsequently general procedure B. Molecular Formula: C₂₄H₂₄N₂O₄. ¹H NMR (600 MHz, CDCl₃) δ 10.81 (s, 1H), 7.88 (d, J=8.3 Hz, 1H), 7.61 (s, 1H), 7.53 (d, J=7.6 Hz, 1H), 7.43 (t, J=7.6 Hz, 1H), 7.39 (d, J=7.6 Hz, 1H), 7.20 (d, J=1.7 Hz, 1H), 7.16 (t, J=7.8 Hz, 1H), 7.11 (dd, J=8.3, 1.7 Hz, 1H), 6.60 (d, J=7.9 Hz, 2H), 6.53 (s, 1H), 5.41 (s, 1H), 4.40 (s, 2H), 3.97 (s, 3H), 3.49 (s, 2H), 2.71 (d, J=4.9 Hz, 3H).

### Step 3: 3-hydroxy-3′-(((3-(2-(methylamino)-2-oxoethyl)phenyl)amino)methyl)[1,1′-biphenyl]-4-carboxylic acid (CDD-2920)

The title compound was prepared from the intermediate according to general procedure E. Molecular Formula: C₂₃H₂₂N₂O₄. ¹H NMR (600 MHz, DMSO) δ 7.85-7.78 (m, 2H), 7.70 (s, 1H), 7.55 (d, J=7.3 Hz, 1H), 7.43-7.37 (m, 2H), 7.16 (s, 2H), 6.94 (t, J=7.8 Hz, 1H), 6.55-6.52 (m, 1H), 6.43 (dd, J=7.9, 1.8 Hz, 1H), 6.41 (d, J=7.5 Hz, 1H), 5.76 (s, 1H), 4.31 (s, 2H), 3.21 (s, 2H), 2.53 (d, J=4.6 Hz, 3H). Calc.'d m/z [M+H]⁺: 391.1658; Obs.'d: 391.1648.

### Synthesis of 3′-(((3-(aminomethyl)-5-hydroxyphenyl)amino)methyl)-3-hydroxy-[1,1′-biphenyl]-4-carboxylic acid (CDD-2974)

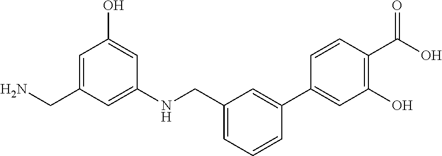 [SMILES: NCc1cc(O)cc(NCc2cccc(-c3ccc(C(=O)O)c(O)c3)c2)c1]

### Step 1: tert-butyl (3-hydroxy-5-nitrobenzyl)carbamate

3-hydroxy-5-nitrobenzaldehyde (85 mg, 0.510 mmol) and tert-butyl carbamate (179 mg, 1.53 mmol) were dissolved in 1:3 dichloromethane:acetonitrile solution (1 mL:3 mL), followed by slow addition of triethylsilane (244 NL, 1.53 mmol). The reaction mixture was stirred vigorously as trifluoroacetic acid (79 μL, 1.02 mmol) was slowly added dropwise. The reaction mixture was left stirring at room temperature overnight. The reaction was monitored by TLC (Rf=0.18 at 10% methanol/dichloromethane). The reaction mixture was diluted with ethyl acetate and washed with brine. The organic layer was dried over anhydrous sodium sulfate and dried in vacuo. The crude product was purified by normal phase silica gel chromatography using a methanol/dichloromethane gradient. Product fractions were eluted at 20% methanol/dichloromethane. Product fractions were collected and dried in vacuo, yielding the desired product (74 mg, 54%). Molecular Formula: C₁₂H₁₆N₂O₅. ¹H NMR (600 MHz, CDCl₃) δ 7.59 (s, 1H), 7.42 (s, 1H), 6.99 (s, 11H), 5.25 (s, 11H), 4.32 (d, J=6.1 Hz, 2H), 1.50 (s, 9H).

### Step 2: tert-butyl (3-amino-5-hydroxybenzyl)carbamate

Intermediate (33 mg, 0.123 mmol) was dissolved in 2 mL ethanol and added zinc powder (32 mg, 0.493 mmol) and acetic acid (14 μL, 0.246 mmol). Reaction mixture was stirred at room temperature overnight. The reaction was monitored with TLC (Rf=0.46 at 10% methanol/dichloromethane). Upon completion, the reaction mixture was filtered using a 0.45 μm PTFE syringe filter and died in vacuo. The crude product was purified using normal phase silica gel chromatography using a methanol/dichloromethane gradient. Product fractions were collected and dried in vacuo (9 mg, 30%). Molecular Formula: C₁₂H₁₈N₂O₃. ¹H NMR (600 MHz, CDCl₃) δ 6.20-6.13 (m, 2H), 6.09 (s, 11H), 5.30 (s, 1H), 4.84 (s, 1H), 4.15 (d, J=5.1 Hz, 2H), 1.46 (s, 9H).

### Step 3: methyl 3′-(((3-(((tert-butoxycarbonyl)amino)methyl)-5-hydroxyphenyl)amino)methyl)-3-hydroxy-[1,1′-biphenyl]-4-carboxylate

The title compound was prepared from the intermediate according to general procedure B. Molecular Formula: C₂₇H₃₀N₂O₆. ¹H NMR (600 MHz, CDCl₃) δ 10.81 (s, 1H), 7.88 (d, J=8.3 Hz, 1H), 7.58 (s, 1H), 7.52 (d, J=7.6 Hz, 1H), 7.42 (t, J=7.6 Hz, 1H), 7.37 (d, J=7.5 Hz, 1H), 7.19 (d, J=1.2 Hz, 1H), 7.11 (dd, J=8.3, 1.4 Hz, 1H), 6.15 (d, J=15.6 Hz, 2H), 6.05-6.01 (m, 1H), 4.34 (s, 2H), 4.16 (d, J=5.4 Hz, 2H), 3.97 (s, 3H), 1.44 (s, 9H).

### Step 4: 3′-(((3-(aminomethyl)-5-hydroxyphenyl)amino)methyl)-3-hydroxy-[1,1′-biphenyl]-4-carboxylic acid (CDD-2974)

The title compound was prepared according to general procedure D using TFA, followed by general experimental procedure E. Molecular Formula: C₂₁H₂₀N₂O₄. ¹H NMR (600 MHz, DMSO): δ 9.16 (s, 1H), 8.04 (s, 3H), 7.70 (d, J=7.7 Hz, 1H), 7.60 (s, 1H), 7.47 (d, J=7.7 Hz, 1H), 7.37 (t, J=7.6 Hz, 1H), 7.30 (d, J=7.6 Hz, 1H), 6.86 (d, J=10.3 Hz, 2H), 6.40 (s, 1H), 6.18 (s, 1H), 6.05-5.98 (m, 2H), 4.29 (d, J=3.0 Hz, 2H), 3.76 (s, 2H). ¹³C NMR (151 MHz, DMSO) δ 172.21, 161.91, 158.86, 150.52, 147.74, 145.01, 141.60, 139.12, 135.73, 131.39, 129.51, 127.89, 126.18, 125.81, 118.21, 115.18, 104.63, 104.15, 99.58, 46.73, 43.19, 26.63. Calc.'d m/z [M+H]⁺: 365.1501; Obs.'d: 365.1494.

### Synthesis of 3′-(((5-(aminomethyl)pyridin-3-yl)amino)methyl)-3-hydroxy-[1,1′-biphenyl]-4-carboxylic acid (CDD-2975)

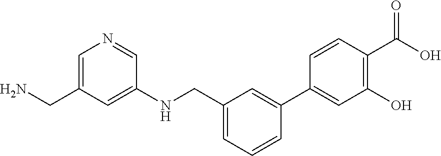 [SMILES: NCc1cncc(NCc2cccc(-c3ccc(C(=O)O)c(O)c3)c2)c1]

### Step 1: methyl 3′-(((5-(((tert-butoxycarbonyl)amino)methyl)pyridin-3-yl)amino)methyl)-3-hydroxy-[1,1′-biphenyl]-4-carboxylate

The title compound was prepared according to general procedure B. Molecular Formula: C₂₆H₂₉N₃O₅. ¹H NMR (600 MHz, CDCl₃) δ 10.81 (s, 1H), 8.00 (bs, 1H), 7.96-7.87 (m, 2H), 7.60 (s, 1H), 7.54 (d, J=6.7 Hz, 1H), 7.49-7.42 (m, 1H), 7.42-7.35 (m, 1H), 7.27 (s, 3H), 7.20 (s, 1H), 7.11 (d, J=8.1 Hz, 1H), 6.89 (s, 1H), 4.85 (s, 1H), 4.41 (s, 2H), 4.25 (s, 3H), 3.97 (d, J=3.1 Hz, 3H), 1.44 (s, 9H).

### Step 2: 3′-(((5-(aminomethyl)pyridin-3-yl)amino)methyl)-3-hydroxy-[1,1′-biphenyl]-4-carboxylic acid (CDD-2975)

The title compound was prepared from the intermediate according to general procedure D using TFA, followed by general experimental procedure E. Molecular Formula: C₂₀H₁₉N₃O₃. ¹H NMR (600 MHz, DMSO) δ 10.09 (s, 1H), 8.07 (s, 2H), 8.01 (d, J=2.5 Hz, 1H), 7.81 (d, J=0.8 Hz, 1H), 7.73 (d, J=8.1 Hz, 1H), 7.65 (s, 1H), 7.52 (d, J=6.8 Hz, 1H), 7.40 (t, J=7.6 Hz, 1H), 7.35 (d, J=7.0 Hz, 1H), 7.06-7.02 (m, 1H), 6.94 (bs, 2H), 6.77 (t, J=6.2 Hz, 1H), 4.38 (d, J=6.2 Hz, 2H), 3.92 (s, 2H). Calc.'d m/z [M+H]⁺: 350.1505; Obs.'d: 350.1494.

### Synthesis of 3′-((3-(aminomethyl)phenyl)carbamoyl)-3-hydroxy-[1,1′-biphenyl]-4-carboxylic acid (CDD-2985)

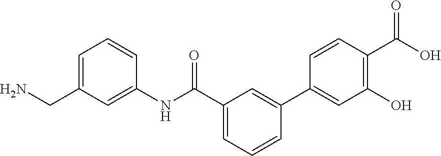 [SMILES: NCc1cccc(NC(=O)c2cccc(-c3ccc(C(=O)O)c(O)c3)c2)c1]

### Step 1: methyl 3′-((3-(((tert-butoxycarbonyl)amino)methyl)phenyl)carbamoyl)-3-hydroxy-[1,1′-biphenyl]-4-carboxylate

The title compound was prepared according to general procedure B. Molecular Formula: C₂₇H₂₈N₂O₆. ¹H NMR (600 MHz, CDCl₃) δ 10.84 (s, 1H), 8.10 (t, J=1.5 Hz, 1H), 7.95-7.90 (m, 2H), 7.88-7.86 (m, 1H), 7.80-7.77 (m, 1H), 7.60-7.56 (m, 3H), 7.35 (t, J=8.0 Hz, 1H), 7.25 (d, J=1.7 Hz, 1H), 7.17 (dd, J=8.3, 1.8 Hz, 1H), 7.10 (d, J=7.5 Hz, 1H), 4.92 (s, 1H), 4.33 (d, J=5.6 Hz, 2H), 3.99 (s, 3H), 1.46 (s, 9H).

### Step 2: 3′-((3-(aminomethyl)phenyl)carbamoyl)-3-hydroxy-[1,1′-biphenyl]-4-carboxylic acid (CDD-2985)

The title compound was prepared from the intermediate according to general procedure D using TFA, followed by general procedure E. Molecular Formula: C₂₁H₁₈N₂O₄. ¹H NMR (600 MHz, DMSO) δ 10.51 (s, 1H), 8.30 (bs, 2H), 8.22 (s, 1H), 8.02 (s, 1H), 7.91 (d, J=7.7 Hz, 1H), 7.85 (d, J=7.8 Hz, 1H), 7.74 (dd, J=16.2, 8.1 Hz, 2H), 7.59 (t, J=7.7 Hz, 1H), 7.43 (t, J=7.9 Hz, 1H), 7.23 (d, J=7.6 Hz, 1H), 7.05 (d, J=1.5 Hz, 1H), 6.99 (dd, J=7.9, 1.5 Hz, 1H), 4.06 (s, 2H). ¹³C NMR (151 MHz, DMSO) δ 171.20, 165.48, 163.48, 142.36, 140.48, 139.37, 135.65, 135.20, 130.53, 129.81, 129.03, 128.88, 126.92, 125.57, 123.93, 120.89, 120.42, 119.95, 114.59, 114.13, 42.84, 39.52. Calc.'d m/z [M+H]⁺: 363.1345; Obs.'d: 363.1340.

### Synthesis of 3′-(((3-(aminomethyl)phenyl)amino)methyl)-3-hydroxy-5′-methyl-[1,1′-biphenyl]-4-carboxylic acid (CDD-2997)

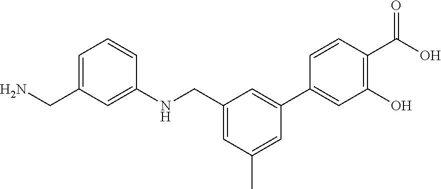 [SMILES: Cc1cc(CNc2cccc(CN)c2)cc(-c2ccc(C(=O)O)c(O)c2)c1]

### Step 1: methyl 3′-formyl-3-hydroxy-5′-methyl-[1,1′-biphenyl]-4-carboxylate

The title compound was prepared according to general procedure C. Molecular Formula: C₁₆H₁₄O₄. ¹H NMR (600 MHz, CDCl₃) δ 10.83 (s, 1H), 10.06 (s, 1H), 7.93-7.90 (m, 2H), 7.73-7.71 (m, 1H), 7.70-7.68 (m, 1H), 7.24 (d, J=1.7 Hz, 1H), 7.15 (dd, J=8.2, 1.8 Hz, 1H), 3.98 (s, 3H), 2.51 (s, 3H).

### Step 2: 3′-(((3-(aminomethyl)phenyl)amino)methyl)-3-hydroxy-5′-methyl-[1,1′-biphenyl]-4-carboxylic acid

The title compound was prepared from the intermediate according to general procedure B. Molecular Formula: C₂₂H₂₂N₂O₃. ¹H NMR (600 MHz, CDCl₃) δ 10.79 (s, 1H), 7.87 (d, J=8.3 Hz, 1H), 7.41 (s, 1H), 7.34 (s, 1H), 7.22 (s, 1H), 7.19 (d, J=1.7 Hz, 1H), 7.13 (t, J=7.8 Hz, 1H), 7.10 (dd, J=8.3, 1.7 Hz, 1H), 6.64 (d, J=7.4 Hz, 1H), 6.59 (s, 1H), 6.55 (dd, J=8.0, 2.0 Hz, 1H), 4.34 (s, 2H), 4.24 (d, J=5.4 Hz, 2H), 3.97 (s, 3H), 2.41 (s, 3H), 1.45 (s, 9H).

### Step 3: 3′-(((3-(aminomethyl)phenyl)amino)methyl)-3-hydroxy-5′-methyl-[1,1′-biphenyl]-4-carboxylic acid (CDD-2997)

The title compound was prepared from the intermediate according to general procedure D using TFA, followed by general procedure E. Molecular Formula: C₂₂H₂₂N₂O₃. ¹H NMR (600 MHz, DMSO) δ 8.14 (bs, 3H), 7.69 (d, J=7.7 Hz, 1H), 7.40 (s, 1H), 7.30 (s, 1H), 7.13 (s, 1H), 7.08 (t, J=7.8 Hz, 1H), 6.88-6.80 (m, 2H), 6.71 (s, 1H), 6.62-6.55 (m, 2H), 6.48-6.40 (m, 1H), 4.29 (d, J=5.5 Hz, 2H), 3.87 (s, 2H), 2.33 (s, 3H). ¹³C NMR (151 MHz, DMSO) δ 171.25, 163.29, 148.97, 143.31, 140.51, 140.34, 137.78, 134.68, 130.33, 129.19, 126.95, 125.76, 122.67, 119.50, 115.76, 114.41, 113.81, 112.39, 112.15, 46.22, 42.70, 21.11. Calc.'d m/z [M+H]⁺: 363.1709; Obs.'d: 363.1702.

### Synthesis of 4-(5-(((3-(aminomethyl)phenyl)amino)methyl)pyridin-3-yl)-2-hydroxybenzoic acid (CDD-2998)

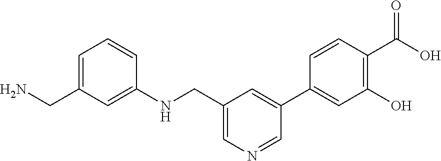 [SMILES: NCc1cccc(NCc2cncc(-c3ccc(C(=O)O)c(O)c3)c2)c1]

### Step 1: methyl 3′-formyl-3-hydroxy-5′-methyl-[1,1′-biphenyl]-4-carboxylate

The title compound was prepared according to general procedure C. Molecular Formula: C₁₆H₁₄O₄. ¹H NMR (600 MHz, CDCl₃) δ 10.89 (s, 1H), 10.21 (s, 1H), 9.09 (t, J=2.0 Hz, 2H), 8.37 (t, J=2.1 Hz, 1H), 7.98 (d, J=8.2 Hz, 1H), 7.16 (dd, J=8.2, 1.8 Hz, 1H), 4.00 (s, 3H).

### Step 2: 4-(5-(((3-(aminomethyl)phenyl)amino)methyl)pyridin-3-yl)-2-hydroxybenzoic acid

The title compound was prepared from the intermediate according to general procedure B. Molecular Formula: C₂₀H₁₉N₃O₃. ¹H NMR (600 MHz, CDCl₃) δ 10.84 (s, 1H), 8.77 (s, 1H), 8.63 (s, 1H), 7.91 (d, J=8.2 Hz, 1H), 7.89 (s, 1H), 7.18 (d, J=1.7 Hz, 1H), 7.13 (t, J=7.8 Hz, 1H), 7.09 (dd, J=8.2, 1.8 Hz, 1H), 6.66 (d, J=7.5 Hz, 1H), 6.59 (s, 1H), 6.53 (dd, J=8.0, 2.0 Hz, 1H), 4.83 (s, 1H), 4.43 (s, 2H), 4.23 (d, J=5.5 Hz, 2H), 3.98 (s, 3H), 2.04 (s, 1H), 1.44 (s, 9H).

### Step 3: 4-(5-(((3-(aminomethyl)phenyl)amino)methyl)pyridin-3-yl)-2-hydroxybenzoic acid (CDD-2998)

The title compound was prepared from the intermediate according to general procedure D using TFA, followed by general experimental procedure E. Molecular Formula: C₂₀H₁₉N₃O₃. ¹H NMR (600 MHz, DMSO) δ 8.70 (s, 1H), 8.54 (s, 1H), 8.22-8.04 (m, 3H), 7.98 (s, 1H), 7.73 (d, J=7.6 Hz, 1H), 7.10 (t, J=7.8 Hz, 1H), 6.92 (s, 1H), 6.89 (d, J=7.7 Hz, 1H), 6.73 (s, 1H), 6.66-6.58 (m, 2H), 6.52 (t, J=5.9 Hz, 1H), 4.37 (d, J=5.8 Hz, 2H), 3.88 (s, 2H). ¹³C NMR (151 MHz, DMSO) δ 170.99, 163.62, 148.59, 147.68, 145.97, 139.92, 135.42, 135.33, 134.77, 132.95, 130.61, 129.28, 120.14, 116.15, 114.34, 114.02, 112.58, 112.28, 43.69, 42.64. Calc.'d m/z [M+H]⁺: 350.1505; Obs.'d: 350.1495.

### Synthesis of 4-(2-(3-(aminomethyl)phenyl)isoindolin-5-yl)-2-hydroxybenzoic acid (CDD-3080)

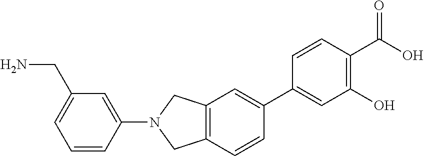 [SMILES: NCc1cccc(N2Cc3ccc(-c4ccc(C(=O)O)c(O)c4)cc3C2)c1]

### Step 1: tert-butyl 5-(3-hydroxy-4-(methoxycarbonyl)phenyl)isoindoline-2-carboxylate

The title compound was prepared according to general procedure C. Chemical Formula: C₂₁H₂₃NO₅. ¹H NMR (600 MHz, CDCl₃) δ 10.81 (d, J=7.2 Hz, 1H), 7.89 (dd, J=8.3, 1.9 Hz, 1H), 7.54-7.43 (m, 2H), 7.33 (dd, J=30.9, 8.2 Hz, 1H), 7.19 (dd, J=5.6, 1.5 Hz, 1H), 7.13-7.07 (m, 1H), 4.74 (d, J=9.5 Hz, 2H), 4.70 (d, J=6.0 Hz, 2H), 3.97 (s, 3H), 1.53 (s, 9H).

### Step 2: methyl 4-(2-(3-(((tert-butoxycarbonyl)amino)methyl)phenyl)isoindolin-5-yl)-2-hydroxybenzoate

The title compound was prepared according to general procedure D using TFA to afford an intermediate which was used in the next reaction without further purification. In a microwave vial, methyl 2-hydroxy-4-(isoindolin-5-yl)benzoate (30 mg, 0.112 mmol), tert-butyl (3-bromobenzyl)carbamate (38 mg, 0.134), potassium tert-butoxide (25 mg, 0.224 mmol), and BrettPhos Pd G4 (11 mg, 0.0112 mmol) were dissolved in 1 mL 1,4-dioxane. The reaction mixture was thoroughly degassed under N₂ gas and heated to 100° C. overnight. The reaction was monitored with TLC (Rf=0.8 at 70% ethyl acetate/hexanes). The reaction mixture was filtered using a 0.45 μm PTFE syringe filter and washed with brine and ethyl acetate. The organic layer was dried over anhydrous sodium sulfate and dried in vacuo. The crude product was purified by normal phase silica gel chromatography using a hexanes/ethyl acetate gradient. Product fractions were eluted at 50% ethyl acetate. Product fractions were collected and dried in vacuo, yielding the desired product (34 mg, 64%). Chemical Formula: C₂₈H₃₀N₂O₅. ¹H NMR (600 MHz, CDCl₃) δ 10.83 (s, 1H), 7.90 (d, J=8.2 Hz, 1H), 7.59-7.54 (m, 2H), 7.42 (d, J=7.7 Hz, 1H), 7.29 (t, J=7.8 Hz, 1H), 7.22 (d, J=1.7 Hz, 1H), 7.13 (dd, J=8.3, 1.7 Hz, 1H), 6.74 (d, J=7.2 Hz, 1H), 6.72-6.67 (m, 2H), 4.87 (s, 1H), 4.73 (d, J=3.0 Hz, 4H), 4.35-4.28 (m, 1H), 3.98 (s, 3H), 3.80 (s, 1H), 1.48-1.46 (m, 9H).

### Step 3: 4-(2-(3-(aminomethyl)phenyl)isoindolin-5-yl)-2-hydroxybenzoic acid (CDD-3080)

The title compound was prepared from the intermediate according to general procedure D using TFA, followed by general procedure E. Molecular Formula: C₂₂H₂₀N₂O₃. ¹H NMR (600 MHz, DMSO) δ 15.83 (s, 1H), 8.60 (s, 3H), 7.88 (d, J=7.9 Hz, 1H), 7.57 (d, J=8.0 Hz, 1H), 7.52 (s, 1H), 7.42 (d, J=7.9 Hz, 1H), 7.26 (t, J=7.8 Hz, 1H), 7.09 (s, 1H), 7.06 (s, 1H), 7.03 (dd, J=8.0, 1.3 Hz, 1H), 6.73 (d, J=7.4 Hz, 1H), 6.67 (d, J=7.8 Hz, 1H), 4.59 (s, 2H), 4.55 (s, 2H), 4.06 (s, 2H). ¹³C NMR (151 MHz, DMSO) δ 172.22, 162.86, 147.29, 143.29, 139.13, 138.25, 137.02, 135.41, 130.71, 129.29, 125.92, 123.08, 120.35, 119.10, 116.38, 114.85, 113.70, 112.02, 111.64, 53.44, 53.10, 48.58, 42.68, 39.52. Calc.'d m/z [M+H]⁺: 361.1552; Obs.'d: 361.1541.

### Synthesis of 4-(2-(((3-(aminomethyl)phenyl)amino)methyl)pyridin-4-yl)-2-hydroxybenzoic acid (CDD-3059)

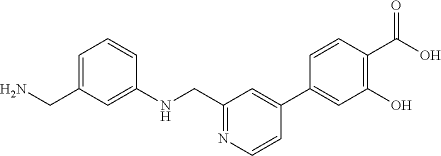 [SMILES: NCc1cccc(NCc2cc(-c3ccc(C(=O)O)c(O)c3)ccn2)c1]

### Step 1: methyl 4-(2-formylpyridin-4-yl)-2-hydroxybenzoate

The title compound was prepared according to general procedure C. Chemical Formula: C₁₄H₁₁NO₄. ¹H NMR (600 MHz, CDCl₃) δ 10.87 (s, 1H), 10.15 (s, 1H), 8.87 (d, J=5.0 Hz, 1H), 8.20 (s, 1H), 7.98 (d, J=8.2 Hz, 1H), 7.74 (dd, J=5.0, 1.6 Hz, 1H), 7.30 (d, J=1.2 Hz, 1H), 7.22-7.19 (m, 1H), 4.00 (s, 3H).

### Step 2: methyl 4-(2-(((3-(((tert-butoxycarbonyl)amino)methyl)phenyl)amino)methyl)pyridin-4-yl)-2-hydroxybenzoate

The title compound was prepared from the intermediate according to general procedure B. Chemical Formula: C₂₆H₂₉N₃O₅. ¹H NMR (600 MHz, CDCl₃) δ 10.84 (s, 1H), 8.65 (d, J=5.2 Hz, 1H), 7.93 (d, J=8.2 Hz, 1H), 7.58 (s, 1H), 7.44 (dd, J=5.2, 1.4 Hz, 1H), 7.22 (d, J=1.7 Hz, 1H), 7.15-7.11 (m, 2H), 6.64 (d, J=7.9 Hz, 2H), 6.61-6.56 (m, 1H), 4.81 (s, 1H), 4.55 (s, 2H), 4.23 (d, J=5.4 Hz, 2H), 3.98 (s, 3H), 1.45 (s, 9H).

### Step 3: 4-(2-(((3-(aminomethyl)phenyl)amino)methyl)pyridin-4-yl)-2-hydroxybenzoic acid (CDD-3059)

The title compound was prepared according to general procedure D using TFA, followed by general procedure E. Chemical Formula: C₂₀H₁₉N₃O₃. ¹H NMR (600 MHz, DMSO) δ 8.54 (d, J=5.2 Hz, 1H), 7.99 (bs, 3H), 7.73 (d, J=7.9 Hz, 1H), 7.61 (s, 1H), 7.52 (dd, J=5.2, 1.6 Hz, 1H), 7.09 (t, J=7.8 Hz, 1H), 6.93 (d, J=1.7 Hz, 1H), 6.91 (dd, J=7.9, 1.8 Hz, 11H), 6.72 (s, 1H), 6.62-6.59 (m, 2H), 6.55 (s, 1H), 4.42 (d, J=6.2 Hz, 2H), 3.87 (s, 2H), 3.17 (d, J=2.3 Hz, 11H). ¹³C NMR (151 MHz, DMSO) δ 171.05, 163.19, 156.20, 150.35, 149.28, 148.59, 141.55, 134.81, 130.16, 129.30, 121.00, 118.74, 116.18, 114.22, 113.83, 112.50, 112.18, 45.31, 42.63, 39.52. Calc.'d m/z [M+H]⁺: 360.1505; Obs.'d: 350.1498.

### Synthesis of 4-(4-(((3-(aminomethyl)phenyl)amino)methyl)pyridin-2-yl)-2-hydroxybenzoic acid (CDD-3060)

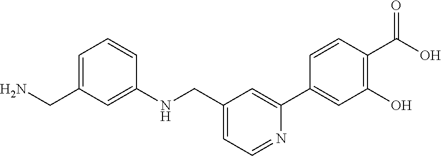 [SMILES: NCc1cccc(NCc2ccnc(-c3ccc(C(=O)O)c(O)c3)c2)c1]

### Step 1: methyl 4-(4-formylpyridin-2-yl)-2-hydroxybenzoate

The title compound was prepared according to general procedure C. Chemical Formula: C₁₄H₁₁NO₄. ¹H NMR (600 MHz, CDCl₃) δ 10.83 (s, 1H), 10.15 (s, 1H), 8.97 (d, J=4.8 Hz, 11H), 8.16 (s, 1H), 7.97 (d, J=8.3 Hz, 1H), 7.75-7.67 (m, 2H), 7.61 (d, J=8.3 Hz, 1H), 3.99 (s, 3H).

### Step 2: methyl 4-(4-(((3-(((tert-butoxycarbonyl)amino)methyl)phenyl)amino)methyl)pyridin-2-yl)-2-hydroxybenzoate

The title compound was prepared from the intermediate according to general procedure B. Chemical Formula: C₂₆H₂₉N₃O₅. ¹H NMR (600 MHz, CDCl₃) δ 10.79 (s, 1H), 8.64 (d, J=5.0 Hz, 1H), 7.93-7.89 (m, 1H), 7.75 (s, 1H), 7.56-7.52 (m, 2H), 7.29 (d, J=4.7 Hz, 1H), 7.12 (t, J=7.8 Hz, 1H), 6.66 (d, J=7.5 Hz, 1H), 6.56 (s, 1H), 6.48 (dd, J=8.0, 1.7 Hz, 1H), 4.80 (s, 1H), 4.43 (s, 2H), 4.22 (d, J=5.4 Hz, 2H), 3.97 (d, J=2.5 Hz, 3H), 1.43 (s, 9H).

### Step 3: 4-(4-(((3-(aminomethyl)phenyl)amino)methyl)pyridin-2-yl)-2-hydroxybenzoic acid (CDD-3060)

The title compound was prepared from the intermediate according to general procedure D using TFA, followed by general procedure E. Chemical Formula: C₂₀H₁₉N₃O₃. 1H NMR (600 MHz, DMSO) δ 8.54 (d, J=5.0 Hz, 1H), 8.13 (bs, 311), 7.85 (s, 11H), 7.72 (d, J=7.7 Hz, 1H), 7.28 (s, 2H), 7.26 (s, 1H), 7.09 (t, J=7.8 Hz, 1H), 6.70 (s, 11H), 6.64-6.56 (m, 3H), 4.38 (d, J=6.2 Hz, 2H), 3.87 (s, 2H). ¹³C NMR (151 MHz, DMSO) δ 171.05, 163.19, 156.20, 150.35, 149.28, 148.59, 141.55, 134.81, 130.16, 129.30, 121.00, 118.74, 116.18, 114.22, 113.83, 112.50, 112.18, 45.31, 42.63, 39.52. Calc.'d m/z [M+H]⁺: 360.1505; Obs.'d: 350.1497.

### Synthesis of 3′-(((3-(aminomethyl)phenyl)amino)methyl)-3,5′-dihydroxy-[1,1′-biphenyl]-4-carboxylic acid (CDD-3061)

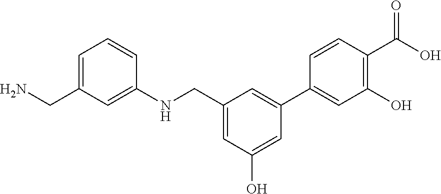 [SMILES: NCc1cccc(NCc2cc(O)cc(-c3ccc(C(=O)O)c(O)c3)c2)c1]

### Step 1: methyl 3′-formyl-3,5′-dihydroxy-[1,1′-biphenyl]-4-carboxylate

The title compound was prepared according to general procedure C. Chemical Formula: C₁₅H₁₂O₅. ¹H NMR (600 MHz, CDCl₃) δ 10.84 (s, 1H), 10.02 (s, 1H), 7.92 (d, J=8.2 Hz, 1H), 7.69 (s, 1H), 7.36 (s, 2H), 7.23 (s, 1H), 7.14 (d, J=8.3 Hz, 1H), 5.38 (s, 1H), 3.99 (s, 3H).

### Step 2: methyl 3′-(((3-(((tert-butoxycarbonyl)amino)methyl)phenyl)amino)methyl)-3,5′-dihydroxy-[1,1′-biphenyl]-4-carboxylate

The title compound was prepared from the intermediate according to general procedure B. Chemical Formula: C₂₇H₃₀N₂O₆. ¹H NMR (600 MHz, CDCl₃) δ 10.78 (s, 1H), 7.83 (d, J=8.3 Hz, 1H), 7.13 (d, J=1.5 Hz, 1H), 7.11-7.09 (m, 2H), 7.05 (dd, J=8.3, 1.5 Hz, 1H), 6.98 (s, 1H), 6.88 (s, 1H), 6.67-6.63 (m, 1H), 6.60 (s, 1H), 6.54 (d, J=8.0 Hz, 1H), 4.93 (s, 1H), 4.27 (s, 2H), 4.22 (d, J=5.0 Hz, 2H), 3.96 (s, 3H), 1.45 (s, 9H).

### Step 3: 3′-(((3-(aminomethyl)phenyl)amino)methyl)-3,5′-dihydroxy-[1,1′-biphenyl]-4-carboxylic acid (CDD-3061)

The title compound was prepared from the intermediate according to general procedure D using TFA, followed by general procedure E. Chemical Formula: C₂₁H₂₀N₂O₄. ¹H NMR (600 MHz, DMSO) δ 9.43 (s, 1H), 7.79 (s, 2H), 7.66 (d, J=8.2 Hz, 1H), 7.07 (t, J=7.8 Hz, 1H), 7.04 (s, 1H), 6.83 (s, 1H), 6.76 (d, J=6.2 Hz, 2H), 6.72 (s, 1H), 6.67 (s, 1H), 6.60-6.55 (m, 2H), 6.43 (t, J=6.2 Hz, 1H), 4.25 (d, J=6.1 Hz, 2H), 3.84 (s, 2H). ¹³C NMR (151 MHz, DMSO) δ 169.71, 158.18, 149.47, 142.52, 135.15, 135.08, 130.81, 130.77, 129.70, 116.79, 116.15, 114.67, 114.22, 113.63, 112.81, 112.63, 112.28, 46.67, 43.21.

Calc.'d m/z [M+H]⁺: 365.1501; Obs.'d: 365.1496.

### Example 4: KPC-2 Inhibitors and their Biochemical Activities

Several hits were identified from the qDOS38_1 library against the KPC-2 enzyme. All compounds were synthesized without linker C1a. Among the hit compounds, CDD-2312 had the highest enrichment score of 5.9 but a lower count number of 115 compared to other libraries screened at the same time. CDD-2312 had the most potent binding affinity (Kᵢ=657±123 μM) compared to other hit compounds from the library (Table 2).

Without wishing to be bound by theory, it was hypothesized that the binding energy and/or affinity was derived from the carboxylic moiety. Further, results of earlier studies indicated that incorporation of di-carboxylate functionality within similar compounds can improve potency against β-lactamase enzymes. Thus, CDD-2311 was synthesized as a derivative with two carboxylate groups on the molecule. CDD-2311 showed a slight improvement in inhibition. All hit compounds obtained from the initial KPC-2 screening were found to have a high μM to low mM potency.

TABLE 2
Compounds identified in KPC-2 screening

| Compound | KPC-2 Kᵢ (μM) | Compound | KPC-2 Kᵢ (μM) |
|---|---|---|---|
| CDD-2308 | 3575 ± 1151 | CDD-2312 | 657 ± 123 |
| CDD-2310 | 1244 ± 532 | CDD-2311 | 522 ± 113 |
| CDD-2309 | 980 ± 180 | | |

### Example 5: Identification, Structural Analysis, and Biochemical Activity of OXA-48 Inhibitors

Several hits were identified from the qDOS38_1 library against the OXA-48 enzyme. Potential binders to the target enzymes were identified by next-generation sequencing of the DNA barcodes of the compounds retained after affinity selection (FIGS. 3B-3C). The compounds were ranked using an enrichment score, calculated based on the counts retrieved from the sequencing data and normalized by the population of the synthesized compounds and total decoded samples. Compounds with the strongest enrichment with the target enzyme that did not enrich with the non-target control from each sub-library were selected for synthesis in the absence of a DNA tag (FIG. 3B). Particularly, compounds that show structure-enrichment relationships in the same library were prioritized for synthesis.

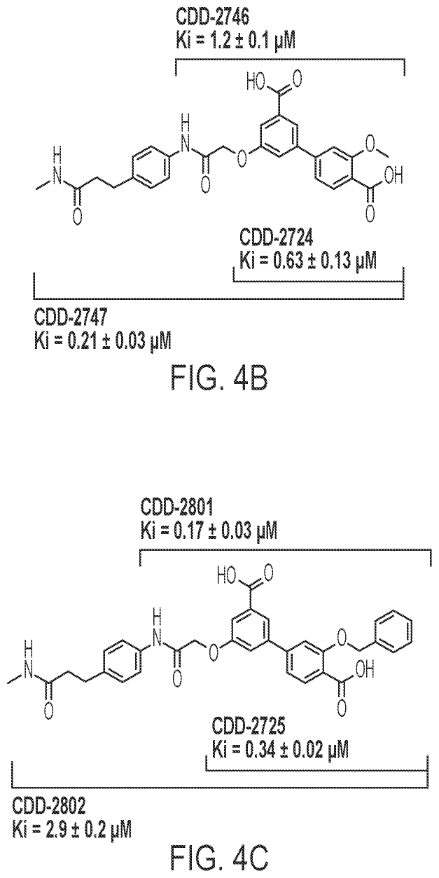

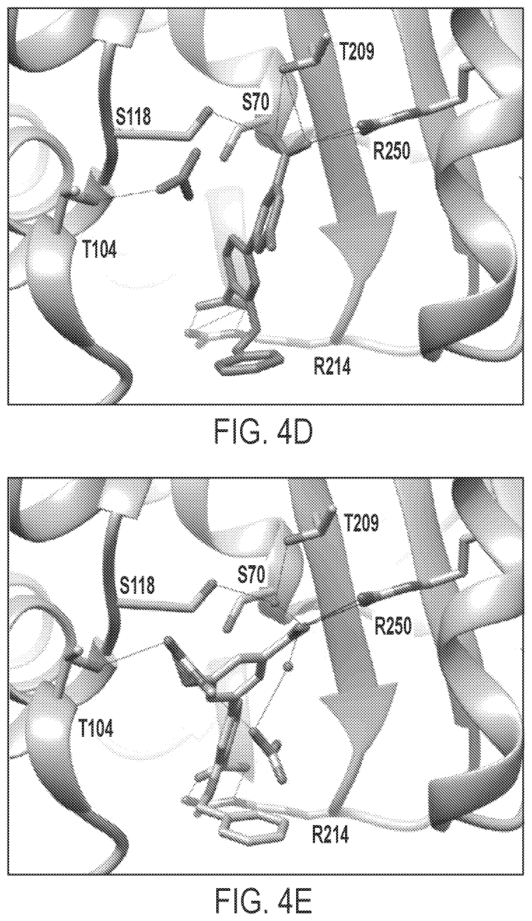

Among the several hits identified as potential OXA-48 inhibitors based on enrichment scores, a total of four compounds were examined in an enzyme inhibition assay using nitrocefin, a colorimetric cephalosporin, as a substrate paired with increasing concentrations of compounds. The nitrocefin hydrolysis rate was plotted against inhibitor concentrations to determine the inhibition constant (Kᵢ). All four compounds tested exhibited inhibition potency, with values ranging from 127 μM to 0.17 μM (FIG. 4A). CDD-2801 was the most potent inhibitor for OXA-48 with a Kᵢ of 0.17 M (FIG. 4A). Notably, the two most potent compounds (CDD-2746 and CDD-2801) contain a carboxylic acid attached to C1b in addition to the designed-in carboxylic acid on C2. There have been previous reports of di-acidic OXA-48 inhibitors, which is consistent with the high potency of these compounds.

Due to their high potency, an initial structure-activity relationship (SAR) study was performed on compounds CDD-2746 and CDD-2801. The role of the linkers for inhibition potency was first examined, and then further truncated the compounds to identify the smallest functional pharmacophore. The linker contributes to the potency of CDD-2427 in that its removal (CDD-2746) results in a 6-fold increase in the Kᵢ. However, further truncation of CDD-2746 (CDD-2724) enhanced the potency by 2-fold (FIG. 4B). In contrast, for CDD-2801, the linker (CDD-2802) reduces potency as its removal decreases the Kᵢ by 17-fold. Further, truncation of the methyl amide from C₁ (CDD-2725) reduced the potency by 2-fold, suggesting that this group contributes to the binding energy (FIG. 4C).

The crystal structure of OXA-48 in complex with CDD-2725 and CDD-2801 to determine the binding interactions of the potent inhibitors in the active site (FIGS. 4D-4E). A carboxylic acid from CDD-2725 is found in the carboxylate-binding pocket, forming hydrogen bonds with the R250, T209, and S118 sidechains. Unexpectedly, the carboxylic acid attached to the C1b instead of the terminal carboxylic acid on C2, which was included as part of the library design, is located in the carboxylic binding pocket (FIG. 4D). The carboxylic acid on C2 of CDD-2725 forms additional hydrogen bonds with the guanidinium group of R214 with its benzyloxy group solvent-exposed (FIG. 4D). A carbonate ion from the buffer solution is also present in a binding pocket, making hydrogen bonding interactions with T104 (FIG. 4D). However, the carbonate ion does not directly interact with the inhibitor CDD-2725.

In the crystal structure of the OXA-48/CDD-2801 complex, as in OXA-48/CDD-2725 structure, the carboxylic acid on C1b is located in the carboxylate-binding pocket, interacting with R250 (FIG. 4E). In addition, the carboxylate interacts with S118 and T209 mediated by a water molecule. This positions the carbonyl group on the methyl amide to interact with T104 (FIG. 4E). The carboxylic acid on C2 forms two hydrogen bonding interactions with R214 (FIG. 4E). The carbonate ion from the buffer solution is no longer able to occupy the space near T104 and is solvent exposed, interacting with the methyl amide on C1b and the carboxylic acid on C1b through a water molecule (FIG. 4E).

TABLE 3
Compounds identified in OXA-48 screening

| Compound | OXA-48 Ki (μM) | Compound | OXA-48 Ki (μM) |
|---|---|---|---|
| CDD-2724 | 0.63 ± 0.13 | CDD-2802 | 2.9 ± 0.2 |
| CDD-2746 | 1.2 ± 0.1 | CDD-2820 | 127 ± 86 |
| CDD-2747 | 0.21 ± 0.03 | CDD-2819 | 88 ± 4.4 |
| CDD-2725 | 0.34 ± 0.02 | CDD-2836 | 7.6 ± 1.0 |
| CDD-2801 | 0.17 ± 0.03 | | |

### Example 6: Identification of NDM-1 Inhibitors and their Biochemical Activities

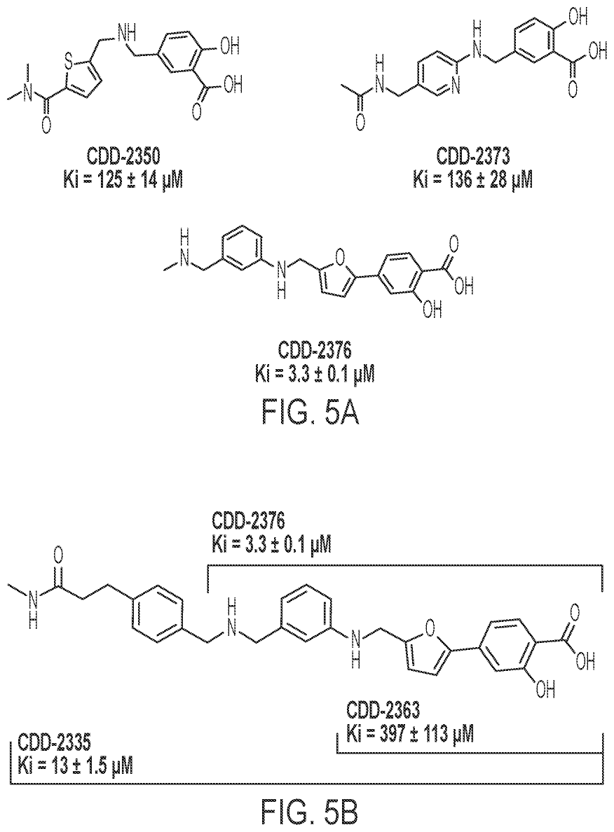

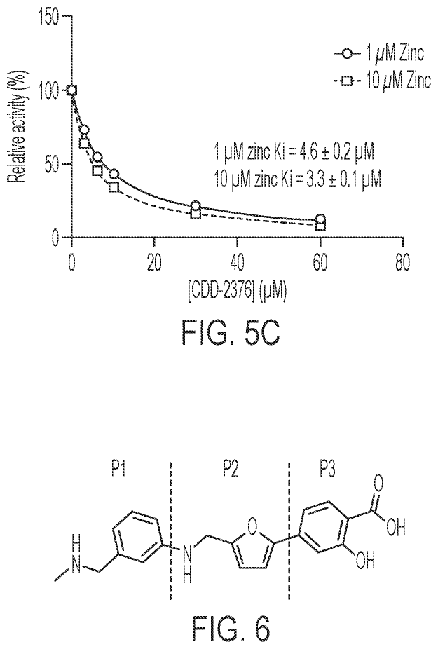

All NDM-1 β-lactamase hit molecules were identified from the qDOS38_2 library. As previously noted, NDM-1 is a class B metallo-β-lactamase with a different structure and active site chemistry compared to OXA-48. Nevertheless, it also contains a binding pocket for the β-lactam carboxylate group. Many of the compounds enriched by affinity selection against NDM-1 contained the designed carboxylic acid in the form of salicylic acid. Without wishing to be bound by any theory, salicylic acids are known to chelate metal ions, suggesting these compounds utilize active site zinc for binding affinity. A total of three highly-enriched compounds were re-synthesized in the absence of the DNA barcode and tested for inhibition using an NDM-1 assay with imipenem as the reporter substrate. The assay buffer also contained 10 M of zinc ions as required for NDM-1 activity. CDD-2350 and CDD-2373 were weak inhibitors with their Kᵢ values greater than 100 μM. In contrast, CDD-2376 was more potent, with a Kᵢ of 3.3 μM (FIG. 5A). CDD-2376 containing the linker (CDD-2335) was synthesized and it was found that that this modification decreased potency by 4-fold. CDD-2363, which lacks both the linker and the C1 building block, showed weak potency, indicating the C1 building block is important for high affinity (FIG. 5B).

Due to the metal chelating property of the salicylic acid group, CDD-2376 inhibits NDM-1 with one of two or more potential mechanisms: 1) binding to the active site of NDM-1 to inhibit the enzyme, or 2) stripping the zinc ions from the active site, thereby inactivating the enzyme. To verify that CDD-2376 was not simply stripping zinc, inhibition potency was tested under 1 μM and 10 μM zinc concentrations. There was no significant difference in the inhibition constants between the zinc concentrations (FIG. 5C), suggesting the compound is not simply stripping zinc. This was further supported by the synthesis of CDD-2463, which lacks the hydroxyl group in salicylic acid (Table 4). The metal chelating properties of salicylic acid comes from the ortho-connectivity of the carboxylic acid and hydroxyl group. Removal of the hydroxyl group would decrease the number of functional groups available for metal chelation. CDD-2463 retained significant potency (Kᵢ=8.5 μM), suggesting that its inhibition activity is not simply due to stripping the active site of zinc (Table 4).

TABLE 4
Compounds identified in NDM-1 β-lactamase
screening and selected analogues thereof

| Compound | Ki (μM) | Compound | Ki (μM) |
|---|---|---|---|
| CDD-2376 | 3.3 ± 0.1 | CDD-2653 | 1.1 ± 0.1 |
| CDD-2463 | 8.5 ± 1.1 | CDD-2997 | 0.53 ± 0.37 |
| CDD-2508 | 369 ± 70 | CDD-3061 | 2.1 ± 0.3 |
| CDD-2511 | 8.1 ± 0.8 | CDD-2919 | >100 |
| CDD-2522 | 17 ± 1.5 | CDD-2907 | >100 |
| CDD-2600 | 1.6 ± 0.3 | CDD-2985 | 29 ± 15 |
| CDD-2616 | 0.9 ± 0.44 | CDD-3080 | 43 ± 24 |
| CDD-2897 | 71 ± 37 | CDD-2336 | 144 ± 16 |
| CDD-2350 | 125 ± 14 | CDD-2363 | 397 ± 113 |
| CDD-2371 | 217 ± 46 | CDD-2395 | 153 ± 28 |
| CDD-2373 | 136 ± 28 | CDD-2335 | 13 ± 1.5 |

### Example 7: Hit Optimization of CDD-2376

As noted above, CDD-2376 was the most potent inhibitor against NDM-1. The ability of CDD-2376 to synergize with imipenem in minimum inhibitory concentration (MIC) assays against E. coli expressing NDM-1 was next evaluated. The addition of CDD-2376 resulted in a 2-fold reduction in imipenem MIC at 16 μg/mL of inhibitor concentration (Table 5).

TABLE 5
Minimum inhibitory concentrations of imipenem
in the presence of NDM-1 inhibitors

| | |
|---|---|
| [inhibitor] | Imipenem MIC (μg/mL) |

| (μg/mL) | 0 | 8 | 16 | 32 | 64 | 128 |
|---|---|---|---|---|---|---|
| CDD-2335 | 8 | NT | 8 | 8 | 8 | 8 |
| CDD-2376 | 8 | NT | 4 | 4 | 4 | 4 |
| CDD-2511 | 8 | NT | 4 | 4 | 4 | 4 |
| CDD-2522 | 8 | NT | 8 | 8 | 8 | 4 |
| CDD-2567 | 8 | NT | 8 | 4 | 4 | 4 |
| CDD-2600 | 8 | NT | 4 | 4 | 4 | 4 |
| CDD-2616 | 8 | 4 | 4 | 4 | 4 | 2 |
| CDD-2653 | 8 | NT | 4 | 4 | 4 | 2 |
| CDD-2906 | 8 | NT | 4 | 4 | 4 | 4 |
| CDD-2974 | 8 | NT | 4 | 4 | 4 | 4 |
| CDD-2985 | 8 | NT | 8 | 8 | 8 | 8 |
| CDD-2997 | 8 | 4 | 4 | 4 | Ins | Ins |
| CDD-2998 | 8 | NT | 8 | 8 | 8 | 4 |
| CDD-3059 | 8 | NT | 8 | 8 | 4 | 4 |
| CDD-3060 | 8 | NT | 8 | 8 | 4 | 4 |
| CDD-3061 | 8 | NT | 4 | 4 | 4 | 4 |
| CDD-3080 | 8 | NT | 8 | 8 | 8 | 8 |

NT = not tested; Ins = insoluble.

Therefore, the compound has weak synergistic activity when coupled with imipenem. An extensive SAR study was conducted to increase the in vitro potency and biological activity, also measured as accumulation. To this end, the compound was divided into three parts (P1, P2, and P3) for separate optimization based on the ease of synthesis of analogs (FIG. 6).

To improve in vitro potency, first, the salicylic acid moiety (P3) was modified to exclude the carboxylic acid or hydroxyl group and tested for inhibition potency against NDM-1. As expected, the removal of the carboxylic acid (CDD-2508) essentially eliminated the potency (Table 4). This validates the premise of the focused library targeting the carboxylate-binding pocket and the importance of the carboxylic acid for inhibition of the NDM-1 enzyme. The hydroxyl group is important, but not essential, for inhibition of NDM-1 as CDD-2463 reduced potency by 2.5 fold (Table 4). Because a free amine has been reported to enhance the accumulation of compounds into bacteria, the methyl amine on PT was converted to a free amine (CDD-2511). This resulted in a 2.5-fold loss in potency. However, to retain a free amine, CDD-2511 was utilized as the basis for subsequent SAR studies.

The P2 ring was next converted from furan to thiophene (CDD-2600) and from furan to benzene (CDD-2616) to mimic the size of the thiophene. These changes resulted in compounds with 2- and 4-fold improvement in potency, respectively (Table 4). CDD-2616 was thus used as the lead compound for a subsequent set of compounds for P2 optimization. CDD-2897, CDD-2653, CDD-2997, and CDD-3061 were synthesized with different substituents on the P2 benzene ring. The addition of a methyl group (CDD-2997) improved the potency 2-fold compared to CDD-2616 or 6-fold compared to the parent compound CDD-2376. The addition of a hydroxyl group (CDD-3061) resulted in similar potency compared to CDD-2376 (Table 4). The addition of a carboxylic acid (CDD-2897) as a larger substituent on the P2 ring reduced the potency more than 10-fold compared to CDD-2376 (Table 4). Without wishing to be limited by any theory, the reduced potency observed for CDD-2897 versus the modest potency changes for CDD-2997 and CDD-3061 suggests that the active site of NDM-1 around P2 accommodates a small hydrophobic substituent. Changing the benzene to a pyridine (CDD-2653) did not improve potency compared to CDD-2616, suggesting no other interactions with active site residues were made with the additional nitrogen.

Modifications of the free amine in P1 to a smaller (CDD-2907) or larger (CDD-2919) group resulted in a near-complete loss of potency against NDM-1 (Table 4). The lack of potency observed upon removal of the amine on P1 suggests that the amine plays an essential role in inhibition. Extensive SAR with the modification on P1 thus was not further pursued. A complete list of NDM-1 inhibitor modifications and associated Kᵢ values are provided herein (Table 6).

TABLE 6
Exemplary CDD-2376 analogues

| Compound | NDM-1 Ki (μM) | Compound | NDM-1 Ki (μM) |
|---|---|---|---|
| CDD-2376 | 3.3 ± 0.1 | CDD-2919 | >100 |
| CDD-2463 | 8.5 ± 1.1 | CDD-2907 | >100 |
| CDD-2508 | 369 ± 70 | CDD-2974 | 3.5 ± 1.4 |
| CDD-2602 | 46 ± 11 | CDD-2975 | 25 ± 14 |
| CDD-2510 | 60 ± 10 | CDD-2897 | 71 ± 37 |
| CDD-2522 | 17 ± 15 | CDD-2653 | 1.1 ± 0.1 |
| CDD-2511 | 8.1 ± 0.8 | CDD-2997 | 0.53 ± 0.37 |
| CDD-2567 | 24 ± 1.5 | CDD-2998 | 1.9 ± 0.6 |
| CDD-2600 | 1.6 ± 0.3 | CDD-2985 | 29 ± 15 |
| CDD-2615 | 14 ± 1.1 | CDD-3080 | 43 ± 24 |
| CDD-2616 | 0.9 ± 0.44 | CDD-3059 | 4.8 ± 0.4 |
| CDD-2906 | 11 ± 2 | CDD-3060 | 8.1 ± 2.0 |
| CDD-2920 | 87 ± 53 | CDD-3061 | 2.1 ± 0.3 |

### Example 8: Molecular Docking of NDM-1 Inhibitors

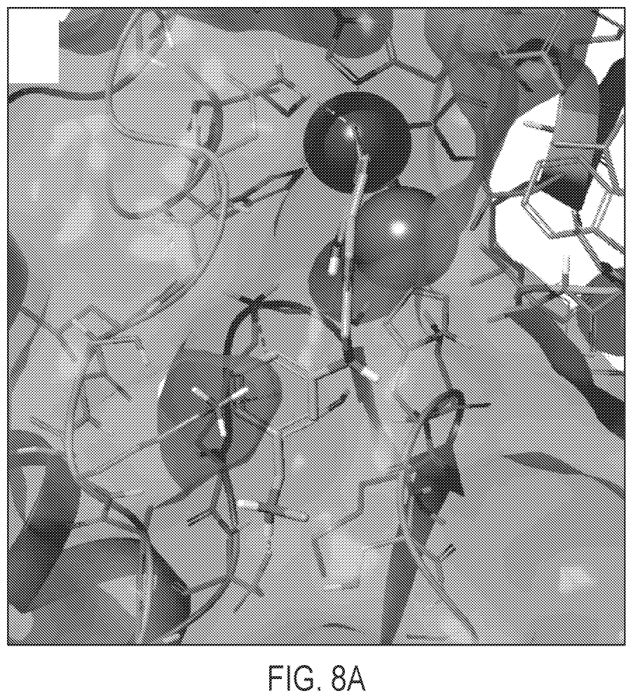

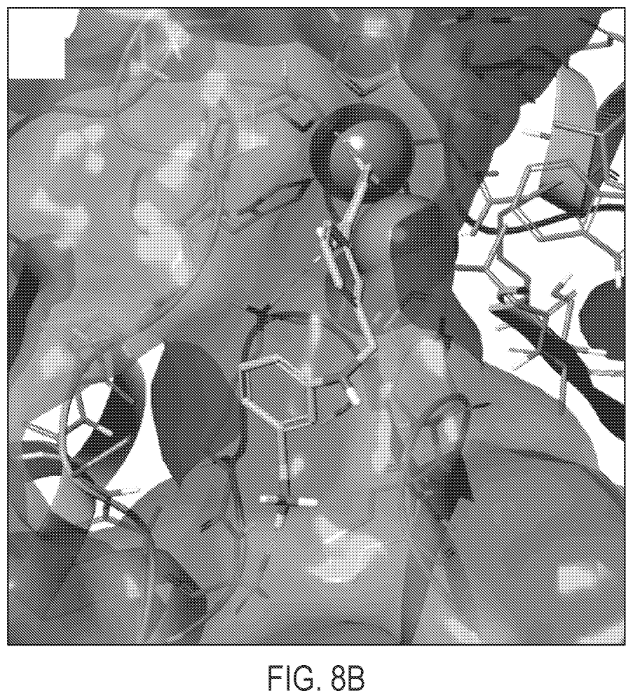

A published structure of NDM-1 β-lactamase (PDB: 4EYL) was used to dock hit compounds obtained from the NDM-1 screening experiments described above. The original NDM-1 structure has hydrolyzed ampicillin as the substrate this was used to define the grid around the active site. The protein and ligands were prepared to remove any unnecessary water molecules in the enzyme and to calculate the stable poses of bound ligands. The Scrödinger software suite induced-fit docking tool was used to dock the structures inside the active site in poses that have low binding energy. Compounds CDD-2376 and CDD-2616 were docked, and for both structures, the carboxylic acid on salicylic acid has interactions with zinc ions as speculated from the structure and also forms an H-bond with Asn220 (FIGS. 8A-8B).

This position (i.e. Asn220) is thought to correspond to the position within the catalytic pocket in which the carboxylic acid from the hydrolyzed β-lactam ring resides. Further, the hydroxyl group on salicylic acid interacts with Lys211 in the acid-binding pocket, which is thought to be the position where carboxylate group on β-lactam antibiotics normally binds.

### Example 9: Biological Activity Improvement

Optimization of compounds to improve accumulation into bacteria was conducted according to guidelines known to those of ordinary skill in the art (see Richter et al). CDD-2376 lowered the imipenem MIC by 2-fold at the lowest inhibitor concentration tested (16 g/mL), and an improvement of this property was sought. One of the guidelines for accumulation is the presence of an unhindered free amine. As noted, CDD-2511 and subsequent compounds discussed above contained a free amine. CDD-2511, however, exhibited similar synergy with imipenem as observed for CDD-2376 (Table 5). An additional guideline for accumulation is to decrease the number of rotatable bonds. CDD-2522, CDD-2985, and CDD-3080 were synthesized to reduce the total number of rotatable bonds. These compounds exhibited reduced synergy compared to CDD-2376 and CDD-2511 (Table 5). This could be due to the relatively weak inhibition potency of these compounds (Table 4).

Beyond compounds specifically designed to follow accumulation guidelines, all compounds that exhibited a Kᵢ of less than 10 μM for synergy with imipenem against E. coli expressing NDM-1 were tested. All compounds with a Kᵢ of less than 10 μM showed at least a 2-fold reduction of imipenem MIC at the highest inhibitor concentration tested (128 μg/mL). Two of the most potent compounds CDD-2616 and CDD-2997 had a 2-fold reduction in imipenem MIC at 8 μg/mL of inhibitor concentration. Two of the three most potent compounds CDD-2616 and CDD-2653 had a 4-fold reduction in imipenem MIC at 128 μg/mL of inhibitor while CDD-2997 could not be tested above 64 μg/mL due to its low solubility in aqueous solution (Table 7). After extensive SAR studies to improve both in vitro potency and biological activity, several promising inhibitors such as CDD-2616, CDD-2653, CDD-2997, and CDD-3061 were identified, and CDD-2616 was determined to be the key lead compound with a sub-micromolar inhibition potency and measurable biological activity.

While most compounds had some biological activity when paired with imipenem, the effect is still modest compared to what is considered clinically relevant. This could be partially due to the low accumulation of compounds into the periplasm and/or due to modest in vitro inhibition potency (Kᵢ). To discern between the possibilities, the accumulation of inhibitors into bacteria was quantified using mass spectrometry. Compounds were pre-incubated with E. coli inoculum before cells were washed, lysed, and analyzed via mass spectrometry. Tetracycline and clindamycin were used as positive and negative controls, respectively. CDD-2376, CDD-2616, and CDD-2653 were chosen for accumulation measurement as they represent the original hit compound and two potent compounds that display biological activity. CDD-2997, the most potent compound, was not tested due to low solubility in buffer at the concentration used for the accumulation assay (50 μM).

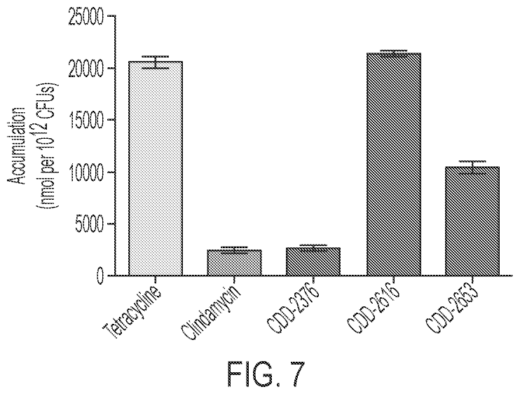

Accumulation of CDD-2376 was the lowest of all three compounds tested and comparable to that of the negative control (FIG. 7). Compared to CDD-2376, however, accumulation of CDD-2653 in bacteria was much higher and accumulation of CDD-2616 was comparable to that of the positive control (FIG. 7). This result concurs with the MIC results where CDD-2616 and CDD-2653 showed the largest reduction in imipenem MIC at the inhibitor concentration of 128 μg/mL. Note, however, that CDD-2616 accumulates at a level similar to the positive control but requires a high concentration of inhibitor to show a biological effect.

### Example 10: Hit Compound Synthesis for PBP-3 Inhibitors

While the focused library was designed for β-lactamase enzymes as targets, the core structure that distinguishes the focused library from other libraries is the terminal carboxylate. Any enzymes comprising a carboxylate binding pocket could be targeted by the β-lactamase-focused library. With respect to β-lactam resistance, penicillin-binding proteins (PBPs) are considered important bacterial proteins, as these enzymes catalyze bacterial cell wall biosynthesis and are the main target of β-lactam antibiotics. It has been proposed that identification of an inhibitor of the PBP enzyme(s) could lead to a novel antibiotic that can be used alone instead of paired with another drug. Further, PBPs and β-lactamase are thought to share a carboxylate-binding pocket, as these enzymes both comprise active sites which are similar in structure and function.

Similarly to KPC-2 and NDM-1, PBP-3 was screened against the β-lactamase-focused library and several hits were identified from qDOS38_2 and qDOS38_4 (Table 7). Since PBP-3 is not a β-lactamase enzyme, the inhibition assay cannot be used to determine the biochemical potency of inhibitor against PBP-3. Instead, in vivo potency was determined directly against E. coli and A. baumanii. Since all bacteria express PBP-3 naturally, other modifications to wild-type bacteria were not necessary.

Unlike β-lactamase inhibitor MIC determinations, which measure the synergy between inhibitor and antibiotic, PBP-3 inhibitor MIC determination only measures a single dimension concentration gradient of the inhibitor. CDD-2396 from the qDOS38_2 library had an MIC of 256 μg/mL against E. coli, which is where DMSO solvent for the inhibitor started showing an effect on bacterial growth. CDD-2507 of qDOS38_4 had an MIC of 128 g/mL but showed a slower growth of bacteria even at 16 μg/mL against E. coli, suggesting these compounds may inhibit PBP-3 function. Neither of the compounds had any significant effect against A. baumanii.

CDD-2507 was the only compound that was identified from the focused library that did not contain a terminal acid on the molecule, which may be a result of the failure to achieve urea formation at C2 of qDOS38_4 library. Instead, when the reaction was validated on DNA, it was found that amide formation was favored over urea formation. Thus, CDD-2507 was synthesized with amide coupling reaction instead of urea formation reaction between C1 and C2 building blocks and, as described elsewhere herein, this molecule exhibits growth inhibition effects versus E. coli.

TABLE 7
Compounds identified in penicillin
binding protein 3 (PBP-3) screening

| | E. coli minimum inhibitory |
|---|---|
| Compound | concentration (MIC) (μg/mL) |
| CDD-2396 | 256 |
| CDD-2507 | 128 |

### ENUMERATED EMBODIMENTS

The following exemplary embodiments are provided, the numbering of which is not to be construed as designating levels of importance:

Embodiment 1 provides a compound of formula (I), or a salt, solvate, stereoisomer, isotopologue, or tautomer thereof, or any mixtures thereof:

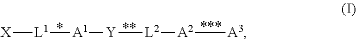

wherein:
R^{1a} and R^{1b} are each independently selected from the group consisting of H, optionally substituted C₁-C₆ alkyl, optionally substituted C₃-C₁₂ cycloalkyl, optionally substituted C₇-C₁₂ aralkyl, optionally substituted C₂-C₁₂ heterocyclyl, optionally substituted C₆-C₁₂ aryl, and C(═O)(optionally substituted C₁-C₆ alkyl);
A¹ and A² are each independently selected from the group consisting of a bond, optionally substituted phenylenyl and optionally substituted C₂-C₉ heteroarylenyl;
A³ is selected from the group consisting of optionally substituted C₆-C₁₂ aryl and optionally substituted C₂-C₁₂heteroaryl;
L¹ and L² are each independently selected from the group consisting of a bond, optionally substituted C₁-C₃ alkylenyl, —C(═O), —C(═O)(optionally substituted C₁-C₃ alkylenyl)-O—, —C(═O)(optionally substituted C₁-C₃ alkylenyl)-, (optionally substituted C₁-C₃ alkylenyl)C(═O)—, —C(═O)(optionally substituted C₁-C₃ alkylenyl)C(═O)—, optionally substituted C₃-C₈ heterocycloalkylenyl, —C(═O)(optionally substituted C₃-C₈ heterocycloalkylenyl)(optionally substituted C₁-C₃ alkylenyl)C(═O)—, and —C(═O)(optionally substituted C₃-C₈ cycloalkylenyl)C(═O)—;
R² is selected from the group consisting of H and optionally substituted C₁-C₆ alkyl,
   wherein R² can combine with A² to form an optionally substituted C₃-C₈ heterocycloalkyl;
X is selected from the group consisting of N(R^{1a})(R^{1b}) and optionally substituted C₂-C₁₂ heterocyclyl;
Y is selected from the group consisting of a bond, —N(R²)—, —(CH₂)₁₋₃N(R²)—**, —O—, and —S—;
* indicates a bond between L¹ and A¹;
** indicates a bond between Y and L²;
*** indicates a bond between A² and A³; and
each occurrence of R^{A} and R^{B} is independently selected from the group consisting of H, —C(═O)(optionally substituted C₁-C₆ alkyl), optionally substituted C₁-C₆ alkyl, optionally substituted C₃-C₅ cycloalkyl, optionally substituted C₇-C₁₂ aralkyl, optionally substituted phenyl, optionally substituted naphthyl, and optionally substituted heteroaryl.

Embodiment 2 provides the compound of Embodiment 1, wherein at least one of the following applies:
(a) A¹ is a bond;
(b) A² is a bond;
(c) L¹ is a bond;
(d) X is

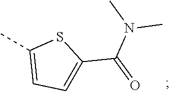

and
(e) Y is a bond or —NH—.

Embodiment 3 provides the compound of Embodiment 1, which is a compound of formula (Ia):

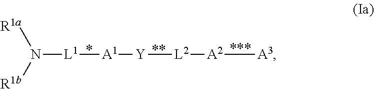

wherein:
A¹ and A² are each independently selected from the group consisting of optionally substituted phenylenyl and optionally substituted C₂-C₉ heteroarylenyl;
A³ is selected from the group consisting of optionally substituted C₆-C₁₂ aryl and optionally substituted C₂-C₁₂heteroaryl;
L¹ and L² are each independently selected from the group consisting of a bond, optionally substituted C₁-C₃ alkylenyl, —C(═O), —C(═O)(optionally substituted C₁-C₃ alkylenyl)-O—, —C(═O)(optionally substituted C₁-C₃ alkylenyl)-, (optionally substituted C₁-C₃ alkylenyl)C(═O)—, —C(═O)(optionally substituted C₁-C₃ alkylenyl)C(═O)—, optionally substituted C₃-C₅ heterocycloalkylenyl, —C(═O)(optionally substituted C₃-C₈ heterocycloalkylenyl)(optionally substituted C₁-C₃ alkylenyl)C(═O)—, and —C(═O)(optionally substituted C₃-C₈ cycloalkylenyl)C(═O)—;
R^{1a} and R^{1b} are each independently selected from the group consisting of H, optionally substituted C₁-C₆ alkyl, optionally substituted C₃-C₁₂ cycloalkyl, optionally substituted C₇-C₁₂ aralkyl, optionally substituted C₂-C₁₂ heterocyclyl, optionally substituted C₆-C₁₂ aryl, and C(═O)(optionally substituted C₁-C₆ alkyl);
R² is selected from the group consisting of H and optionally substituted C₁-C₆ alkyl,
   wherein R² can combine with A² to form an optionally substituted C₃-C₅ heterocycloalkyl;
Y is selected from the group consisting of —N(R²)—, —(CH₂)₁₋₃N(R²)—**, —O—, and —S—;
* indicates a bond between L¹ and A¹;
** indicates a bond between Y and L²;
*** indicates a bond between A² and A³; and
each occurrence of R^{A} and R^{B} is independently selected from the group consisting of H, —C(═O)(optionally substituted C₁-C₆ alkyl), optionally substituted C₁-C₆ alkyl, optionally substituted C₃-C₈ cycloalkyl, optionally substituted C₇-C₁₂ aralkyl, optionally substituted phenyl, optionally substituted naphthyl, and optionally substituted heteroaryl.

Embodiment 4 provides the compound of any one of Embodiments 1-3, wherein A¹ is selected from the group consisting of:

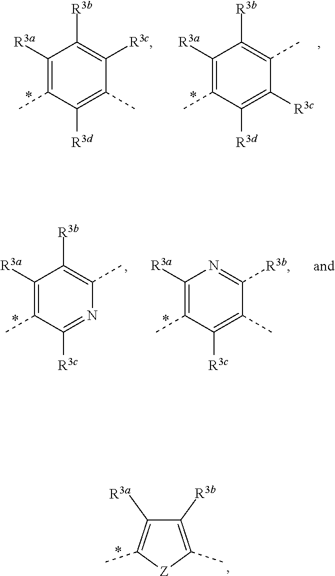

wherein:
R^{3a}, R^{3b}, R^{3c}, and R^{3d}, if present, are each independently selected from the group consisting of H, halogen, OR^{A}, C(═O)OR^{A}, CN, NO₂, optionally substituted C₁-C₆ alkyl, and optionally substituted C₃-C₈ cycloalkyl.

Embodiment 5 provides the compound of Embodiment 4, wherein at least one of the following applies:
(a) at least one selected from R^{3a}, R^{3b}, R^{3c}, and R^{3d}, is H;
(b) at least two selected from R^{3a}, R^{3b}, R^{3c}, and R^{3d} are H; and
(c) at least three selected from R^{3a}, R^{3b}, R^{3c}, and R^{3d} are H.

Embodiment 6 provides the compound of any one of Embodiments 1-5, wherein A¹ is selected from the group consisting of:

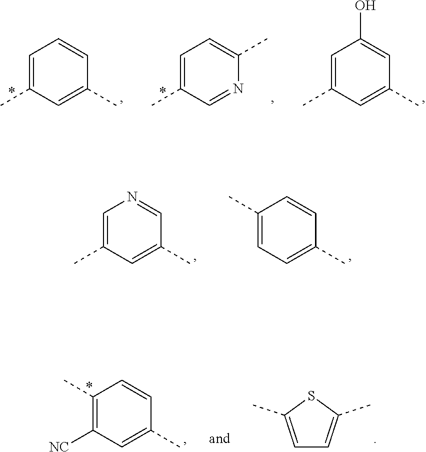

Embodiment 7 provides the compound of any one of Embodiments 1-6, wherein A² is selected from the group consisting of:

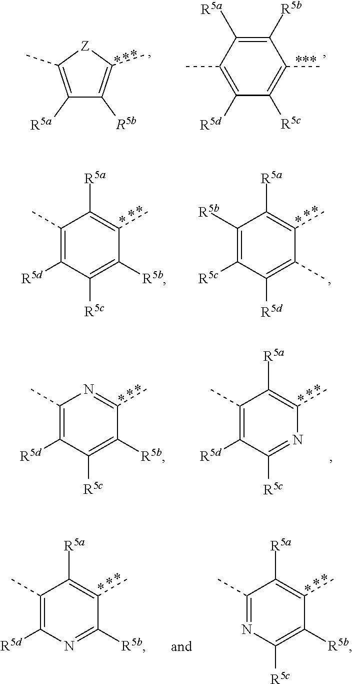

wherein:
Z is selected from the group consisting of O, S, and NR⁴;
R⁴ is selected from the H and optionally substituted C₁-C₆ alkyl; and
R^{5a}, R^{5b}, R^{5c}, and R^{5d}, if present, are each independently selected from the group consisting of H, halogen, OR^{A}, C(═O)OR^{A}, CN, NO₂, optionally substituted C₁-C₆ alkyl, and optionally substituted C₃-C₅ cycloalkyl.

Embodiment 8 provides the compound of Embodiment 7, wherein at least one of the following applies:
(a) at least one selected from R^{5a}, R^{5b}, R^{5c}, and R^{5d} is H;
(b) at least two selected from R^{5a}, R^{5b}, R^{5c}, and R^{5d} are H; and
(c) at least three selected from R^{5a}, R^{5b}, R^{5c}, and R^{5d} are H.

Embodiment 9 provides the compound of any one of Embodiments 1-8, wherein A² is selected from the group consisting of:

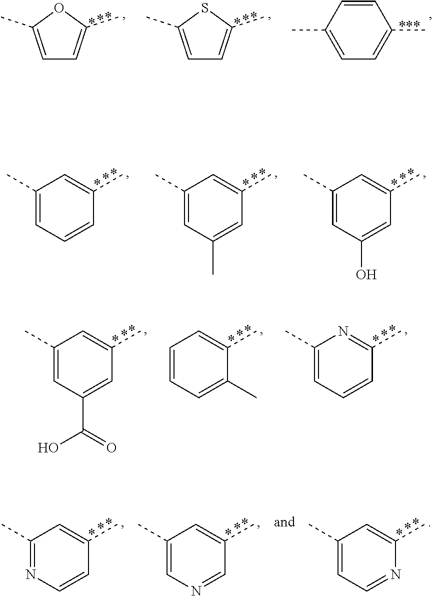

Embodiment 10 provides the compound of any one of Embodiments 1-9, wherein A³ is selected from the group consisting of:

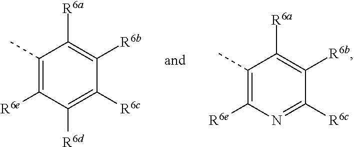

wherein:
R^{6a}, R^{6b}, R^{6c}, R^{6d}, and R^{6e}, if present, are each independently selected from the group consisting of H, halogen, OR^{A}, C(═O)OR^{A}, (optionally substituted C₁-C₆ alkylenyl)C(═O)OR^{A}. CN, NO₂, optionally substituted C₁-C₆ alkyl, and optionally substituted C₃-C₈ cycloalkyl.

Embodiment 11 provides the compound of Embodiment 10, wherein R^{6a}, R^{6b}, R^{6c}, R^{6d}, and R^{6e} are each independently selected from the group consisting of H, C(═O)OH, OH, and CH₂C(═O)OH.

Embodiment 12 provides the compound of Embodiment 10 or 11, wherein at least one of the following applies:
(a) at least one selected from R^{6a}, R^{6b}, R^{6c}, R^{6d}, and R^{6e} is H;
(b) at least two selected from R^{6a}, R^{6b}, R^{6c}, R^{6d}, and R^{6e} are H;
(c) at least three selected from R^{6a}, R^{6b}, R^{6c}, R^{6d}, and R^{6e} are H; and
(d) at least four selected from R^{6a}, R^{6b}, R^{6c}, R^{6d}, and R^{6e} are H.

Embodiment 13 provides the compound of any one of Embodiments 10-12, wherein at least one selected from R^{6a}, R^{6b}, R^{6c}, R^{6d}, and R^{6e} is selected from the group consisting of C(═O)OH, CH₂C(═O)OH, OMe, OBn, and OH.

Embodiment 14 provides the compound of any one of Embodiments 1-13, wherein A³ is selected from the group consisting of:

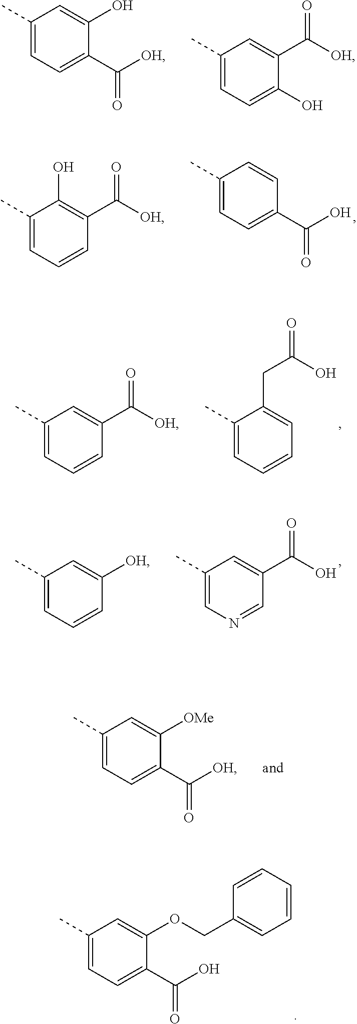

Embodiment 15 provides the compound of any one of Embodiments 1-14, wherein L¹ is selected from the group consisting of —CH₂—, —CH₂C(═O)—, —CH₂CH₂C(═O)—, C(═O)CH₂CH₂O—,

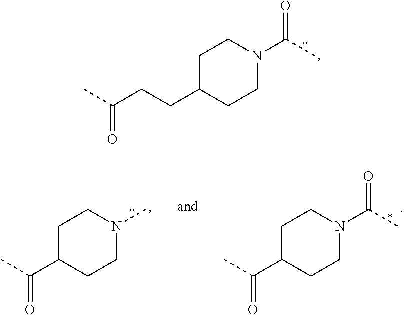

Embodiment 16 provides the compound of any one of Embodiments 1-15, wherein L² is selected from the group consisting of —CH₂—, —C(═O)—, and —C(═O)CH₂O—.

Embodiment 17 provides the compound of any one of Embodiments 1-16, wherein R^{1a} and R^{1b} are each independently selected from the group consisting of H, CH₃, C(═O)CH₃, and

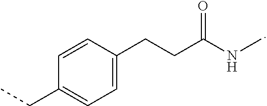

Embodiment 18 provides the compound of any one of Embodiments 1-17, wherein one of the following applies:
(a) R^{1a} is H and R^{1b} is H;
(b) R^{1a} is CH₃ and R^{1b} is H;
(c) R^{1a} is C(═O)CH₃ and R^{1b} is H; or
(d) R^{1a} is

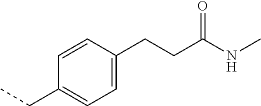

and R^{1b} is H.

Embodiment 19 provides the compound of any one of Embodiments 1-18, wherein R² is H.

Embodiment 20 provides the compound of any one of Embodiments 1-19, wherein Y is selected from the group consisting of —NH—, —CH₂NH—**, and —(CH₂)₃NH—**.

Embodiment 21 provides the compound of any one of Embodiments 1-20, which is selected from the group consisting of:
2-hydroxy-4-(5-(((3-((methylamino)methyl)phenyl)amino)methyl)furan-2-yl)benzoic acid;
2-(4′-(((3-(((4-(3-(methylamino)-3-oxopropyl)benzyl)amino)methyl)phenyl)amino)methyl)-[1,1′-biphenyl]-2-yl)acetic acid;
5-((((5-(dimethylcarbamoyl)thiophen-2-yl)methyl)amino)methyl)-2-hydroxybenzoic acid;
5-(((5-(acetamidomethyl)pyridin-2-yl)amino)methyl)-2-hydroxybenzoic acid;
2-hydroxy-4-(5-(((3-(((4-(3-(methylamino)-3-oxopropyl)benzyl)amino)methyl)phenyl)amino)methyl)furan-2-yl)benzoic acid;
2-hydroxy-4-(5-((methylamino)methyl)furan-2-yl)benzoic acid;
4-(5-(((3-((methylamino)methyl)phenyl)amino)methyl)furan-2-yl)benzoic acid;
3-(5-(((3-((methylamino)methyl)phenyl)amino)methyl)furan-2-yl)phenol;
2-hydroxy-5-(5-(((3-((methylamino)methyl)phenyl)amino)methyl)furan-2-yl)benzoic acid;
2-hydroxy-3-(5-(((3-((methylamino)methyl)phenyl)amino)methyl)furan-2-yl)benzoic acid;
3-(5-(((3-((methylamino)methyl)phenyl)amino)methyl)furan-2-yl)benzoic acid;
4-(5-(((3-(aminomethyl)phenyl)amino)methyl)furan-2-yl)-2-hydroxybenzoic acid;
3-(5-(((3-(aminomethyl)phenyl)amino)methyl)furan-2-yl)-2-hydroxybenzoic acid;
4-(5-(((3-(aminomethyl)phenyl)amino)methyl)thiophen-2-yl)-2-hydroxybenzoic acid;
4′-(((3-(aminomethyl)phenyl)amino)methyl)-3-hydroxy-[1,1′-biphenyl]-4-carboxylic acid;
3′-(((3-(aminomethyl)phenyl)amino)methyl)-3-hydroxy-[1,1′-biphenyl]-4-carboxylic acid;
5-(4-(((3-aminophenyl)amino)methyl)phenyl)nicotinic acid;
5-(((3-(aminomethyl)phenyl)amino)methyl)-3′-hydroxy-[1,1′-biphenyl]-3,4′-dicarboxylic acid;
4-(6-(((3-(aminomethyl)phenyl)amino)methyl)pyridin-2-yl)-2-hydroxybenzoic acid;
3′-(((3-(aminomethyl)phenyl)amino)methyl)-3-hydroxy-5′-methyl-[1,1′-biphenyl]-4-carboxylic acid;
3′-(((3-(aminomethyl)phenyl)amino)methyl)-3,5′-dihydroxy-[1,1′-biphenyl]-4-carboxylic acid;
3′-((3-(aminomethyl)phenyl)carbamoyl)-3-hydroxy-[1,1′-biphenyl]-4-carboxylic acid;
4-(2-(3-(aminomethyl)phenyl)isoindolin-5-yl)-2-hydroxybenzoic acid;
3-hydroxy-3′-(((3-((methylamino)methyl)phenyl)amino)methyl)-[1,1′-biphenyl]-4-carboxylic acid;
3-hydroxy-3′-(((3-(2-(methylamino)-2-oxoethyl)phenyl)amino)methyl)-[1,1′-biphenyl]-4-carboxylic acid;
3′-(((3-(aminomethyl)-5-hydroxyphenyl)amino)methyl)-3-hydroxy-[1,1′-biphenyl]-4-carboxylic acid;
3′-(((5-(aminomethyl)pyridin-3-yl)amino)methyl)-3-hydroxy-[1,1′-biphenyl]-4-carboxylic acid;
4-(5-(((3-(aminomethyl)phenyl)amino)methyl)pyridin-3-yl)-2-hydroxybenzoic acid;
4-(2-(((3-(aminomethyl)phenyl)amino)methyl)pyridin-4-yl)-2-hydroxybenzoic acid;
4-(4-(((3-(aminomethyl)phenyl)amino)methyl)pyridin-2-yl)-2-hydroxybenzoic acid;
3′-methoxy-5-(2-(methylamino)-2-oxoethoxy)-[1,1′-biphenyl]-3,4′-dicarboxylic acid;
3′-(benzyloxy)-5-(2-(methylamino)-2-oxoethoxy)-[1,1′-biphenyl]-3,4′-dicarboxylic acid;
3′-(benzyloxy)-5-(2-((4-(3-(methylamino)-3-oxopropyl)phenyl)amino)-2-oxoethoxy)-[1,1′-biphenyl]-3,4′-dicarboxylic acid;
2′-(((3-(3-aminophenyl)propyl)amino)methyl)-[1,1′-biphenyl]-3-carboxylic acid;
2′-(((3-(3-((4-(3-(methylamino)-3-oxopropyl)benzyl)amino)phenyl)propyl)amino)methyl)-[1,1′-biphenyl]-3-carboxylic acid;
2′-(((3-cyano-4-(4-(dimethylcarbamoyl)piperidin-1-yl)benzyl)amino)methyl)-[1,1′-biphenyl]-3-carboxylic acid;
2-hydroxy-5-((((5-(4-(3-(methylamino)-3-oxopropyl)piperidine-1-carbonyl)thiophen-2-yl)methyl)amino)methyl)benzoic acid; and
2-hydroxy-5-(((5-((4-(methylcarbamoyl)cyclohexane-1-carboxamido)methyl)pyridin-2-yl)amino)methyl)benzoic acid.

Embodiment 22 provides the compound of formula (II), or a salt, solvate, stereoisomer, isotopologue, or tautomer thereof, or any mixtures thereof:

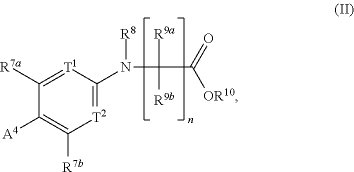

wherein:
A⁴ is selected from the group consisting of optionally substituted C₆-C₁₀ aryl and optionally substituted C₂-C₉ heteroaryl;
R^{7a}, R^{7b}, R^{7c}, and R^{7d}, if present, are each independently selected from the group consisting of H, halogen, OR^{A}, C(═O)OR^{A}, CN, NO₂, optionally substituted C₁-C₆ alkyl, and optionally substituted C₃-C₈ cycloalkyl;
R⁸ is selected from the group consisting of H and optionally substituted C₁-C₆ alkyl;
each occurrence of R^{9a} and R^{9b} is independently selected from the group consisting of H and optionally substituted C₁-C₆ alkyl;
R¹⁰ is selected from the group consisting of H, optionally substituted C₁-C₆ alkyl, optionally substituted C₃-C₁₂ cycloalkyl, optionally substituted C₇-C₁₂ aralkyl, optionally substituted C₂-C₁₂ heterocyclyl, optionally substituted C₆-C₁₂ aryl;
T¹ is selected from the group consisting of N and CR^{7c};
T² is selected from the group consisting of N and CR^{7d};
n is an integer selected from the group consisting of 1, 2, and 3; and
each occurrence of R^{A} and R^{B} is independently selected from the group consisting of H, —C(═O)(optionally substituted C₁-C₆ alkyl), optionally substituted C₁-C₆ alkyl, optionally substituted C₃-C₈ cycloalkyl, optionally substituted C₇-C₁₂ aralkyl, optionally substituted phenyl, optionally substituted naphthyl, and optionally substituted heteroaryl.

Embodiment 23 provides the compound of Embodiment 22, wherein A⁴ is selected from the group consisting of:

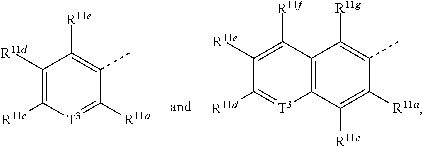

wherein:
R^{11a}, R^{11b}, R^{11c}, R^{11d}, R^{11e}, R^{11f}, and R^{11g}, if present, are each independently selected from the group consisting of H, halogen, OR^{A}, O(optionally substituted C₁-C₃ alkylenyl)C(═O)OR^{A}. O(optionally substituted C₁-C₃ alkylenyl)C(═O)N(R^{A})(R^{B}), C(═O)R^{A} C(═O)OR^{A}, C(═O)N(R^{A})(R^{B}), CN, NO₂, optionally substituted C₁-C₆ alkyl, and optionally substituted C₃-C₈ cycloalkyl; and
T³ is selected from the group consisting of N and CR^{11b}.

Embodiment 24 provides the compound of Embodiment 22 or 23, wherein A⁴ is selected from the group consisting of:

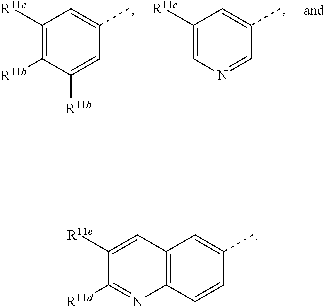

Embodiment 25 provides the compound of Embodiment 23 or 24, wherein R^{11a}, R^{11b}, R^{11c}, R^{11d}, R^{11e}, R^{11f}, and R^{11g}, if present, are each independently selected from the group consisting of H, Me, OMe, F, C(═O)NH(Me), C(═O)H, OCH₂C(═O)OH, and OCH₂C(═O)NMe₂.

Embodiment 26 provides the compound of any one of Embodiments 22-25, wherein A⁴ is selected from the group consisting of:

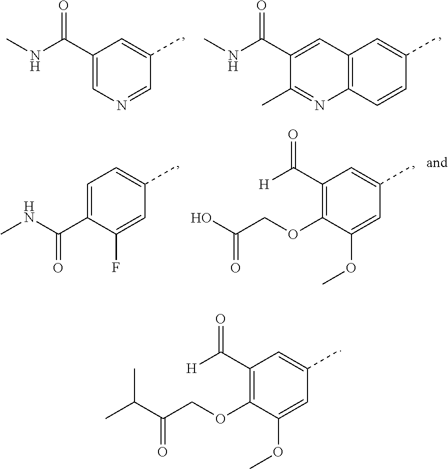

Embodiment 27 provides the compound of any one of Embodiments 22-26, wherein at least one of the following applies:
(a) T¹ is N;
(b) T² is N;
(c) R^{7a} is H; and
(d) R^{7b} is H.

Embodiment 28 provides the compound of any one of Embodiments 22-27, wherein R⁸ is H.

Embodiment 29 provides the compound of any one of Embodiments 22-28, wherein R^{9a} is H and R^{9b} is H.

Embodiment 30 provides the compound of any one of Embodiments 22-29, wherein n is 1.

Embodiment 31 provides the compound of any one of Embodiments 22-30, wherein R¹⁰ is H.

Embodiment 32 provides the compound of any one of Embodiments 22-31, which is selected from the group consisting of
(5-(5-(methylcarbamoyl)pyridin-3-yl)pyrimidin-2-yl)glycine;
(5-(2-methyl-3-(methylcarbamoyl)quinolin-6-yl)pyrimidin-2-yl)glycine;
(5-(3-fluoro-4-(methylcarbamoyl)phenyl)pyrimidin-2-yl)glycine;
(5-(4-(2-(dimethylamino)-2-oxoethoxy)-3-formyl-5-methoxyphenyl)pyrimidin-2-yl)glycine; and
(5-(4-(carboxymethoxy)-3-formyl-5-methoxyphenyl)pyrimidin-2-yl)glycine.

Embodiment 33 provides a compound of formula (III), or a salt, solvate, stereoisomer, isotopologue, or tautomer thereof, or any mixtures thereof:

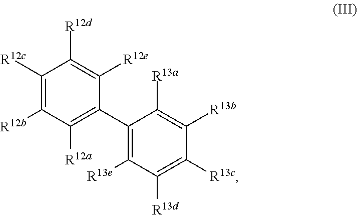

wherein:
R^{12a}, R^{12b}, R^{12c}, R^{12d}, and R^{12e} are each independently selected from the group consisting of H, halogen, OR^{A}, C(═O)R^{A}, C(═O)OR^{A}, C(═O)N(R^{A})(R^{B}), CN, NO₂, optionally substituted C₁-C₆ alkyl, and optionally substituted C₃-C₈ cycloalkyl,
   wherein at least one selected from the group consisting of R^{1a}, R^{12b}, R^{12c}, R^{12d}, and R^{12e} is C(═O)OH;
R^{13a}, R^{13b}, R^{13c}, R^{13d}, and R^{13e} are each independently selected from the group consisting of H, halogen, OR^{A}, C(═O)R^{A}, C(═O)OR^{A}, C(═O)N(R^{A})(R^{B}), CN, NO₂, optionally substituted C₁-C₆ alkyl, and optionally substituted C₃-C₈ cycloalkyl,
   wherein at least one selected from the group consisting of R^{13a}, R^{13b}, R^{13c}, R^{13d}, and R^{13e} is C(═O)OH; and
each occurrence of R^{A} and R^{B} is independently selected from the group consisting of H, —C(═O)(optionally substituted C₁-C₆ alkyl), optionally substituted C₁-C₆ alkyl, optionally substituted C₃-C₈ cycloalkyl, optionally substituted C₇-C₁₂ aralkyl, optionally substituted phenyl, optionally substituted naphthyl, and optionally substituted heteroaryl.

Embodiment 34 provides the compound of Embodiment 33, wherein R^{12a}, R^{12b}, R^{12c}, R^{12d}, and R^{12e} are each independently selected from the group consisting of H, OH, and C(═O)OH.

Embodiment 35 provides the compound of Embodiment 33 or 34, wherein R^{13a}, R^{13b}, R^{13c}, R^{13d}, and R^{13e} are each independently selected from the group consisting of H, OMe, OCH₂Ph, and C(═O)OH.

Embodiment 36 provides the compound of any one of Embodiments 33-35, wherein the compound is:

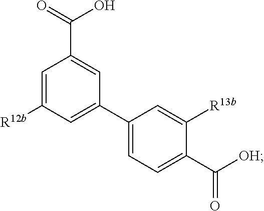

Embodiment 37 provides the compound of any one of Embodiments 33-36, which is selected from the group consisting of
5-hydroxy-3′-methoxy-[1,1′-biphenyl]-3,4′-dicarboxylic acid; and
3′-(benzyloxy)-5-hydroxy-[1,1′-biphenyl]-3,4′-dicarboxylic acid.

Embodiment 38 provides a compound of formula (IV), or a salt, solvate, stereoisomer, isotopologue, or tautomer thereof, or any mixtures thereof:

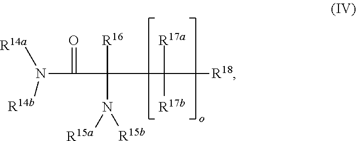

wherein:
R^{14a} and R^{14b} are each independently selected from the group consisting of H, optionally substituted C₁-C₆ alkyl, optionally substituted C₃-C₈ cycloalkyl, and optionally substituted C₇-C₁₂ aralkyl;
R^{15a} and R^{15b} are each independently selected from the group consisting of H and optionally substituted C₁-C₆ alkyl;
R¹⁶ is selected from the group consisting of H and optionally substituted C₁-C₆ alkyl;
R^{17a} and R^{17b} are each independently selected from the group consisting of H and optionally substituted C₁-C₆ alkyl;
R¹⁸ is optionally substituted aryl;
o is an integer selected from the group consisting of 0, 1, 2, and 3; and
each occurrence of R^{A} and R^{B} is independently selected from the group consisting of H, —C(═O)(optionally substituted C₁-C₆ alkyl), optionally substituted C₁-C₆ alkyl, optionally substituted C₃-C₈ cycloalkyl, optionally substituted C₇-C₁₂ aralkyl, optionally substituted phenyl, optionally substituted naphthyl, and optionally substituted heteroaryl.

Embodiment 39 provides the compound of Embodiment 38, wherein R^{14a} and R^{14b} are each independently selected from the group consisting of H and CH₂CH₂Ph.

Embodiment 40 provides the compound of Embodiment 38 or 39, wherein R^{15a} and R^{15b} are each independently H.

Embodiment 41 provides the compound of any one of Embodiments 38-40, wherein R¹⁶ is H.

Embodiment 42 provides the compound of any one of Embodiments 38-41, wherein R^{17a} and R^{17b} are each independently H.

Embodiment 43 provides the compound of any one of Embodiments 38-42, wherein o is 1.

Embodiment 44 provides the compound of any one of Embodiments 38-43, wherein R¹⁸ is phenyl optionally substituted with at least one hydroxyl.

Embodiment 45 provides the compound of any one of Embodiments 38-44, wherein R¹⁸ is

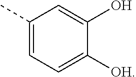

Embodiment 46 provides the compound of any one of Embodiments 38-45, which is 2-amino-3-(3,4-dihydroxyphenyl)-N-phenethylpropanamide.

Embodiment 47 provides the compound of any one of Embodiments 1-46, wherein each occurrence of alkyl, cycloalkyl, aralkyl, alkylenyl, phenylenyl, heteroarylenyl, heterocyclyl, heteroaryl, phenyl, naphthyl, and aryl is independently optionally substituted with at least one substituent C₁-C₆ alkyl, C₃-C₅ cycloalkyl, C₁-C₆ hydroxyalkyl, halogen, CN, NO₂ OR^{I}, N(R^{I})(R^{II}), C₁-C₆ alkoxy, C₃-C₈ cycloalkoxy, C₁-C₆ haloalkoxy, C₃-C₈ halocycloalkoxy, phenyl, heteroaryl, heterocyclyl, (C₁-C₆ alkylenyl)C(═O)N(R^{I})(R^{II}), (C₁-C₆ alkylenyl)C(═O)OR, O(C₁-C₃ alkylenyl)C(═O)OR^{II}, O(C₁-C₃ alkylenyl)C(═O)N(R^{I})(R^{II}), C(═O)R^{I}, C(═O)OR^{I}, OC(═O)R, OC(═O)OR^{I}, SR^{I}, S(═O)R^{I}, S(═O)₂R′, S(═O)₂N(R^{I})(R^{II}), S(═O)₂NR^{I}C(═O)NHR^{II}, N(R^{I})S(═O)₂R^{II}, N(R^{I})C(═O)R^{II}, and C(═O)NR^{I}R^{II}, wherein R^{I} and R^{II} is independently selected from the group consisting of H, —C(═O)(C₁-C₆ alkyl), C₁-C₆ alkyl, C₃-C₈ cycloalkyl, C₇-C₁₂ aralkyl, phenyl, naphthyl, and heteroaryl.

Embodiment 48 provides a pharmaceutical composition comprising at least one compound of any one of Embodiments 1-47 and a pharmaceutically acceptable carrier.

Embodiment 49 provides the pharmaceutical composition of Embodiment 48, further comprising at least one additional antibiotic agent.

Embodiment 50 provides the pharmaceutical composition of Embodiment 49, wherein the at least one additional antibiotic agent is one or more selected from the group consisting of benzathine, benzylpenicillin (penicillin G), benzathine penicillin G, benzathine penicillin V, phenoxymethylpenicillin (penicillin V), procaine penicillin, pheneticillin, cloxacillin, dicloxacillin, flucloxacillin, methicillin, nafcillin, oxacillin, temocillin, amoxicillin, ampicillin, mecillinam, piperacillin, carbenicillin, ticarcillin, azlocillin, mezlocillin, cefazolin, cephalexin, cephalosporin C, cephalothin, cefapirin, cefuroxime, cefaclor, cefprozil, cefaclor, cefamandole, cefuroxime, cefotetan, cefoxitin, cefixime, cefotaxime, cefpodoxime, ceftazidime, ceftriaxone, cefdinir, cefepime, cepirome, ceftaroline, ceftobiprole, biapenem, doripenem, ertapenem, faropenem, Imipenem, meropenem, panipenem, razupenem, tebipenem, thienamycin, aztreonam, tigemonam, nocardicin A, and tabtoxinine.

Embodiment 51 provides a method of treating, preventing, and/or ameliorating a bacterial infection in a subject, the method comprising administering to the subject a therapeutically effective amount of at least one compound of any one of Embodiments 1-47 and/or at least one pharmaceutical composition of any one of Embodiments 48-50.

Embodiment 52 provides the method of Embodiment 51, wherein the subject is further administered at least one additional antibiotic agent.

Embodiment 53 provides the method of Embodiment 52, wherein the subject is co-administered the at least one compound and/or composition and the at least one additional antibiotic agent.

Embodiment 54 provides the method of Embodiment 52 or 53, wherein the at least one compound and/or composition and the at least one additional antibiotic agent are coformulated.

Embodiment 55 provides the method of any one of Embodiments 52-54, wherein the at least one additional antibiotic agent is one or more selected from the group consisting of benzathine, benzylpenicillin (penicillin G), benzathine penicillin G, benzathine penicillin V, phenoxymethylpenicillin (penicillin V), procaine penicillin, pheneticillin, cloxacillin, dicloxacillin, flucloxacillin, methicillin, nafcillin, oxacillin, temocillin, amoxicillin, ampicillin, mecillinam, piperacillin, carbenicillin, ticarcillin, azlocillin, mezlocillin, cefazolin, cephalexin, cephalosporin C, cephalothin, cefapirin, cefuroxime, cefaclor, cefprozil, cefaclor, cefamandole, cefuroxime, cefotetan, cefoxitin, cefixime, cefotaxime, cefpodoxime, ceftazidime, ceftriaxone, cefdinir, cefepime, cepirome, ceftaroline, ceftobiprole, biapenem, doripenem, ertapenem, faropenem, Imipenem, meropenem, panipenem, razupenem, tebipenem, thienamycin, aztreonam, tigemonam, nocardicin A, and tabtoxinine.

Embodiment 56 provides a method of inhibiting a β-lactamase and/or penicillin binding protein (PBP) in a subject, the method comprising administering to the subject a therapeutically effective amount of at least one compound of any one of Embodiments 1-47 and/or at least one pharmaceutical composition of any one of Embodiments 48-50.

Embodiment 57 provides the method of Embodiment 56, wherein the β-lactamase is selected from the group consisting of NDM-1, KPC-2, and OXA-48.

Embodiment 58 provides the method of Embodiment 56, wherein the PBP is PBP-3.

Embodiment 59 provides the method of any one of Embodiments 56-58, wherein the subject is further administered at least one additional antibiotic agent.

Embodiment 60 provides the method of Embodiment 59, wherein the subject is co-administered the at least one compound and/or composition and the at least one additional antibiotic agent.

Embodiment 61 provides the method of Embodiment 59 or 60, wherein the at least one compound and/or composition and the at least one additional antibiotic agent are coformulated.

Embodiment 62 provides the method of any one of Embodiments 59-61, wherein the at least one additional antibiotic agent is one or more selected from the group consisting of benzathine, benzylpenicillin (penicillin G), benzathine penicillin G, benzathine penicillin V, phenoxymethylpenicillin (penicillin V), procaine penicillin, pheneticillin, cloxacillin, dicloxacillin, flucloxacillin, methicillin, nafcillin, oxacillin, temocillin, amoxicillin, ampicillin, mecillinam, piperacillin, carbenicillin, ticarcillin, azlocillin, mezlocillin, cefazolin, cephalexin, cephalosporin C, cephalothin, cefapirin, cefuroxime, cefaclor, cefprozil, cefaclor, cefamandole, cefuroxime, cefotetan, cefoxitin, cefixime, cefotaxime, cefpodoxime, ceftazidime, ceftriaxone, cefdinir, cefepime, cepirome, ceftaroline, ceftobiprole, biapenem, doripenem, ertapenem, faropenem, Imipenem, meropenem, panipenem, razupenem, tebipenem, thienamycin, aztreonam, tigemonam, nocardicin A, and tabtoxinine.

Embodiment 63 provides the method of any one of Embodiments 51-62, wherein the subject is a mammal.

Embodiment 64 provides the method of Embodiment 63, wherein the mammal is a human.

The terms and expressions employed herein are used as terms of description and not of limitation, and there is no intention in the use of such terms and expressions of excluding any equivalents of the features shown and described or portions thereof, but it is recognized that various modifications are possible within the scope of the embodiments of the present application. Thus, it should be understood that although the present application describes specific embodiments and optional features, modification and variation of the compositions, methods, and concepts herein disclosed may be resorted to by those of ordinary skill in the art, and that such modifications and variations are considered to be within the scope of embodiments of the present application.

1. A compound of formula (I), or a salt, solvate, stereoisomer, isotopologue, or tautomer thereof, or any mixtures thereof: 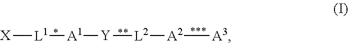
wherein:
R^{1a} and R^{1b} are each independently selected from the group consisting of H, optionally substituted C₁-C₆ alkyl, optionally substituted C₃-C₁₂ cycloalkyl, optionally substituted C₇-C₁₂ aralkyl, optionally substituted C₂-C₁₂ heterocyclyl, optionally substituted C₆-C₁₂ aryl, and C(═O)(optionally substituted C₁-C₆ alkyl);
A¹ and A² are each independently selected from the group consisting of a bond, optionally substituted phenylenyl and optionally substituted C₂-C₉ heteroarylenyl;
A³ is selected from the group consisting of optionally substituted C₆-C₁₂ aryl and optionally substituted C₂-C₁₂heteroaryl;
L¹ and L² are each independently selected from the group consisting of a bond, optionally substituted C₁-C₃ alkylenyl, —C(═O), —C(═O)(optionally substituted C₁-C₃ alkylenyl)-O—, —C(═O)(optionally substituted C₁-C₃ alkylenyl)-, (optionally substituted C₁-C₃ alkylenyl)C(═O)—, —C(═O)(optionally substituted C₁-C₃ alkylenyl)C(═O)—, optionally substituted C₃-C₈ heterocycloalkylenyl, —C(═O)(optionally substituted C₃-C₈ heterocycloalkylenyl)(optionally substituted C₁-C₃ alkylenyl)C(═O)—, and —C(═O)(optionally substituted C₃-C₈ cycloalkylenyl)C(═O)—;
R² is selected from the group consisting of H and optionally substituted C₁-C₆ alkyl,
wherein R² can combine with A² to form an optionally substituted C₃-C₈ heterocycloalkyl;
X is selected from the group consisting of N(R^{1a})(R^{1b}) and optionally substituted C₂-C₁₂ heterocyclyl;
Y is selected from the group consisting of a bond, —N(R²)—, —(CH₂)₁₋₃N(R²)—**, —O—, and —S—;
* indicates a bond between L¹ and A¹;
** indicates a bond between Y and L²;
*** indicates a bond between A² and A³; and
each occurrence of R^{A} and R^{B} is independently selected from the group consisting of H, —C(═O)(optionally substituted C₁-C₆ alkyl), optionally substituted C₁-C₆ alkyl, optionally substituted C₃-C₈ cycloalkyl, optionally substituted C₇-C₁₂ aralkyl, optionally substituted phenyl, optionally substituted naphthyl, and optionally substituted heteroaryl.

2. The compound of claim 1, wherein at least one of the following applies:
(a) A¹ is a bond;
(b) A² is a bond;
(c) L¹ is a bond;
(d) X is 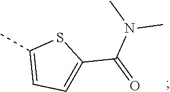
and
(e) Y is a bond or —NH—.

3. The compound of claim 1, which is a compound of formula (Ia): 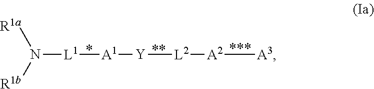
wherein:
A¹ and A² are each independently selected from the group consisting of optionally substituted phenylenyl and optionally substituted C₂-C₉ heteroarylenyl;
A³ is selected from the group consisting of optionally substituted C₆-C₁₂ aryl and optionally substituted C₂-C₁₂heteroaryl;
L¹ and L² are each independently selected from the group consisting of a bond, optionally substituted C₁-C₃ alkylenyl, —C(═O), —C(═O)(optionally substituted C₁-C₃ alkylenyl)-O—, —C(═O)(optionally substituted C₁-C₃ alkylenyl)-, (optionally substituted C₁-C₃ alkylenyl)C(═O)—, —C(═O)(optionally substituted C₁-C₃ alkylenyl)C(═O)—, optionally substituted C₃-C₈ heterocycloalkylenyl, —C(═O)(optionally substituted C₃-C₈ heterocycloalkylenyl)(optionally substituted C₁-C₃ alkylenyl)C(═O)—, and —C(═O)(optionally substituted C₃-C₈ cycloalkylenyl)C(═O)—;
R^{1a} and R^{1b} are each independently selected from the group consisting of H, optionally substituted C₁-C₆ alkyl, optionally substituted C₃-C₁₂ cycloalkyl, optionally substituted C₇-C₁₂ aralkyl, optionally substituted C₂-C₁₂ heterocyclyl, optionally substituted C₆-C₁₂ aryl, and C(═O)(optionally substituted C₁-C₆ alkyl);
R² is selected from the group consisting of H and optionally substituted C₁-C₆ alkyl,
wherein R² can combine with A² to form an optionally substituted C₃-C₈ heterocycloalkyl;
Y is selected from the group consisting of —N(R²)—, —(CH₂)₁₋₃N(R²)—**, —O—, and —S—;
* indicates a bond between L¹ and A¹;
** indicates a bond between Y and L²;
*** indicates a bond between A² and A³; and
each occurrence of R^{A} and R^{B} is independently selected from the group consisting of H, —C(═O)(optionally substituted C₁-C₆ alkyl), optionally substituted C₁-C₆ alkyl, optionally substituted C₃-C₈ cycloalkyl, optionally substituted C₇-C₁₂ aralkyl, optionally substituted phenyl, optionally substituted naphthyl, and optionally substituted heteroaryl.

4. The compound of claim 1, wherein A¹ is selected from the group consisting of: 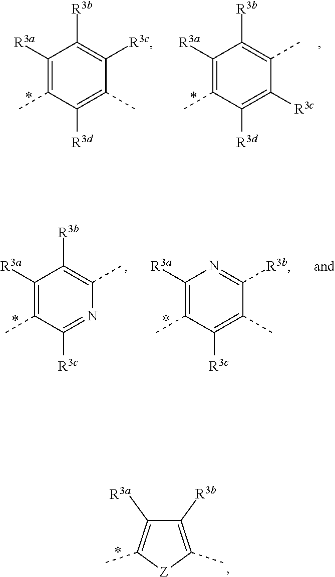
wherein:
R^{3a}, R^{3b}, R^{3c}, and R^{3d}, if present, are each independently selected from the group consisting of H, halogen, OR^{A}, C(═O)OR^{A}, CN, NO₂, optionally substituted C₁-C₆ alkyl, and optionally substituted C₃-C₈ cycloalkyl.

5. The compound of claim 4, wherein at least one of the following applies:
(a) at least one selected from R^{3a}, R^{3b}, R^{3c}, and R^{3d}, is H;
(b) at least two selected from R^{3a}, R^{3b}, R^{3c}, and R^{3d} are H; and
(c) at least three selected from R^{3a}, R^{3b}, R^{3c}, and R^{3d} are H.

6. The compound of claim 1, wherein A¹ is selected from the group consisting of:

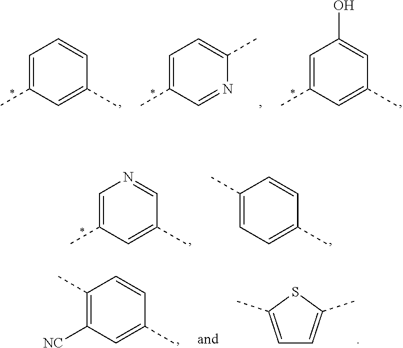

7. The compound of claim 1, wherein A² is selected from the group consisting of: 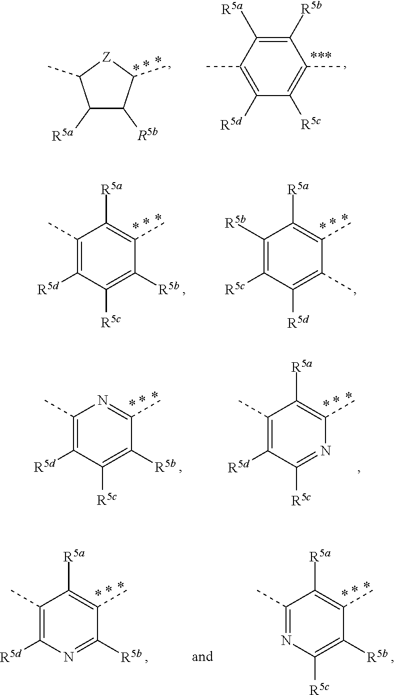
wherein:
Z is selected from the group consisting of O, S, and NR⁴;
R⁴ is selected from the H and optionally substituted C₁-C₆ alkyl; and
R^{5a}, R^{5b}, R^{5c}, and R^{5d}, if present, are each independently selected from the group consisting of H, halogen, OR^{A}, C(═O)OR^{A}, CN, NO₂, optionally substituted C₁-C₆ alkyl, and optionally substituted C₃-C₈ cycloalkyl.

8. The compound of claim 7, wherein at least one of the following applies:
(a) at least one selected from R^{5a}, R^{5b}, R^{5c}, and R^{5d} is H;
(b) at least two selected from R^{5a}, R^{5b}, R^{5c}, and R^{5d} are H; and
(c) at least three selected from R^{5a}, R^{5b}, R^{5c}, and R^{5d} are H.

9. The compound of claim 1, wherein A² is selected from the group consisting of

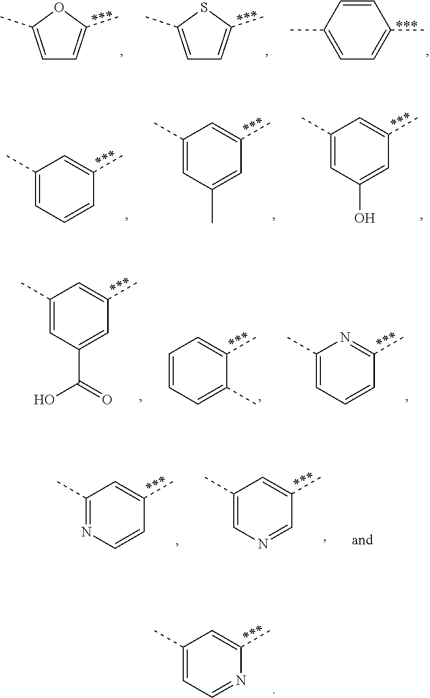

10. The compound of claim 1, wherein A³ is selected from the group consisting of: 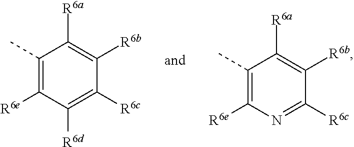
wherein:
R^{6a}, R^{6b}, R^{6c}, R^{6d}, and R^{6e}, if present, are each independently selected from the group consisting of H, halogen, OR^{A}, C(═O)OR^{A}, (optionally substituted C₁-C₆ alkylenyl)C(═O)OR^{A}, CN, NO₂, optionally substituted C₁-C₆ alkyl, and optionally substituted C₃-C₈ cycloalkyl.

11. The compound of claim 10, wherein at least one of the following applies:
(a) R^{6a}, R^{6b}, R^{6c}, R^{6d}, and R^{6e} are each independently selected from the group consisting of H, C(═O)OH, OH, and CH₂C(═O)OH;
(b) at least one selected from R^{6a}, R^{6b}, R^{6c}, R^{6d}, and R^{6e} is H;
(c) at least two selected from R^{6a}, R^{6b}, R^{6c}, R^{6d}, and R^{6e} are H;
(d) at least three selected from R^{6a}, R^{6b}, R^{6c}, R^{6d}, and R^{6e} are H;
(e) at least four selected from R^{6a}, R^{6b}, R^{6c}, R^{6d}, and R^{6e} are H; and
(f) at least one selected from R^{6a}, R^{6b}, R^{6c}, R^{6d}, and R^{6e} is selected from the group consisting of C(═O)OH, CH₂C(═O)OH, OMe, OBn, and OH.

12-13. (canceled)

14. The compound of claim 1, wherein A³ is selected from the group consisting of

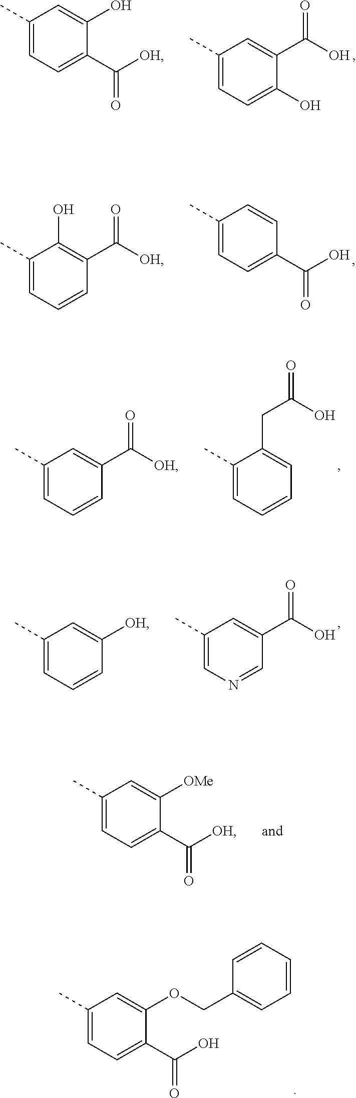

15. The compound of claim 1, wherein L¹ is selected from the group consisting of —CH₂—, —CH₂C(═O)—, —CH₂CH₂C(═O)—, C(═O)CH₂CH₂O—,

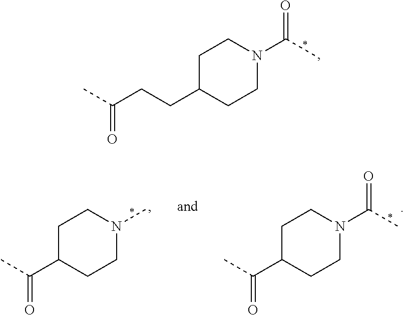

16. The compound of claim 1, wherein at least one of the following applies:
(a) L² is selected from the group consisting of —CH₂—, —C(═O)—, and —C(═O)CH₂O—;
(b) R^{1a} and R^{1b} are each independently selected from the group consisting of H, CH₃, C(═O)CH₃, and 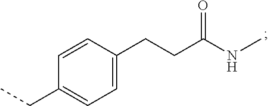
(c) R² is H; and
(d) Y is selected from the group consisting of —NH—, —CH₂NH—**, and —(CH₂)₃NH—**.

17. (canceled)

18. The compound of claim 1, wherein one of the following applies:
(a) R^{1a} is H and R^{1b} is H;
(b) R^{1a} is CH₃ and R^{1b} is H;
(c) R^{1a} is C(═O)CH₃ and R^{1b} is H; or
(d) R^{1a} is 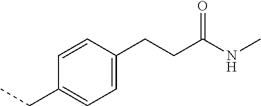
and R^{1b} is H.

19-20. (canceled)

21. The compound of claim 1, which is selected from the group consisting of:
2-hydroxy-4-(5-(((3-((methylamino)methyl)phenyl)amino)methyl)furan-2-yl)benzoic acid;
2-(4′-(((3-(((4-(3-(methylamino)-3-oxopropyl)benzyl)amino)methyl)phenyl)amino)methyl)-[1,1′-biphenyl]-2-yl)acetic acid;
5-((((5-(dimethylcarbamoyl)thiophen-2-yl)methyl)amino)methyl)-2-hydroxybenzoic acid;
5-(((5-(acetamidomethyl)pyridin-2-yl)amino)methyl)-2-hydroxybenzoic acid;
2-hydroxy-4-(5-(((3-(((4-(3-(methylamino)-3-oxopropyl)benzyl)amino)methyl)phenyl)amino)methyl)furan-2-yl)benzoic acid;
2-hydroxy-4-(5-((methylamino)methyl)furan-2-yl)benzoic acid;
4-(5-(((3-((methylamino)methyl)phenyl)amino)methyl)furan-2-yl)benzoic acid;
3-(5-(((3-((methylamino)methyl)phenyl)amino)methyl)furan-2-yl)phenol;
2-hydroxy-5-(5-(((3-((methylamino)methyl)phenyl)amino)methyl)furan-2-yl)benzoic acid;
2-hydroxy-3-(5-(((3-((methylamino)methyl)phenyl)amino)methyl)furan-2-yl)benzoic acid;
3-(5-(((3-((methylamino)methyl)phenyl)amino)methyl)furan-2-yl)benzoic acid;
4-(5-(((3-(aminomethyl)phenyl)amino)methyl)furan-2-yl)-2-hydroxybenzoic acid;
3-(5-(((3-(aminomethyl)phenyl)amino)methyl)furan-2-yl)-2-hydroxybenzoic acid;
4-(5-(((3-(aminomethyl)phenyl)amino)methyl)thiophen-2-yl)-2-hydroxybenzoic acid;
4′-(((3-(aminomethyl)phenyl)amino)methyl)-3-hydroxy-[1,1′-biphenyl]-4-carboxylic acid;
3′-(((3-(aminomethyl)phenyl)amino)methyl)-3-hydroxy-[1,1′-biphenyl]-4-carboxylic acid;
5-(4-(((3-aminophenyl)amino)methyl)phenyl)nicotinic acid;
5-(((3-(aminomethyl)phenyl)amino)methyl)-3′-hydroxy-[1,1′-biphenyl]-3,4′-dicarboxylic acid;
4-(6-(((3-(aminomethyl)phenyl)amino)methyl)pyridin-2-yl)-2-hydroxybenzoic acid;
3′-(((3-(aminomethyl)phenyl)amino)methyl)-3-hydroxy-5′-methyl-[1,1′-biphenyl]-4-carboxylic acid;
3′-(((3-(aminomethyl)phenyl)amino)methyl)-3,5′-dihydroxy-[1,1′-biphenyl]-4-carboxylic acid;
3′-((3-(aminomethyl)phenyl)carbamoyl)-3-hydroxy-[1,1′-biphenyl]-4-carboxylic acid;
4-(2-(3-(aminomethyl)phenyl)isoindolin-5-yl)-2-hydroxybenzoic acid;
3-hydroxy-3′-(((3-((methylamino)methyl)phenyl)amino)methyl)-[1,1′-biphenyl]-4-carboxylic acid;
3-hydroxy-3′-(((3-(2-(methylamino)-2-oxoethyl)phenyl)amino)methyl)-[1,1′-biphenyl]-4-carboxylic acid;
3′-(((3-(aminomethyl)-5-hydroxyphenyl)amino)methyl)-3-hydroxy-[1,1′-biphenyl]-4-carboxylic acid;
3′-(((5-(aminomethyl)pyridin-3-yl)amino)methyl)-3-hydroxy-[1,1′-biphenyl]-4-carboxylic acid;
4-(5-(((3-(aminomethyl)phenyl)amino)methyl)pyridin-3-yl)-2-hydroxybenzoic acid;
4-(2-(((3-(aminomethyl)phenyl)amino)methyl)pyridin-4-yl)-2-hydroxybenzoic acid;
4-(4-(((3-(aminomethyl)phenyl)amino)methyl)pyridin-2-yl)-2-hydroxybenzoic acid;
3′-methoxy-5-(2-(methylamino)-2-oxoethoxy)-[1,1′-biphenyl]-3,4′-dicarboxylic acid;
3′-(benzyloxy)-5-(2-(methylamino)-2-oxoethoxy)-[1,1′-biphenyl]-3,4′-dicarboxylic acid;
3′-(benzyloxy)-5-(2-((4-(3-(methylamino)-3-oxopropyl)phenyl)amino)-2-oxoethoxy)-[1,1′-biphenyl]-3,4′-dicarboxylic acid;
2′-(((3-(3-aminophenyl)propyl)amino)methyl)-[1,1′-biphenyl]-3-carboxylic acid;
2′-(((3-(3-((4-(3-(methylamino)-3-oxopropyl)benzyl)amino)phenyl)propyl)amino)methyl)-[1,1′-biphenyl]-3-carboxylic acid;
2′-(((3-cyano-4-(4-(dimethylcarbamoyl)piperidin-1-yl)benzyl)amino)methyl)-[1,1′-biphenyl]-3-carboxylic acid;
2-hydroxy-5-((((5-(4-(3-(methylamino)-3-oxopropyl)piperidine-1-carbonyl)thiophen-2-yl)methyl)amino)methyl)benzoic acid; and
2-hydroxy-5-(((5-((4-(methylcarbamoyl)cyclohexane-1-carboxamido)methyl)pyridin-2-yl)amino)methyl)benzoic acid.

22. A compound of formula (II), or a salt, solvate, stereoisomer, isotopologue, or tautomer thereof, or any mixtures thereof: 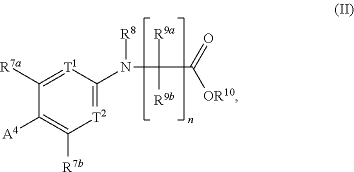
wherein:
A⁴ is selected from the group consisting of optionally substituted C₆-C₁₀ aryl and optionally substituted C₂-C₉ heteroaryl;
R^{7a}, R^{7b}, R^{7c}, and R^{7d}, if present, are each independently selected from the group consisting of H, halogen, OR^{A}, C(═O)OR^{A}, CN, NO₂, optionally substituted C₁-C₆ alkyl, and optionally substituted C₃-C₈ cycloalkyl;
R⁸ is selected from the group consisting of H and optionally substituted C₁-C₆ alkyl;
each occurrence of R^{9a} and R^{9b} is independently selected from the group consisting of H and optionally substituted C₁-C₆ alkyl;
R¹⁰ is selected from the group consisting of H, optionally substituted C₁-C₆ alkyl, optionally substituted C₃-C₁₂ cycloalkyl, optionally substituted C₇-C₁₂ aralkyl, optionally substituted C₂-C₁₂ heterocyclyl, optionally substituted C₆-C₁₂ aryl;
T¹ is selected from the group consisting of N and CR^{7c};
T² is selected from the group consisting of N and CR^{7d};
n is an integer selected from the group consisting of 1, 2, and 3; and
each occurrence of R^{A} and R^{B} is independently selected from the group consisting of H, —C(═O)(optionally substituted C₁-C₆ alkyl), optionally substituted C₁-C₆ alkyl, optionally substituted C₃-C₈ cycloalkyl, optionally substituted C₇-C₁₂ aralkyl, optionally substituted phenyl, optionally substituted naphthyl, and optionally substituted heteroaryl.

23. The compound of claim 22, wherein A⁴ is selected from the group consisting of: 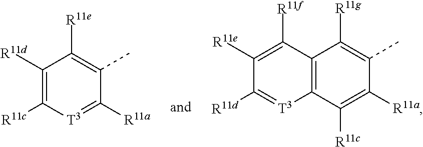
wherein:
R^{11a}, R^{11b}, R^{11c}, R^{11d}, R^{11e}, R^{11f}, and R^{11g}, if present, are each independently selected from the group consisting of H, halogen, OR^{A}, O(optionally substituted C₁-C₃ alkylenyl)C(═O)OR^{A}, O(optionally substituted C₁-C₃ alkylenyl)C(═O)N(R^{A})(R^{B}), C(═O)R^{A}, C(═O)OR^{A}, C(═O)N(R^{A})(R^{B}), CN, NO₂, optionally substituted C₁-C₆ alkyl, and optionally substituted C₃-C₈ cycloalkyl; and
T³ is selected from the group consisting of N and CR^{11b};
optionally wherein A⁴ is selected from the group consisting of

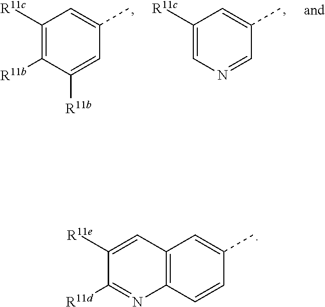

24. (canceled)

25. The compound of claim 23, wherein at least one of the following applies:
(a) R^{11a}, R^{11b}, R^{11c}, R^{11d}, R^{11e}, R^{11f}, and R^{11g}, if present, are each independently selected from the group consisting of H, Me, OMe, F, C(═O)NH(Me), C(═O)H, OCH₂C(═O)OH, and OCH₂C(═O)NMe₂; and
(b) A⁴ is selected from the group consisting of

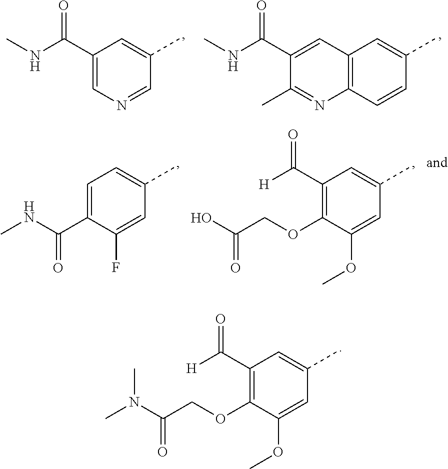

26. (canceled)

27. The compound of claim 22, wherein at least one of the following applies:
(a) T¹ is N;
(b) T² is N;
(c) R^{7a} is H; and
(d) R^{7b} is H.

28. The compound of claim 22, wherein at least one of the following applies:
(a) R⁸ is H;
(b) R^{9a} is H and R^{9b} is H;
(c) n is 1;
(d) R¹⁰ is H; and
(e) the compound is selected from the group consisting of:
(5-(5-(methylcarbamoyl)pyridin-3-yl)pyrimidin-2-yl)glycine;
(5-(2-methyl-3-(methylcarbamoyl)quinolin-6-yl)pyrimidin-2-yl)glycine:
(5-(3-fluoro-4-(methylcarbamoyl)phenyl)pyrimidin-2-yl)glycine;
(5-(4-(2-(dimethylamino)-2-oxoethoxy)-3-formyl-5-methoxyphenyl)pyrimidin-2-yl)glycine; and
(5-(4-(carboxymethoxy)-3-formyl-5-methoxyphenyl)pyrimidin-2-yl)glycine.

29-32. (canceled)

33. A compound of formula (III), or a salt, solvate, stereoisomer, isotopologue, or tautomer thereof, or any mixtures thereof: 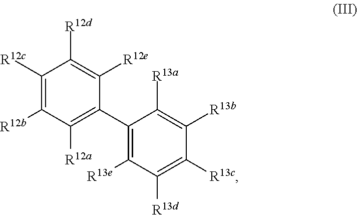
wherein:
R^{12a}, R^{12b}, R^{12c}, R^{12d}, and R^{12e} are each independently selected from the group consisting of H, halogen, OR^{A}, C(═O)R^{A}, C(═O)OR^{A}, C(═O)N(R^{A})(R^{B}), CN, NO₂, optionally substituted C₁-C₆ alkyl, and optionally substituted C₃-C₈ cycloalkyl,
wherein at least one selected from the group consisting of R^{12a}, R^{12b}, R^{12c}, R^{12d}, and R^{12e} is C(═O)OH;
R^{13a}, R^{13b}, R^{13c}, R^{13d}, and R^{13e} are each independently selected from the group consisting of H, halogen, OR^{A}, C(═O)R^{A}, C(═O)OR^{A}, C(═O)N(R^{A})(R^{B}), CN, NO₂, optionally substituted C₁-C₆ alkyl, and optionally substituted C₃-C₈ cycloalkyl,
wherein at least one selected from the group consisting of R^{13a}, R^{13b}, R^{13c}, R^{13d}, and R^{13e} is C(═O)OH; and
each occurrence of R^{A} and R^{B} is independently selected from the group consisting of H, —C(═O)(optionally substituted C₁-C₆ alkyl), optionally substituted C₁-C₆ alkyl, optionally substituted C₃-C₈ cycloalkyl, optionally substituted C₇-C₁₂ aralkyl, optionally substituted phenyl, optionally substituted naphthyl, and optionally substituted heteroaryl.

34. The compound of claim 33, wherein at least one of the following applies:
(a) R^{12a}, R^{12b}, R^{12c}, R^{12d}, and R^{12e} are each independently selected from the group consisting of H, OH, and C(═O)OH;
(b) R^{13a}, R^{13b}, R^{13c}, R^{13d}, and R^{13e} are each independently selected from the group consisting of H, OMe, OCH₂Ph, and C(═O)OH;
(c) the compound is 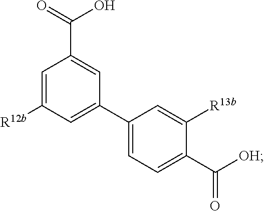
(d) the compound is 5-hydroxy-3′-methoxy-[1,1′-biphenyl]-3,4′-dicarboxylic acid; and
(e) the compound is 3′-(benzyloxy)-5-hydroxy-[1,1′-biphenyl]-3,4′-dicarboxylic acid.

35-37. (canceled)

38. A compound of formula (IV), or a salt, solvate, stereoisomer, isotopologue, or tautomer thereof, or any mixtures thereof: 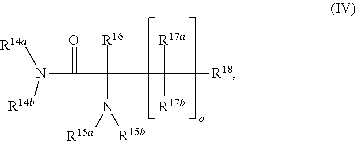
R^{14a} and R^{14b} are each independently selected from the group consisting of H, optionally substituted C₁-C₆ alkyl, optionally substituted C₃-C₈ cycloalkyl, and optionally substituted C₇-C₁₂ aralkyl;
R^{15a} and R^{15b} are each independently selected from the group consisting of H and optionally substituted C₁-C₆ alkyl;
R¹⁶ is selected from the group consisting of H and optionally substituted C₁-C₆ alkyl;
R^{17a} and R^{17b} are each independently selected from the group consisting of H and optionally substituted C₁-C₆ alkyl;
R¹⁸ is optionally substituted aryl;
o is an integer selected from the group consisting of 0, 1, 2, and 3; and
each occurrence of R^{A} and R^{B} is independently selected from the group consisting of H, —C(═O)(optionally substituted C₁-C₆ alkyl), optionally substituted C₁-C₆ alkyl, optionally substituted C₃-C₈ cycloalkyl, optionally substituted C₇-C₁₂ aralkyl, optionally substituted phenyl, optionally substituted naphthyl, and optionally substituted heteroaryl.

39. The compound of claim 38, wherein at least one of the following applies:
(a) R^{14a} and R^{14b} are each independently selected from the group consisting of H and CH₂CH₂Ph;
(b) R^{15a} and R^{15b} are each independently H;
(c) R¹⁶ is H;
(d) R^{17a} and R^{17b} are each independently H;
(e) o is 1;
(f) R¹⁸ is phenyl optionally substituted with at least one hydroxyl;
(g) R¹⁸ is 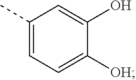
and
(h) the compound is 2-amino-3-(3,4-dihydroxyphenyl)-N-phenethylpropanamide.

40-46. (canceled)

47. The compound of claim 1, wherein each occurrence of alkyl, cycloalkyl, aralkyl, alkylenyl, phenylenyl, heteroarylenyl, heterocyclyl, heteroaryl, phenyl, naphthyl, and aryl is independently optionally substituted with at least one substituent C₁-C₆ alkyl, C₃-C₈ cycloalkyl, C₁-C₆ hydroxyalkyl, halogen, CN, NO₂ OR^{I}, N(R^{I})(R^{II}), C₁-C₆ alkoxy, C₃-C₈ cycloalkoxy, C₁-C₆ haloalkoxy, C₃-C₈ halocycloalkoxy, phenyl, heteroaryl, heterocyclyl, (C₁-C₆ alkylenyl)C(═O)N(R^{I})(R^{II}), (C₁-C₆ alkylenyl)C(═O)OR^{I}, O(C₁-C₃ alkylenyl)C(═O)OR^{II}, O(C₁-C₃ alkylenyl)C(═O)N(R^{I})(R^{II}), C(═O)R^{I}, C(═O)OR^{I}, OC(═O)R^{I}, OC(═O)OR^{I}, SR^{I}, S(═O)R^{I}, S(═O)₂R^{I}, S(═O)₂N(R^{I})(R^{II}), S(═O)₂NR^{I}C(═O)NHR^{II}, N(R^{I})S(═O)₂R^{II}, N(R^{I})C(═O)R^{II}, and C(═O)NR^{I}R^{II}, wherein R^{I} and R^{II} is independently selected from the group consisting of H, —C(═O)(C₁-C₆ alkyl), C₁-C₆ alkyl, C₃-C₈ cycloalkyl, C₇-C₁₂ aralkyl, phenyl, naphthyl, and heteroaryl.

48. A pharmaceutical composition comprising at least one compound of claim 1 and a pharmaceutically acceptable carrier;
wherein optionally the pharmaceutical composition further comprises at least one additional antibiotic agent, optionally wherein the at least one additional antibiotic agent is one or more selected from the group consisting of benzathine, benzylpenicillin (penicillin G), benzathine penicillin G, benzathine penicillin V, phenoxymethylpenicillin (penicillin V), procaine penicillin, pheneticillin, cloxacillin, dicloxacillin, flucloxacillin, methicillin, nafcillin, oxacillin, temocillin, amoxicillin, ampicillin, mecillinam, piperacillin, carbenicillin, ticarcillin, azlocillin, mezlocillin, cefazolin, cephalexin, cephalosporin C, cephalothin, cefapirin, cefuroxime, cefaclor, cefprozil, cefaclor, cefamandole, cefuroxime, cefotetan, cefoxitin, cefixime, cefotaxime, cefpodoxime, ceftazidime, ceftriaxone, cefdinir, cefepime, cepirome, ceftaroline, ceftobiprole, biapenem, doripenem, ertapenem, faropenem, Imipenem, meropenem, panipenem, razupenem, tebipenem, thienamycin, aztreonam, tigemonam, nocardicin A, and tabtoxinine.

49-50. (canceled)

51. A method of treating, preventing, or ameliorating a bacterial infection in a subject, the method comprising administering to the subject a therapeutically effective amount of at least one compound of claim 1.

52. The method of claim 51, wherein the subject is further administered at least one additional antibiotic agent,
optionally wherein the at least one additional antibiotic agent is one or more selected from the group consisting of benzathine, benzylpenicillin (penicillin G), benzathine penicillin G, benzathine penicillin V, phenoxymethylpenicillin (penicillin V), procaine penicillin, pheneticillin, cloxacillin, dicloxacillin, flucloxacillin, methicillin, nafcillin, oxacillin, temocillin, amoxicillin, ampicillin, mecillinam, piperacillin, carbenicillin, ticarcillin, azlocillin, mezlocillin, cefazolin, cephalexin, cephalosporin C, cephalothin, cefapirin, cefuroxime, cefaclor, cefprozil, cefaclor, cefamandole, cefuroxime, cefotetan, cefoxitin, cefixime, cefotaxime, cefpodoxime, ceftazidime, ceftriaxone, cefdinir, cefepime, cepirome, ceftaroline, ceftobiprole, biapenem, doripenem, ertapenem, faropenem, Imipenem, meropenem, panipenem, razupenem, tebipenem, thienamycin, aztreonam, tigemonam, nocardicin A, and tabtoxinine;
optionally wherein the subject is co-administered the at least one compound and the at least one additional antibiotic agent or the at least one compound and the at least one additional antibiotic agent are coformulated.

53-55. (canceled)

56. A method of inhibiting a β-lactamase and/or penicillin binding protein (PBP) in a subject, the method comprising administering to the subject a therapeutically effective amount of at least one compound of claim 1.

57. The method of claim 56, wherein at least one of the following applies:
(a) the β-lactamase is selected from the group consisting of NDM-1, KPC-2, and OXA-48;
(b) the PBP is PBP-3;
(c) the subject is further administered at least one additional antibiotic agent, optionally wherein the at least one additional antibiotic agent is one or more selected from the group consisting of benzathine, benzylpenicillin (penicillin G), benzathine penicillin G, benzathine penicillin V, phenoxymethylpenicillin (penicillin V), procaine penicillin, pheneticillin, cloxacillin, dicloxacillin, flucloxacillin, methicillin, nafcillin, oxacillin, temocillin, amoxicillin, ampicillin, mecillinam, piperacillin, carbenicillin, ticarcillin, azlocillin, mezlocillin, cefazolin, cephalexin, cephalosporin C, cephalothin, cefapirin, cefuroxime, cefaclor, cefprozil, cefaclor, cefamandole, cefuroxime, cefotetan, cefoxitin, cefixime, cefotaxime, cefpodoxime, ceftazidime, ceftriaxone, cefdinir, cefepime, cepirome, ceftaroline, ceftobiprole, biapenem, doripenem, ertapenem, faropenem, Imipenem, meropenem, panipenem, razupenem, tebipenem, thienamycin, aztreonam, tigemonam, nocardicin A, and tabtoxinine;
optionally wherein the subject is co-administered the at least one compound and the at least one additional antibiotic agent or the at least one compound and the at least one additional antibiotic agent are coformulated; and
(d) the subject is a mammal, optionally the mammal is a human.

58-64. (canceled)
